SEQUENCE LISTING (325 sequences)

SEQ ID NO: 1 - protein, 5 aa, Artificial Sequence
  Description of Artificial Sequence Synthetic peptide
SYWIG

SEQ ID NO: 2 - protein, 17 aa, Artificial Sequence
  Description of Artificial Sequence Synthetic peptide
IIYPGDSDTRYSPSFQG

SEQ ID NO: 3 - protein, 14 aa, Artificial Sequence
  Description of Artificial Sequence Synthetic peptide
HGRGYNGYEGAFDI

SEQ ID NO: 4 - protein, 11 aa, Artificial Sequence
  Description of Artificial Sequence Synthetic peptide
RASQGISSALA

SEQ ID NO: 5 - protein, 7 aa, Artificial Sequence
  Description of Artificial Sequence Synthetic peptide
DASSLES

SEQ ID NO: 6 - protein, 10 aa, Artificial Sequence
  Description of Artificial Sequence Synthetic peptide
CQQFNSYPLT

SEQ ID NO: 7 - protein, 120 aa, Artificial Sequence
  Description of Artificial Sequence Synthetic polypeptide
EVQLVESGGGLVQPGGSLRLSCAASGFTFSDYAMSWVRQAPGKGLEWVAVISENGSDTYYADSVKGRFTISRDDSKNTLYLQMNSLRAEDTAVYYCARDRGGAVSYFDVWGQGTLVTVSS

SEQ ID NO: 8 - protein, 109 aa, Artificial Sequence
  Description of Artificial Sequence Synthetic polypeptide
DIQMTQSPSSLSASVGDRVTITCRASQDVSSYLAWYQQKPGKAPKLLIYAASSLESGVPSRFSGSGSGTDFTLTISSLQPEDFATYYCQQYNSLPYTFGQGTKVEIKRT

SEQ ID NO: 9 - protein, 121 aa, Artificial Sequence
  Description of Artificial Sequence Synthetic polypeptide
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSS

SEQ ID NO: 10 - protein, 107 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYIAPYTFGGGTKVEIK

SEQ ID NO: 11 - protein, 9 aa, Artificial Sequence
  Description of Artificial Sequence Synthetic peptide
FTFSDADMD

SEQ ID NO: 12 - protein, 19 aa, Artificial Sequence
  Description of Artificial Sequence Synthetic peptide
RTRNKAGSYTTEYAASVKG

SEQ ID NO: 13 - protein, 12 aa, Artificial Sequence
  Description of Artificial Sequence Synthetic peptide
AREPKYWIDFDL

SEQ ID NO: 14 - protein, 11 aa, Artificial Sequence
  Description of Artificial Sequence Synthetic peptide
RASQSISSYLN

SEQ ID NO: 15 - protein, 7 aa, Artificial Sequence
  Description of Artificial Sequence Synthetic peptide
AASSLQS

SEQ ID NO: 16 - protein, 9 aa, Artificial Sequence
  Description of Artificial Sequence Synthetic peptide
QQSYIAPYT

SEQ ID NO: 17 - DNA, 363 bp, Artificial Sequence
  Description of Artificial Sequence Synthetic polynucleotide
gaggtgcagctggtggagtctgggggaggcttggtccagcctggagggtccctgagactctcctgtgcagcctctggattcaccttcagtgacgccgacatggactgggtccgccaggctccagggaaggggctggagtgggttggccgtactagaaacaaagcaggaagttacaccacagaatacgccgcgtctgtgaaaggcagattcaccatctcaagagatgattcaaagaactcactgtatctgcaaatgaacagcctgaaaaccgaggacacggcggtgtactactgcgccagagagcctaaatactggatcgacttcgacctatgggggagaggtaccttggtcaccgtctcctca

SEQ ID NO: 18 - DNA, 321 bp, Artificial Sequence
  Description of Artificial Sequence Synthetic polynucleotide
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaagctacatcgccccttacacttttggcggagggaccaaggttgagatcaaa

SEQ ID NO: 19 - protein, 119 aa, Artificial Sequence
  Description of Artificial Sequence Synthetic polypeptide
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSS

SEQ ID NO: 20 - protein, 106 aa, Artificial Sequence
  Description of Artificial Sequence Synthetic polypeptide
DIQMTQSPSSLSASVGDRVTITCRASQSINSYLNWYQQKPGKAPKLLIYAASSLQSGVPSRFSGSGSGTDFTLTISSLQPEDFATYYCQQGVSDITFGGGTKVEIK

SEQ ID NO: 21 - protein, 9 aa, Artificial Sequence
  Description of Artificial Sequence Synthetic peptide
GTFRIYAIS

SEQ ID NO: 22 - protein, 17 aa, Artificial Sequence
  Description of Artificial Sequence Synthetic peptide
GIIPDFGVANYAQKFQG

SEQ ID NO: 23 - protein, 12 aa, Artificial Sequence
  Description of Artificial Sequence Synthetic peptide
ARGGLDTDEFDL

SEQ ID NO: 24 - protein, 11 aa, Artificial Sequence
  Description of Artificial Sequence Synthetic peptide
RASQSINSYLN

SEQ ID NO: 25 - protein, 7 aa, Artificial Sequence
  Description of Artificial Sequence Synthetic peptide
AASSLQS

SEQ ID NO: 26 - protein, 8 aa, Artificial Sequence
  Description of Artificial Sequence Synthetic peptide
QQGVSDIT

SEQ ID NO: 27 - DNA, 357 bp, Artificial Sequence
  Description of Artificial Sequence Synthetic polynucleotide
caggtgcagctggtgcagtctggggctgaggtgaagaagcctgggtcctcggtgaaggtctcctgcaaggcttctggaggcaccttccgaatctatgctatcagctgggtgcgacaggcccctggacaagggcttgagtggatgggagggatcatccctgacttcggtgtagcaaactacgcacagaagttccagggcagagtcacgattaccgcggacgaatccacgagcacagcctacatggagctgagcagcctgagatctgaggacacggcggtgtactactgcgccagaggtggattggacacagacgagttcgacctatgggggagaggtaccttggtcaccgtctcctca

SEQ ID NO: 28 - DNA, 318 bp, Artificial Sequence
  Description of Artificial Sequence Synthetic polynucleotide
gacatccagatgacccagtctccatcctccctgtctgcatctgtaggagacagagtcaccatcacttgccgggcaagtcagagcattaacagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaggagtcagtgacatcacttttggcggagggaccaaggttgagatcaaa

SEQ ID NO: 29 - protein, 119 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGGLDTDEFDLWGRGTLVTVSS

SEQ ID NO: 30 - protein, 106 aa, Artificial Sequence; identical to SEQ ID NO: 20
  Description of Artificial Sequence Synthetic polypeptide

SEQ ID NO: 31 - protein, 9 aa, Artificial Sequence
  Description of Artificial Sequence Synthetic peptide
GTFSSYAIS

SEQ ID NO: 32 - protein, 17 aa, Artificial Sequence
  Description of Artificial Sequence Synthetic peptide
GIIPIFGTANYAQKFQG

SEQ ID NO: 33 - protein, 12 aa, Artificial Sequence
  Description of Artificial Sequence Synthetic peptide
ARGGLDTDEFDL

SEQ ID NO: 34 - protein, 11 aa, Artificial Sequence
  Description of Artificial Sequence Synthetic peptide
RASQSINSYLN

SEQ ID NO: 35 - protein, 7 aa, Artificial Sequence
  Description of Artificial Sequence Synthetic peptide
AASSLQS

SEQ ID NO: 36 - protein, 8 aa, Artificial Sequence
  Description of Artificial Sequence Synthetic peptide
QQGVSDIT

SEQ ID NO: 37 - DNA, 357 bp, Artificial Sequence
  Description of Artificial Sequence Synthetic polynucleotide
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggcggtgtactactgcgccagaggtggattggacacagacgagttcgacctatgggggagaggtaccttggtcaccgtctcctca

SEQ ID NO: 38 - DNA, 318 bp, Artificial Sequence; identical to SEQ ID NO: 28
  Description of Artificial Sequence Synthetic polynucleotide

SEQ ID NO: 39 - protein, 119 aa, Artificial Sequence
  Description of Artificial Sequence Synthetic polypeptide
QVQLVQSGAEVKKPGSSVKVSCKASGGTFSLYAISWVRQAPGQGLEWMGGIIPAFGTANYAQKFQGRVTITADESTSTAYMELSSLRSEDTAVYYCARGGLDTDEFDLWGRGTLVTVSS

SEQ ID NO: 40 - protein, 106 aa, Artificial Sequence; identical to SEQ ID NO: 20
  Description of Artificial Sequence Synthetic polypeptide

SEQ ID NO: 41 - protein, 9 aa, Artificial Sequence
  Description of Artificial Sequence Synthetic peptide
GTFSLYAIS

SEQ ID NO: 42 - protein, 17 aa, Artificial Sequence
  Description of Artificial Sequence Synthetic peptide
GIIPAFGTANYAQKFQG

SEQ ID NO: 43 - protein, 12 aa, Artificial Sequence
  Description of Artificial Sequence Synthetic peptide
ARGGLDTDEFDL

SEQ ID NO: 44 - protein, 11 aa, Artificial Sequence
  Description of Artificial Sequence Synthetic peptide
RASQSINSYLN

SEQ ID NO: 45 - protein, 7 aa, Artificial Sequence
  Description of Artificial Sequence Synthetic peptide
AASSLQS

SEQ ID NO: 46 - protein, 8 aa, Artificial Sequence
  Description of Artificial Sequence Synthetic peptide
QQGVSDIT

SEQ ID NO: 47 - DNA, 357 bp, Artificial Sequence
  Description of Artificial Sequence Synthetic polynucleotide
caggtgcagctggtgcagtctggggctgaggtgaagaagcctgggtcctcggtgaaggtctcctgcaaggcttctggaggcaccttcagcctctatgctatctcctgggtgcgacaggcccctggacaagggcttgagtggatgggagggatcatccctgccttcggtaccgcaaactacgcacagaagttccagggcagagtcacgattaccgcggacgaatccacgagcacagcctacatggagctgagcagcctgagatctgaggacacggcggtgtactactgcgccagaggtggattggacacagacgagttcgacctatgggggagaggtaccttggtcaccgtctcctca

SEQ ID NO: 48 - DNA, 318 bp, Artificial Sequence; identical to SEQ ID NO: 28
  Description of Artificial Sequence Synthetic polynucleotide

SEQ ID NO: 49 - protein, 119 aa, Artificial Sequence
  Description of Artificial Sequence Synthetic polypeptide
QVQLVQSGAEVKKPGSSVKVSCKASGGTFSLYAISWVRQAPGQGLEWMGGIIPHFGLANYAQKFQGRVTITADESTSTAYMELSSLRSEDTAVYYCARGGLDTDEFDLWGRGTLVTVSS

SEQ ID NO: 50 - protein, 106 aa, Artificial Sequence; identical to SEQ ID NO: 20
  Description of Artificial Sequence Synthetic polypeptide

SEQ ID NO: 51 - protein, 9 aa, Artificial Sequence
  Description of Artificial Sequence Synthetic peptide
GTFSLYAIS

SEQ ID NO: 52 - protein, 17 aa, Artificial Sequence
  Description of Artificial Sequence Synthetic peptide
GIIPHFGLANYAQKFQG

SEQ ID NO: 53 - protein, 12 aa, Artificial Sequence
  Description of Artificial Sequence Synthetic peptide
ARGGLDTDEFDL

SEQ ID NO: 54 - protein, 11 aa, Artificial Sequence
  Description of Artificial Sequence Synthetic peptide
RASQSINSYLN

SEQ ID NO: 55 - protein, 7 aa, Artificial Sequence
  Description of Artificial Sequence Synthetic peptide
AASSLQS

SEQ ID NO: 56 - protein, 8 aa, Artificial Sequence
  Description of Artificial Sequence Synthetic peptide
QQGVSDIT

SEQ ID NO: 57 - DNA, 357 bp, Artificial Sequence
  Description of Artificial Sequence Synthetic polynucleotide
caggtgcagctggtgcagtctggggctgaggtgaagaagcctgggtcctcggtgaaggtctcctgcaaggcttctggaggcaccttctccctctatgctatcagctgggtgcgacaggcccctggacaagggcttgagtggatgggagggatcatccctcacttcggtctcgcaaactacgcacagaagttccagggcagagtcacgattaccgcggacgaatccacgagcacagcctacatggagctgagcagcctgagatctgaggacacggcggtgtactactgcgccagaggtggattggacacagacgagttcgacctatgggggagaggtaccttggtcaccgtctcctca

SEQ ID NO: 58 - DNA, 318 bp, Artificial Sequence; identical to SEQ ID NO: 28
  Description of Artificial Sequence Synthetic polynucleotide

SEQ ID NO: 59 - protein, 123 aa, Artificial Sequence
  Description of Artificial Sequence Synthetic polypeptide
EVQLLESGGGLVQPGGSLRLSCAASGFTFSNYAMSWVRQAPGKGLEWVSAISGSGGSTYYADSVKGRFTISRDNSKNTLYLQMNSLRAEDTAVYYCAKGPPTYHTNYYYMDVWGKGTTVTVSS

SEQ ID NO: 60 - protein, 107 aa, Artificial Sequence
  Description of Artificial Sequence Synthetic polypeptide
DIQMTQSPSSVSASVGDRVTITCRASQGISSWLAWYQQKPGKAPKLLIYAASSLQSGVPSRFSGSGSGTDFTLTISSLQPEDFATYYCQQTNSFPYTFGGGTKVEIK

SEQ ID NO: 61 - protein, 9 aa, Artificial Sequence
  Description of Artificial Sequence Synthetic peptide
FTFSNYAMS

SEQ ID NO: 62 - protein, 17 aa, Artificial Sequence
  Description of Artificial Sequence Synthetic peptide
AISGSGGSTYYADSVKG

SEQ ID NO: 63 - protein, 16 aa, Artificial Sequence
  Description of Artificial Sequence Synthetic peptide
AKGPPTYHTNYYYMDV

SEQ ID NO: 64 - protein, 11 aa, Artificial Sequence
  Description of Artificial Sequence Synthetic peptide
RASQGISSWLA

SEQ ID NO: 65 - protein, 7 aa, Artificial Sequence
  Description of Artificial Sequence Synthetic peptide
AASSLQS

SEQ ID NO: 66 - protein, 9 aa, Artificial Sequence
  Description of Artificial Sequence Synthetic peptide
QQTNSFPYT

SEQ ID NO: 67 - DNA, 369 bp, Artificial Sequence
  Description of Artificial Sequence Synthetic polynucleotide
gaggtgcagctgttggagtctgggggaggcttggtacagcctggggggtccctgagactctcctgtgcagcctctggattcacctttagcaattatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggcggtgtactactgcgccaagggccctcctacataccacacaaactactactacatggacgtatggggcaagggtacaactgtcaccgtctcctca

SEQ ID NO: 68 - DNA, 321 bp, Artificial Sequence
  Description of Artificial Sequence Synthetic polynucleotide
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttattactgtcagcaaacaaatagtttcccttacacttttggcggagggaccaaggttgagatcaaa

SEQ ID NO: 69 - protein, 123 aa, Artificial Sequence
  Description of Artificial Sequence Synthetic polypeptide
EVQLLESGGGLVQPGGSLRLSCAASGFTFSSYVMIWVRQAPGKGLEWVSSISGDSVTTYYADSVKGRFTISRDNSKNTLYLQMNSLRAEDTAVYYCAKGPPTYHTNYYYMDVWGKGTTVTVSS

SEQ ID NO: 70 - protein, 107 aa, Artificial Sequence; identical to SEQ ID NO: 60
  Description of Artificial Sequence Synthetic polypeptide

SEQ ID NO: 71 - protein, 9 aa, Artificial Sequence
  Description of Artificial Sequence Synthetic peptide
FTFSSYVMI

SEQ ID NO: 72 - protein, 17 aa, Artificial Sequence
  Description of Artificial Sequence Synthetic peptide
SISGDSVTTYYADSVKG

SEQ ID NO: 73 - protein, 16 aa, Artificial Sequence
  Description of Artificial Sequence Synthetic peptide
AKGPPTYHTNYYYMDV

SEQ ID NO: 74 - protein, 11 aa, Artificial Sequence
  Description of Artificial Sequence Synthetic peptide
RASQGISSWLA

SEQ ID NO: 75 - protein, 7 aa, Artificial Sequence
  Description of Artificial Sequence Synthetic peptide
AASSLQS

SEQ ID NO: 76 - protein, 9 aa, Artificial Sequence
  Description of Artificial Sequence Synthetic peptide
QQTNSFPYT

SEQ ID NO: 77 - DNA, 369 bp, Artificial Sequence
  Description of Artificial Sequence Synthetic polynucleotide
gaggtgcagctgttggagtctgggggaggcttggtacagcctggggggtccctgagactctcctgtgcagcctctggattcacctttagcagctatgtcatgatctgggtccgccaggctccagggaaggggctggagtgggtctcaagcattagtggtgacagcgtaacaacatactacgcagactccgtgaagggccggttcaccatctccagagacaattccaagaacacgctgtatctgcaaatgaacagcctgagagccgaggacacggcggtgtactactgcgccaagggccctcctacataccacacaaactactactacatggacgtatggggcaagggtacaactgtcaccgtctcctca

SEQ ID NO: 78 - DNA, 321 bp, Artificial Sequence; identical to SEQ ID NO: 68
  Description of Artificial Sequence Synthetic polynucleotide

SEQ ID NO: 79 - protein, 121 aa, Artificial Sequence
  Description of Artificial Sequence Synthetic polypeptide
EVQLVESGGGLVQPGGSLRLSCAASGFTFSDHYMDWVRQAPGKGLEWVGRTRNKASSYTTEYAASVKGRFTISRDDSKNSLYLQMNSLKTEDTAVYYCAREPKYWIDFDLWGRGTLVTVSS

SEQ ID NO: 80 - protein, 107 aa, Artificial Sequence; identical to SEQ ID NO: 10
  Description of Artificial Sequence Synthetic polypeptide

SEQ ID NO: 81 - protein, 9 aa, Artificial Sequence
  Description of Artificial Sequence Synthetic peptide
FTFSDHYMD

SEQ ID NO: 82 - protein, 19 aa, Artificial Sequence
  Description of Artificial Sequence Synthetic peptide
RTRNKASSYTTEYAASVKG

SEQ ID NO: 83 - protein, 12 aa, Artificial Sequence
  Description of Artificial Sequence Synthetic peptide
AREPKYWIDFDL

SEQ ID NO: 84 - protein, 11 aa, Artificial Sequence
  Description of Artificial Sequence Synthetic peptide
RASQSISSYLN

SEQ ID NO: 85 - protein, 7 aa, Artificial Sequence
  Description of Artificial Sequence Synthetic peptide
AASSLQS

SEQ ID NO: 86 - protein, 9 aa, Artificial Sequence
  Description of Artificial Sequence Synthetic peptide
QQSYIAPYT

SEQ ID NO: 87 - DNA, 363 bp, Artificial Sequence
  Description of Artificial Sequence Synthetic polynucleotide
gaggtgcagctggtggagtctgggggaggcttggtccagcctggagggtccctgagactctcctgtgcagcctctggattcaccttcagtgaccactacatggactgggtccgccaggctccagggaaggggctggagtgggttggccgtactagaaacaaagctagtagttacaccacagaatacgccgcgtctgtgaaaggcagattcaccatctcaagagatgattcaaagaactcactgtatctgcaaatgaacagcctgaaaaccgaggacacggcggtgtactactgcgccagagagcctaaatactggatcgacttcgacctatgggggagaggtaccttggtcaccgtctcctca

SEQ ID NO: 88 - DNA, 321 bp, Artificial Sequence; identical to SEQ ID NO: 18
  Description of Artificial Sequence Synthetic polynucleotide

SEQ ID NO: 89 - protein, 121 aa, Artificial Sequence
  Description of Artificial Sequence Synthetic polypeptide
EVQLVESGGGLVQPGRSLRLSCTASGFTFSDHDMNWVRQAPGKGLEWVGRTRNAAGSYTTEYAASVKGRFTISRDDSKNSLYLQMNSLKTEDTAVYYCAREPKYWIDFDLWGRGTLVTVSS

SEQ ID NO: 90 - protein, 107 aa, Artificial Sequence; identical to SEQ ID NO: 10
  Description of Artificial Sequence Synthetic polypeptide

SEQ ID NO: 91 - protein, 9 aa, Artificial Sequence
  Description of Artificial Sequence Synthetic peptide
FTFSDHDMN

SEQ ID NO: 92 - protein, 19 aa, Artificial Sequence
  Description of Artificial Sequence Synthetic peptide
RTRNAAGSYTTEYAASVKG

SEQ ID NO: 93 - protein, 12 aa, Artificial Sequence
  Description of Artificial Sequence Synthetic peptide
AREPKYWIDFDL

SEQ ID NO: 94 - protein, 11 aa, Artificial Sequence
  Description of Artificial Sequence Synthetic peptide
RASQSISSYLN

SEQ ID NO: 95 - protein, 7 aa, Artificial Sequence
  Description of Artificial Sequence Synthetic peptide
AASSLQS

SEQ ID NO: 96 - protein, 9 aa, Artificial Sequence
  Description of Artificial Sequence Synthetic peptide
QQSYIAPYT

SEQ ID NO: 97 - DNA, 363 bp, Artificial Sequence
  Description of Artificial Sequence Synthetic polynucleotide
gaggtgcagctggtggagtctgggggaggcttggtacagccagggcggtccctgagactctcctgtacagcttctggattcaccttcagtgaccacgacatgaactgggtccgccaggctccagggaaggggctggagtgggttggccgtactagaaacgccgctggaagttacaccacagaatacgccgcgtctgtgaaaggcagattcaccatctcaagagatgattcaaagaactcactgtatctgcaaatgaacagcctgaaaaccgaggacacggcggtgtactactgcgccagagagcctaaatactggatcgacttcgacctatgggggagaggtaccttggtcaccgtctcctca

SEQ ID NO: 98 - DNA, 321 bp, Artificial Sequence; identical to SEQ ID NO: 18
  Description of Artificial Sequence Synthetic polynucleotide

SEQ ID NO: 99 - protein, 121 aa, Artificial Sequence
  Description of Artificial Sequence Synthetic polypeptide
EVQLVESGGGLVQPGGSLRLSCAASGFTFVDHDMDWVRQAPGKGLEWVGRTRNKLGSYTTEYAASVKGRFTISRDDSKNSLYLQMNSLKTEDTAVYYCAREPKYWIDFDLWGRGTLVTVSS

SEQ ID NO: 100 - protein, 107 aa, Artificial Sequence; identical to SEQ ID NO: 10
  Description of Artificial Sequence Synthetic polypeptide

SEQ ID NO: 101 - protein, 9 aa, Artificial Sequence
  Description of Artificial Sequence Synthetic peptide
FTFVDHDMD

SEQ ID NO: 102 - protein, 19 aa, Artificial Sequence
  Description of Artificial Sequence Synthetic peptide
RTRNKLGSYTTEYAASVKG

SEQ ID NO: 103 - protein, 12 aa, Artificial Sequence
  Description of Artificial Sequence Synthetic peptide
AREPKYWIDFDL

SEQ ID NO: 104 - protein, 11 aa, Artificial Sequence
  Description of Artificial Sequence Synthetic peptide
RASQSISSYLN

SEQ ID NO: 105 - protein, 7 aa, Artificial Sequence
  Description of Artificial Sequence Synthetic peptide
AASSLQS

SEQ ID NO: 106 - protein, 9 aa, Artificial Sequence
  Description of Artificial Sequence Synthetic peptide
QQSYIAPYT

SEQ ID NO: 107 - DNA, 363 bp, Artificial Sequence
  Description of Artificial Sequence Synthetic polynucleotide
gaggtgcagctggtggagtctgggggaggcttggtccagcctggagggtccctgagactctcctgtgcagcctctggattcaccttcgtagaccacgacatggactgggtccgccaggctccagggaaggggctggagtgggttggccgtactagaaacaaactaggaagttacaccacagaatacgccgcgtctgtgaaaggcagattcaccatctcaagagatgattcaaagaactcactgtatctgcaaatgaacagcctgaaaaccgaggacacggcggtgtactactgcgccagagagcctaaatactggatcgacttcgacctatgggggagaggtaccttggtcaccgtctcctca

SEQ ID NO: 108 - DNA, 321 bp, Artificial Sequence; identical to SEQ ID NO: 18
  Description of Artificial Sequence Synthetic polynucleotide

SEQ ID NO: 109 - protein, 214 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYIAPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 451 aa, Artificial Sequence
  Description of Artificial Sequence Synthetic polypeptide
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 451 aa, Artificial Sequence
  Description of Artificial Sequence Synthetic polypeptide
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 451 aa, Artificial Sequence
  Description of Artificial Sequence Synthetic polypeptide
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 451 aa, Artificial Sequence
  Description of Artificial Sequence Synthetic polypeptide
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 114 - protein, 451 aa, Artificial Sequence
  Description of Artificial Sequence Synthetic polypeptide
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 115 - protein, 213 aa, Artificial Sequence
  Description of Artificial Sequence Synthetic polypeptide
DIQMTQSPSSLSASVGDRVTITCRASQSINSYLNWYQQKPGKAPKLLIYAASSLQSGVPSRFSGSGSGTDFTLTISSLQPEDFATYYCQQGVSDITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 449 aa, Artificial Sequence
  Description of Artificial Sequence Synthetic polypeptide
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 449 aa, Artificial Sequence
  Description of Artificial Sequence Synthetic polypeptide
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 449 aa, Artificial Sequence
  Description of Artificial Sequence Synthetic polypeptide
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 449 aa, Artificial Sequence
  Description of Artificial Sequence Synthetic polypeptide
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 120 - protein, 449 aa, Artificial Sequence
  Description of Artificial Sequence Synthetic polypeptide
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 121 - protein, 107 aa, Artificial Sequence [sequence omitted]

SEQ ID NO: 122 - protein, 330 aa, Homo sapiens [sequence omitted]

SEQ ID NO: 123 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 126 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 127 - protein, 9 aa, Artificial Sequence
  Description of Artificial Sequence Synthetic peptide
  MOD_RES (4)..(4): Ser or Arg
  MOD_RES (5)..(5): Ser, Ile or Leu
GTFXXYAIS

SEQ ID NO: 128 - protein, 17 aa, Artificial Sequence
  Description of Artificial Sequence Synthetic peptide
  MOD_RES (5)..(5): Ile, Asp, Ala or His
  MOD_RES (8)..(8): Thr, Val or Leu
GIIPXFGXANYAQKFQG

SEQ ID NO: 129 - protein, 12 aa, Artificial Sequence
  Description of Artificial Sequence Synthetic peptide
ARGGLDTDEFDL

SEQ ID NO: 130 - protein, 11 aa, Artificial Sequence
  Description of Artificial Sequence Synthetic peptide
RASQSINSYLN

SEQ ID NO: 131 - protein, 7 aa, Artificial Sequence
  Description of Artificial Sequence Synthetic peptide
AASSLQS

SEQ ID NO: 132 - protein, 8 aa, Artificial Sequence
  Description of Artificial Sequence Synthetic peptide
QQGVSDIT

SEQ ID NO: 133 - protein, 9 aa, Artificial Sequence
  Description of Artificial Sequence Synthetic peptide
  MOD_RES (5)..(5): Asn or Ser
  MOD_RES (7)..(7): Ala or Val
  MOD_RES (9)..(9): Ser or Ile
FTFSXYXMX

SEQ ID NO: 134 - protein, 17 aa, Artificial Sequence
  Description of Artificial Sequence Synthetic peptide
  MOD_RES (1)..(1): Ala or Ser
  MOD_RES (5)..(5): Ser or Asp
  MOD_RES (6)..(6): Gly or Ser
  MOD_RES (7)..(7): Gly or Val
  MOD_RES (8)..(8): Ser or Thr
XISGXXXXTYYADSVKG

SEQ ID NO: 135 - protein, 16 aa, Artificial Sequence
  Description of Artificial Sequence Synthetic peptide
AKGPPTYHTNYYYMDV

SEQ ID NO: 136 - protein, 11 aa, Artificial Sequence
  Description of Artificial Sequence Synthetic peptide
RASQGISSWLA

SEQ ID NO: 137 - protein, 7 aa, Artificial Sequence
  Description of Artificial Sequence Synthetic peptide
AASSLQS

SEQ ID NO: 138 - protein, 9 aa, Artificial Sequence
  Description of Artificial Sequence Synthetic peptide
QQTNSFPYT

SEQ ID NO: 139 - protein, 9 aa, Artificial Sequence
  Description of Artificial Sequence Synthetic peptide
  MOD_RES (4)..(4): Ser or Val
  MOD_RES (6)..(6): His or Ala
  MOD_RES (7)..(7): Tyr or Asp
  MOD_RES (9)..(9): Asp or Asn
FTFXDXXMX

SEQ ID NO: 140 - protein, 19 aa, Artificial Sequence
  Description of Artificial Sequence Synthetic peptide
  MOD_RES (5)..(5): Lys or Ala
  MOD_RES (6)..(6): Ala or Leu
  MOD_RES (7)..(7): Ser or Gly
RTRNXXXSYTTEYAASVKG

SEQ ID NO: 141 - protein, 12 aa, Artificial Sequence
  Description of Artificial Sequence Synthetic peptide
AREPKYWIDFDL

SEQ ID NO: 142 - protein, 11 aa, Artificial Sequence
  Description of Artificial Sequence Synthetic peptide
RASQSISSYLN

SEQ ID NO: 143 - protein, 7 aa, Artificial Sequence
  Description of Artificial Sequence Synthetic peptide
AASSLQS

SEQ ID NO: 144 - protein, 9 aa, Artificial Sequence
  Description of Artificial Sequence Synthetic peptide
QQSYIAPYT

SEQ ID NO: 145 - protein, 976 aa, Homo sapiens [sequence omitted]

SEQ ID NO: 146 - protein, 972 aa, Homo sapiens
MRGARGAWDFLCVLLLLLRVQTGSSQPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKEQIHPHTLFTPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 147 - protein, 123 aa, Artificial Sequence
  Description of Artificial Sequence Synthetic polypeptide
QVQLVQSGAAVKKPGESLKISCKGSGYRFTTYWIGWVRQMPGKGLEWMGIIYPGDSDTRYSPSFQGQVTISAGKSISTAYLQWSSLKASDTAMYYCARHGRGYNGYEGAFDIWGQGTMVTVSS

SEQ ID NO: 148 - protein, 107 aa, Artificial Sequence
  Description of Artificial Sequence Synthetic polypeptide
AIQLTQSPSSLSASVGDRVTITCRASQGVSSALAWYQQKPGKAPKLLIYDASSLESGVPSRFSGSGSGTDFTLTISSLQPEDFATYYCQQFNSYPLTFGGGTKVEIK

SEQ ID NO: 149 - protein, 107 aa, Artificial Sequence
  Description of Artificial Sequence Synthetic polypeptide
DIQLTQSPSSLSASVGDRVTITCRASQGIRTDLGWYQQKPGKAPKLLIYDASSLESGVPSRFSGSGSGTDFTLTISSLQPEDFATYYCQQFNSYPLTFGGGTKVEIK

SEQ ID NO: 150 - protein, 107 aa, Artificial Sequence
  Description of Artificial Sequence Synthetic polypeptide
AIRMTQSPSSLSASVGDRVTITCRASQGIRNDLAWYQQKPGKTPKLLIYDASSLESGVPSRFSGSGSGTDFTLTISSLQPEDFATYYCQQFNSYPLTFGGGTKVEIK

SEQ ID NO: 151 - protein, 107 aa, Artificial Sequence
  Description of Artificial Sequence Synthetic polypeptide
AIQMTQSPSSLSASVGDRVTITCRASQGIRNDLGWYQQKPGKAPKLLIYDASSLESGVPSRFSGSGSGTDFTLTISSLQPEDFATYYCQQFNSYPLTFGGGTKVDIK

SEQ ID NO: 152 - protein, 107 aa, Artificial Sequence
  Description of Artificial Sequence Synthetic polypeptide
NIQMTQSPSSLSASVGDRVTITCRASQAISDYLAWFQQKPGKAPKLLIYDASNLETGVPSRFSGSGSGTDFTLTISSLQPEDFATYYCQQLNSYPLTFGGGTKVEIK

SEQ ID NO: 153 - protein, 107 aa, Artificial Sequence
  Description of Artificial Sequence Synthetic polypeptide
AIRMTQSPSSLSASVGDRVIIACRASQGIGGALAWYQQKPGNAPKVLVYDASTLESGVPSRFSGGGSGTDFTLTISSLQPEDFATYYCQQFNSYPLTFGGGTKLEIK

SEQ ID NO: 154 - protein, 107 aa, Artificial Sequence
  Description of Artificial Sequence Synthetic polypeptide
DIAMTQSPPSLSAFVGDRVTITCRASQGIISSLAWYQQKPGKAPKLLIYDASSLESGVPSRFSGSGSGTDFTLTIRSLQPEDFATYYCQQFNSYPLTFGGGTKLEIK

SEQ ID NO: 155 - protein, 107 aa, Artificial Sequence
  Description of Artificial Sequence Synthetic polypeptide
DIQMTQSPSSLSASVGDRVTITCRASQGISSALAWYQQKAGKAPKVLISDASSLESGVPSRFSGSGSGTDFTLSISSLQPEDFATYYCQQFNGYPLTFGGGTKVDIK

SEQ ID NO: 156 - protein, 107 aa, Artificial Sequence
  Description of Artificial Sequence Synthetic polypeptide
AIRMTQSPSSLSASVGDRVTITCQASQGIRNDLGWYQQKPGKAPKLLIYDASNLETGVPSRFSGSGSGTDFTFTISSLQPEDIATYYCQQFNSYPLTFGGGTKLEIK

SEQ ID NO: 157 - protein, 107 aa, Artificial Sequence
  Description of Artificial Sequence Synthetic polypeptide
NIQMTQSPSSLSTSVGDRVTITCRASQGIGTSLAWYQQKPGKPPKLLIYDASSLESGVPSRLSGSGSGTDFTLTISSLQPEDFATYYCQQSNSYPITFGQGTRLEIK

SEQ ID NO: 158 - protein, 107 aa, Artificial Sequence
  Description of Artificial Sequence Synthetic polypeptide
AIQLTQSPSSLSASVGDRVTITCRASQSIGDYLTWYQQKPGKAPKVLIYGASSLQSGVPPRFSGSGSGTDFTLTVSSLQPEDFATYYCQQLNSYPLTFGGGTKLEIK

SEQ ID NO: 159 - protein, 107 aa, Artificial Sequence
  Description of Artificial Sequence Synthetic polypeptide
DIQLTQSPSSLSASVGDRVTITCRASQGVRSTLAWYQQKPGKAPKLLIYDASILESGVPSRFSGSGSGTDFTLTISSLQPEDFATYYCQQFNGYPLTFGQGTRLEIK

SEQ ID NO: 160 - protein, 107 aa, Artificial Sequence
  Description of Artificial Sequence Synthetic polypeptide
DIVMTQSPSSLSASVGDRVTITCRASQGIRNDLGWYQQKPGKAPKLLIYDASSLESGVPSRFSGSGSGTDFTLTISSLQPEDFATYYCQQFNSYPLTFGGGTKLEIK

SEQ ID NO: 161 - protein, 107 aa, Artificial Sequence
  Description of Artificial Sequence Synthetic polypeptide
DIQLTQSPSSLSASVGDRVTITCRASQGISSFLAWYQQKPGKAPKLLIYDASTLQSGVPSRFSGSASGTDFTLTISSLQPEDFATYYCQQLNGYPLTFGGGTKVEIK

SEQ ID NO: 162 - protein, 107 aa, Artificial Sequence
  Description of Artificial Sequence Synthetic polypeptide
AIQLTQSPSSLSASVGDRVTITCRASQGIGSALAWYQQKPGIGPKLLIYDASTLESGVPARFSGSGSRTDFTLTITSLQPEDFATYYCQQFNGYPLTFGGGTKLEIK

SEQ ID NO: 163 - protein, 107 aa, Artificial Sequence
  Description of Artificial Sequence Synthetic polypeptide
AIQLTQSPSSLSASVGDRVTITCRASQGITSALAWYQEKPGKAPNLLIYDASSLESGVPSRFSGSGYGTDFTLTISSLQPEDFATYYCQQLNSYPLTFGGGTKVDIK

SEQ ID NO: 164 - protein, 121 aa, Artificial Sequence
  Description of Artificial Sequence Synthetic polypeptide
QIQLVQSGPELRKPGESVKISCKASGYTFTDYAMYWVKQAPGKGLKWMGWINTYTGKPTYADDFKGRFVFSLEASANTANLQISNLKNEDTATYFCARARGLVDDYVMDAWGQGTSVTVSS

SEQ ID NO: 165 - protein, 108 aa, Artificial Sequence
  Description of Artificial Sequence Synthetic polypeptide
SYELIQPPSASVTLGNTVSLTCVGDELSKRYAQWYQQKPDKTIVSVIYKDSERPSGISDRFSGSSSGTTATLTIHGTLAEDEADYYCLSTYSDDNLPVFGGGTKLTVL

SEQ ID NO: 166 - protein, 117 aa, Artificial Sequence
  Description of Artificial Sequence Synthetic polypeptide
EVQLQQYGAELGKPGTSVRLSCKVSGYNIRNTYIHWVNQRPGEGLEWIGRIDPTNGNTISAEKFKTKATLTADTSSHTAYLQFSQLKSDDTAIYFCALNYEGYADYWGQGVMVTGSS

SEQ ID NO: 167 - protein, 106 aa, Artificial Sequence
  Description of Artificial Sequence Synthetic polypeptide
DIQMTQSPSFLSASVGDRVTINCKASQNINKYLNWYQQKVGEAPKRLIFKTNSLQTGIPSRFSGSGSGTDYTLTISSLQTEDVATYFCFQYNIGYTFGAGTKVELK

SEQ ID NO: 168 - protein, 124 aa, Artificial Sequence
  Description of Artificial Sequence Synthetic polypeptide
EVQLQESGPGLVKPSQSLSLTCSVTGYSISSNYRWNWIRKFPGNKVEWMGYINSAGSTNYNPSLKSRISMTRDTSKNQFFLQVNSVTTEDTATYYCARSLRGYITDYSGFFDYWGQGVMVTVSS

SEQ ID NO: 169 - protein, 107 aa, Artificial Sequence
  Description of Artificial Sequence Synthetic polypeptide
DIRMTQSPASLSASLGETVNIECLASEDIFSDLAWYQQKPGKSPQLLIYNANSLQNGVPSRFSGSGSGTRYSLKINSLQSEDVATYFCQQYKNYPLTFGSGTKLEIK

SEQ ID NO: 170 - protein, 117 aa, Artificial Sequence
  Description of Artificial Sequence Synthetic polypeptide
EVQLQQYGAELGKPGTSVRLSCKLSGYKIRNTYIHWVNQRPGKGLEWIGRIDPANGNTIYAEKFKSKVTLTADTSSNTAYMQLSQLKSDDTALYFCAMNYEGYEDYWGQGVMVTVSS

SEQ ID NO: 171 - protein, 106 aa, Artificial Sequence
  Description of Artificial Sequence Synthetic polypeptide
DIQMTQSPSFLSASVGDSVTINCKASQNINKYLNWYQQKLGEAPKRLIHKTDSLQTGIPSRFSGSGSGTDYTLTISSLQPEDVATYFCFQYKSGFMFGAGTKLELK

SEQ ID NO: 172 - protein, 120 aa, Artificial Sequence
  Description of Artificial Sequence Synthetic polypeptide
QIQLVQSGPELKKPGESVKISCKASGYTFTDYAVYWVIQAPGKGLKWMGWINTYTGKPTYADDFKGRFVFSLETSASTANLQISNLKNEDTATYFCARGAGMTKDYVMDAWGRGVLVTVS

SEQ ID NO: 173 - protein, 108 aa, Artificial Sequence
  Description of Artificial Sequence Synthetic polypeptide
SYELIQPPSASVTLGNTVSLTCVGDELSKRYAQWYQQKPDKTIVSVIYKDSERPSDISDRFSGSSSGTTATLTIHGTLAEDEADYYCLSTYSDDNLPVFGGGTKLTVL

SEQ ID NO: 174 - protein, 116 aa, Artificial Sequence
  Description of Artificial Sequence Synthetic polypeptide
QVQLKESGPGLVQPSQTLSLTCTVSGFSLTSYLVHWVRQPPGKTLEWVGLMWNDGDTSYNSALKSRLSISRDTSKSQVFLKMHSLQAEDTATYYCARESNLGFTYWGHGTLVTVSS

SEQ ID NO: 175 - protein, 107 aa, Artificial Sequence
  Description of Artificial Sequence Synthetic polypeptide
DIQMTQSPASLSASLEEIVTITCKASQGIDDDLSWYQQKPGKSPQLLIYDVTRLADGVPSRFSGSRSGTQYSLKISRPQVADSGIYYCLQSYSTPYTFGAGTKLELK

SEQ ID NO: 176 - protein, 117 aa, Artificial Sequence
  Description of Artificial Sequence Synthetic polypeptide
EVQLQQYGAELGKPGTSVRLSCKVSGYNIRNTYIHWVHQRPGEGLEWIGRIDPTNGNTISAEKFKSKATLTADTSSNTAYMQFSQLKSDDTAIYFCAMNYEGYADYWGQGVMVTVSS

SEQ ID NO: 177 - protein, 106 aa, Artificial Sequence
  Description of Artificial Sequence Synthetic polypeptide
DIQMTQSPSFLSASVGDRLTINCKASQNINKYLNWYQQKLGEAPKRLIFKTNSLQTGIPSRFSGSGSGTDYTLTISSLQPEDVATYFCFQYNIGFTFGAGTKLELK

SEQ ID NO: 178 - protein, 124 aa, Artificial Sequence
  Description of Artificial Sequence Synthetic polypeptide
EVQLVESGGGLVQSGRSLKLSCAASGFTVSDYYMAWVRQAPTKGLEWVATINYDGSTTYHRDSVKGRFTISRDNAKSTLYLQMDSLRSEDTATYYCARHGDYGYHYGAYYFDYWGQGVMVTVSS

SEQ ID NO: 179 - protein, 109 aa, Artificial Sequence
  Description of Artificial Sequence Synthetic polypeptide
DIVLTQSPALAVSLGQRATISCRASQTVSLSGYNLIHWYQQRTGQQPKLLIYRASNLAPGIPARFSGSGSGTDFTLTISPVQSDDIATYYCQQSRESWTFGGGTNLEMK

SEQ ID NO: 180 - protein, 116 aa, Artificial Sequence
  Description of Artificial Sequence Synthetic polypeptide
QIQLVQSGPELKKPGESVKISCKASGYTFTDYAIHWVKQAPGQGLRWMAWINTETGKPTYADDFKGRFVFSLEASASTAHLQISNLKNEDTATFFCAGGSHWFAYWGQGTLVTVSS

SEQ ID NO: 181 - protein, 108 aa, Artificial Sequence
  Description of Artificial Sequence Synthetic polypeptide
SYELIQPPSASVTLENTVSITCSGDELSNKYAHWYQQKPDKTILEVIYNDSERPSGISDRFSGSSSGTTAILTIRDAQAEDEADYYCLSTFSDDDLPIFGGGTKLTVL

SEQ ID NO: 182 - protein, 108 aa, Artificial Sequence
  Description of Artificial Sequence Synthetic polypeptide
SYELIQPPSTSVTLGNTVSLTCVGNELPKRYAYWFQQKPDQSIVRLIYDDDRRPSGISDRFSGSSSGTTATLTIRDAQAEDEAYYYCHSTYTDDKVPIFGGGTKLTVL

SEQ ID NO: 183 - protein, 123 aa, Artificial Sequence
  Description of Artificial Sequence Synthetic polypeptide
EVQLVESGGGLVQPGRSMKLSCKASGFTFSNYDMAWVRQAPTRGLEWVASISYDGITAYYRDSVKGRFTISRENAKSTLYLQLVSLRSEDTATYYCTTEGGYVYSGPHYFDYWGQGVMVTVSS

SEQ ID NO: 184 - protein, 107 aa, Artificial Sequence
  Description of Artificial Sequence Synthetic polypeptide
DIQMTQSPSSMSVSLGDTVTITCRASQDVGIFVNWFQQKPGRSPRRMIYRATNLADGVPSRFSGSRSGSDYSLTISSLESEDVADYHCLQYDEFPRTFGGGTKLELK

SEQ ID NO: 185 - protein, 117 aa, Artificial Sequence
  Description of Artificial Sequence Synthetic polypeptide
EVQLQQYGAELGKPGTSVRLSCKVSGYKIRNTYIHWVNQRPGKGLEWIGRIDPANGNTIYAEKFKSKVTLTADTSSNTAYMQLSQLKSDDTALYFCAMNYEGYEDYWGQGVMVTVSS

SEQ ID NO: 186 - protein, 106 aa, Artificial Sequence
  Description of Artificial Sequence Synthetic polypeptide
DIQMTQSPSFLSASVGDSVTINCKASQNINKYLNWYQQKLGEAPKRLIHKTNSLQPGFPSRFSGSGSGTDYTLTISSLQPEDVAAYFCFQYNSGFTFGAGTKLELK

SEQ ID NO: 187 - protein, 125 aa, Artificial Sequence
  Description of Artificial Sequence Synthetic polypeptide
QVQLVQSGAEVKKPGASVKVSCKASGYTFTDYYIHWVRQAPGQGLEWMGWMNPHSGDTGYAQKFQGRVTMTRDTSTSTVYMELSSLRSEDTAVYYCARHGRGYNGYEGAFDIWGQGTLVTVSSAS

SEQ ID NO: 188 - protein, 107 aa, Artificial Sequence
  Description of Artificial Sequence Synthetic polypeptide
DIQMTQSPSSLSASVGDRVTITCRASQGIGNELGWYQQKPGKAPKLLIYAASNLQSGVPSRFSGSGSGTDFTLTISSLQPEDFATYYCQQYDNLPLTFGQGTKVEIK

SEQ ID NO: 189 - protein, 125 aa, Artificial Sequence
  Description of Artificial Sequence Synthetic polypeptide
QVQLVQSGAEVKKPGASVKVSCKASGYTFTGYYLHWVRQAPGQGLEWMGWINPNSGDTNYAQNFQGRVTMTRDTSTSTVYMELSSLRSEDTAVYYCARHGRGYNGYEGAFDIWGQGTLVTVSSAS

SEQ ID NO: 190 - protein, 107 aa, Artificial Sequence
  Description of Artificial Sequence Synthetic polypeptide
DIQMTQSPSSLSASVGDRVTITCRASQGIRNDLGWYQQKPGKAPKLLIYDASSLESGVPSRFSGSGSGTDFTLTISSLQPEDFATYYCQQLNGYPLTFGGGTKVEIK

SEQ ID NO: 191 - protein, 125 aa, Artificial Sequence
  Description of Artificial Sequence Synthetic polypeptide
QVQLVQSGAEVKKPGASVKVSCKASGYTFTGYYLHWVRQAPGQGLEWMGWINPNSGGTNYAQKFQGRVTMTRDTSTSTVYMELSSLRSEDTAVYYCARHGRGYEGYEGAFDIWGQGTLVTVSSAS

SEQ ID NO: 192 - protein, 107 aa, Artificial Sequence
  Description of Artificial Sequence Synthetic polypeptide
DIQMTQSPSSLSASVGDRVTITCRASQGIRNDLGWYQQKPGKAPKLLIYDASELETGVPSRFSGSGSGTDFTLTISSLQPEDFATYYCQQLNGYPITFGQGTKVEIK

SEQ ID NO: 193 - protein, 125 aa, Artificial Sequence
  Description of Artificial Sequence Synthetic polypeptide
QVQLVQSGAEVKKPGASVKVSCKASGYTFTSYYIHWVRQAPGQGLEWMGWLNPSGGGTSYAQKFQGRVTMTRDTSTSTVYMELSSLRSEDTAVYYCARHGRGYDGYEGAFDIWGQGTLVTVSSAS

SEQ ID NO: 194 - protein, 107 aa, Artificial Sequence
  Description of Artificial Sequence Synthetic polypeptide
DIQMTQSPSSLSASVGDRVTITCRASQGIRNDLGWYQQKPGKAPKLLIYDASNLETGVPSRFSGSGSGTDFTLTISSLQPEDFATYYCQQLNGYPLTFGGGTKVEIK

SEQ ID NO: 195 - protein, 125 aa, Artificial Sequence
  Description of Artificial Sequence Synthetic polypeptide
QVQLVQSGAEVKKPGASVKVSCKASGYTFSTYYMHWVRQAPGQGLEWMGIINPSGGSTSYAQKFQGRVTMTRDTSTSTVYMKLSSLRSEDTAVYYCARHGRGYEGYEGAFDIWGQGTLVTVSSAS

SEQ ID NO: 196 - protein, 107 aa, Artificial Sequence
  Description of Artificial Sequence Synthetic polypeptide
DIQMTQSPSSLSASVGDRVTITCRASQGIRDDLGWYQQKPGKAPKLLIYDASNLETGVPSRFSGSGSGTDFTLTISSLQPEDFATYYCQQANGFPLTFGGGTKVEIK

SEQ ID NO: 197 - protein, 125 aa, Artificial Sequence
  Description of Artificial Sequence Synthetic polypeptide
QVQLVQSGAEVKKPGASVKVSCKASGYTFTGYYIHWVRQAPGQGLEWMGIINPSGGNTNYAQNFQGRVTMTRDTSTSTVYMELSSLRSEDTAVYYCARHGRGYNAYEGAFDIWGQGTLVTVSSAS

SEQ ID NO: 198 - protein, 107 aa, Artificial Sequence
  Description of Artificial Sequence Synthetic polypeptide
DIQMTQSPSSLSASVGDRVTITCRASQGIRNDLGWYQQKPGKAPKLLIYDASNLETGVPSRFSGSGSGTDFTLTISSLQPEDFATYYCQQVNGYPLTFGGGTKVEIK

SEQ ID NO: 199 - protein, 125 aa, Artificial Sequence
  Description of Artificial Sequence Synthetic polypeptide
QVQLVQSGAEVKKPGASVKVSCKASGGTFSSYAISWVRQAPGQGLEWMGVINPTVGGANYAQKFQGRVTMTRDTSTSTVYMELSSLRSEDTAVYYCARHGRGYNEYEGAFDIWGQGTLVTVSSAS

SEQ ID NO: 200 - protein, 107 aa, Artificial Sequence
  Description of Artificial Sequence Synthetic polypeptide
DIQMTQSPSSLSASVGDRVTITCQASQDISDYLNWYQQKPGKAPKLLIYDASNLETGVPSRFSGSGSGTDFTLTISSLQPEDFATYYCQQGNSFPLTFGGGTKLEIK

SEQ ID NO: 201 - protein, 125 aa, Artificial Sequence
  Description of Artificial Sequence Synthetic polypeptide
QVQLVQSGAEVKKLGASVKVSCKASGYTFSSYYMHWVRQAPGQGLEWMGVINPNGAGTNFAQKFQGRVTMTRDTSTSTVYMELSSLRSEDTAVYYCARHGRGYEGYEGAFDIWGQGTLVTVSSAS

SEQ ID NO: 202 - protein, 125 aa, Artificial Sequence
  Description of Artificial Sequence Synthetic polypeptide
QVQLVQSGAEVKKPGASVKVSCKASGYTFTTYYMHWVRQAPGQGLEWMGWINPTGGGTNYAQNFQGRVTMTRDTSTSTVYMELSSLRSEDTAVYYCARHGRGYEGYEGAFDIWGQGTLVTVSSAS

SEQ ID NO: 203 - protein, 107 aa, Artificial Sequence
  Description of Artificial Sequence Synthetic polypeptide
DIQMTQSPSSLSASVGDRVTITCRASQGIRNDVSWYQQKPGKAPKLLIYDASNLETGVPSRFSGSGSGTDFTLTISSLQPEDFATYYCQQLSGYPITFGQGTKLEIK

SEQ ID NO: 204 - protein, 125 aa, Artificial Sequence
  Description of Artificial Sequence Synthetic polypeptide
QVQLVQSGAEVKKPGASVKVSCKASGYTFTSYYIHWVRQAPGQGLEWMGMINPSGGSTNYAQKFQGRVTMTRDTSTSTVYMELSSLRSEDTAVYYCARHGRGYNDYEGAFDIWGQGTLVTVSSAS

SEQ ID NO: 205 - protein, 107 aa, Artificial Sequence
  Description of Artificial Sequence Synthetic polypeptide
DIQMTQSPSSLSASVGDRVTITCRASQSISDWLAWYQQKPGKAPKLLIYEASNLEGGVPSRFSGSGSGTDFTLTISSLQPEDFATYYCQQANSFPYTFGQGTKVEIK

SEQ ID NO: 206 - protein, 125 aa, Artificial Sequence
  Description of Artificial Sequence Synthetic polypeptide
QVQLVQSGAEVKKPGASVKVSCKASGYIFSAYYIHWVRQAPGQGLEWMGIINPSGGSTRYAQKFQGRVTMTRDTSTSTVYMELSSLRSEDTAVYYCARHGRGYGGYEGAFDIWDQGTLVTVSSAS

SEQ ID NO: 207 - protein, 107 aa, Artificial Sequence
  Description of Artificial Sequence Synthetic polypeptide
DIQMTQSPSSLSASVGDRVTITCRASQGIGDYVAWYQQKPGKAPKLLIYDASNLETGVPSRFSGSGSGTDFTLTISSLQPEDFATYYCQQLNGYPITFGQGTRLEIK

SEQ ID NO: 208 - protein, 125 aa, Artificial Sequence
  Description of Artificial Sequence Synthetic polypeptide
EVQLVQSGAEVKKPGESLKISCKGSGYRFTSYWIGWVRQMPGKGLEWMGIIYPDDSDTRYSPSFQGQVTISVDKSNSTAYLQWSSLKASDTAMYYCARHGRGYNGYEGAFDIWGQGTLVTVSSAS

SEQ ID NO: 209 - protein, 107 aa, Artificial Sequence
  Description of Artificial Sequence Synthetic polypeptide
DIQMTQSPSSLSASVGDRVTITCRASQGISSYLAWYQQKPGKAPKLLIYDASNLETGVPSRFSGSGSGTYFTLTISSLQPEDFATYYCQQGASFPITFGQGTKVEIK

SEQ ID NO: 210 - protein, 125 aa, Artificial Sequence
  Description of Artificial Sequence Synthetic polypeptide
EVQLVQSGAEVKKPGESLKISCKGSGSSFPNSWIAWVRQMPGKGLEWMGIIYPSDSDTRYSPSFQGQVTISADKSISTAYLQWSSLEASDTAMYYCARHGRGYNGYEGAFDIWGQGTLVTVSSAS

SEQ ID NO: 211 - protein, 107 aa, Artificial Sequence
  Description of Artificial Sequence Synthetic polypeptide
DIQMTQSPSSLSASVGDRVTITCRASQGIRNYLAWYQQKPGKAPKLLIYDASSLQSGVPSRFSGSGSGTDFTLTISSLQPEDFATYYCQQLNSYPLTFGGGTKVEIK

SEQ ID NO: 212 - protein, 125 aa, Artificial Sequence
  Description of Artificial Sequence Synthetic polypeptide
EVQLVQSGAEVKKPGESLKISCKGSGYSFDSYWIGWVRQMPGKGLEWMGIMYPGDSDTRYSPSFQGQVTISADKSISTAYLQWSSLKASDTAMYYCARHGRGYNAYEGAFDIWGQGTLVTVSSAS

SEQ ID NO: 213 - protein, 107 aa, Artificial Sequence
  Description of Artificial Sequence Synthetic polypeptide
DIQMTQSPSSLSASVGDRVTITCRASQSINNWLAWYQQKPGKAPKLLIYDAFILQSGVPSRFSGSGSGTDFTLTISSLQPEDFATYYCLQLNSYPLTFGPGTKVDIK

SEQ ID NO: 214 - protein, 124 aa, Artificial Sequence
  Description of Artificial Sequence Synthetic polypeptide
EVQLVQSGAEVKKPGESLKISCKGSGYSFTNWIAWVRQMPGKGLEWMGIIYPGDSETRYSPSFQGQVTISADKSISTAYLQWSSLKASDTAMYYCARHGRGYYGYEGAFDIWGQGTLVTVSSAS

SEQ ID NO: 215 - protein, 107 aa, Artificial Sequence
  Description of Artificial Sequence Synthetic polypeptide
DIQMTQSPSSLSASVGDRVTITCRASQGISDNLNWYQQKPGKAPKLLIYDASNLETGVPSRFSGSGSGTDFTLTISSLQPEDFATYYCQQAISFPLTFGQGTKVEIK

SEQ ID NO: 216 - protein, 125 aa, Artificial Sequence
  Description of Artificial Sequence Synthetic polypeptide
EVQLVQSGAEVKKPGESLKISCKGSGYNFTSYWIGWVRQMPGKGLEWMGVIYPDDSETRYSPSFQGQVTISADKSISTAYLQWSSLKASDTAMYYCARHGRGYNGYEGAFDIWGQGTLVTVSSAS

SEQ ID NO: 217 - protein, 107 aa, Artificial Sequence
  Description of Artificial Sequence Synthetic polypeptide
DIQMTQSPSSLSASVGDRVTITCRASRDIRDDLGWYQQKPGKAPKLLIYDASNLETGVPSRFSGSGSGTDFTLTISSLQPEDFATYYCQQANSFPLTFGGGTKVEIK

SEQ ID NO: 218 - protein, 124 aa, Artificial Sequence
  Description of Artificial Sequence Synthetic polypeptide
EVQLVQSGAEVKKPGESLKISCKGSGYTFNTYIGWVRQMPGKGLEWMGIIYPGDSGTRYSPSFQGQVTISADKAISTAYLQWSSLKASDTAMYYCARHSRGYNGYEGAFDIWGQGTLVTVSSAS

SEQ ID NO: 219 - protein, 107 aa, Artificial Sequence
  Description of Artificial Sequence Synthetic polypeptide
DIQMTQSPSSLSASVGDRVTITCRASQGISNYLAWYQQKPGKAPKLLIYDASNLETGVPSRFSGSGSGTDFTLTISSLQPEDFATYYCQQANSFPVTFGQGTKVEIK

SEQ ID NO: 220 - protein, 125 aa, Artificial Sequence
  Description of Artificial Sequence Synthetic polypeptide
EVQLVQSGAEVKKPGESLKISCKGSGYNFTTYWIGWVRQMPGKGLEWMGIIHPADSDTRYNPSFQGQVTISADKSISTAYLQWSSLKASDTAMYYCARHGRGYNGYEGAFDIWGQGTLVTVSSAS

SEQ ID NO: 221 - protein, 107 aa, Artificial Sequence
  Description of Artificial Sequence Synthetic polypeptide
DIQMTQSPSSLSASVGDRVTITCRVSQGISSYLAWYQQKPGKAPKLLIYDASNLETGVPSRFSGSGSGTDFTLTISSLQPEDFATYYCQQANSFPLTFGGGTKVEIK

SEQ ID NO: 222 - protein, 125 aa, Artificial Sequence
  Description of Artificial Sequence Synthetic polypeptide
EVQLVQSGAEVKKPGESLKISCKGSGYRFSNYWIAWVRQMPGKGLEWMGIIYPDNSDTRYSPSFQGQVTISADKSISTAYLQWSSLKASDTAMYYCARHGRGYDGYEGAFDIWGQGTLVTVSSAS

SEQ ID NO: 223 - protein, 107 aa, Artificial Sequence
  Description of Artificial Sequence Synthetic polypeptide
DIQMTQSPSSLSASVGDRVTITCRASQGIRSDLAWYQQKPGKAPKLLIYGASSLQSGVPSRFSGSGSGTDFTLTISSLQPEDFATYYCQQANSFPLSFGQGTKVEIK

SEQ ID NO: 224 - protein, 124 aa, Artificial Sequence
  Description of Artificial Sequence Synthetic polypeptide
EVQLVQSGAEVKKPGESLKISCKGSGYRFASYWIGWVRQMPGKGLEWMGITYPGDSETRYNPSQGQVTISADKSISTAYLQWSSLKASDTAMYYCARHGRGYGGYEGAFDIWGQGTLVTVSSAS

SEQ ID NO: 225 - protein, 107 aa, Artificial Sequence
  Description of Artificial Sequence Synthetic polypeptide
DIQMTQSPSSLSASVGDRVTITCRASQGIRNDLGWYQQKPGKAPKLLIYDASNLETGVPSRFSGSGSGTDFTLTISSLQPEDFATYYCQQANSFPLTFGGGTKVEIK

SEQ ID NO: 226 - protein, 125 aa, Artificial Sequence
  Description of Artificial Sequence Synthetic polypeptide
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARHGRGYNGYEGAFDIWGQGTLVTVSSAS

SEQ ID NO: 227 - protein, 107 aa, Artificial Sequence
  Description of Artificial Sequence Synthetic polypeptide
DIQMTQSPSSLSASVGDRVTITCRASQSISNWLAWYQQKPGKAPKLLIYDASNLETGVPSRFSGSGSGTDFTLTISSLQPEDFATYYCQQTNSFPLTFGQGTRLEIK

SEQ ID NO: 228 - protein, 107 aa, Artificial Sequence
  Description of Artificial Sequence Synthetic polypeptide
DIQLTQSPSSLSASVGDRVTITCRASQGVISALAWYQQKPGKAPKLLIYDASSLESGVPSRFSGSGSGTDFTLTISSLQPEDFATYYCQQFNSYPLTFGGGTKVEIK

SEQ ID NO: 229 - protein, 107 aa, Artificial Sequence
  Description of Artificial Sequence Synthetic polypeptide
DIQLTQSPSSLSASVGDRVTITCRASQGIRSALAWYQQKPGKAPKLLIYDASSLESGVPSRFSGSGSGTDFTLTISSLQPEDFATYYCQQFNSYPLTFGGGTKVEIK

SEQ ID NO: 230 - protein, 107 aa, Artificial Sequence
  Description of Artificial Sequence Synthetic polypeptide
DIQLTQSPSSLSASVGDRVTITCRASQGVGSALAWYQQKPGKAPKLLIYDASSLESGVPSRFSGSGSGTDFTLTISSLQPEDFATYYCQQFNSYPLTFGGGTKVEIK

SEQ ID NO: 231 - protein, 107 aa, Artificial Sequence
  Description of Artificial Sequence Synthetic polypeptide
DIQLTQSPSSLSASVGDRVTITCRASQGVISALAWYQQKPGKAPKLLIYDASILESGVPSRFSGSGSGTDFTLTISSLQPEDFATYYCQQFNSYPLTFGGGTKVEIK

SEQ ID NO: 232 - protein, 107 aa, Artificial Sequence
  Description of Artificial Sequence Synthetic polypeptide
DIQLTQSPSSLSASVGDRVTITCRASQGIRSALAWYQQKPGKAPKLLIYDASILESGVPSRFSGSGSGTDFTLTISSLQPEDFATYYCQQFNSYPLTFGGGTKVEIK

SEQ ID NO: 233 - protein, 107 aa, Artificial Sequence
  Description of Artificial Sequence Synthetic polypeptide
DIQLTQSPSSLSASVGDRVTITCRASQGVGSALAWYQQKPGKAPKLLIYDASILESGVPSRFSGSGSGTDFTLTISSLQPEDFATYYCQQFNSYPLTFGGGTKVEIK

SEQ ID NO: 234 - protein, 107 aa, Artificial Sequence
  Description of Artificial Sequence Synthetic polypeptide
DIQLTQSPSSLSASVGDRVTITCRASQGISSALAWYQQKPGKAPKLLIYDASILESGVPSRFSGSGSGTDFTLTISSLQPEDFATYYCQQFNSYPLTFGGGTKVEIK

SEQ ID NO: 235 - protein, 107 aa, Artificial Sequence
  Description of Artificial Sequence Synthetic polypeptide
DIQLTQSPSSLSASVGDRVTITCRASQGVISALAWYQQKPGKAPKLLIYDASTLESGVPSRFSGSGSGTDFTLTISSLQPEDFATYYCQQFNSYPLTFGGGTKVEIK

SEQ ID NO: 236 - protein, 107 aa, Artificial Sequence
  Description of Artificial Sequence Synthetic polypeptide
DIQLTQSPSSLSASVGDRVTITCRASQGIRSALAWYQQKPGKAPKLLIYDASTLESGVPSRFSGSGSGTDFTLTISSLQPEDFATYYCQQFNSYPLTFGGGTKVEIK

SEQ ID NO: 237 - protein, 107 aa, Artificial Sequence
  Description of Artificial Sequence Synthetic polypeptide
DIQLTQSPSSLSASVGDRVTITCRASQGVGSALAWYQQKPGKAPKLLIYDASTLESGVPSRFSGSGSGTDFTLTISSLQPEDFATYYCQQFNSYPLTFGGGTKVEIK

SEQ ID NO: 238 - protein, 123 aa, Artificial Sequence
  Description of Artificial Sequence Synthetic polypeptide
EVQLVQSGAEVKKPGESLKISCKGSGYRFTTSWIGWVRQMPGKGLEWMGIIYPGDSDTRYSPSFQGQVTISADKSISTAYLQWSSLKASDTAMYYCARHGLGYNGYEGAFDIWGQGTLVTVSS

SEQ ID NO: 239 - protein, 107 aa, Artificial Sequence
  Description of Artificial Sequence Synthetic polypeptide
DIQMTQSPSSLSASVGDRVTITCRASQGIGSALAWYQQKPGKAPKLLIYDASTLESGVPSRFSGSGSGTDFTLTISSLQPEDFATYYCQQFNGYPLTFGQGTRLEIK

SEQ ID NO: 240 - protein, 107 aa, Artificial Sequence
  Description of Artificial Sequence Synthetic polypeptide
DIQMTQSPSSLSASVGDRVTITCRASRGISDYLAWYQQKPGKAPKLLIYDASNLETGVPSRFSGSGSGTDFTLTISSLQPEDFATYYCQQANSFPITFGQGTRLEIK

SEQ ID NO: 241 - protein, 107 aa, Artificial Sequence
  Description of Artificial Sequence Synthetic polypeptide
DIQMTQSPSSLSASVGDRVTITCRASQGIGSALAWYQQKPGKAPKLLIYDASTLESGVPSRFSGSGSGTDFTLTISSLQPEDFATYYCQQLNGYPLTFGQGTRLEIK

SEQ ID NO: 242 - protein, 107 aa, Artificial Sequence
  Description of Artificial Sequence Synthetic polypeptide
DIQMTQSPSSLSASVGDRVTITCRASQGIGSALAWYQQKPGKAPKLLIYDASNLETGVPSRFSGSGSGTDFTLTISSLQPEDFATYYCQQLNGYPLTFGQGTRLEIK

SEQ ID NO: 243 - protein, 123 aa, Artificial Sequence
  Description of Artificial Sequence Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSS

SEQ ID NO: 244 - protein, 107 aa, Artificial Sequence
  Description of Artificial Sequence Synthetic polypeptide
DIQMTQSPSSLSASVGDRVTITCRSSQGIRSDLGWYQQKPGKAPKLLIYDASNLETGVPSRFSGSGSGTDFTLTISSLQPEDFATYYCQQANGFPLTFGGGTKVEIK

SEQ ID NO: 245 - protein, 5 aa, Artificial Sequence
  Description of Artificial Sequence Synthetic peptide
NYWIG

SEQ ID NO: 246 - protein, 17 aa, Artificial Sequence
  Description of Artificial Sequence Synthetic peptide
IINPRDSDTRYRPSFQG

SEQ ID NO: 247 - protein, 14 aa, Artificial Sequence
  Description of Artificial Sequence Synthetic peptide
HGRGYEGYEGAFDI

SEQ ID NO: 248 - protein, 11 aa, Artificial Sequence
  Description of Artificial Sequence Synthetic peptide
RSSQGIRSDLG

SEQ ID NO: 249 - protein, 7 aa, Artificial Sequence
  Description of Artificial Sequence Synthetic peptide
DASNLET

SEQ ID NO: 250 - protein, 9 aa, Artificial Sequence
  Description of Artificial Sequence Synthetic peptide
QQANGFPLT

SEQ ID NO: 251 - protein, 123 aa, Artificial Sequence
  Description of Artificial Sequence Synthetic polypeptide
EVQLVQSGAEVKKPGESLKISCKGSGYSFTNYWIGWVRQMPGKGLEWMGIIYPGDSDIRYSPSLQGQVTISVDTSTSTAYLQWNSLKPSDTAMYYCARHGRGYNGYEGAFDIWGQGTLVTVSS

SEQ ID NO: 252 - protein, 107 aa, Artificial Sequence
  Description of Artificial Sequence Synthetic polypeptide
DIQMTQSPSSLSASVGDRVTITCRASQGIGDSLAWYQQKPGKAPKLLIYDASNLETGVPSRFSGSGSGTDFTLTISSLQPEDFATYYCQQLNGYPITFGQGTKVEIK

SEQ ID NO: 253 - protein, 17 aa, Artificial Sequence
  Description of Artificial Sequence Synthetic peptide
IIYPGDSDIRYSPSLQG

SEQ ID NO: 254 - protein, 11 aa, Artificial Sequence
  Description of Artificial Sequence Synthetic peptide
RASQGIGDSLA

SEQ ID NO: 255 - protein, 9 aa, Artificial Sequence
  Description of Artificial Sequence Synthetic peptide
QQLNGYPIT

SEQ ID NO: 256 - protein, 107 aa, Artificial Sequence
  Description of Artificial Sequence Synthetic polypeptide
DIQMTQSPSSLSASVGDRVTITCRASQGIRNDLGWYQQKPGKAPKLLIYDASSLESGVPSRFSGSGSGTDFTLTISSLQPEDFATYYCQQLNGYPITFGQGTKVEIK

SEQ ID NO: 257 - protein, 11 aa, Artificial Sequence
  Description of Artificial Sequence Synthetic peptide
RASQGIRNDLG

SEQ ID NO: 258 - protein, 123 aa, Artificial Sequence
  Description of Artificial Sequence Synthetic polypeptide
EVQLVQSGAEVKKPGESLKISCKGSGYSFTNYWIGWVRQMPGKGLEWMGIIYPGDSLTRYSPSFQGQVTISADKSISTAYLQWSSLKASDTAMYYCARHGRGYNGYEGAFDIWGQGTLVTVSS

SEQ ID NO: 259 - protein, 17 aa, Artificial Sequence
  Description of Artificial Sequence Synthetic peptide
IIYPGDSLTRYSPSFQG

SEQ ID NO: 260 - protein, 123 aa, Artificial Sequence
  Description of Artificial Sequence Synthetic polypeptide
EVQLVQSGAEVKKPGESLKISCKGSGYSFTNYWIGWVRQMPGKGLEWMGIIYPGDSDTRYSPSFQGQVTISADKSISTAYLQWSSLKASDTAMYYCARHGRGYNGYEGAFDIWGQGTLVTVSS

SEQ ID NO: 261 - protein, 123 aa, Artificial Sequence
  Description of Artificial Sequence Synthetic polypeptide
QVQLVQSGAAVKKPGESLKISCKGSGYRFTSYWIGWVRQMPGKGLEWMGIIYPGDSDTRYSPSFQGQVTISAGKSISTAYLQWSSLKASDTAMYYCARHGRGYNGYEGAFDIWGQGTMVTVSS

SEQ ID NO: 262 - protein, 107 aa, Artificial Sequence [sequence omitted]

SEQ ID NO: 263 - protein, 5 aa, Artificial Sequence
  Description of Artificial Sequence Synthetic peptide
TYWIG

SEQ ID NO: 264 - protein, 11 aa, Artificial Sequence
  Description of Artificial Sequence Synthetic peptide
RASQGVISALA

SEQ ID NO: 265 - protein, 7 aa, Artificial Sequence
  Description of Artificial Sequence Synthetic peptide
DASILES

SEQ ID NO: 266 - protein, 9 aa, Artificial Sequence
  Description of Artificial Sequence Synthetic peptide
QQFNSYPLT

SEQ ID NO: 267 - protein, 11 aa, Artificial Sequence
  Description of Artificial Sequence Synthetic peptide
RASQGVGSALA

SEQ ID NO: 268 - protein, 7 aa, Artificial Sequence
  Description of Artificial Sequence Synthetic peptide
DASTLES

SEQ ID NO: 269 - protein, 330 aa, Homo sapiens [sequence omitted]

SEQ ID NO: 270 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 271 - protein, 330 aa, Artificial Sequence; identical to SEQ ID NO: 123
  Description of Artificial Sequence Synthetic polypeptide

SEQ ID NO: 272 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 273 - protein, 330 aa, Artificial Sequence; identical to SEQ ID NO: 124
  Description of Artificial Sequence Synthetic polypeptide

SEQ ID NO: 274 - protein, 330 aa, Artificial Sequence; identical to SEQ ID NO: 126
  Description of Artificial Sequence Synthetic polypeptide

SEQ ID NO: 275 - protein, 453 aa, Artificial Sequence
  Description of Artificial Sequence Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 276 - protein, 453 aa, Artificial Sequence
  Description of Artificial Sequence Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 277 - protein, 453 aa, Artificial Sequence
  Description of Artificial Sequence Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 278 - protein, 453 aa, Artificial Sequence
  Description of Artificial Sequence Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 279 - protein, 453 aa, Artificial Sequence
  Description of Artificial Sequence Synthetic polypeptide
EVQLVQSGAEVKKPGESLKISCKGSGYRFTTSWIGWVRQMPGKGLEWMGIIYPGDSDTRYSPSFQGQVTISADKSISTAYLQWSSLKASDTAMYYCARHGLGYNGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 280 - protein, 453 aa, Artificial Sequence
  Description of Artificial Sequence Synthetic polypeptide
EVQLVQSGAEVKKPGESLKISCKGSGYRFTTSWIGWVRQMPGKGLEWMGIIYPGDSDTRYSPSFQGQVTISADKSISTAYLQWSSLKASDTAMYYCARHGLGYNGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 281 - protein, 453 aa, Artificial Sequence
  Description of Artificial Sequence Synthetic polypeptide
EVQLVQSGAEVKKPGESLKISCKGSGYRFTTSWIGWVRQMPGKGLEWMGIIYPGDSDTRYSPSFQGQVTISADKSISTAYLQWSSLKASDTAMYYCARHGLGYNGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 282 - protein, 453 aa, Artificial Sequence
  Description of Artificial Sequence Synthetic polypeptide
EVQLVQSGAEVKKPGESLKISCKGSGYRFTTSWIGWVRQMPGKGLEWMGIIYPGDSDTRYSPSFQGQVTISADKSISTAYLQWSSLKASDTAMYYCARHGLGYNGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 283 - protein, 107 aa, Artificial Sequence [sequence omitted]

SEQ ID NO: 284 - protein, 214 aa, Artificial Sequence
  Description of Artificial Sequence Synthetic polypeptide
DIQMTQSPSSLSASVGDRVTITCRSSQGIRSDLGWYQQKPGKAPKLLIYDASNLETGVPSRFSGSGSGTDFTLTISSLQPEDFATYYCQQANGFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 285 - protein, 214 aa, Artificial Sequence
  Description of Artificial Sequence Synthetic polypeptide
DIQMTQSPSSLSASVGDRVTITCRASQGIGSALAWYQQKPGKAPKLLIYDASNLETGVPSRFSGSGSGTDFTLTISSLQPEDFATYYCQQLNGYPLTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 286 - protein, 5 aa, Artificial Sequence
  Description of Artificial Sequence Synthetic peptide
TSWIG

SEQ ID NO: 287 - protein, 14 aa, Artificial Sequence
  Description of Artificial Sequence Synthetic peptide
HGLGYNGYEGAFDI

SEQ ID NO: 288 - protein, 11 aa, Artificial Sequence
  Description of Artificial Sequence Synthetic peptide
RASQGIGSALA

SEQ ID NO: 289 - protein, 10 aa, Artificial Sequence
  Description of Artificial Sequence Synthetic peptide
CQQLNGYPLT

SEQ ID NO: 290 - protein, 293 aa, Escherichia coli
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 291 - protein, 293 aa, Shigella dysenteriae
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 292 - protein, 297 aa, Escherichia coli
DEFTVDFSSQKSYVDSLNSIRSAISTPLGNISQGGVSVSVINHVLGGNYISLNVRGLDPYSERFNHLRLIMERNNLYVAGFINTETNIFYRFSDFSHISVPDVITVSMTTDSSYSSLQRIADLERTGMQIGRHSLVGSYLDLMEFRGRSMTRASSRAMLRFVTVIAEALRFRQIQRGFRPALSEASPLYTMTAQDVDLTLNWGRISNVLPEYRGEEGVRIGRISFNSLSAILGSVAVILNCHSTGSYSVRSVSQKQKTECQIVGDRAAIKVNNVLWEANTIAALLNRKPQDLTEPNQ

SEQ ID NO: 293 - protein, 351 aa, Homo sapiens
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 294 - protein, 5 aa, Artificial Sequence
  Description of Artificial Sequence Synthetic peptide
EYYMY

SEQ ID NO: 295 - protein, 17 aa, Artificial Sequence
  Description of Artificial Sequence Synthetic peptide
RIDPEDGSIDYVEKFKK

SEQ ID NO: 296 - protein, 9 aa, Artificial Sequence
  Description of Artificial Sequence Synthetic peptide
GKFNYRFAY

SEQ ID NO: 297 - protein, 16 aa, Artificial Sequence
  Description of Artificial Sequence Synthetic peptide
RSSQSLLHSSGNTYLN

SEQ ID NO: 298 - protein, 7 aa, Artificial Sequence
  Description of Artificial Sequence Synthetic peptide
LVSKLES

SEQ ID NO: 299 - protein, 9 aa, Artificial Sequence
  Description of Artificial Sequence Synthetic peptide
MQFTHYPYT

SEQ ID NO: 300 - protein, 118 aa, Artificial Sequence
  Description of Artificial Sequence Synthetic polypeptide
QVQLVQSGAEVKKPGASVKVSCKASGYTFTEYYMYWVRQAPGQGLELMGRIDPEDGSIDYVEKFKKKVTLTADTSSSTAYMELSSLTSDDTAVYYCARGKFNYRFAYWGQGTLVTVSS

SEQ ID NO: 301 - protein, 112 aa, Artificial Sequence
  Description of Artificial Sequence Synthetic polypeptide
DVVMTQSPPSLLVTLGQPASISCRSSQSLLHSSGNTYLNWLLQRPGQSPQPLIYLVSKLESGVPDRFSGSGSGTDFTLKISGVEAEDVGVYYCMQFTHYPYTFGQGTKLEIK

SEQ ID NO: 302 - protein, 7 aa, Artificial Sequence
  Description of Artificial Sequence Synthetic peptide
GFTFSSY

SEQ ID NO: 303 - protein, 5 aa, Artificial Sequence
  Description of Artificial Sequence Synthetic peptide
SGGGF

SEQ ID NO: 304 - protein, 9 aa, Artificial Sequence
  Description of Artificial Sequence Synthetic peptide
SSYGEIMDY

SEQ ID NO: 305 - protein, 11 aa, Artificial Sequence
  Description of Artificial Sequence Synthetic peptide
RASQRIGTSIH

SEQ ID NO: 306 - protein, 7 aa, Artificial Sequence
  Description of Artificial Sequence Synthetic peptide
YASESIS

SEQ ID NO: 307 - protein, 10 aa, Artificial Sequence
  Description of Artificial Sequence Synthetic peptide
QQSHGWPFTF

SEQ ID NO: 308 - protein, 117 aa, Artificial Sequence
  Description of Artificial Sequence Synthetic polypeptide
EVKLVESGGGLVKPGGSLKLSCAASGFTFSSYDMSWVRQTPEKRLEWVASISGGGFLYYLDSVKGRFTISRDNARNILYLHMTSLRSEDTAMYYCARSSYGEIMDYWGQGTSVTVSS

SEQ ID NO: 309 - protein, 107 aa, Artificial Sequence
  Description of Artificial Sequence Synthetic polypeptide
DILLTQSPAILSVSPGERVSFSCRASQRIGTSIHWYQQRTTGSPRLLIKYASESISGIPSRFSGSGSGTDFTLSINSVESEDVADYYCQQSHGWPFTFGGGTKLEIE

SEQ ID NO: 310 - protein, 9 aa, Artificial Sequence
  Description of Artificial Sequence Synthetic peptide
SSYGELMDY

SEQ ID NO: 311 - protein, 117 aa, Artificial Sequence
  Description of Artificial Sequence Synthetic polypeptide
EVKLVESGGGLVKPGGSLKLSCAASGFTFSSYDMSWVRQTPEKRLEWVASISGGGFLYYLDSVKGRFTISRDNARNILYLHMTSLRSEDTAMYYCARSSYGELMDYWGQGTSVTVSS

SEQ ID NO: 312 - protein, 495 aa, Homo sapiens
MPMGSLQPLATLYLLGMLVASCLGRLSWYDPDFQARLTRSNSKCQGQLEVYLKDGWHMVCSQSWGRSSKQWEDPSQASKVCQRLNCGVPLSLGPFLVTYTPQSSIICYGQLGSFSNCSHSRNDMCHSLGLTCLEPQKTTPPTTRPPPTTTPEPTAPPRLQLVAQSGGQHCAGVVEFYSGSLGGTISYEAQDKTQDLENFLCNNLQCGSFLKHLPETEAGRAQDPGEPREHQPLPIQWKIQNSSCTSLEHCFRKIKPQKSGRVLALLCSGFQPKVQSRLVGGSSICEGTVEVRQGAQWAALCDSSSARSSLRWEEVCREQQCGSVNSYRVLDAGDPTSRGLFCPHQKLSQCHELWERNSYCKKVFVTCQDPNPAGLAAGTVASIILALVLLVVLLVVCGPLAYKKLVKKFRQKKQRQWIGPTGMNQNMSFHRNHTATVRSHAENPTASHVDNEYSQPPRNSHLSAYPALEGALHRSSMQPDNSSDSDYDLHGAQRL

SEQ ID NO: 313 - protein, 120 aa, Artificial Sequence
  Description of Artificial Sequence Synthetic polypeptide
QVTLKESGPVLVKPTETLTLTCTFSGFSLSTSGMGVGWIRQAPGKGLEWVAHIWWDDDVYYNPSLKSRLTITKDASKDQVSLKLSSVTAADTAVYYCVRRRATGTGFDYWGQGTLVTVSS

SEQ ID NO: 314 - protein, 106 aa, Artificial Sequence
  Description of Artificial Sequence Synthetic polypeptide
NIVMTQSPSSLSASVGDRVTITCQASQDVGTAVAWYQQKPDQSPKLLIYWTSTRHTGVPDRFTGSGSGTDFTLTISSLQPEDIATYFCHQYNSYNTFGSGTKLEIK

SEQ ID NO: 315 - protein, 9 aa, Artificial Sequence
  Description of Artificial Sequence Synthetic peptide
FSLSTSGMG

SEQ ID NO: 316 - protein, 5 aa, Artificial Sequence
  Description of Artificial Sequence Synthetic peptide
WWDDD

SEQ ID NO: 317 - protein, 10 aa, Artificial Sequence
  Description of Artificial Sequence Synthetic peptide
RRATGTGFDY

SEQ ID NO: 318 - protein, 6 aa, Artificial Sequence
  Description of Artificial Sequence Synthetic peptide
QDVGTA

SEQ ID NO: 319 - protein, 7 aa, Artificial Sequence
  Description of Artificial Sequence Synthetic peptide
WTSTRHT

SEQ ID NO: 320 - protein, 6 aa, Artificial Sequence
  Description of Artificial Sequence Synthetic peptide
YNSYNT

SEQ ID NO: 321 - protein, 9 aa, Unknown
  Description of Unknown “LAGLIDADG” family motif peptide
LAGLIDADG

SEQ ID NO: 322 - protein, 9 aa, Human immunodeficiency virus 1
RKKRRQRRR

SEQ ID NO: 323 - protein, 12 aa, Human immunodeficiency virus 1
RQARRNRRRRWR

SEQ ID NO: 324 - protein, 7 aa, Simian virus 40
PKKKRKV

SEQ ID NO: 325 - protein, 8 aa, Unknown
  Description of Unknown Amatoxin sequence
  MOD_RES (7)..(7): Asn or Asp
IWGIGCXP

(19) United States
(12) Patent Application Publication
(10) Pub. No.: US 2022/0175946 A1
(43) Pub. Date: Jun. 9, 2022

(54) CONDITIONING METHODS FOR GENE THERAPY

(71) Applicant: Magenta Therapeutics, Inc., Cambridge, MA (US)

(72) Inventors: Anthony BOITANO, Newton, MA (US); Michael Cooke, Boston, MA (US); Rahul Palchaudhuri, Somerville, MA (US); Bradley R. Pearse, Watertown, MA (US)

(21) Appl. No.: 17/507,456

(22) Filed: Oct. 21, 2021

Related U.S. Application Data
(63) Continuation of application No. PCT/US2020/029934, filed on Apr. 24, 2020.
(60) Provisional application No. 62/944,925, filed on Dec. 6, 2019.
(60) Provisional application No. 62/838,278, filed on Apr. 24, 2019.

(51) Int. Cl. A61K 47/68; A61K 35/28; C07K 16/28

(52) U.S. Cl. CPC A61K 47/6825; A61K 35/28; C07K 16/2803; A61K 48/00; C07K 2317/92; C07K 2317/21; A61K 47/6849

(57) ABSTRACT

The disclosure provides compositions and methods useful for the depletion of a specific population of endogenous hematopoietic stem cells and/or immune cells from a subject prior to transplantation with genetically modified stem cells to improve the engraftment of the transplanted stem cells and provide gene therapy. The disclosure provides compositions and methods for the treatment of various hematopoietic diseases, metabolic disorders, cancers, and autoimmune diseases, among others. Described herein are antibodies, antigen-binding fragments, and conjugates thereof that can be applied to effect the treatment of these conditions, for instance, by depleting a population of CD117+ or CD45+ cells in a patient, such as a human.

### RELATED APPLICATIONS

This application is a continuation of PCT Appln. No. PCT/US2020/029934, filed on Apr. 24, 2020, which claims priority to U.S. Provisional Application No. 62/838,278, filed on Apr. 24, 2019 and U.S. Provisional Application No. 62/944,925, filed on Dec. 6, 2019. The contents of each of the priority applications are incorporated by reference herein.

### SEQUENCE LISTING

The instant application contains a Sequence Listing which has been submitted electronically in ASCII format and is hereby incorporated by reference in its entirety. Said ASCII copy, created on Oct. 20, 2021, is named M103034_2070US_C1_SL.txt and is 341,247 bytes in size.

### BACKGROUND

Hematopoietic stem cells (HSCs) have significant therapeutic potential for addressing various diseases. More recently, HSC-based therapies include the use of powerful gene-editing methods that allow genetic modification of stem cells. Genetically modified HSCs can be delivered to patients suffering from diseases of a particular blood cell (e.g., sickle cell disease), metabolic disorders (e.g., mucopolysaccharidosis), cancers, and autoimmune conditions (e.g., chronic granulomatosis disease), among others, to correct a defective gene. For many patients, HSC-based therapy remains the only curative treatment.

Hematopoietic stem cell transplant (HSCT) requires a conditioning of the subject's tissues (e.g., bone marrow tissue) prior to engraftment. Current non-targeted conditioning methods, which include, for example, irradiation (e.g., total body irradiation or TBI) and DNA alkylating/modifying agents, are highly toxic to multiple organ systems, hematopoietic and non-hematopoietic cells and the hematopoietic microenvironment. These harsh conditioning regimens effectively kill the host subject's immune and niche cells and adversely affect multiple organ systems, frequently leading to life-threatening complications. For example, while recent advances in gene editing methods have enabled the development of genetically modified stem cells for sickle cell disease, the current treatment methods that include harsh conditioning regimens have proved unsuccessful, with patients developing life-threatening or long-term complications such as cancer (e.g., secondary malignancies) and infertility. Thus, while HSCs have significant therapeutic potential, such limitations have hindered their use in the clinic.

There is currently a need for methods that promote the engraftment of genetically modified HSC grafts such that the multi-potency and hematopoietic functionality of the HSCs and their corrected or altered genes are preserved in the patient following transplantation.

### FIELD

The present disclosure relates to the use of genetically modified stem cells for the treatment of patients suffering from various pathologies, such as blood diseases, metabolic disorders, cancers, and autoimmune diseases, among others, in conjunction with a conditioning method using an antibody drug conjugate (ADC), wherein the ADC is capable of binding molecules (e.g., CD117 or CD45) on hematopoietic stem cells and/or immune cells.

### SUMMARY

Described herein is a method of providing stem cell gene therapy, comprising methods of administering genetically modified stem cells to a subject in need thereof in conjunction with a conditioning method comprising the use of an antibody-drug conjugate (ADC). The antibodies of the ADCs described herein target and deplete a specific population of endogenous hematopoietic stem cells and/or immune cells from the subject prior to transplantation with genetically modified stem cells.

In some aspects, the present disclosure provides a method of administering genetically modified stem cells to a human subject in need thereof comprising: a) administering to the human subject an antibody-drug conjugate (ADC) that binds to a cell surface molecule expressed on hematopoietic stem cells (HSC) and/or immune cells, thereby depleting HSCs and/or immune cells from the human subject; and b) administering to the human subject a transplant to the human subject comprising a population of genetically modified stem cells. In some embodiments, the ADC binds to a cell surface molecule expressed on HSCs and/or immune cells to be depleted.

In some aspects, the present disclosure provides a method of treating a human subject with genetically modified cells, the method comprising administering a transplant comprising a population of genetically modified stem cells to the human subject in need thereof, wherein the human subject has received a conditioning treatment comprising an antibody-drug conjugate (ADC) that binds to a cell surface molecule expressed on hematopoietic stem cells (HSC) and/or immune cells.

In some embodiments, the genetically modified stem cells are autologous stem cells. In some embodiments, the genetically modified stem cells are allogeneic stem cells.

In some embodiments, the genetically modified stem cells are HSCs.

In some embodiments, the genetically modified stem cells are CD34+ HSCs.

In some embodiments, the subject is suffering from any one or more of a cancer, a hemoglobinopathy disorder, myelodysplastic disorder, immunodeficiency disorder, or a metabolic disorder.

In some embodiments, the hemoglobinopathy disorder is selected from any one or more of sickle cell anemia, thalassemia, Fanconi anemia, aplastic anemia, or Wiskott-Aldrich syndrome.

In some embodiments, the immunodeficiency disorder is a congenital immunodeficiency or an acquired immunodeficiency.

In some embodiments, the acquired immunodeficiency is human immunodeficiency virus or acquired immune deficiency syndrome (AIDS).

In some embodiments, the metabolic disorder is selected from any one or more of glycogen storage diseases, mucopolysaccharidosis, Gaucher's Disease, Hurlers Disease, sphingolipidoses, globoid cell leukodystrophy, or metachromatic leukodystrophy.

In some embodiments, the cancer is selected from any one or more of leukemia, lymphoma, multiple myeloma, or neuroblastoma.

In some embodiments, the cancer is a hematological cancer, selected from, e.g., acute myeloid leukemia, acute lymphoid leukemia, chronic myeloid leukemia, chronic lymphoid leukemia, or multiple myeloma.

In some embodiments, the subject is suffering from a disorder selected from any one or more of an adenosine deaminase deficiency, severe combined immunodeficiency, hyper immunoglobulin M syndrome, Chediak-Higashi disease, hereditary lymphohistiocytosis, osteopetrosis, osteogenesis imperfecta, storage diseases, thalassemia major, systemic sclerosis, systemic lupus erythematosus, multiple sclerosis, or juvenile rheumatoid arthritis.

In some embodiments, the subject is suffering from an autoimmune disorder. In some embodiments, the autoimmune disorder is selected from any one or more of multiple sclerosis, human systemic lupus, rheumatoid arthritis, inflammatory bowel disease, treating psoriasis, Type 1 diabetes mellitus, acute disseminated encephalomyelitis, Addison's disease, alopecia universalis, ankylosing spondylitisis, antiphospholipid antibody syndrome, aplastic anemia, autoimmune hemolytic anemia, autoimmune hepatitis, autoimmune inner ear disease, autoimmune lymphoproliferative syndrome, autoimmune oophoritis, Balo disease, Behcet's disease, bullous pemphigoid, cardiomyopathy, Chagas' disease, chronic fatigue immune dysfunction syndrome, chronic inflammatory demyelinating polyneuropathy, Crohn's disease, cicatrical pemphigoid, coeliac sprue-dermatitis herpetiformis, cold agglutinin disease, CREST syndrome, Degos disease, discoid lupus, dysautonomia, endometriosis, essential mixed cryoglobulinemia, fibromyalgia-fibromyositis, Goodpasture's syndrome, Grave's disease, Guillain-Barre syndrome, Hashimoto's thyroiditis, Hidradenitis suppurativa, idiopathic and/or acute thrombocytopenic purpura, idiopathic pulmonary fibrosis, IgA neuropathy, interstitial cystitis, juvenile arthritis, Kawasaki's disease, lichen planus, Lyme disease, Meniere disease, mixed connective tissue disease, myasthenia gravis, neuromyotonia, opsoclonus myoclonus syndrome, optic neuritis, Ord's thyroiditis, pemphigus vulgaris, pernicious anemia, polychondritis, polymyositis and dermatomyositis, primary biliary cirrhosis, polyarteritis nodosa, polyglandular syndromes, polymyalgia rheumatica, primary agammaglobulinemia, Raynaud phenomenon, Reiter's syndrome, rheumatic fever, sarcoidosis, scleroderma, Sjögren's syndrome, stiff person syndrome, Takayasu's arteritis, temporal arteritis, ulcerative colitis, uveitis, vasculitis, vitiligo, vulvodynia, chronic granulomatosis disease, or Wegener's granulomatosis.

In some embodiments, the population of stem cells has been genetically modified to alter a target gene. In some embodiments, the target gene is selected from one or more of beta-globin, gamma-globin, adenosine deaminase, arylsulfatase A, WASp gene, phagocyte NADPH oxidase, galatosyla ceramidase, beta-galactosidase, beta-hexosaminidase, alpha-L iduronidase, ATM serine/threonine kinase, Ribosome maturation protein SBDS, or CCR5.

In some embodiments, the transplant comprising a population of genetically modified stem cells has been altered using a gene editing system. In some embodiments, the gene editing system is a CRISPR/Cas system.

In some embodiments, the ADC comprises an antibody or antigen-binding fragment thereof that binds one or more cell surface molecule selected from CD2, CD5, CD7, CDwl2, CD13, CD15, CD19, CD21, CD22, CD29, CD30, CD33, CD34, CD36, CD38, CD40, CD41, CD42a, CD42b, CD42c, CD42d, CD43, CD45, CD45RA, CD45RB, CD45RC, CD45RO, CD48, CD49b, CD49d, CD49e, CD49f, CD50, CD53, CD55, CD64a, CD68, CD71, CD72, CD73, CD81, CD82, CD85A, CD85K, CD90, CD99, CD104, CD105, CD109, CD110, CD111, CD112, CD114, CD115, CD117, CD123, CD124, CD126, CD127, CD130, CD131, CD133, CD135, CD137, CD138, CD151, CD157, CD162, CD164, CD168, CD172a, CD173, CD174, CD175, CD175s, CD176, CD183, CD191, CD200, CD201, CD205, CD217, CD220, CD221, CD222, CD223, CD224, CD225, CD226, CD227, CD228, CD229, CD230, CD235a, CD235b, CD236, CD236R, CD238, CD240, CD242, CD243, CD277, CD292, CDw293, CD295, CD298, CD309, CD318, CD324, CD325, CD338, CD344, CD349, or CD350.

In some embodiments, the ADC comprises an antibody or antigen-binding fragment thereof that binds CD117.

In some embodiments, the ADC is administered in an amount sufficient to deplete a population of CD117+ cells in the subject.

In some embodiments, the CD117 is GNNK+ CD117.

In some embodiments, the anti-CD117 antibody or antigen-binding fragment thereof comprises: (a) a heavy chain variable region comprising a CDR1 domain comprising the amino acid sequence as set forth in SEQ ID NO: 31, a CDR2 domain comprising the amino acid sequence as set forth in SEQ ID NO:32, and a CDR3 domain comprising the amino acid sequence as set forth in SEQ ID NO: 33; and a light chain variable region comprising a CDR1 domain comprising the amino acid sequence as set forth in SEQ ID NO: 34, a CDR2 domain comprising the amino acid sequence as set forth in SEQ ID NO:35, and a CDR3 domain comprising the amino acid sequence as set forth in SEQ ID NO: 36. In some embodiments, the anti-CD117 antibody or antigen-binding fragment thereof comprises: a heavy chain variable region comprising a CDR1 domain comprising the amino acid sequence as set forth in SEQ ID NO: 21, a CDR2 domain comprising the amino acid sequence as set forth in SEQ ID NO:22, and a CDR3 domain comprising the amino acid sequence as set forth in SEQ ID NO: 23; and a light chain variable region comprising a CDR1 domain comprising the amino acid sequence as set forth in SEQ ID NO: 24, a CDR2 domain comprising the amino acid sequence as set forth in SEQ ID NO:25, and a CDR3 domain comprising the amino acid sequence as set forth in SEQ ID NO: 26. In some embodiments, the anti-CD117 antibody or antigen-binding fragment thereof comprises: a heavy chain variable region comprising a CDR1 domain comprising the amino acid sequence as set forth in SEQ ID NO: 41, a CDR2 domain comprising the amino acid sequence as set forth in SEQ ID NO:42, and a CDR3 domain comprising the amino acid sequence as set forth in SEQ ID NO: 43; and a light chain variable region comprising a CDR1 domain comprising the amino acid sequence as set forth in SEQ ID NO: 44, a CDR2 domain comprising the amino acid sequence as set forth in SEQ ID NO:45, and a CDR3 domain comprising the amino acid sequence as set forth in SEQ ID NO: 46. In some embodiments, the anti-CD117 antibody or antigen-binding fragment thereof comprises: a heavy chain variable region comprising a CDR1 domain comprising the amino acid sequence as set forth in SEQ ID NO: 51, a CDR2 domain comprising the amino acid sequence as set forth in SEQ ID NO:52, and a CDR3 domain comprising the amino acid sequence as set forth in SEQ ID NO: 53; and a light chain variable region comprising a CDR1 domain comprising the amino acid sequence as set forth in SEQ ID NO: 54, a CDR2 domain comprising the amino acid sequence as set forth in SEQ ID NO:55, and a CDR3 domain comprising the amino acid sequence as set forth in SEQ ID NO: 56. In some embodiments, the anti-CD117 antibody or antigen-binding fragment thereof comprises: a heavy chain variable region comprising a CDR1 domain comprising the amino acid sequence as set forth in SEQ ID NO: 61, a CDR2 domain comprising the amino acid sequence as set forth in SEQ ID NO:62, and a CDR3 domain comprising the amino acid sequence as set forth in SEQ ID NO: 63; and a light chain variable region comprising a CDR1 domain comprising the amino acid sequence as set forth in SEQ ID NO: 64, a CDR2 domain comprising the amino acid sequence as set forth in SEQ ID NO:65, and a CDR3 domain comprising the amino acid sequence as set forth in SEQ ID NO: 66. In some embodiments, the anti-CD117 antibody or antigen-binding fragment thereof comprises: a heavy chain variable region comprising a CDR1 domain comprising the amino acid sequence as set forth in SEQ ID NO: 71, a CDR2 domain comprising the amino acid sequence as set forth in SEQ ID NO:72, and a CDR3 domain comprising the amino acid sequence as set forth in SEQ ID NO: 73; and a light chain variable region comprising a CDR1 domain comprising the amino acid sequence as set forth in SEQ ID NO: 74, a CDR2 domain comprising the amino acid sequence as set forth in SEQ ID NO:75, and a CDR3 domain comprising the amino acid sequence as set forth in SEQ ID NO: 76. In some embodiments, the anti-CD117 antibody or antigen-binding fragment thereof comprises: a heavy chain variable region comprising a CDR1 domain comprising the amino acid sequence as set forth in SEQ ID NO: 81, a CDR2 domain comprising the amino acid sequence as set forth in SEQ ID NO:82, and a CDR3 domain comprising the amino acid sequence as set forth in SEQ ID NO: 83; and a light chain variable region comprising a CDR1 domain comprising the amino acid sequence as set forth in SEQ ID NO: 84, a CDR2 domain comprising the amino acid sequence as set forth in SEQ ID NO:85, and a CDR3 domain comprising the amino acid sequence as set forth in SEQ ID NO: 86. In some embodiments, the anti-CD117 antibody or antigen-binding fragment thereof comprises: a heavy chain variable region comprising a CDR1 domain comprising the amino acid sequence as set forth in SEQ ID NO: 11, a CDR2 domain comprising the amino acid sequence as set forth in SEQ ID NO:12, and a CDR3 domain comprising the amino acid sequence as set forth in SEQ ID NO: 13; and a light chain variable region comprising a CDR1 domain comprising the amino acid sequence as set forth in SEQ ID NO: 14, a CDR2 domain comprising the amino acid sequence as set forth in SEQ ID NO:15, and a CDR3 domain comprising the amino acid sequence as set forth in SEQ ID NO: 16. In some embodiments, the anti-CD117 antibody or antigen-binding fragment thereof comprises: a heavy chain variable region comprising a CDR1 domain comprising the amino acid sequence as set forth in SEQ ID NO: 91, a CDR2 domain comprising the amino acid sequence as set forth in SEQ ID NO:92, and a CDR3 domain comprising the amino acid sequence as set forth in SEQ ID NO: 93; and a light chain variable region comprising a CDR1 domain comprising the amino acid sequence as set forth in SEQ ID NO: 94, a CDR2 domain comprising the amino acid sequence as set forth in SEQ ID NO:95, and a CDR3 domain comprising the amino acid sequence as set forth in SEQ ID NO: 96. In some embodiments, the anti-CD117 antibody or antigen-binding fragment thereof comprises: a heavy chain variable region comprising a CDR1 domain comprising the amino acid sequence as set forth in SEQ ID NO: 101, a CDR2 domain comprising the amino acid sequence as set forth in SEQ ID NO:102, and a CDR3 domain comprising the amino acid sequence as set forth in SEQ ID NO: 103; and a light chain variable region comprising a CDR1 domain comprising the amino acid sequence as set forth in SEQ ID NO: 104, a CDR2 domain comprising the amino acid sequence as set forth in SEQ ID NO:105, and a CDR3 domain comprising the amino acid sequence as set forth in SEQ ID NO: 106. In some embodiments, the anti-CD117 antibody or antigen-binding fragment thereof comprises: a heavy chain variable region comprising a CDR1 domain comprising the amino acid sequence as set forth in SEQ ID NO: 245, a CDR2 domain comprising the amino acid sequence as set forth in SEQ ID NO:246, and a CDR3 domain comprising the amino acid sequence as set forth in SEQ ID NO: 247; and a light chain variable region comprising a CDR1 domain comprising the amino acid sequence as set forth in SEQ ID NO: 248, a CDR2 domain comprising the amino acid sequence as set forth in SEQ ID NO:249, and a CDR3 domain comprising the amino acid sequence as set forth in SEQ ID NO: 250.

In some embodiments, the anti-CD117 antibody or antigen-binding fragment thereof comprises a heavy chain variable region comprising a CDR1 domain comprising the amino acid sequence as set forth in SEQ ID NO: 127, a CDR2 domain comprising the amino acid sequence as set forth in SEQ ID NO:128, and a CDR3 domain comprising the amino acid sequence as set forth in SEQ ID NO: 129; and comprising a light chain variable region comprising a CDR1 domain comprising the amino acid sequence as set forth in SEQ ID NO: 130, a CDR2 domain comprising the amino acid sequence as set forth in SEQ ID NO:131, and a CDR3 domain comprising the amino acid sequence as set forth in SEQ ID NO: 132.

In some embodiments, the anti-CD117 antibody or antigen-binding fragment thereof comprises: (a) a heavy chain variable region comprising a CDR1 domain comprising the amino acid sequence as set forth in SEQ ID NO: 133, a CDR2 domain comprising the amino acid sequence as set forth in SEQ ID NO:134, and a CDR3 domain comprising the amino acid sequence as set forth in SEQ ID NO: 135; and comprising a light chain variable region comprising a CDR1 domain comprising the amino acid sequence as set forth in SEQ ID NO: 136, a CDR2 domain comprising the amino acid sequence as set forth in SEQ ID NO:137, and a CDR3 domain comprising the amino acid sequence as set forth in SEQ ID NO: 138. In some embodiments, the anti-CD117 antibody or antigen-binding fragment thereof comprises a heavy chain variable region comprising a CDR1 domain comprising the amino acid sequence as set forth in SEQ ID NO: 139, a CDR2 domain comprising the amino acid sequence as set forth in SEQ ID NO:140, and a CDR3 domain comprising the amino acid sequence as set forth in SEQ ID NO: 141; and comprising a light chain variable region comprising a CDR1 domain comprising the amino acid sequence as set forth in SEQ ID NO: 142, a CDR2 domain comprising the amino acid sequence as set forth in SEQ ID NO:143, and a CDR3 domain comprising the amino acid sequence as set forth in SEQ ID NO: 144.

In some embodiments, the anti-CD117 antibody or antigen-binding fragment thereof comprises: a heavy chain variable region comprising the amino acid sequence as set forth in SEQ ID NO: 29, and a light chain variable region comprising the amino acid sequence as set forth in SEQ ID NO: 30. In some embodiments, the anti-CD117 antibody or antigen-binding fragment thereof comprises: a heavy chain variable region comprising the amino acid sequence as set forth in SEQ ID NO: 19, and a light chain variable region comprising the amino acid sequence as set forth in SEQ ID NO: 20. In some embodiments, the anti-CD117 antibody or antigen-binding fragment thereof comprises: a heavy chain variable region comprising the amino acid sequence as set forth in SEQ ID NO: 39, and a light chain variable region comprising the amino acid sequence as set forth in SEQ ID NO: 40. In some embodiments, the anti-CD117 antibody or antigen-binding fragment thereof comprises: a heavy chain variable region comprising the amino acid sequence as set forth in SEQ ID NO: 49, and a light chain variable region comprising the amino acid sequence as set forth in SEQ ID NO: 50. In some embodiments, the anti-CD117 antibody or antigen-binding fragment thereof comprises: a heavy chain variable region comprising the amino acid sequence as set forth in SEQ ID NO: 59, and a light chain variable region comprising the amino acid sequence as set forth in SEQ ID NO: 60.

In some embodiments, the anti-CD117 antibody or antigen-binding fragment thereof comprises: a heavy chain variable region comprising the amino acid sequence as set forth in SEQ ID NO: 69, and a light chain variable region comprising the amino acid sequence as set forth in SEQ ID NO: 70. In some embodiments, the anti-CD117 antibody or antigen-binding fragment thereof comprises: a heavy chain variable region comprising the amino acid sequence as set forth in SEQ ID NO: 79, and a light chain variable region comprising the amino acid sequence as set forth in SEQ ID NO: 80. In some embodiments, the anti-CD117 antibody or antigen-binding fragment thereof comprises: a heavy chain variable region comprising the amino acid sequence as set forth in SEQ ID NO: 9, and a light chain variable region comprising the amino acid sequence as set forth in SEQ ID NO: 10. In some embodiments, the anti-CD117 antibody or antigen-binding fragment thereof comprises: a heavy chain variable region comprising the amino acid sequence as set forth in SEQ ID NO: 89, and a light chain variable region comprising the amino acid sequence as set forth in SEQ ID NO: 90. In some embodiments, the anti-CD117 antibody or antigen-binding fragment thereof comprises: a heavy chain variable region comprising the amino acid sequence as set forth in SEQ ID NO: 99, and a light chain variable region comprising the amino acid sequence as set forth in SEQ ID NO: 100. In some embodiments, the anti-CD117 antibody or antigen-binding fragment thereof comprises: a heavy chain variable region comprising the amino acid sequence as set forth in SEQ ID NO: 243, and a light chain variable region comprising the amino acid sequence as set forth in SEQ ID NO: 244.

In some embodiments, the anti-CD117 antibody, or antigen binding fragment thereof has a dissociation rate (K_{OFF}) of 1×10⁻² to 1×10⁻³, 1×10⁻³ to 1×10⁻⁴, 1×10⁻⁵ to 1×10⁻⁶, 1×10⁻⁶ to 1×10⁻⁷ or 1×10⁻⁷ to 1×10⁻⁸ as measured by bio-layer interferometry (BLI).

In some embodiments, the anti-CD117 antibody or antigen-binding fragment thereof binds CD117 with a K_{D} of about 100 nM or less, about 90 nM or less, about 80 nM or less, about 70 nM or less, about 60 nM or less, about 50 nM or less, about 40 nM or less, about 30 nM or less, about 20 nM or less, about 10 nM or less, about 8 nM or less, about 6 nM or less, about 4 nM or less, about 2 nM or less, about 1 nM or less as determined by a Bio-Layer Interferometry (BLI) assay.

In some embodiments, the antibody or antigen-binding fragment thereof is human. In some embodiments, the antibody or antigen-binding fragment thereof is an intact antibody. In some embodiments, the antibody or antigen-binding fragment thereof is an IgG. In some embodiments, the antibody or antigen-binding fragment thereof is an IgG1 or an IgG4. In some embodiments, the antibody or antigen-binding fragment thereof is a monoclonal antibody.

In some embodiments, the antibody or antigen-binding fragment thereof comprises a heavy chain constant region having an amino acid sequence as set forth as SEQ ID NO: 122 and/or a light chain constant region comprising an amino acid sequence as set forth in SEQ ID NO: 121.

In some embodiments, the antibody or antigen-binding fragment thereof comprises an Fc region comprising at least one amino acid substitution selected from the group consisting of D265C, H435A, L234A, and L235A (numbering according to the EU index). In some embodiments, the Fc region comprises amino acid substitutions D265C, L234A, and L235A (numbering according to the EU index).

In some embodiments, the anti-CD117 antibody or antigen-binding fragment thereof comprises a light chain comprising an amino acid sequence as set forth in SEQ ID NO: 109, and a heavy chain comprising an amino acid sequence selected from the group consisting of SEQ ID NO: 110, SEQ ID NO: 111, SEQ ID NO: 112, SEQ ID NO: 113, and SEQ ID NO: 114.

In some embodiments, the anti-CD117 antibody or antigen-binding fragment thereof comprises a light chain comprising an amino acid sequence as set forth in SEQ ID NO: 115, and a heavy chain comprising an amino acid sequence selected from the group consisting of SEQ ID NO: 116, SEQ ID NO: 117, SEQ ID NO: 118, SEQ ID NO: 119, and SEQ ID NO: 120.

In some embodiments, the anti-CD117 antibody or antigen-binding fragment thereof comprises a light chain comprising an amino acid sequence as set forth in SEQ ID NO: 284, and a heavy chain comprising an amino acid sequence selected from the group consisting of SEQ ID NO: 275, SEQ ID NO: 276, SEQ ID NO: 277, and SEQ ID NO: 278.

In some embodiments, the anti-CD117 antibody or antigen-binding fragment thereof comprises a heavy chain comprising an HC-CDR1, an HC-CDR2, and an HC-CDR3 or a variable region sequence from the heavy chain variable region of Ab55, Ab54, Ab56, Ab57, Ab58, Ab61, Ab66, Ab67, Ab68, Ab69, Ab85, Ab86, Ab87, Ab88, Ab89, Ab77, Ab79, Ab81, Ab85, or Ab249, and a light chain comprising an LC-CDR1, an LC-CDR2, and an LC-CDR3 or a variable region sequence from the light chain variable region of Ab55, Ab54, Ab56, Ab57, Ab58, Ab61, Ab66, Ab67, Ab68, Ab69, Ab85, Ab86, Ab87, Ab88, Ab89, Ab77, Ab79, Ab81, Ab85, or Ab249. In some embodiments, the anti-CD117 antibody or antigen-binding fragment thereof comprises a heavy chain comprising an HC-CDR1, an HC-CDR2, and an HC-CDR3 or a variable region from the heavy chain variable region amino acid sequence of SEQ ID NO: 147, 164, 166, 168, 170, 172, 174, 176, 178, 180, 183, 185, 187, 189, 191, 193, 195, 197, 199, 201, 202, 204, 206, 208, 210, 212, 214, 216, 218, 220, 222, 224, 226, 238, or 243, and a light chain comprising an LC-CDR1, an LC-CDR2, and an LC-CDR3 or a variable region from the light chain variable region amino acid sequence of SEQ ID NO: 148, 149, 150, 151, 152, 153, 154, 155, 156, 157, 158, 159, 160, 161, 162, 163, 165, 167, 169, 171, 173, 175, 177, 179, 181, 182, 184, 186, 188, 190, 192, 194, 196, 198, 200, 203, 205, 207, 209, 211, 213, 215, 217, 219, 221, 223, 225, 227, 228, 229, 230, 231, 232, 233, 234, 235, 236, 237, 239, 240, 241, 242, or 244.

In some embodiments, the ADC is represented by the formula Ab-(Z-L-Cy)ₙ, wherein: Ab is the antibody or antigen-binding fragment thereof; L is a linker; Z is a chemical moiety formed by a coupling reaction between a reactive substituent Z′ present on L and a reactive substituent present within the antibody or antigen-binding fragment thereof, Cy is a cytotoxin selected from the group consisting of an amatoxin, pseudomonas exotoxin A, deBouganin, diphtheria toxin, saporin, maytansine, a maytansinoid, a pyrrolobenzodiazepine, a pyrrolobenzodiazepine dimer, an indolinobenzodiazepine, an indolinobenzodiazepine dimer, an indolinobenzodiazepine pseudodimer, a calicheamicin, an auristatin, and an anthracycline; and n is an integer from about 1 to about 20, which represents the average number of cytotoxins per antibody. In some embodiments, n is an integer of about 1, about 2, about 3, about 4, about 5, about 6, about 7, about 8, about 9, about 10, about 11, about 12, about 13, about 14, about 15, about 16, about 17, about 18, about 19, or about 20. In some embodiments, n is 2.

In some embodiments, the cytotoxin is an amatoxin.

In some embodiments, the ADC is represented by formula (I):

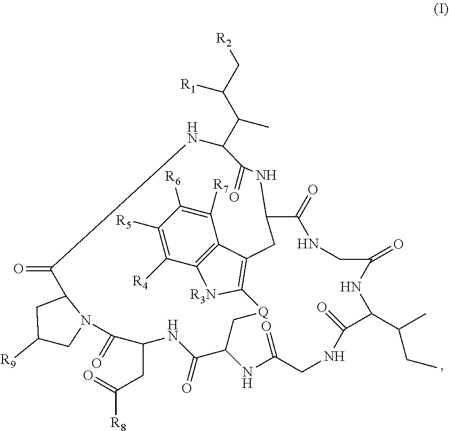

wherein:

Q is —S—, —S(O)—, or —SO₂—;

R₁ is H, OH, OR_{A}, or OR_{D};

R₂ is H, OH, OR_{B}, or OR_{D};

R_{A} and R_{B}, when present, together with the oxygen atoms to which they are bound, combine to form an optionally substituted 5-membered heterocycloalkyl group;

R₃ is H, R_{C}, or R_{D};

R₄ is H, OH, OR_{C}, OR_{D}, R_{C}, or R_{D};

R₅ is H, OH, OR_{C}, OR_{D}, R_{C}, or R_{D};

R₆ is H, OH, OR_{C}, OR_{D}, R_{C}, or R_{D};

R₇ is H, OH, OR_{C}, OR_{D}, R_{C}, or R_{D};

R₈ is OH, NH₂, OR_{C}, OR_{D}, NHR_{D}, or NR_{C}R_{D};

R₉ is H, OH, OR_{C}, or OR_{D};

R_{C} is C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, cycloalkyl, heterocycloalkyl, aryl, heteroaryl, or a combination thereof, wherein each of said C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, cycloalkyl, heterocycloalkyl, aryl, or heteroaryl may optionally be substituted with from 1 to 5 substituents, independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro;

R_{D} is -L-Z-Ab, wherein the ADC of formula (I) contains exactly one R_{D} substituent;

L comprises one or more of a hydrazine, a disulfide, a thioether, an amino acid, a peptide consisting of up to 10 amino acids, a p-aminobenzyl (PAB) group, a heterocyclic self-immolative group, C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, C₃-C₆ cycloalkyl, heterocycloalkyl, aryl, heteroaryl, a —(C═O)— group, a —C(O)NH— group, an —OC(O)NH— group, or a —(CH₂CH₂O)ₚ— group where p is an integer from 1-6;

wherein each C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, C₃-C₆ cycloalkyl, heterocycloalkyl, aryl, or heteroaryl group may be optionally substituted with from 1 to 5 (e.g., 1, 2, 3, 4, or 5) substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro; and

each C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, C₃-C₆ cycloalkyl, heterocycloalkyl, aryl, or heteroaryl group may optionally be interrupted by one or more heteroatoms selected from O, S and N.

In some embodiments, the ADC of formula (is represented by formula (Ia):

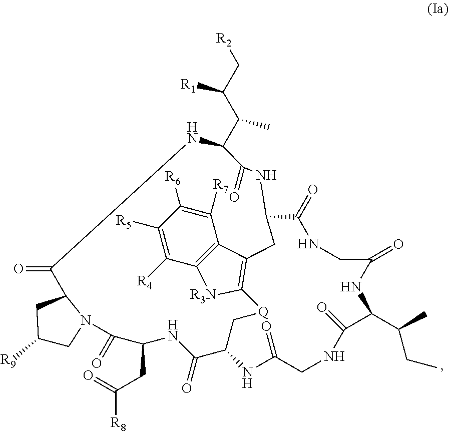

wherein each of Q, R₁-R₉, R_{A}, R_{B}, R_{C}, R_{D}, L, and Z are as previously defined for formula (I).

In some embodiments, R₁ is OR_{A}; R₂ is OR_{B}; and R_{A} and R_{B}, together with the oxygen atoms to which they are bound, combine to form:

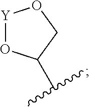

wherein:

Y is —(C═O)—, —(C═S)—, —(C═NH)—, —(CH)₂—, or CR_{E}R_{E′}, and

R_{E} and R_{E′} are each independently selected from H, C₁-C₆ alkylene-R_{D}, C₁-C₆ heteroalkylene-R_{D}, C₂-C₆ alkenylene-R_{D}, C₂-C₆ heteroalkenylene-R_{D}, C₂-C₆ alkynylene-R_{D}, C₂-C₆ heteroalkynylene-R_{D}, cycloalkylene-R_{D}, heterocycloalkylene-R_{D}, arylene-R_{D}, and heteroarylene-R_{D}, wherein each of said C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, cycloalkylene, heterocycloalkylene, arylene, or heteroarylene may optionally be substituted with from 1 to 5 (e.g., 1, 2, 3, 4, or 5) substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

In some embodiments, Y is C═O, represented by the formula:

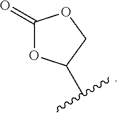 [SMILES: *C1COC(=O)O1]

In some embodiments, the linker comprises one or more of a peptide, oligosaccharide, —(CH₂)ₚ—, —(CH₂CH₂O)ₚ—, —(C═O)—, —(C═O)(CH₂)ₚ—, PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB, wherein p is an integer from 1-6 (e.g., 1, 2, 3, 4, 5, or 6).

In some embodiments, the linker comprises PAB-Ala-Val-propionyl, represented by the formula:

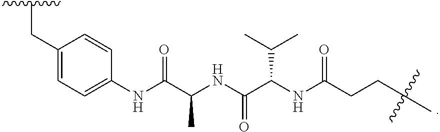 [SMILES: *CCC(=O)N[C@H](C(=O)N[C@@H](C)C(=O)Nc1ccc(C*)cc1)C(C)C]

In some embodiments, the linker comprises PAB-Cit-Val-propionyl, represented by the formula:

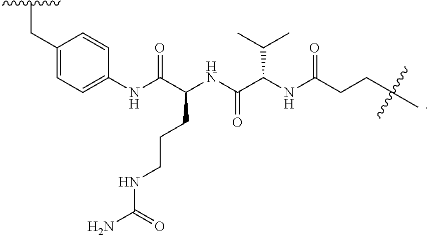 [SMILES: *CCC(=O)N[C@H](C(=O)N[C@@H](CCCNC(N)=O)C(=O)Nc1ccc(C*)cc1)C(C)C]

In some embodiments, the linker-antibody conjugate, taken together as L-Z-Ab, has the structure:

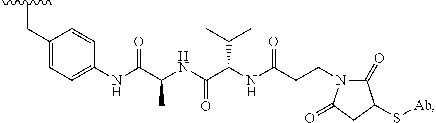

where S is a sulfur atom which represents a reactive substituent present within the antibody or antigen-binding fragment thereof.

In some embodiments, L-Z-Ab, has the structure:

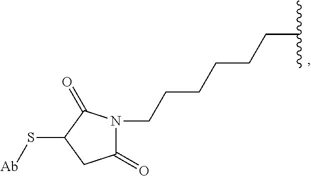

where S is a sulfur atom which represents a reactive substituent present within the antibody or antigen-binding fragment thereof.

In some embodiments, the ADC of formula (Ia) is selected from the group consisting of:

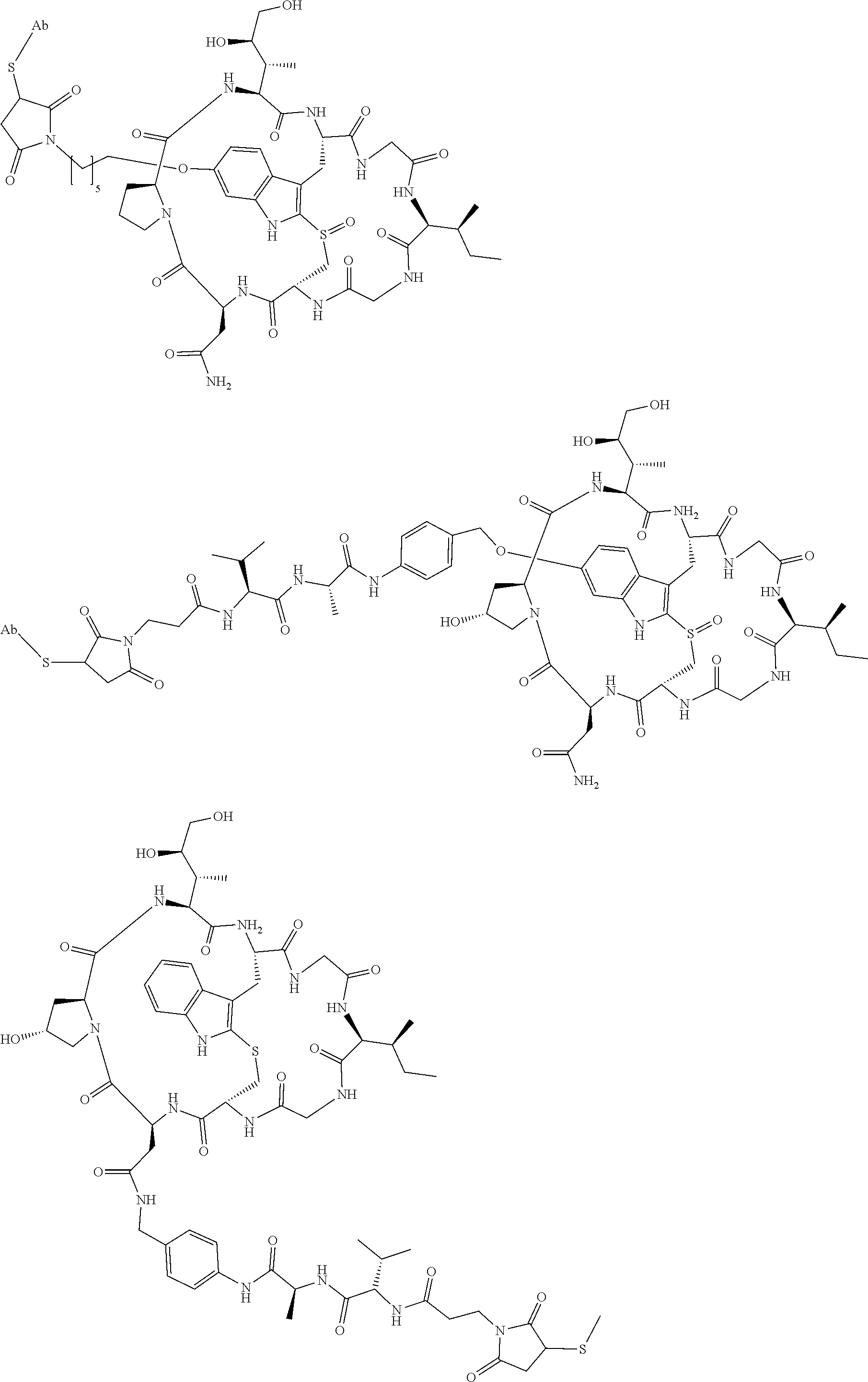

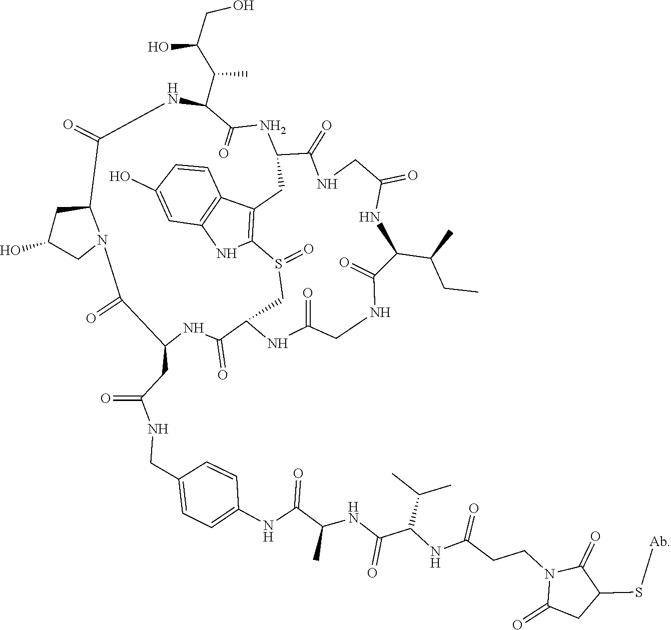

### BRIEF DESCRIPTION OF THE FIGURES

FIGS. 1A and 1B graphically depicts the results of in vitro cell killing assays that show Kasumi-1 cell viability as measured in luminescence (RLU) by Celltiter Glo as a function of the indicated anti-CD117 ADO or control concentration. The results for Ab54 (FIG. 1A), Ab55 (FIG. 1A), Ab56 (FIG. 1A), Ab57 (FIG. 1A), Ab58 (FIG. 1A), Ab61 (FIG. 1A), Ab66 (FIG. 1B), Ab67 (FIG. 1B), Ab68 (FIG. 1B), and Ab69 (FIG. 1B) are shown.

FIG. 2 graphically depicts a quantification of the area under the killing curve in the in vitro cell killing assays depicted in FIGS. 1A and 1B.

FIGS. 3-6 show the 00117-ADO is potent on primary human and non-human primate (NHP) CD34+ cells and is selective for HSCs. FIG. 3 graphically depicts the results of an in vitro cell killing assay using human and non-human primate (NHP) HSCs that shows the CD117 ADO efficiently depletes HSCs and progenitor cells in comparison to the human isotype and NHP isotype controls. FIG. 4 describes the results of a single dose of the CD117-ADC administered to Rhesus primates analyzed using flow cytotmetry showing significant HSC depletion while the lymphocytes was maintained. The data demonstrated that a single dose of an CD117-ADC led to significant colony forming cell depletion in the bone marrow 7 days after dosing (FIG. 5). FIG. 6 graphically depicts the rapid clearance of the CD117-ADC which enables graft infusion within days post-dosing.

FIGS. 7-11 show the CD117-ADC is sufficient to enable autologous gene modified transplant therapy in Rhesus primates. FIG. 7A depicts the treatment scheme for gene-marked autologous transplant in (single-dose administered) CD117 ADC-conditioned primates. FIG. 7B depicts the treatment scheme for the multi-dose administration of busulfan. FIG. 8 graphically depicts the results a single dose administration of the CD117 ADC on neutrophil count (10³/μl) as a function of days post-transplant. FIG. 9 graphically depicts the results a single dose administration of the CD117 ADC on platelet count (10⁵/μl) as a function of days post-transplant. FIG. 10 graphically depicts the results a single dose administration of the CD117 ADC on lymphocyte count (10³/μl) as a function of days post-transplant. FIG. 11 graphically depicts the results of an assay showing the peripheral granulocyte p-globin vector copy number (VCN) as a function of days post-transplant. The data shows that peripheral granulocyte vector copy number is stable over time and comparable to historical data with busulfan conditioning. The shaded box represents VCN range with busulfan conditioning.

FIG. 12 graphically depicts the results of an assay showing the aspartate aminotransferase (AST) levels (U/L) as a function days post-transplant.

### DETAILED DESCRIPTION

Described herein is a method of providing stem cell gene therapy, comprising methods of administering a transplant comprising a population of genetically modified stem cells (e.g., hematopoietic stem cells) to a subject in need thereof. The genetically modified stem cells are administered to a subject who has received a conditioning treatment, which method comprises administering an antibody-drug conjugate (ADC) that targets and depletes a specific population of endogenous hematopoietic stem cells and/or immune cells from the subject (conditioning). As described herein, the genetically modified stem cells can be used to deliver a correction of a defective gene (e.g., a mutant gene that causes a genetic disorder) to a subject in need of treatment. The present disclosure provides methods of combining stem cell gene therapy with a conditioning method that enhances engraftment, and thereby allows gene correction.

By way of example, autologous stem cells from a sickle cell anemia patient can be genetically modified to correct the defective gene (e.g., mutation(s) in the beta globin gene—HBB gene) ex vivo and administered to the patient. Various methods of genetically modifying stem cells (i.e., gene editing) are known and available in the art, and include, for example, zinc finger nucleases (e.g., U.S. Pat. No. 9,834,787), transcription-activator like effector nucleases (TALENs), viral-mediated gene editing, or the CRISPR/Cas system (e.g., US 2019/0010495 A1). Further, various genetically modified stem cells are known in the art (see, e.g., reviewed in Yong et al. “Recent challenges and advances in genetically-engineered cell therapy” J. Pharm. Investig. 48(2):199-208, 2018, and the references cited herein, incorporated herein by reference in their entireties), any of which can be used in the present method.

The ADCs for use in the present disclosure can target specific molecules on hemoatopoietic stem cells and/or immune cells, including, e.g., CD2, CD5, CD7, CDwl2, CD13, CD15, CD19, CD21, CD22, CD29, CD30, CD33, CD34, CD36, CD38, CD40, CD41, CD42a, CD42b, CD42c, CD42d, CD43, CD45, CD45RA, CD45RB, CD45RC, CD45RO, CD47, CD48, CD49b, CD49d, CD49e, CD49f, CD50, CD53, CD55, CD64a, CD68, CD71, CD72, CD73, CD81, CD82, CD85A, CD85K, CD90, CD99, CD104, CD105, CD109, CD110, CD111, CD112, CD114, CD115, CD117, CD123, CD124, CD126, CD127, CD130, CD131, CD133, CD134, CD135, CD137, CD138, CD151, CD157, CD162, CD164, CD168, CD172a, CD173, CD174, CD175, CD175s, CD176, CD183, CD191, CD200, CD201, CD205, CD217, CD220, CD221, CD222, CD223, CD224, CD225, CD226, CD227, CD228, CD229, CD230, CD235a, CD235b, CD236, CD236R, CD238, CD240, CD242, CD243, CD252, CD277, CD292, CDw293, CD295, CD298, CD309, CD318, CD324, CD325, CD338, CD344, CD349, or CD350. For example, described herein are ADCs comprising isolated anti-CD117 human antibodies that bind to human CD117. Also described herein are ADCs comprising isolated anti-CD45 human antibodies that bind to human CD45. The antibodies provided herein have many characteristics making them advantageous for therapy, including methods of conditioning human patients for genetically modified stem cell transplantation. For example, antibodies disclosed herein cross react with rhesus CD117 and are able to internalize. Both of these features also make them advantageous for use in conjugates for delivering cytotoxins to CD117 expressing cells.

The antibodies described herein include both antagonist antibodies and neutral antibodies. Specifically, provided herein are anti-CD117 antibodies Antibody 54 (Ab54), Antibody 55 (Ab55), Antibody 56 (Ab56), Antibody 57 (Ab57), Antibody 58 (Ab58), Antibody 61 (Ab61), Antibody 66 (Ab66), Antibody 67 (Ab67), Antibody 68 (Ab68), and Antibody 69 (Ab69) which are each human anti-CD117 antibodies that specifically bind to the ectodomain of human CD117. The binding regions of Ab54, Ab55, Ab56, Ab57, Ab58, Ab61, Ab66, Ab67, Ab68, and Ab69 are described below, including in Table 9. The anti-CD117 antibodies disclosed herein can be included in anti-CD117 antibody drug conjugates (ADCs; also referred to herein as conjugates).

Genetically modified stem cells in combination with conditioning methods that include the ADCs (e.g., anti-CD117 or anti-CD45 antibody drug conjugates (ADCs)) described herein can be used in methods to treat a variety of disorders, such as diseases of a cell type in the hematopoietic lineage, cancers, autoimmune diseases, metabolic disorders, and stem cell disorders, among others. The ADC compositions and methods described herein deplete a population of endogenous hematopoietic stem cells so as to promote the engraftment of the transplanted genetically modified hematopoietic stem cells by providing a niche to which the transplanted cells may home. The foregoing activities can be achieved by administration of an ADC, antibody, or antigen-binding fragment thereof, capable of binding an antigen (e.g., CD117 or CD45) expressed by a hematopoietic stem cell. This administration can cause the selective depletion of a population of endogenous hematopoietic stem cells, thereby creating a vacancy in the hematopoietic tissue, such as the bone marrow, that can subsequently be filled by transplanted, genetically modified hematopoietic stem cells. This selective depletion is also referred to as “conditioning”. The present methods are based, in part, on the observation that ADCs, antibodies, or antigen-binding fragments thereof, capable of binding, e.g., CD117 (such as GNNK+CD117) or CD45 can be administered to a patient as a conditioning agent. ADCs, antibodies, or antigen-binding fragments thereof, that bind, e.g., CD117 or CD45, can be administered to a patient suffering from a cancer, such as leukemia, or autoimmune disease to directly deplete a population of cancerous cells or autoimmune cells, and can also be administered to a patient in need of hematopoietic stem cell gene therapy in order to promote the survival and engraftment potential of transplanted genetically modified hematopoietic stem cells, thereby ensuring that the corrected or altered genes are preserved in the patient following transplantation.

Engraftment of genetically modified hematopoietic stem cell transplants due to the administration of, e.g., anti-CD117 or anti-CD45 ADC, can manifest in a variety of empirical measurements. For instance, engraftment of transplanted genetically modified hematopoietic stem cells can be evaluated by assessing the quantity of competitive repopulating units (CRU) present within the bone marrow of a patient following administration of an ADC described herein and subsequent administration of a genetically modified hematopoietic stem cell transplant. Additionally, one can observe engraftment of a genetically modified hematopoietic stem cell transplant by incorporating a reporter gene, such as an enzyme that catalyzes a chemical reaction yielding a fluorescent, chromophoric, or luminescent product, into a vector with which the transplant comprising the genetically modified hematopoietic stem cells have been transfected and subsequently monitoring the corresponding signal in a tissue into which the transplanted hematopoietic stem cells have homed, such as the bone marrow. One can also observe hematopoietic stem cell engraftment by evaluation of the quantity and survival of hematopoietic stem and progenitor cells, for instance, as determined by fluorescence activated cell sorting (FACS) analysis methods known in the art. Engraftment can also be determined by measuring white blood cell counts in peripheral blood during a post-transplant period, and/or by measuring recovery of marrow cells by donor cells in a bone marrow aspirate sample. Engraftment can also be determined by detecting the presence of a corrected or altered gene sequence. For example, in a treatment for sickle cell disease, engraftment can be determined by detecting the presence of the corrected HBB gene sequence.

The sections that follow provide a description of ADCs, antibodies, or antigen-binding fragments thereof, and genetically modified stem cells that can be administered to a patient, such as a patient suffering from a cancer or autoimmune disease, or a patient in need of hematopoietic stem cell transplant therapy in order to promote engraftment of genetically modified hematopoietic stem cell grafts, as well as methods of administering such therapeutics to a patient (e.g., conditioning and administration of the genetically modified HSCs).

### A. Definitions

As used herein, the term “about” refers to a value that is within 10% above or below the value being described. For example, the term “about 5 nM” indicates a range of from 4.5 nM to 5.5 nM.

As used herein, the term “antibody” refers to an immunoglobulin molecule that specifically binds to, or is immunologically reactive with, a particular antigen. An antibody includes, but is not limited to, monoclonal antibodies, multispecific antibodies (e.g., bispecific antibodies), genetically engineered, and otherwise modified forms of antibodies, including but not limited to chimeric antibodies, humanized antibodies, heteroconjugate antibodies (e.g., bi- tri- and quad-specific antibodies, diabodies, triabodies, and tetrabodies), and antibody fragments (i.e., antigen binding fragments of antibodies), including, for example, Fab′, F(ab′)₂, Fab, Fv, rIgG, and scFv fragments, so long as they exhibit the desired antigen-binding activity.

The term “monoclonal antibody” (mAb) is meant to include both intact molecules, as well as antibody fragments (including, for example, Fab and F(ab′)₂ fragments) that are capable of specifically binding to a target protein. A monoclonal antibody refers to an antibody that is derived from a single clone, by any means available or known in the art, and is not limited to antibodies produced through hybridoma technology. Monoclonal antibodies useful with the present disclosure can be prepared using a wide variety of techniques known in the art including the use of hybridoma, recombinant, and phage display technologies, or a combination thereof. As used herein, the Fab and F(ab′)₂ fragments refer to antibody fragments that lack the Fc fragment of an intact antibody. Examples of these antibody fragments are described herein.

The antibodies of the present disclosure are generally isolated or recombinant. “Isolated,” when used herein generally refers to a polypeptide, e.g., an antibody, that has been identified and separated and/or recovered from a cell or cell culture from which it was expressed. Ordinarily, an isolated antibody will be prepared by at least one purification step. Thus, an “isolated antibody,” refers to an antibody which is substantially free of other antibodies having different antigenic specificities. For instance, an isolated antibody that specifically binds to CD117 is substantially free of antibodies that specifically bind antigens other than CD117.

The term “antigen-binding fragment,” as used herein, refers to a fragment or portion of an antibody that retains the ability to specifically bind to a target antigen. The antigen-binding function of an antibody can be performed by a fragment of a full-length antibody. An antibody fragment can be, for example, a Fab, F(ab′)₂, scFv, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂ fragment, a bivalent fragment containing two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb including V_{H} and V_{L} domains; (vi) a dAb fragment that consists of a V_{H} domain (see, e.g., Ward et al., Nature 341:544-546, 1989); (vii) a dAb which consists of a V_{H} or a V_{L} domain; (viii) an isolated complementarity determining region (CDR); and (ix) a combination of two or more (e.g., two, three, four, five, or six) isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single chain Fv (scFv); see, for example, Bird et al., Science 242:423-426, 1988 and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art.

As used herein, the term “anti-CD117 antibody” or “an antibody that binds to CD117” refers to an antibody that is capable of binding CD117 with sufficient affinity such that the antibody is useful as a diagnostic and/or therapeutic agent in targeting CD117. The amino acid sequences of the two main isoforms of human CD117 are provided in SEQ ID NO: 145 (isoform 1) and SEQ ID NO: 146 (isoform 2).

As used herein, the term “anti-CD45 antibody” or “an antibody that binds to CD45” refers to an antibody that is capable of binding CD45 with sufficient affinity such that the antibody is useful as a diagnostic and/or therapeutic agent in targeting CD45.

As used herein, the term “anti-CD2 antibody” or “an antibody that binds to CD2” or an “anti-CD2 ADC” or “an ADC that binds to CD2” refers to an antibody or ADC that specifically binds to human CD2 as CD2 is found on the cell surface of cells, such as T cells.

As used herein, the term “anti-CD5 antibody” or “an antibody that binds to CD5” or an “anti-CD5 ADC” or “an ADC that binds to CD5” refers to an antibody or ADC that specifically binds to human CD5 as CD5 is found on the cell surface of cells, such as T cells.

As used herein, the term “anti-CD137 antibody” or an “antibody that binds to CD137” refers to an antibody that is capable of binding CD137 with sufficient affinity such that the antibody is useful as a diagnostic and/or therapeutic agent in targeting CD137.

As used herein, the term “bispecific antibody” refers to an antibody, for example, a monoclonal, often a human or humanized antibody, that is capable of binding at least two different antigens or two different epitopes that can be on the same or different antigens. For instance, one of the binding specificities can be directed towards an epitope on a hematopoietic stem cell surface antigen, such as CD117 (e.g., CD117 such as GNNK+CD117) or such as CD45, and the other can specifically bind an epitope on a different hematopoietic stem cell surface antigen or another cell surface protein, such as a receptor or receptor subunit involved in a signal transduction pathway that potentiates cell growth, among others. In some embodiments, the binding specificities can be directed towards unique, non-overlapping epitopes on the same target antigen (i.e., a biparatopic antibody).

As used herein, the term “complementarity determining region” (CDR) refers to a hypervariable region found both in the light chain and the heavy chain variable domains of an antibody. The more highly conserved portions of variable domains are referred to as framework regions (FRs). The amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The antibodies described herein may contain modifications in these hybrid hypervariable positions. The variable domains of native heavy and light chains each contain four framework regions that primarily adopt a p-sheet configuration, connected by three CDRs, which form loops that connect, and in some cases form part of, the p-sheet structure. The CDRs in each chain are held together in close proximity by the framework regions in the order FR1-CDR1-FR2-CDR2-FR3-CDR3-FR4 and, with the CDRs from the other antibody chains, contribute to the formation of the target binding site of antibodies (see Kabat et al., Sequences of Proteins of Immunological Interest, National Institute of Health, Bethesda, Md., 1987). In certain embodiments, numbering of immunoglobulin amino acid residues is performed according to the immunoglobulin amino acid residue numbering system of Kabat et al., unless otherwise indicated (although any antibody numbering scheme, including, but not limited to IMGT and Chothia, can be utilized).

As used herein, the terms “condition” and “conditioning” refer to a process or processes by which a patient is prepared for receipt of a transplant, e.g., a transplant containing genetically modified hematopoietic stem cells (HSCs). Such procedures promote the engraftment of a hematopoietic stem cell transplant (for instance, as inferred from a sustained increase in the quantity of viable hematopoietic stem cells within a blood sample isolated from a patient following a conditioning procedure and subsequent hematopoietic stem cell transplantation). According to the methods described herein, a patient may be conditioned for genetically modified HSC transplant therapy by administration to the patient of an ADC capable of binding a molecule, e.g., an antigen, expressed by hematopoietic stem cells and/or immune cells, such as CD117 (e.g., GNNK+CD117) or CD45. As described herein, the antibody may be covalently conjugated to a cytotoxin so as to form a drug-antibody conjugate (also referred to as an antibody drug conjugate (ADC)). Administration of an ADC, antibody, or antigen-binding fragment thereof, capable of binding one or more of the HSC-expressed antigens disclosed herein to a patient in need of hematopoietic stem cell transplant (HSCT) therapy can promote the engraftment of a HSC graft, for example, by selectively depleting endogenous HSCs, thereby creating a vacancy filled by a genetically modified HSC transplant.

As used herein, the term “conjugate”, “antibody drug conjugate” or “drug antibody conjugate” or “ADC”, refers to an antibody, or fragment thereof, which is linked to a cytotoxin. An ADC is formed by the chemical bonding of a reactive functional group of an antibody or antigen-binding fragment thereof, with an appropriately reactive functional group of another molecule, such as a cytotoxin described herein. Conjugates may include a linker between the two molecules bound to one another, e.g., between an antibody and a cytotoxin. Examples of linkers that can be used for the formation of a conjugate include peptide-containing linkers, such as those that contain naturally occurring or non-naturally occurring amino acids, such as D-amino acids. Linkers can be prepared using a variety of strategies described herein and known in the art. Depending on the reactive components therein, a linker may be cleaved, for example, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012). As described above, the term “conjugate” (when referring to a compound) is also referred to interchangeably herein as a “drug conjugate”, “drug antibody conjugate,” “antibody drug conjugate” or “ADC”.

As used herein, the term “coupling reaction” refers to a chemical reaction in which two or more substituents suitable for reaction with one another react so as to form a chemical moiety that joins (e.g., covalently) the molecular fragments bound to each substituent. Coupling reactions include those in which a reactive substituent bound to a fragment that is a cytotoxin, such as a cytotoxin known in the art or described herein, reacts with a suitably reactive substituent bound to a fragment that is an antibody, or antigen-binding fragment thereof, such as an antibody, antigen-binding fragment thereof, or specific anti-CD117 antibody that binds CD117 (such as GNNK+CD117) known in the art or described herein. Examples of suitably reactive substituents include a nucleophile/electrophile pair (e.g., a thiol/haloalkyl pair, an amine/carbonyl pair, or a thiol/α,β-unsaturated carbonyl pair, among others), a diene/dienophile pair (e.g., an azide/alkyne pair, among others), and the like. Coupling reactions include, without limitation, thiol alkylation, hydroxyl alkylation, amine alkylation, amine condensation, amidation, esterification, disulfide formation, cycloaddition (e.g., [4+2] Diels-Alder cycloaddition, [3+2] Huisgen cycloaddition, among others), nucleophilic aromatic substitution, electrophilic aromatic substitution, and other reactive modalities known in the art or described herein.

As used herein, “CRU (competitive repopulating unit)” refers to a unit of measure of long-term engrafting stem cells, which can be detected after in-vivo transplantation.

As used herein, the term “diabody” refers to a bivalent antibody containing two polypeptide chains, in which each polypeptide chain includes V_{H} and V_{L} domains joined by a linker that is too short (e.g., a linker composed of five amino acids) to allow for intramolecular association of V_{H} and V_{L} domains on the same peptide chain. This configuration forces each domain to pair with a complementary domain on another polypeptide chain so as to form a homodimeric structure. Accordingly, the term “triabody” refers to trivalent antibodies containing three peptide chains, each of which contains one V_{H} domain and one V_{L} domain joined by a linker that is exceedingly short (e.g., a linker composed of 1-2 amino acids) to permit intramolecular association of V_{H} and V_{L} domains within the same peptide chain. In order to fold into their native structures, peptides configured in this way typically trimerize so as to position the V_{H} and V_{L} domains of neighboring peptide chains spatially proximal to one another (see, for example, Holliger et al., Proc. Natl. Acad. Sci. USA 90:6444-48, 1993).

As used herein, “drug-to-antibody ratio” or “DAR” refers to the number of drugs, e.g., amatoxin, attached to the antibody of a conjugate. The DAR of an ADC can range from 1 to 8, although higher loads are also possible depending on the number of linkage sites on an antibody. In certain embodiments, the conjugate has a DAR of 1, 2, 3, 4, 5, 6, 7, or 8.

As used herein, the term “endogenous” describes a substance, such as a molecule, cell, tissue, or organ (e.g., a hematopoietic stem cell or a cell of hematopoietic lineage, such as a megakaryocyte, thrombocyte, platelet, erythrocyte, mast cell, myeloblast, basophil, neutrophil, eosinophil, microglial cell, granulocyte, monocyte, osteoclast, antigen-presenting cell, macrophage, dendritic cell, natural killer cell, T-lymphocyte, or B-lymphocyte) that is found naturally in a particular organism, such as a human patient.

As used herein, the term “engraftment potential” is used to refer to the ability of hematopoietic stem and progenitor cells to repopulate a tissue, whether such cells are naturally circulating or are provided by transplantation. The term encompasses all events surrounding or leading up to engraftment, such as tissue homing of cells and colonization of cells within the tissue of interest. The engraftment efficiency or rate of engraftment can be evaluated or quantified using any clinically acceptable parameter as known to those of skill in the art and can include, for example, assessment of competitive repopulating units (CRU); incorporation or expression of a marker in tissue(s) into which stem cells have homed, colonized, or become engrafted; or by evaluation of the progress of a subject through disease progression, survival of hematopoietic stem and progenitor cells, or survival of a recipient. Engraftment can also be determined by measuring white blood cell counts in peripheral blood during a post-transplant period. Engraftment can also be assessed by measuring recovery of marrow cells by donor cells in a bone marrow aspirate sample.

As used herein, the term “exogenous” describes a substance, such as a molecule, cell, tissue, or organ (e.g., a genetically modified hematopoietic stem cell or a cell of hematopoietic lineage, such as a megakaryocyte, thrombocyte, platelet, erythrocyte, mast cell, myeloblast, basophil, neutrophil, eosinophil, microglial cell, granulocyte, monocyte, osteoclast, antigen-presenting cell, macrophage, dendritic cell, natural killer cell, T-lymphocyte, or B-lymphocyte) that is not found naturally in a particular organism, such as a human patient. In some embodiments, a substance that is exogenous to a recipient organism, e.g., a recipient patient, may be naturally present in a donor organism, e.g., a donor subject, from which the substance is derived. For example, an allogeneic cell transplant contains cells that are exogenous to the recipient, but native to the donor. In some embodiments, an autologous cell transplant contains gene sequence(s) that is exogenous to the recipient (e.g., through correction of a mutation that was present in the recipient), and thus such autologous cell transplant is “exogenous” to the recipient. Exogenous substances include those that are provided from an external source to an organism or to cultured matter extracted therefrom.

The terms “Fc”, “Fc region,” and “Fc domain,” as used herein refer to the portion of an IgG antibody molecule that correlates to a crystallizable fragment obtained by papain digestion of an IgG molecule. The Fc region comprises the C-terminal half of two heavy chains of an IgG molecule that are linked by disulfide bonds. It has no antigen binding activity but contains the carbohydrate moiety and binding sites for complement and Fc receptors, including the FcRn receptor (see below). For example, an Fc region contains the second constant domain CH2 (e.g., residues at EU positions 231-340 of IgG1) and the third constant domain CH3 (e.g., residues at EU positions 341-447 of human IgG1). As used herein, the Fc region or domain includes the “lower hinge region” (e.g., residues at EU positions 233-239 of IgG1).

Fc can refer to this region in isolation, or this region in the context of an antibody, antibody fragment, or Fc fusion protein. Polymorphisms have been observed at a number of positions in Fc domains, including but not limited to EU positions 270, 272, 312, 315, 356, and 358, and thus slight differences between the sequences presented in the instant application and sequences known in the art can exist. Thus, a “wild type IgG Fc domain” or “WT IgG Fc domain” refers to any naturally occurring IgG Fc region (i.e., any allele). The sequences of the heavy chains of human IgG1, IgG2, IgG3 and IgG4 can be found in a number of sequence databases, for example, at the Uniprot database (www.uniprot.org) under accession numbers P01857 (IGHG1_HUMAN), P01859 (IGHG2_HUMAN), P01860 (IGHG3_HUMAN), and P01861 (IGHG1_HUMAN), respectively. An example of a “WT” Fc region is provided in SEQ ID NO: 122 (which provides a heavy chain constant region containing an Fc region).

The terms “modified Fc region” or “variant Fc region” as used herein refers to an IgG Fc domain comprising one or more amino acid substitutions, deletions, insertions or modifications introduced at any position within the Fc region. In certain aspects a variant IgG Fc domain comprises one or more amino acid substitutions resulting in decreased or ablated binding affinity for an Fc gamma R and/or C1q as compared to the wild type Fc domain not comprising the one or more amino acid substitutions. Further, Fc binding interactions are essential for a variety of effector functions and downstream signaling events including, but not limited to, antibody dependent cell-mediated cytotoxicity (ADCC) and complement dependent cytotoxicity (CDC). Accordingly, in certain aspects, an antibody comprising a variant Fc domain (e.g., an antibody, fusion protein or conjugate) can exhibit altered binding affinity for at least one or more Fc ligands (e.g., Fc gamma Rs) relative to a corresponding antibody otherwise having the same amino acid sequence but not comprising the one or more amino acid substitution, deletion, insertion or modifications such as, for example, an unmodified Fc region containing naturally occurring amino acid residues at the corresponding position in the Fc region.

Variant Fc domains are defined according to the amino acid modifications that compose them. For all amino acid substitutions discussed herein in regard to the Fc region, numbering is always according to the EU index as in Kabat. Thus, for example, D265C is an Fc variant with the aspartic acid (D) at EU position 265 substituted with cysteine (C) relative to the parent Fc domain. It is noted that the order in which substitutions are provided is arbitrary. Likewise, e.g., D265C/L234A/L235A defines a variant Fc variant with substitutions at EU positions 265 (D to C), 234 (L to A), and 235 (L to A) relative to the parent Fc domain. A variant can also be designated according to its final amino acid composition in the mutated EU amino acid positions. For example, the L234A/L235A mutant can be referred to as “LALA”. As a further example, the E233P.L234V.L235A.delG236 (deletion of 236) mutant can be referred to as “EPLVLAdelG”. As yet another example, the I253A.H310A.H435A mutant can be referred to as “IHH”. It is noted that the order in which substitutions are provided is arbitrary.

The terms “Fc gamma receptor” or “Fc gamma R” as used herein refer to any member of the family of proteins that bind the IgG antibody Fc region and are encoded by the Fc gamma R genes. In humans this family includes but is not limited to Fc gamma RI (CD64), including isoforms Fc gamma RIa, Fc gamma RIb, and Fc gamma RIc; Fc gamma RII (CD32), including isoforms Fc gamma RIIa (including allotypes H131 and R131), Fc gamma RIIb (including Fc gamma RIIb-1 and Fc gamma RIIb-2), and Fc gamma RIIc; and Fc gamma RIII (CD16), including isoforms Fc gamma RIIIa (including allotypes V158 and F158) and Fc gamma RIIIb (including allotypes Fc gamma RIIIb-NA1 and Fc gamma RIIIb-NA2), as well as any undiscovered human Fc gamma Rs or Fc gamma R isoforms or allotypes. An Fc gamma R can be from any organism, including but not limited to humans, mice, rats, rabbits, and monkeys. Mouse Fc gamma Rs include but are not limited to Fc gamma RI (CD64), Fc gamma RII (CD32), Fc gamma RIII (CD16), and Fc gamma RIII-2 (CD16-2), as well as any undiscovered mouse Fc gamma Rs or Fc gamma R isoforms or allotypes.

The term “effector function” as used herein refers to a biochemical event that results from the interaction of an Fc domain with an Fc receptor. Effector functions include but are not limited to ADCC, ADCP, and CDC. By “effector cell” as used herein is meant a cell of the immune system that expresses or one or more Fc receptors and mediates one or more effector functions. Effector cells include but are not limited to monocytes, macrophages, neutrophils, dendritic cells, eosinophils, mast cells, platelets, B cells, large granular lymphocytes, Langerhans' cells, natural killer (NK) cells, and gamma delta T cells, and can be from any organism included but not limited to humans, mice, rats, rabbits, and monkeys.

The term “silent”, “silenced”, or “silencing” as used herein refers to an antibody having a modified Fc region described herein that has decreased binding to an Fc gamma receptor (FcγR) relative to binding of an identical antibody comprising an unmodified Fc region to the FcγR (e.g., a decrease in binding to a FcγR by at least 70%, at least 80%, at least 90%, at least 95%, at least 98%, at least 99%, or 100% relative to binding of the identical antibody comprising an unmodified Fc region to the FcγR as measured by, e.g., BLI). In some embodiments, the Fc silenced antibody has no detectable binding to an FcγR. Binding of an antibody having a modified Fc region to an FcγR can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE™ analysis or Octet™ analysis (forteBIO)), and other methods such as indirect binding assays, competitive binding assays fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions. One example of a competitive binding assay is a radioimmunoassay comprising the incubation of labeled antigen with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound in the presence of increasing amounts of an unlabeled second antibody.

As used herein, the term “identical antibody comprising an unmodified Fc region” refers to an antibody that lacks the recited amino acid substitutions (e.g., D265C, H435A, L234A, and/or L235A), but otherwise has the same amino acid sequence as the Fc modified antibody to which it is being compared.

The terms “antibody-dependent cell-mediated cytotoxicity” or “ADCC” refer to a form of cytotoxicity in which a polypeptide comprising an Fc domain, e.g., an antibody, bound onto Fc receptors (FcRs) present on certain cytotoxic cells (e.g., primarily NK cells, neutrophils, and macrophages) and enables these cytotoxic effector cells to bind specifically to an antigen-bearing “target cell” and subsequently kill the target cell with cytotoxins. (Hogarth et al., Nature review Drug Discovery 2012, 11:313) It is contemplated that, in addition to antibodies and fragments thereof, other polypeptides comprising Fc domains, e.g., Fc fusion proteins and Fc conjugate proteins, having the capacity to bind specifically to an antigen-bearing target cell will be able to effect cell-mediated cytotoxicity.

For simplicity, the cell-mediated cytotoxicity resulting from the activity of a polypeptide comprising an Fc domain is also referred to herein as ADCC activity. The ability of any particular polypeptide of the present disclosure to mediate lysis of the target cell by ADCC can be assayed. To assess ADCC activity, a polypeptide of interest (e.g., an antibody) is added to target cells in combination with immune effector cells, resulting in cytolysis of the target cell. Cytolysis is generally detected by the release of label (e.g., radioactive substrates, fluorescent dyes or natural intracellular proteins) from the lysed cells. Useful effector cells for such assays include peripheral blood mononuclear cells (PBMC) and Natural Killer (NK) cells. Specific examples of in vitro ADCC assays are described in Bruggemann et al., J. Exp. Med. 166:1351 (1987); Wilkinson et al., J. Immunol. Methods 258:183 (2001); Patel et al., J. Immunol. Methods 184:29 (1995). Alternatively, or additionally, ADCC activity of the antibody of interest can be assessed in vivo, e.g., in an animal model such as that disclosed in Clynes et al., Proc. Natl. Acad. Sci. USA 95:652 (1998).

The terms “full length antibody” and “intact antibody” are used herein interchangeably to refer to an antibody in its substantially intact form, and not an antibody fragment as defined herein. Thus, for an IgG antibody, an intact antibody comprises two heavy chains each comprising a variable region, a constant region and an Fc region, and two light chains each comprising a variable region and a constant region. More specifically, an intact IgG comprises two light chains each comprising a light chain variable region (VL) and a light chain constant region (CL), and comprises two heavy chains each comprising a heavy chain variable region (VH) and three heavy chain constant regions (CH1, CH2, and CH3). CH2 and CH3 represent the Fc region of the heavy chain. In certain embodiments, the ADCs used in the methods described herein comprising an intact antibody that binds to an antigen expressed on the surface of a stem cell, e.g., human CD117 (hCD117) or human CD45 (hCD45).

As used herein, the term “framework region” or “FW region” includes amino acid residues that are adjacent to the CDRs of an antibody or antigen-binding fragment thereof. FW region residues may be present in, for example, human antibodies, humanized antibodies, monoclonal antibodies, antibody fragments, Fab fragments, single chain antibody fragments, scFv fragments, antibody domains, and bispecific antibodies, among others.

Also provided are “conservative sequence modifications” of the sequences set forth in SEQ ID NOs described herein, i.e., nucleotide and amino acid sequence modifications which do not abrogate the binding of the antibody encoded by the nucleotide sequence or containing the amino acid sequence, to the antigen. Such conservative sequence modifications include conservative nucleotide and amino acid substitutions, as well as, nucleotide and amino acid additions and deletions. For example, modifications can be introduced into SEQ ID NOs described herein by standard techniques known in the art, such as site-directed mutagenesis and PCR-mediated mutagenesis. Conservative sequence modifications include conservative amino acid substitutions, in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in an anti-CD117 antibody is preferably replaced with another amino acid residue from the same side chain family. Methods of identifying nucleotide and amino acid conservative substitutions that do not eliminate antigen binding are well-known in the art (see, e.g., Brummell et al., Biochem. 32:1180-1187 (1993); Kobayashi et al. Protein Eng. 12(10):879-884 (1999); and Burks et al. Proc. Natl. Acad. Sci. USA 94:412-417 (1997)).

As used herein, the term “half-life” refers to the time it takes for the plasma concentration of the antibody drug in the body to be reduced by one half or 50% in a subject, e.g., a human subject. This 50% reduction in serum concentration reflects the amount of drug circulating.

As used herein, the term “stem cells” refers to multipotent stem cells (e.g., hematopoietic stem cells). The term can also refer to totipotent or pluripotent stem cells.

As used herein, the term “hematopoietic stem cells” (“HSCs”) refers to immature blood cells having the capacity to self-renew and to differentiate into mature blood cells containing diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Such cells may include CD34⁺ cells. CD34⁺ cells are immature cells that express the CD34 cell surface marker. In humans, CD34+ cells are believed to include a subpopulation of cells with the stem cell properties defined above, whereas in mice, HSCs are CD34−. In addition, HSCs also refer to long term repopulating HSCs (LT-HSC) and short term repopulating HSCs (ST-HSC). LT-HSCs and ST-HSCs are differentiated, based on functional potential and on cell surface marker expression. For example, human HSCs are CD34+, CD38−, CD45RA−, CD90+, CD49F+, and lin−(negative for mature lineage markers including CD2, CD3, CD4, CD7, CD8, CD10, CD11B, CD19, CD20, CD56, CD235A). In mice, bone marrow LT-HSCs are CD34−, SCA-1+, C-kit+, CD135−, Slamfl/CD150+, CD48−, and lin−(negative for mature lineage markers including Ter119, CD11b, Gr1, CD3, CD4, CD8, B220, IL7ra), whereas ST-HSCs are CD34+, SCA-1+, C-kit+, CD135−, Slamfl/CD150+, and lin−(negative for 25 mature lineage markers including Ter119, CD11 b, Gr1, CD3, CD4, CD8, B220, IL7ra). In addition, ST-HSCs are less quiescent and more proliferative than LT-HSCs under homeostatic conditions. However, LT-HSC have greater self-renewal potential (i.e., they survive throughout adulthood, and can be serially transplanted through successive recipients), whereas ST-HSCs have limited self-renewal (i.e., they survive for only a limited period of time, and do not possess serial transplantation potential). Any of these HSCs can be used in the methods described herein. ST-HSCs are particularly useful because they are highly proliferative and thus, can more quickly give rise to differentiated progeny.

As used herein, the term “genetically modified stem cell” or “genetically modified HSC” refers to a cell or cells that have undergone gene editing so as to alter a target gene in the cell's genome, or have been altered such that an exogenous gene or exogenous gene sequence is expressed in the cell.

As used herein, the term “target gene” refers to gene sequence or a portion of a gene sequence of the stem cell genome. In some embodiments, the target gene sequence is a coding sequence. In some embodiments, the target gene sequence is a non-coding sequence (e.g., a regulatory sequence). In some embodiments, the target gene sequence is a non-wildtype gene sequence (e.g., a mutation) that gives rise to a disorder or illness. In some embodiments, altering a target gene includes, e.g., 1) use of a gene editing system to correct one or more mutations in the target gene sequence (e.g., by codon-specific editing; or replacement of the entire or a portion of the gene), thereby restoring function of the gene (e.g., produce a functional protein or a functional regulatory sequence), 2) insertion of a functional gene sequence (e.g., a wild-type or a functional variant sequence of a gene) into the stem cell genome, or 3) insertion of a functional gene (e.g., a wild-type or a functional variant sequence of a gene), and disruption (e.g., silencing) of a target gene sequence. Various gene editing systems for correcting and/or inserting gene sequences are known in the art. By way of example, one or more mutations in the HBB gene that causes sickle cell disease can be corrected by site-specific editing of the mutation(s) or by replacement of the gene, in whole or in part. By way of example, the mutant HBB gene can be silenced by gene-editing methods, and a functional HBB gene can be inserted into the stem cell genome. In some embodiments, the target gene is a wild-type gene (e.g., CCR5), that can be replaced with a variant sequence, or edited to encode a variant sequence, that confers a therapeutic benefit (e.g., CCR5(delta)32 for HIV therapy).

As used herein, the term “hematopoietic stem cell functional potential” refers to the functional properties of hematopoietic stem cells which include 1) multi-potency (which refers to the ability to differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), 2) self-renewal (which refers to the ability of hematopoietic stem cells to give rise to daughter cells that have equivalent potential as the mother cell, and further that this ability can repeatedly occur throughout the lifetime of an individual without exhaustion), and 3) the ability of hematopoietic stem cells or progeny thereof to be reintroduced into a transplant recipient whereupon they home to the hematopoietic stem cell niche and re-establish productive and sustained hematopoiesis.

As used herein, the term “human antibody” is intended to include antibodies having variable and constant regions derived from human germline immunoglobulin sequences. A human antibody may include amino acid residues not encoded by human germline immunoglobulin sequences (e.g., mutations introduced by random or site-specific mutagenesis in vitro or during gene rearrangement or by somatic mutation in vivo). However, the term “human antibody”, as used herein, is not intended to include antibodies in which CDR sequences derived from the germline of another mammalian species, such as a mouse, have been grafted onto human framework sequences. A human antibody can be produced in a human cell (for example, by recombinant expression) or by a non-human animal or a prokaryotic or eukaryotic cell that is capable of expressing functionally rearranged human immunoglobulin (such as heavy chain and/or light chain) genes. When a human antibody is a single chain antibody, it can include a linker peptide that is not found in native human antibodies. For example, an Fv can contain a linker peptide, such as two to about eight glycine or other amino acid residues, which connects the variable region of the heavy chain and the variable region of the light chain. Such linker peptides are considered to be of human origin. Human antibodies can be made by a variety of methods known in the art including phage display methods using antibody libraries derived from human immunoglobulin sequences. Human antibodies can also be produced using transgenic mice that are incapable of expressing functional endogenous immunoglobulins, but which can express human immunoglobulin genes (see, for example, PCT Publication Nos. WO 1998/24893; WO 1992/01047; WO 1996/34096; WO 1996/33735; U.S. Pat. Nos. 5,413,923; 5,625,126; 5,633,425; 5,569,825; 5,661,016; 5,545,806; 5,814,318; 5,885,793; 5,916,771; and 5,939,598).

A “humanized” antibody refers to an antibody that contains minimal sequences derived from non-human immunoglobulin. Thus, “humanized” forms of non-human (e.g., murine) antibodies are chimeric antibodies that contain minimal sequence derived from the non-human antibody. All or substantially all of the FW regions may be those of a human immunoglobulin sequence. In general, a humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. A humanized antibody can also comprise at least a portion of an immunoglobulin constant region (Fc), typically that of a human immunoglobulin consensus sequence. Methods of antibody humanization are known in the art and have been described, for example, in Riechmann et al., Nature 332:323-7, 1988; U.S. Pat. Nos. 5,530,101; 5,585,089; 5,693,761; 5,693,762; and U.S. Pat. No. 6,180,370 to Queen et al.; EP239400; PCT publication WO 91/09967; U.S. Pat. No. 5,225,539; EP592106; EP519596; Padlan, 1991, Mol. Immunol., 28:489-498; Studnicka et al., 1994, Prot. Eng. 7:805-814; Roguska et al., 1994, Proc. Natl. Acad. Sci. 91:969-973; and U.S. Pat. No. 5,565,332.

As used herein, patients that are “in need of” a genetically modified hematopoietic stem cell transplant include patients that, e.g., exhibit a defect or deficiency in one or more blood cell types, as well as patients having a stem cell disorder, autoimmune disease, cancer, or other pathology described herein. In some embodiments, patients that are in need of a genetically modified HSC transplant include patients that carry a defective gene that causes a disorder (e.g., sickle cell anemia, thalassemia, Wiskott-Aldrich syndrome, adenosine deaminase deficiency). Hematopoietic stem cells generally exhibit 1) multi-potency, and can thus differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), 2) self-renewal, and can thus give rise to daughter cells that have equivalent potential as the mother cell, and 3) the ability to be reintroduced into a transplant recipient whereupon they home to the hematopoietic stem cell niche and re-establish productive and sustained hematopoiesis. Hematopoietic stem cells can thus be administered to a patient defective or deficient in one or more cell types of the hematopoietic lineage (e.g., due to a mutant gene) in order to re-constitute the defective or deficient population of cells in vivo, for example, by altering the target gene (e.g., mutant gene). Additionally, or alternatively, the patient may be suffering from a hemoglobinopathy (e.g., a non-malignant hemoglobinopathy), such as sickle cell anemia, thalassemia, Fanconi anemia, aplastic anemia, and Wiskott-Aldrich syndrome. The subject may be one that is suffering from adenosine deaminase severe combined immunodeficiency (ADA SCID), HIV/AIDS, metachromatic leukodystrophy, Diamond-Blackfan anemia, and Schwachman-Diamond syndrome. The subject may have or be affected by an inherited blood disorder (e.g., sickle cell anemia) or an autoimmune disorder. Additionally, or alternatively, the subject may have or be affected by a malignancy, such as neuroblastoma or a hematologic cancer. For instance, the subject may have a leukemia, lymphoma, or myeloma. In some embodiments, the subject has acute myeloid leukemia, acute lymphoid leukemia, chronic myeloid leukemia, chronic lymphoid leukemia, multiple myeloma, diffuse large B-cell lymphoma, or non-Hodgkin's lymphoma. In some embodiments, the subject has myelodysplastic syndrome. In some embodiments, the subject has an autoimmune disease, such as scleroderma, multiple sclerosis, ulcerative colitis, Crohn's disease, Type 1 diabetes, or another autoimmune pathology described herein. In some embodiments, the subject is in need of chimeric antigen receptor T-cell (CART) therapy. In some embodiments, the subject has or is otherwise affected by a metabolic storage disorder. The subject may suffer or otherwise be affected by a metabolic disorder selected from the group consisting of glycogen storage diseases, mucopolysaccharidosis, Gaucher's Disease, Hurlers Disease, sphingolipidoses, metachromatic leukodystrophy, or any other diseases or disorders which may benefit from the treatments and therapies disclosed herein and including, without limitation, severe combined immunodeficiency, Wiscott-Aldrich syndrome, hyper immunoglobulin M (IgM) syndrome, Chediak-Higashi disease, hereditary lymphohistiocytosis, osteopetrosis, osteogenesis imperfecta, storage diseases, thalassemia major, sickle cell disease, systemic sclerosis, systemic lupus erythematosus, multiple sclerosis, juvenile rheumatoid arthritis and those diseases, or disorders described in “Bone Marrow Transplantation for Non-Malignant Disease,” ASH Education Book, 1:319-338 (2000), the disclosure of which is incorporated herein by reference in its entirety as it pertains to pathologies that may be treated by administration of hematopoietic stem cell transplant therapy.

As used herein a “neutral antibody” refers to an antibody, or an antigen binding fragment thereof, that is not capable of significantly neutralizing, blocking, inhibiting, abrogating, reducing or interfering with the activities of a particular or specified target (e.g., CD117 or CD45), including the binding of receptors to ligands or the interactions of enzymes with substrates. In one embodiment, a neutral anti-CD117 antibody, or fragment thereof, is an anti-CD117 antibody that does not substantially inhibit SCF-dependent cell proliferation and does not cross block SCF binding to CD117. An example of a neutral antibody is Ab67 (or an antibody having the binding regions of Ab67). In contrast, an “antagonist” anti-CD117 antibody inhibits SCF-dependent proliferation and is able to cross block SCF binding to CD117. An example of an antagonist antibody is Ab55 (or an antibody having the binding regions of Ab55).

As used herein, the term “recipient” refers to a patient that receives a transplant, such as a transplant containing a population of genetically modified hematopoietic stem cells. The transplanted cells administered to a recipient may be, e.g., autologous, syngeneic, or allogeneic cells.

As used herein, the term “donor” refers to a human or animal from which one or more cells are isolated prior to administration of the cells, or progeny thereof, into a recipient. The one or more cells may be, for example, a population of hematopoietic stem cells.

As used herein, the term “autologous” refers to cells that are transplanted to a subject that are derived from the same subject. In certain instances, an autologous cell or cells is genetically modified prior to transplantation back to the subject.

As used herein, the term “allogeneic” refers to cells of the same species that differ genetically to the cell in comparison, i.e., cells from different human subjects. For example, a cell from one human subject may be transplanted into a different human subject. Allogeneic is intended to reference the source of the cell relative to the recipient, and not the status of the cell with respect to genetic modifications.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) taken from a subject.

As used herein, the term “scFv” refers to a single chain Fv antibody in which the variable domains of the heavy chain and the light chain from an antibody have been joined to form one chain. scFv fragments contain a single polypeptide chain that includes the variable region of an antibody light chain (V_{L}) (e.g., CDR-L1, CDR-L2, and/or CDR-L3) and the variable region of an antibody heavy chain (V_{H}) (e.g., CDR-H1, CDR-H2, and/or CDR-H3) separated by a linker. The linker that joins the V_{L} and V_{H} regions of a scFv fragment can be a peptide linker composed of proteinogenic amino acids. Alternative linkers can be used to so as to increase the resistance of the scFv fragment to proteolytic degradation (for example, linkers containing D-amino acids), in order to enhance the solubility of the scFv fragment (for example, hydrophilic linkers such as polyethylene glycol-containing linkers or polypeptides containing repeating glycine and serine residues), to improve the biophysical stability of the molecule (for example, a linker containing cysteine residues that form intramolecular or intermolecular disulfide bonds), or to attenuate the immunogenicity of the scFv fragment (for example, linkers containing glycosylation sites). It will also be understood by one of ordinary skill in the art that the variable regions of the scFv molecules described herein can be modified such that they vary in amino acid sequence from the antibody molecule from which they were derived. For example, nucleotide or amino acid substitutions leading to conservative substitutions or changes at amino acid residues can be made (e.g., in CDR and/or framework residues) so as to preserve or enhance the ability of the scFv to bind to the antigen recognized by the corresponding antibody.

The terms “specific binding” or “specifically binding”, as used herein, refers to the ability of an antibody (or ADC) to recognize and bind to a specific protein structure (epitope) rather than to proteins generally. If an antibody is specific for epitope “A”, the presence of a molecule containing epitope A (or free, unlabeled A), in a reaction containing labeled “A” and the antibody, will reduce the amount of labeled A bound to the antibody. By way of example, an antibody “binds specifically” to a target if the antibody, when labeled, can be competed away from its target by the corresponding non-labeled antibody. In one embodiment, an antibody specifically binds to a target, e.g., CD117, if the antibody has a K_{D} for the target of at least about 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less (less meaning a number that is less than 10⁻¹², e.g. 10⁻¹³). Ranges including the aforementioned K_{D} values are also included herein, e.g., 10⁻⁸ M to 10⁻¹² M, 10⁻⁹ M to 10⁻¹² M, or 10⁻¹⁰ M to 10⁻¹² M. In one embodiment, the term “specific binding to CD117” or “specifically binds to CD117,” as used herein, refers to an antibody (or ADC) that binds to CD117 and has a dissociation constant (K_{D}) of 1.0×10⁻⁷ M or less, as determined by surface plasmon resonance. In one embodiment, K_{D} (M) is determined according to standard bio-layer interferometery (BLI). In one embodiment, K_{off} (1/s) is determined according to standard bio-layer interferometery (BLI). It shall be understood, however, that the antibody may be capable of specifically binding to two or more antigens which are related in sequence. For example, in one embodiment, an antibody can specifically bind to both human and a non-human (e.g., mouse or non-human primate) orthologs of, e.g., CD117 or CD45.

As used herein, the terms “subject” and “patient” refer to an organism, such as a human, that receives treatment for a particular disease or condition as described herein.

For instance, a patient, such as a human patient, may receive treatment prior to hematopoietic stem cell transplant therapy in order to promote the engraftment of genetically modified hematopoietic stem cells, which may be autologous or allogeneic.

As used herein, the phrase “substantially cleared from the blood” refers to a point in time following administration of a therapeutic agent (such as an anti-CD117 antibody, or antigen-binding fragment thereof) to a patient when the concentration of the therapeutic agent in a blood sample isolated from the patient is such that the therapeutic agent is not detectable by conventional means (for instance, such that the therapeutic agent is not detectable above the noise threshold of the device or assay used to detect the therapeutic agent). A variety of techniques known in the art can be used to detect antibodies, or antibody fragments, such as ELISA-based detection assays known in the art or described herein. Additional assays that can be used to detect antibodies, or antibody fragments, include immunoprecipitation techniques and immunoblot assays, among others known in the art.

As used herein, the phrase “stem cell disorder” broadly refers to any disease, disorder, or condition that may be treated or cured by conditioning a subject's target tissues, and/or by ablating an endogenous stem cell population in a target tissue (e.g., ablating an endogenous hematopoietic stem or progenitor cell population from a subject's bone marrow tissue), and by engrafting or transplanting genetically modified stem cells in a subject's target tissues. Additional disorders that can be treated using the compositions and methods described herein include, without limitation, sickle cell anemia, thalassemias, Fanconi anemia, aplastic anemia, Wiskott-Aldrich syndrome, ADA SCID, HIV/AIDS, metachromatic leukodystrophy, Diamond-Blackfan anemia, and Schwachman-Diamond syndrome. Additional diseases that may be treated using the patient conditioning and genetically modified hematopoietic stem cell transplant methods described herein include inherited blood disorders (e.g., sickle cell anemia) and autoimmune disorders, such as scleroderma, multiple sclerosis, ulcerative colitis, and Crohn's disease. Additional diseases that may be treated using the conditioning and transplantation methods described herein include a malignancy, such as a neuroblastoma or a hematologic cancer, such as leukemia, lymphoma, and myeloma. For instance, the cancer may be acute myeloid leukemia, acute lymphoid leukemia, chronic myeloid leukemia, chronic lymphoid leukemia, multiple myeloma, diffuse large B-cell lymphoma, or non-Hodgkin's lymphoma. Additional diseases treatable using the conditioning and/or transplantation methods described herein include myelodysplastic syndrome. In some embodiments, the subject has or is otherwise affected by a metabolic storage disorder. For example, the subject may suffer or otherwise be affected by a metabolic disorder selected from the group consisting of glycogen storage diseases, mucopolysaccharidosis, Gaucher's Disease, Hurlers Disease, sphingolipidoses, metachromatic leukodystrophy, or any other diseases or disorders which may benefit from the treatments and therapies disclosed herein and including, without limitation, severe combined immunodeficiency, Wiscott-Aldrich syndrome, hyper immunoglobulin M (IgM) syndrome, Chediak-Higashi disease, hereditary lymphohistiocytosis, osteopetrosis, osteogenesis imperfecta, storage diseases, thalassemia major, sickle cell disease, systemic sclerosis, systemic lupus erythematosus, multiple sclerosis, juvenile rheumatoid arthritis and those diseases, or disorders described in “Bone Marrow Transplantation for Non-Malignant Disease,” ASH Education Book, 1:319-338 (2000), the disclosure of which is incorporated herein by reference in its entirety as it pertains to pathologies that may be treated by administration of hematopoietic stem cell transplant therapy.

As used herein, the term “transfection” refers to any of a wide variety of techniques commonly used for the introduction of exogenous DNA into a prokaryotic or eukaryotic host cell, such as electroporation, lipofection, calcium-phosphate precipitation, DEAE-dextran transfection and the like.

As used herein, the terms “treat” or “treatment” refers to reducing the severity and/or frequency of disease symptoms, eliminating disease symptoms and/or the underlying cause of said symptoms, reducing the frequency or likelihood of disease symptoms and/or their underlying cause, and improving or remediating damage caused, directly or indirectly, by disease, any improvement of any consequence of disease, such as prolonged survival, less morbidity, and/or a lessening of side effects which are the byproducts of an alternative therapeutic modality; as is readily appreciated in the art, full eradication of disease is a preferred but albeit not a requirement for a treatment act. Beneficial or desired clinical results include, but are not limited to, promoting the engraftment of hematopoietic cells in a patient following ADC conditioning therapy as described herein and subsequent genetically modified hematopoietic stem cell transplant therapy. Additional beneficial results include an increase in the cell count or relative concentration of hematopoietic stem cells in a patient in need of a hematopoietic stem cell transplant following conditioning therapy and subsequent administration of a hematopoietic stem cell graft to the patient. Beneficial results of therapy described herein may also include an increase in the cell count or relative concentration of one or more cells of hematopoietic lineage, such as a megakaryocyte, thrombocyte, platelet, erythrocyte, mast cell, myeloblast, basophil, neutrophil, eosinophil, microglial cell, granulocyte, monocyte, osteoclast, antigen-presenting cell, macrophage, dendritic cell, natural killer cell, T-lymphocyte, or B-lymphocyte, following conditioning therapy and subsequent hematopoietic stem cell transplant therapy. Additional beneficial results may include the reduction in quantity of a disease-causing cell population, such as a population of cancer cells (e.g., CD117+ leukemic cells) or autoimmune cells (e.g., CD117+ autoimmune lymphocytes, such as a CD117+ T-cell that expresses a T-cell receptor that cross-reacts with a self-antigen). Additional beneficial results include the presence or detection of a functional protein expressed in the patient as a result of correcting the disease-causing target gene, as described herein. Insofar as the methods of the present disclosure are directed to preventing disorders, it is understood that the term “prevent” does not require that the disease state be completely thwarted. Rather, as used herein, the term preventing refers to the ability of the skilled artisan to identify a population that is susceptible to disorders, such that administration of the compounds of the present disclosure may occur prior to onset of a disease. The term does not imply that the disease state is completely avoided.

As used herein, the terms “variant” and “derivative” are used interchangeably and refer to naturally-occurring, synthetic, and semi-synthetic analogues of a compound, peptide, protein, or other substance described herein. A variant or derivative of a compound, peptide, protein, or other substance described herein may retain or improve upon the biological activity of the original material.

As used herein, the term “vector” includes a nucleic acid vector, such as a plasmid, a DNA vector, a plasmid, a RNA vector, virus, or other suitable replicon. Expression vectors described herein may contain a polynucleotide sequence as well as, for example, additional sequence elements used for the expression of proteins and/or the integration of these polynucleotide sequences into the genome of a mammalian cell. Certain vectors that can be used for the expression of antibodies and antibody fragments of the present disclosure include plasmids that contain regulatory sequences, such as promoter and enhancer regions, which direct gene transcription. Other useful vectors for expression of antibodies and antibody fragments contain polynucleotide sequences that enhance the rate of translation of these genes or improve the stability or nuclear export of the mRNA that results from gene transcription. These sequence elements may include, for example, 5′ and 3′ untranslated regions and a polyadenylation signal site in order to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, and nourseothricin.

The term “acyl” as used herein refers to —C(═O)R, wherein R is hydrogen (“aldehyde”), C₁-C₁₂ alkyl, C₂-C₁₂ alkenyl, C₂-C₁₂ alkynyl, C₃-C₇ carbocyclyl, C₆-C₂₀ aryl, 5-10 membered heteroaryl, or 5-10 membered heterocyclyl, as defined herein. Non-limiting examples include formyl, acetyl, propanoyl, benzoyl, and acryloyl.

The term “C₁-C₁₂ alkyl” as used herein refers to a straight chain or branched, saturated hydrocarbon having from 1 to 12 carbon atoms. Representative C₁-C₁₂ alkyl groups include, but are not limited to, -methyl, -ethyl, -n-propyl, -n-butyl, -n-pentyl, and -n-hexyl; while branched C₁-C₁₂ alkyls include, but are not limited to, -isopropyl, -sec-butyl, -isobutyl, -tert-butyl, -isopentyl, and 2-methylbutyl. A C₁-C₁₂ alkyl group can be unsubstituted or substituted.

The term “alkenyl” as used herein refers to C₂-C₁₂ hydrocarbon containing normal, secondary, or tertiary carbon atoms with at least one site of unsaturation, i.e., a carbon-carbon, sp² double bond. Examples include, but are not limited to: ethylene or vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, and the like. An alkenyl group can be unsubstituted or substituted.

“Alkynyl” as used herein refers to a C₂-C₁₂ hydrocarbon containing normal, secondary, or tertiary carbon atoms with at least one site of unsaturation, i.e., a carbon-carbon, sp triple bond. Examples include, but are not limited to acetylenic and propargyl. An alkynyl group can be unsubstituted or substituted.

“Aryl” as used herein refers to a C₆-C₂₀ carbocyclic aromatic group. Examples of aryl groups include, but are not limited to, phenyl, naphthyl and anthracenyl. An aryl group can be unsubstituted or substituted.

“Arylalkyl” as used herein refers to an acyclic alkyl radical in which one of the hydrogen atoms bonded to a carbon atom, typically a terminal or sp³ carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. The arylalkyl group comprises 6 to 20 carbon atoms, e.g. the alkyl moiety, including alkanyl, alkenyl or alkynyl groups, of the arylalkyl group is 1 to 6 carbon atoms and the aryl moiety is 5 to 14 carbon atoms. An alkaryl group can be unsubstituted or substituted.

“Cycloalkyl” as used herein refers to a saturated carbocyclic radical, which may be mono- or bicyclic. Cycloalkyl groups include a ring having 3 to 7 carbon atoms as a monocycle or 7 to 12 carbon atoms as a bicycle. Examples of monocyclic cycloalkyl groups include cyclopropyl, cyclobutyl, cyclopentyl, cyclohexyl, cycloheptyl, and cyclooctyl. A cycloalkyl group can be unsubstituted or substituted.

“Cycloalkenyl” as used herein refers to an unsaturated carbocyclic radical, which may be mono- or bicyclic. Cycloalkenyl groups include a ring having 3 to 6 carbon atoms as a monocycle or 7 to 12 carbon atoms as a bicycle. Examples of monocyclic cycloalkenyl groups include 1-cyclopent-1-enyl, 1-cyclopent-2-enyl, 1-cyclopent-3-enyl, 1-cyclohex-1-enyl, 1-cyclohex-2-enyl, and 1-cyclohex-3-enyl. A cycloalkenyl group can be unsubstituted or substituted.

“Heteroaralkyl” as used herein refers to an acyclic alkyl radical in which one of the hydrogen atoms bonded to a carbon atom, typically a terminal or sp³ carbon atom, is replaced with a heteroaryl radical. Typical heteroarylalkyl groups include, but are not limited to, 2-benzimidazolylmethyl, 2-furylethyl, and the like. The heteroarylalkyl group comprises 6 to 20 carbon atoms, e.g. the alkyl moiety, including alkanyl, alkenyl or alkynyl groups, of the heteroarylalkyl group is 1 to 6 carbon atoms and the heteroaryl moiety is 5 to 14 carbon atoms and 1 to 3 heteroatoms selected from N, O, P, and S. The heteroaryl moiety of the heteroarylalkyl group may be a monocycle having 3 to 7 ring members (2 to 6 carbon atoms or a bicycle having 7 to 10 ring members (4 to 9 carbon atoms and 1 to 3 heteroatoms selected from N, O, P, and S), for example: a bicyclo[4,5], [5,5], [5,6], or [6,6] system.

“Heteroaryl” and “heterocycloalkyl” as used herein refer to an aromatic or non-aromatic ring system, respectively, in which one or more ring atoms is a heteroatom, e.g. nitrogen, oxygen, and sulfur. The heteroaryl or heterocycloalkyl radical comprises 2 to 20 carbon atoms and 1 to 3 heteroatoms selected from N, O, P, and S. A heteroaryl or heterocycloalkyl may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S), for example: a bicyclo[4,5], [5,5], [5,6], or [6,6] system. Heteroaryl and heterocycloalkyl can be unsubstituted or substituted.

Heteroaryl and heterocycloalkyl groups are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566.

Examples of heteroaryl groups include by way of example and not limitation pyridyl, thiazolyl, tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, benzotriazolyl, benzisoxazolyl, and isatinoyl.

Examples of heterocycloalkyls include by way of example and not limitation dihydroypyridyl, tetrahydropyridyl (piperidyl), tetrahydrothiophenyl, piperidinyl, 4-piperidonyl, pyrrolidinyl, 2-pyrrolidonyl, tetrahydrofuranyl, tetrahydropyranyl, bis-tetrahydropyranyl, tetrahydroquinolinyl, tetrahydroisoquinolinyl, decahydroquinolinyl, octahydroisoquinolinyl, piperazinyl, quinuclidinyl, and morpholinyl.

By way of example and not limitation, carbon bonded heteroaryls and heterocycloalkyls are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heteroaryls and heterocycloalkyls are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or beta-carboline. Still more typically, nitrogen bonded heterocycles include 1-aziridyl, 1-azetedyl, 1-pyrrolyl, 1-imidazolyl, 1-pyrazolyl, and 1-piperidinyl.

“Substituted” as used herein and as applied to any of the above alkyl, alkenyl, alkynyl, aryl, arylalkyl, cycloalkyl, heteroaryl, heterocyclyl, and the like, means that one or more hydrogen atoms are each independently replaced with a substituent. Unless otherwise constrained by the definition of the individual substituent, the foregoing chemical moieties, such as “alkyl”, “alkylene”, “heteroalkyl”, “heteroalkylene”, “alkenyl”, “alkenylene”, “heteroalkenyl”, “heteroalkenylene”, “alkynyl”, “alkynylene”, “heteroalkynyl”, “heteroalkynylene”, “cycloalkyl”, “cycloalkylene”, “heterocyclolalkyl”, heterocycloalkylene”, “aryl,” “arylene”, “heteroaryl”, and “heteroarylene” groups can optionally be substituted. Typical substituents include, but are not limited to, —X, —R, —OH, —OR, —SH, —SR, NH₂, —NHR, —N(R)₂, —N⁺(R)₃, —CX₃, —CN, —OCN, —SCN, —NCO, —NCS, —NO, —NO₂, —N₃, —NC(═O)H, —NC(═O)R, —C(═O)H, —C(═O)R, —C(═O)NH₂, —C(═O)N(R)₂, —SO₃—, —SO₃H, —S(═O)₂R, —OS(═O)₂OR, —S(═O)₂NH₂, —S(═O)₂N(R)₂, —S(═O)R, —OP(═O)(OH)₂, —OP(═O)(OR)₂, —P(═O)(OR)₂, —PO₃, —PO₃H2, —C(═O)X, —C(═S)R, —CO₂H, —CO₂R, —CO₂—, —C(═S)OR, —C(═O)SR, —C(═S)SR, —C(═O)NH₂, —C(═O)N(R)₂, —C(═S)NH₂, —C(═S)N(R)₂, —C(═NH)NH₂, and —C(═NR)N(R)₂; wherein each X is independently selected for each occasion from F, Cl, Br, and I; and each R is independently selected for each occasion from C₁-C₁₂ alkyl, C₆-C₂₀ aryl, C₃-C₁₄ heterocycloalkyl or heteroaryl, protecting group and prodrug moiety. Wherever a group is described as “optionally substituted,” that group can be substituted with one or more of the above substituents, independently for each occasion.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene,” “alkenylene,” “arylene,” “heterocycloalkylene,” and the like.

Wherever a substituent is depicted as a di-radical (i.e., has two points of attachment to the rest of the molecule), it is to be understood that the substituent can be attached in any directional configuration unless otherwise indicated.

“Isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers,” or sometimes “optical isomers.”

A carbon atom bonded to four non-identical substituents is termed a “chiral center.” “Chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116). A mixture containing equal amounts of individual enantiomeric forms of opposite chirality is termed a “racemic mixture.”

The compounds disclosed in this description and in the claims may comprise one or more asymmetric centers, and different diastereomers and/or enantiomers of each of the compounds may exist. The description of any compound in this description and in the claims is meant to include all enantiomers, diastereomers, and mixtures thereof, unless stated otherwise. In addition, the description of any compound in this description and in the claims is meant to include both the individual enantiomers, as well as any mixture, racemic or otherwise, of the enantiomers, unless stated otherwise. When the structure of a compound is depicted as a specific enantiomer, it is to be understood that the present disclosure of the present application is not limited to that specific enantiomer. Accordingly, enantiomers, optical isomers, and diastereomers of each of the structural formulae of the present disclosure are contemplated herein. In the present specification, the structural formula of the compound represents a certain isomer for convenience in some cases, but the present disclosure includes all isomers, such as geometrical isomers, optical isomers based on an asymmetrical carbon, stereoisomers, tautomers, and the like, it being understood that not all isomers may have the same level of activity. The compounds may occur in different tautomeric forms. The compounds according to the disclosure are meant to include all tautomeric forms, unless stated otherwise. When the structure of a compound is depicted as a specific tautomer, it is to be understood that the present disclosure of the present application is not limited to that specific tautomer.

The compounds of any formula described herein include the compounds themselves, as well as their salts, and their solvates, if applicable. A salt, for example, can be formed between an anion and a positively charged group (e.g., amino) on a compound of the disclosure. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate). The term “pharmaceutically acceptable anion” refers to an anion suitable for forming a pharmaceutically acceptable salt. Likewise, a salt can also be formed between a cation and a negatively charged group (e.g., carboxylate) on a compound of the disclosure. Suitable cations include sodium ion, potassium ion, magnesium ion, calcium ion, and an ammonium cation such as tetramethylammonium ion. Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. The compounds of the disclosure also include those salts containing quaternary nitrogen atoms.

Examples of suitable inorganic anions include, but are not limited to, those derived from the following inorganic acids: hydrochloric, hydrobromic, hydroiodic, sulfuric, sulfurous, nitric, nitrous, phosphoric, and phosphorous. Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Additionally, the compounds of the present disclosure, for example, the salts of the compounds, can exist in either hydrated or unhydrated (the anhydrous) form or as solvates with other solvent molecules. Non-limiting examples of hydrates include monohydrates, dihydrates, etc. Non-limiting examples of solvates include ethanol solvates, acetone solvates, etc. “Solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O. A hydrate refers to, for example, a mono-hydrate, a di-hydrate, a tri-hydrate, etc.

In addition, a crystal polymorphism may be present for the compounds or salts thereof represented by the formulae disclosed herein. It is noted that any crystal form, crystal form mixture, or anhydride or hydrate thereof, is included in the scope of the present disclosure.

As used herein, the term “amatoxin” refers to a member of the amatoxin family of peptides produced by Amanita phalloides mushrooms, or a variant or derivative thereof, such as a variant or derivative thereof capable of inhibiting RNA polymerase II activity. Suitable amatoxins and derivatives thereof are further described herein below. As described herein, amatoxins may be conjugated to an antibody, or antigen-binding fragment thereof, for instance, by way of a linker moiety (L), thus forming a conjugate (i.e., an ADC). Exemplary methods of amatoxin conjugation and linkers useful for such processes are described below and are known in the art.

### B. Methods of Stem Cell Gene Therapy

Methods of the present disclosure include administering a population of genetically modified stem cells to a patient suffering from a condition that results from a defective gene (e.g., a mutation). Generally, the present methods relate to stem cell gene therapy, in which the genome of living cells (e.g., stem cells) is modified for therapeutic purposes. In particular, a therapeutic effect can be achieved by correcting a defective gene, as described herein. By way of example, hematopoietic stem cell (HSCs) may be extracted from a patient suffering from a disorder caused by the defective gene (e.g., a sickle cell patient with a defective HBB gene) and purified by selecting for CD34 expressing cells (CD34+). The isolated cells can be treated ex vivo using known methods in the art, and its genome can be modified as desired, e.g., edited to correct the defective target gene into a functional gene. Such modified stem cells are subsequently administered back to the patient. The transplanted stem cells take root in the patient's bone marrow, replicating and creating cells that mature and create normally functioning protein, thereby resolving the problem.

Methods of isolating stem cells from a source and further treatment of the cells ex vivo (e.g., expansion and genome modification) are well known and available in the art. In some embodiments, the stem cells are allogeneic to the mammal to which they are administered. In some embodiments, the stem cells are autologous to the mammal to which they are administered.

In some embodiments, the stem cells are isolated from bone marrow. In some embodiments, the stem cells are isolated from peripheral blood, e.g., mobilized peripheral blood. In some embodiments, the mobilized peripheral blood is isolated from a subject who has been administered a G-CSF. In some embodiments, the mobilized peripheral blood is isolated from a subject who has been administered a mobilization agent other than G-CSF, for example, Plerixafor® (AMD3100). In some embodiments, the stem cells are isolated from umbilical cord blood.

In some embodiments, the isolated stem cells comprise or consist of CD34+ cells. In some embodiments, the cells are substantially free of CD34− cells. In some embodiments, the cells comprise or consist of CD34+/CD90+ stem cells. In some embodiments, the cells comprise or consist of CD34+/CD90− cells. In some embodiments, the cells are a population comprising one or more of the cell types described above or described herein.

In some embodiments, any one or more of known genetically modified stem cells can be used in the present methods, including, e.g., Strimvelis™ (autologous 0034+ enriched cell fraction that contains CD34+ cells transduced with retroviral vector that encodes for the human ADA cDNA sequence).

The genetically modified HSC described herein may be used in genetically modified stem cell therapy, or stem cell gene therapy, which refers to the in vitro gene editing (e.g., by CRISPR/Cas system or by viral transduction) of cells to form genetically modified cells prior to introducing into a patient. Therefore, the genetically modified stem cells described herein are used in methods of gene therapy because they contain the altered or corrected gene, and/or contain an exogenous gene. In particular, the genetically modified stem cells described herein are useful in methods of gene therapy because all or most progeny from the modified stem cells will contain the altered or corrected gene. The modified hematopoietic cells can therefore be used for treatment of a mammalian subject, such as a human subject, suffering from a condition including but not limited to, inherited disorders, cancer, and certain viral infections.

As described herein, the genetically modified stem cells are administered (i.e., transplanted) to a patient that has been conditioned using the ADCs and methods as described herein to ensure or improve engraftment.

1. Stem Cell Genome Modification Methods

Various methods for editing the genome of cells (e.g., stem cells) are known in the art, e.g., as described herein. Stem cells to be manipulated include individual isolated stem cells or stem cells from a stem cell line established from the isolated stem cells, which comprise one or more nucleic acid mutations. Any suitable genetic manipulation method known in the art and those described herein, may be used to edit or alter the genome of the stem cells. In particular embodiments, genetic modification of the genome of the stem cell can correct the nucleic acid mutation in the stem cells. In some embodiments, genetic modification of the genome of the stem cells can introduce an exogenous gene into the stem cell. In certain embodiments, nucleic acid manipulation reagents (e.g., components of a gene editing system) are introduced into the stem cells. In some embodiments, delivery of genetic material (e.g., an exogenous gene to be expressed) and/or nucleic acid manipulation reagents (e.g., components of a gene editing system) can be achieved via a viral vector (e.g., retroviral, adenoviral, AAV, helper-dependent adenoviral systems, hybrid adenoviral systems, herpes simplex, pox virus, lentivirus, and Epstein-Barr virus), and non-viral systems, such as physical systems (naked DNA, DNA bombardment, electroporation, hydrodynamic, ultrasound, and magnetofection), and chemical system (cationic lipids, different cationic polymers, and lipid polymers). In some embodiments, the viral vector is a lentivirus. Nucleic acid manipulation reagents subsequently correct the nucleic acid mutation in the stem cell to form manipulated stem cells. Such reagents work by enabling efficient and precise modification of one or more target polynucleotide sequences (target sequences) in a target cell (e.g., stem cell), and typically comprise a site-directed modifying polypeptide, such as a nucleic acid-guided endonuclease (e.g., RNA-guided endonuclease, such as Cas9, or DNA-guided endonuclease) that recognizes a nucleic acid sequence in the target cell.

The site-directed modifying polypeptides used in the presently disclosed compositions and methods are site-specific, in that the polypeptide itself or an associated molecule recognizes and is targeted to a particular nucleic acid sequence or a set of similar sequences (i.e., target sequence(s)). In some embodiments, the site-directed modifying polypeptide (or its associated molecule) recognizes sequences that are similar in sequence, comprising conserved bases or motifs that can be degenerate at one or more positions.

In particular embodiments, the site-directed modifying polypeptide modifies the polynucleotide at particular location(s) (i.e., modification site(s)) outside of its target sequence. The modification site(s) modified by a particular site-directed modifying polypeptide are also generally specific to a particular sequence or set of similar sequences. In some of these embodiments, the site-directed modifying polypeptide modifies sequences that are similar in sequence, comprising conserved bases or motifs that can be degenerate at one or more positions. In other embodiments, the site-directed modifying polypeptide modifies sequences that are within a particular location relative to the target sequence(s). For example, the site-directed modifying polypeptide may modify sequences that are within a particular number of nucleic acids upstream or downstream from the target sequence(s).

As used herein with respect to site-directed modifying polypeptides, the term “modification” or “alteration” means any insertion, deletion, substitution, or chemical modification of at least one nucleotide the modification site or alternatively, a change in the expression of a gene that is adjacent to the target site. The substitution of at least one nucleotide in the modification site can be the result of the recruitment of a base editing domain, such as a cytidine deaminase or adenine deaminase domain (see, for example, Eid et al. (2018) Biochem J 475(11):1955-1964, which is herein incorporated in its entirety).

The change in expression of a gene adjacent to a target site can result from the recruitment of a transcriptional activation domain or transcriptional repression domain to the promoter region of the gene or the recruitment of an epigenetic modification domain that covalently modifies DNA or histone proteins to alter histone structure and/or chromosomal structure without altering the DNA sequence, leading to changes in gene expression of an adjacent gene. The term “modification” or “alteration” also encompasses the recruitment to a target site of a detectable label that can be conjugated to the site-directed modifying polypeptide or an associated molecule (e.g., gRNA) that allows for the detection of a specific nucleic acid sequence (e.g., a disease-associated sequence).

In some embodiments, the site-directed modifying polypeptide is a nuclease or variant thereof and the agent comprising the nuclease or variant thereof. As used herein a “nuclease” refers to an enzyme which cleaves a phosphodiester bond in the backbone of a polynucleotide chain. Suitable nucleases for the presently disclosed compositions and methods can have endonuclease and/or exonuclease activity. An exonuclease cleaves nucleotides one at a time from the end of a polynucleotide chain. An endonuclease cleaves a polynucleotide chain by cleaving phosphodiester bonds within a polynucleotide chain, other than those at the two ends of a polynucleotide chain. The nuclease can cleave RNA polynucleotide chains (i.e., ribonuclease) and/or DNA polynucleotide chains (i.e., deoxyribonuclease).

Nucleases cleave polynucleotide chains, resulting in a cleavage site. As used herein, the term “cleave” refers to the hydrolysis of phosphodiester bonds within the backbone of a polynucleotide chain. Cleavage by nucleases of the presently disclosure can be single-stranded or double-stranded. In some embodiments, a double-stranded cleavage of DNA is achieved via cleavage with two nucleases wherein each nuclease cleaves a single strand of the DNA. Cleavage by the nuclease can result in blunt ends or staggered ends.

Non-limiting examples of nucleases suitable for the presently disclosed compositions and methods include meganucleases, such as homing endonucleases; restriction endonucleases, such as Type IIS endonucleases (e.g., FokI)); zinc finger nucleases; transcription activator-like effector nucleases (TALENs), and nucleic acid-guided nucleases (e.g., RNA-guided endonuclease, DNA-guided endonuclease, or DNA/RNA-guided endonuclease).

As used herein, a “meganuclease” refers to an endonuclease that binds DNA at a target sequence that is greater than 12 base pairs in length. Meganucleases bind to double-stranded DNA as heterodimers. Suitable meganucleases for the presently disclosed compositions and methods include homing endonucleases, such as those of the LAGLIDADG (SEQ ID NO: 321) family comprising this amino acid motif or a variant thereof.

As used herein, a “zinc finger nuclease” or “ZFN” refers to a chimeric protein comprising a zinc finger DNA-binding domain fused to a nuclease domain from an exonuclease or endonuclease, such as a restriction endonuclease or meganuclease. The zinc finger DNA-binding domain is bound by a zinc ion that serves to stabilize the unique structure.

As used herein, a “transcription activator-like effector nuclease” or “TALEN” refers to a chimeric protein comprising a DNA-binding domain comprising multiple TAL domain repeats fused to a nuclease domain from an exonuclease or endonuclease, such as a restriction endonuclease or meganuclease. TAL domain repeats can be derived from the TALE family of proteins from the Xanthomonas genus of Proteobacteria. TAL domain repeats are 33-34 amino acid sequences with hypervariable 12^{th} and 13^{th} amino acids that are referred to as the repeat variable diresidue (RVD). The RVD imparts specificity of target sequence binding. The TAL domain repeats can be engineered through rational or experimental means to produce variant TALENs that have a specific target sequence specificity (see, for example, Boch et al. (2009) Science 326(5959):1509-1512 and Moscou and Bogdanove (2009) Science 326(5959):1501, each of which is incorporated by reference in its entirety). DNA cleavage by a TALEN requires two DNA target sequences flanking a nonspecific spacer region, wherein each DNA target sequence is bound by a TALEN monomer. In some embodiments, the TALEN comprises a compact TALEN, which refers to an endonuclease comprising a DNA-binding domain with one or more TAL domain repeats fused in any orientation to any portion of a homing endonuclease (e.g., I-TevI, MmeI, EndA, End1, I-BasI, I-TevII, I-TevIII, I-TwoI, MspI, MvaI, NucA, and NucM). Compact TALENs are advantageous in that they do not require dimerization for DNA processing activity, thus only requiring a single target site.

As used herein, a “nucleic acid-guided nuclease” refers to a nuclease that is directed to a specific target sequence based on the complementarity (full or partial) between a guide nucleic acid (i.e., guide RNA or gRNA, guide DNA or gDNA, or guide DNA/RNA hybrid) that is associated with the nuclease and a target sequence. The binding between the guide RNA and the target sequence serves to recruit the nuclease to the vicinity of the target sequence. Non-limiting examples of nucleic acid-guided nucleases suitable for the presently disclosed compositions and methods include naturally-occurring Clustered Regularly Interspaced Short Palindromic Repeats (CRISPR)-associated (Cas) polypeptides from a prokaryotic organism (e.g., bacteria, archaea) or variants thereof. CRISPR sequences found within prokaryotic organisms are sequences that are derived from fragments of polynucleotides from invading viruses and are used to recognize similar viruses during subsequent infections and cleave viral polynucleotides via CRISPR-associated (Cas) polypeptides that function as an RNA-guided nuclease to cleave the viral polynucleotides. As used herein, a “CRISPR-associated polypeptide” or “Cas polypeptide” refers to a naturally-occurring polypeptide that is found within proximity to CRISPR sequences within a naturally-occurring CRISPR system. Certain Cas polypeptides function as RNA-guided nucleases.

There are at least two classes of naturally-occurring CRISPR systems, Class 1 and Class 2. In general, the nucleic acid-guided nucleases of the presently disclosed compositions and methods are Class 2 Cas polypeptides or variants thereof given that the Class 2 CRISPR systems comprise a single polypeptide with nucleic acid-guided nuclease activity, whereas Class 1 CRISPR systems require a complex of proteins for nuclease activity. There are at least three known types of Class 2 CRISPR systems, Type II, Type V, and Type VI, among which there are multiple subtypes (subtype II-A, II-B, II-C, V-A, V-B, V-C, VI-A, VI-B, and VI-C, among other undefined or putative subtypes). In general, Type II and Type V-B systems require a tracrRNA, in addition to crRNA, for activity. In contrast, Type V-A and Type VI only require a crRNA for activity. All known Type II and Type V RNA-guided nucleases target double-stranded DNA, whereas all known Type VI RNA-guided nucleases target single-stranded RNA. The RNA-guided nucleases of Type II CRISPR systems are referred to as Cas9 herein and in the literature.

In some embodiments, the nucleic acid-guided nuclease of the presently disclosed compositions and methods is a Type II Cas9 protein or a variant thereof. Type V Cas polypeptides that function as RNA-guided nucleases do not require tracrRNA for targeting and cleavage of target sequences. The RNA-guided nuclease of Type VA CRISPR systems are referred to as Cpf1; of Type VB CRISPR systems are referred to as C2C1; of Type VC CRISPR systems are referred to as Cas12C or C2C3; of Type VIA CRISPR systems are referred to as C2C2 or Cas13A1; of Type VIB CRISPR systems are referred to as Cas13B; and of Type VIC CRISPR systems are referred to as Cas13A2 herein and in the literature. In certain embodiments, the nucleic acid-guided nuclease of the presently disclosed compositions and methods is a Type VA Cpf1 protein or a variant thereof. Naturally-occurring Cas polypeptides and variants thereof that function as nucleic acid-guided nucleases are known in the art and include, but are not limited to Streptococcus pyogenes Cas9, Staphylococcus aureus Cas9, Streptococcus thermophilus Cas9, Francisella novicida Cpf1, or those described in Shmakov et al. (2017) Nat Rev Microbiol 15(3):169-182; Makarova et al. (2015) Nat Rev Microbiol 13(11):722-736; and U.S. Pat. No. 9,790,490, each of which is incorporated herein in its entirety. Class 2 Type V CRISPR nucleases include Cas12 and any subtypes of Cas12, such as Cas12a, Cas12b, Cas12c, Cas12d, Cas12e, Cas12f, Cas12g, Cas12h, and Cas12i. Class 2 Type VI CRISPR nucleases including Cas13 can be employed in order to cleave RNA target sequences.

The nucleic acid-guided nuclease of the presently disclosed compositions and methods can be a naturally-occurring nucleic acid-guided nuclease (e.g., S. pyogenes Cas9) or a variant thereof. Variant nucleic acid-guided nucleases can be engineered or naturally occurring variants that contain substitutions, deletions, or additions of amino acids that, for example, alter the activity of one or more of the nuclease domains, fuse the nucleic acid-guided nuclease to a heterologous domain that imparts a modifying property (e.g., transcriptional activation domain, epigenetic modification domain, detectable label), modify the stability of the nuclease, or modify the specificity of the nuclease.

In some embodiments, a nucleic acid-guided nuclease includes one or more mutations to improve specificity for a target site and/or stability in the intracellular microenvironment. For example, where the protein is Cas9 (e.g., SpCas9) or a modified Cas9, it may be beneficial to delete any or all residues from N175 to R307 (inclusive) of the Rec2 domain. It may be found that a smaller, or lower-molecular mass, version of the nuclease is more effective. In some embodiments, the nuclease comprises at least one substitution relative to a naturally-occurring version of the nuclease. For example, where the protein is Cas9 or a modified Cas9, it may be beneficial to mutate C80 or C574 (or homologs thereof, in modified proteins with indels). In Cas9, desirable substitutions may include any of C80A, C80L, C80I, C80V, C80K, C574E, C574D, C574N, and C574Q (in any combination). Substitutions may be included to reduce intracellular protein binding of the nuclease and/or increase target site specificity. Additionally, or alternatively, substitutions may be included to reduce off-target toxicity of the composition.

The nucleic acid-guided nuclease is directed to a particular target sequence through its association with a guide nucleic acid (e.g., guideRNA (gRNA), guideDNA (gDNA)). The nucleic acid-guided nuclease is bound to the guide nucleic acid via non-covalent interactions, thus forming a complex. The polynucleotide-targeting nucleic acid provides target specificity to the complex by comprising a nucleotide sequence that is complementary to a sequence of a target sequence. The nucleic acid-guided nuclease of the complex or a domain or label fused or otherwise conjugated thereto provides the site-specific activity. In other words, the nucleic acid-guided nuclease is guided to a target polynucleotide sequence (e.g. a target sequence in a chromosomal nucleic acid; a target sequence in an extrachromosomal nucleic acid, e.g. an episomal nucleic acid, a minicircle; a target sequence in a mitochondrial nucleic acid; a target sequence in a chloroplast nucleic acid; a target sequence in a plasmid) by virtue of its association with the protein-binding segment of the polynucleotide-targeting guide nucleic acid.

Thus, the guide nucleic acid comprises two segments, a “polynucleotide-targeting segment” and a “polypeptide-binding segment.” By “segment” it is meant a segment/section/region of a molecule (e.g., a contiguous stretch of nucleotides in an RNA). A segment can also refer to a region/section of a complex such that a segment may comprise regions of more than one molecule. For example, in some cases the polypeptide-binding segment (described below) of a polynucleotide-targeting nucleic acid comprises only one nucleic acid molecule and the polypeptide-binding segment therefore comprises a region of that nucleic acid molecule. In other cases, the polypeptide-binding segment (described below) of a DNA-targeting nucleic acid comprises two separate molecules that are hybridized along a region of complementarity.

The polynucleotide-targeting segment (or “polynucleotide-targeting sequence” or “guide sequence”) comprises a nucleotide sequence that is complementary (fully or partially) to a specific sequence within a target sequence (for example, the complementary strand of a target DNA sequence). The polypeptide-binding segment (or “polypeptide-binding sequence”) interacts with a nucleic acid-guided nuclease. In general, site-specific cleavage or modification of the target DNA by a nucleic acid-guided nuclease occurs at locations determined by both (i) base-pairing complementarity between the polynucleotide-targeting sequence of the nucleic acid and the target DNA; and (ii) a short motif (referred to as the protospacer adjacent motif (PAM)) in the target DNA.

A protospacer adjacent motif can be of different lengths and can be a variable distance from the target sequence, although the PAM is generally within about 1 to about 10 nucleotides from the target sequence, including about 1, about 2, about 3, about 4, about 5, about 6, about 7, about 8, about 9, or about 10 nucleotides from the target sequence. The PAM can be 5′ or 3′ of the target sequence. Generally, the PAM is a consensus sequence of about 3-4 nucleotides, but in particular embodiments, can be 2, 3, 4, 5, 6, 7, 8, 9, or more nucleotides in length. Methods for identifying a preferred PAM sequence or consensus sequence for a given RNA-guided nuclease are known in the art and include, but are not limited to the PAM depletion assay described by Karvelis et al. (2015) Genome Biol 16:253, or the assay disclosed in Pattanayak et al. (2013) Nat Biotechnol 31(9):839-43, each of which is incorporated by reference in its entirety.

The polynucleotide-targeting sequence (i.e., guide sequence) is the nucleotide sequence that directly hybridizes with the target sequence of interest. The guide sequence is engineered to be fully or partially complementary with the target sequence of interest. In various embodiments, the guide sequence can comprise from about 8 nucleotides to about 30 nucleotides, or more. For example, the guide sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the guide sequence is about 10 to about 26 nucleotides in length, or about 12 to about 30 nucleotides in length. In particular embodiments, the guide sequence is about 30 nucleotides in length. In some embodiments, the degree of complementarity between a guide sequence and its corresponding target sequence, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In particular embodiments, the guide sequence is free of secondary structure, which can be predicted using any suitable polynucleotide folding algorithm known in the art, including but not limited to mFold (see, e.g., Zuker and Stiegler (1981) Nucleic Acids Res. 9:133-148) and RNAfold (see, e.g., Gruber et al. (2008) Cell 106(1):23-24).

In some embodiments, a guide nucleic acid comprises two separate nucleic acid molecules (an “activator-nucleic acid” and a “targeter-nucleic acid”, see below) and is referred to herein as a “double-molecule guide nucleic acid” or a “two-molecule guide nucleic acid.” In other embodiments, the subject guide nucleic acid is a single nucleic acid molecule (single polynucleotide) and is referred to herein as a “single-molecule guide nucleic acid,” a “single-guide nucleic acid,” or an “sgNA.” The term “guide nucleic acid” or “gNA” is inclusive, referring both to double-molecule guide nucleic acids and to single-molecule guide nucleic acids (i.e., sgNAs). In those embodiments wherein the guide nucleic acid is an RNA, the gRNA can be a double-molecule guide RNA or a single-guide RNA. Likewise, in those embodiments wherein the guide nucleic acid is a DNA, the gDNA can be a double-molecule guide DNA or a single-guide DNA.

An exemplary two-molecule guide nucleic acid comprises a crRNA-like (“CRISPR RNA” or “targeter-RNA” or “crRNA” or “crRNA repeat”) molecule and a corresponding tracrRNA-like (“trans-acting CRISPR RNA” or “activator-RNA” or “tracrRNA”) molecule. A crRNA-like molecule (targeter-RNA) comprises both the polynucleotide-targeting segment (single stranded) of the guide RNA and a stretch (“duplex-forming segment”) of nucleotides that forms one half of the dsRNA duplex of the polypeptide-binding segment of the guide RNA, also referred to herein as the CRISPR repeat sequence.

The term “activator-nucleic acid” or “activator-NA” is used herein to mean a tracrRNA-like molecule of a double-molecule guide nucleic acid. The term “targeter-nucleic acid” or “targeter-NA” is used herein to mean a crRNA-like molecule of a double-molecule guide nucleic acid. The term “duplex-forming segment” is used herein to mean the stretch of nucleotides of an activator-NA or a targeter-NA that contributes to the formation of the dsRNA duplex by hybridizing to a stretch of nucleotides of a corresponding activator-NA or targeter-NA molecule. In other words, an activator-NA comprises a duplex-forming segment that is complementary to the duplex-forming segment of the corresponding targeter-NA. As such, an activator-NA comprises a duplex-forming segment while a targeter-NA comprises both a duplex-forming segment and the DNA-targeting segment of the guide nucleic acid. Therefore, a subject double-molecule guide nucleic acid can be comprised of any corresponding activator-NA and targeter-NA pair.

The activator-NA comprises a CRISPR repeat sequence comprising a nucleotide sequence that comprises a region with sufficient complementarity to hybridize to an activator-NA (the other part of the polypeptide-binding segment of the guide nucleic acid). In various embodiments, the CRISPR repeat sequence can comprise from about 8 nucleotides to about 30 nucleotides, or more. For example, the CRISPR repeat sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the degree of complementarity between a CRISPR repeat sequence and the antirepeat region of its corresponding tracr sequence, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more.

A corresponding tracrRNA-like molecule (i.e., activator-NA) comprises a stretch of nucleotides (duplex-forming segment) that forms the other part of the double-stranded duplex of the polypeptide-binding segment of the guide nucleic acid. In other words, a stretch of nucleotides of a crRNA-like molecule (i.e., the CRISPR repeat sequence) are complementary to and hybridize with a stretch of nucleotides of a tracrRNA-like molecule (i.e., the anti-repeat sequence) to form the double-stranded duplex of the polypeptide-binding domain of the guide nucleic acid. The crRNA-like molecule additionally provides the single stranded DNA-targeting segment. Thus, a crRNA-like and a tracrRNA-like molecule (as a corresponding pair) hybridize to form a guide nucleic acid. The exact sequence of a given crRNA or tracrRNA molecule is characteristic of the CRISPR system and species in which the RNA molecules are found. A subject double-molecule guide RNA can comprise any corresponding crRNA and tracrRNA pair.

A trans-activating-like CRISPR RNA or tracrRNA-like molecule (also referred to herein as an “activator-NA”) comprises a nucleotide sequence comprising a region that has sufficient complementarity to hybridize to a CRISPR repeat sequence of a crRNA, which is referred to herein as the anti-repeat region. In some embodiments, the tracrRNA-like molecule further comprises a region with secondary structure (e.g., stem-loop) or forms secondary structure upon hybridizing with its corresponding crRNA. In particular embodiments, the region of the tracrRNA-like molecule that is fully or partially complementary to a CRISPR repeat sequence is at the 5′ end of the molecule and the 3′ end of the tracrRNA-like molecule comprises secondary structure. This region of secondary structure generally comprises several hairpin structures, including the nexus hairpin, which is found adjacent to the anti-repeat sequence. The nexus hairpin often has a conserved nucleotide sequence in the base of the hairpin stem, with the motif UNANNC found in many nexus hairpins in tracrRNAs. There are often terminal hairpins at the 3′ end of the tracrRNA that can vary in structure and number, but often comprise a GC-rich Rho-independent transcriptional terminator hairpin followed by a string of U's at the 3′ end. See, for example, Briner et al. (2014) Molecular Cell 56:333-339, Briner and Barrangou (2016) Cold Spring Harb Protoc; doi: 10.1101/pdb.top090902, and U.S. Publication No. 2017/0275648, each of which is herein incorporated by reference in its entirety.

In various embodiments, the anti-repeat region of the tracrRNA-like molecule that is fully or partially complementary to the CRISPR repeat sequence comprises from about 8 nucleotides to about 30 nucleotides, or more. For example, the region of base pairing between the tracrRNA-like anti-repeat sequence and the CRISPR repeat sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the degree of complementarity between a CRISPR repeat sequence and its corresponding tracrRNA-like anti-repeat sequence, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more.

In various embodiments, the entire tracrRNA-like molecule can comprise from about 60 nucleotides to more than about 140 nucleotides. For example, the tracrRNA-like molecule can be about 60, about 65, about 70, about 75, about 80, about 85, about 90, about 95, about 100, about 105, about 110, about 115, about 120, about 125, about 130, about 135, about 140, or more nucleotides in length. In particular embodiments, the tracrRNA-like molecule is about 80 to about 100 nucleotides in length, including about 80, about 81, about 82, about 83, about 84, about 85, about 86, about 87, about 88, about 89, about 90, about 91, about 92, about 93, about 94, about 95, about 96, about 97, about 98, about 99, and about 100 nucleotides in length.

A subject single-molecule guide nucleic acid (i.e., sgNA) comprises two stretches of nucleotides (a targeter-NA and an activator-NA) that are complementary to one another, are covalently linked by intervening nucleotides (“linkers” or “linker nucleotides”), and hybridize to form the double stranded nucleic acid duplex of the protein-binding segment, thus resulting in a stem-loop structure. The targeter-NA and the activator-NA can be covalently linked via the 3′ end of the targeter-NA and the 5′ end of the activator-NA. Alternatively, the targeter-NA and the activator-NA can be covalently linked via the 5′ end of the targeter-NA and the 3′ end of the activator-NA.

The linker of a single-molecule DNA-targeting nucleic acid can have a length of from about 3 nucleotides to about 100 nucleotides. For example, the linker can have a length of from about 3 nucleotides (nt) to about 90 nt, from about 3 nt to about 80 nt, from about 3 nt to about 70 nt, from about 3 nt to about 60 nt, from about 3 nt to about 50 nt, from about 3 nt to about 40 nt, from about 3 nt to about 30 nt, from about 3 nt to about 20 nt or from about 3 nt to about 10 nt, including but not limited to about 3, about 4, about 5, about 6, about 7, about 8, about 9, about 10, about 11, about 12, about 13, about 14, about 15, about 16, about 17, about 18, about 19, about 20, or more nucleotides. In some embodiments, the linker of a single-molecule DNA-targeting nucleic acid is 4 nt.

An exemplary single-molecule DNA-targeting nucleic acid comprises two complementary stretches of nucleotides that hybridize to form a double-stranded duplex, along with a guide sequence that hybridizes to a specific target sequence.

Appropriate naturally-occurring cognate pairs of crRNAs (and, in some embodiments, tracrRNAs) are known for most Cas proteins that function as nucleic acid-guided nucleases that have been discovered or can be determined for a specific naturally-occurring Cas protein that has nucleic acid-guided nuclease activity by sequencing and analyzing flanking sequences of the Cas nucleic acid-guided nuclease protein to identify tracrRNA-coding sequence, and thus, the tracrRNA sequence, by searching for known antirepeat-coding sequences or a variant thereof. Antirepeat regions of the tracrRNA comprise one-half of the ds protein-binding duplex. The complementary repeat sequence that comprises one-half of the ds protein-binding duplex is called the CRISPR repeat. CRISPR repeat and antirepeat sequences utilized by known CRISPR nucleic acid-guided nucleases are known in the art and can be found, for example, at the CRISPR database on the world wide web at crispr.i2bc.paris-saclay.fr/crispr/.

The single guide nucleic acid or dual-guide nucleic acid can be synthesized chemically or via in vitro transcription. Assays for determining sequence-specific binding between a nucleic acid-guided nuclease and a guide nucleic acid are known in the art and include, but are not limited to, in vitro binding assays between an expressed nucleic acid-guided nuclease and the guide nucleic acid, which can be tagged with a detectable label (e.g., biotin) and used in a pull-down detection assay in which the nucleoprotein complex is captured via the detectable label (e.g., with streptavidin beads). A control guide nucleic acid with an unrelated sequence or structure to the guide nucleic acid can be used as a negative control for non-specific binding of the nucleic acid-guided nuclease to nucleic acids.

In certain embodiments, the site-directed modifying polypeptide of the presently disclosed compositions and methods comprise a nuclease variant that functions as a nickase, wherein the nuclease comprises a mutation in comparison to the wild-type nuclease that results in the nuclease only being capable of cleaving a single strand of a double-stranded nucleic acid molecule, or lacks nuclease activity altogether (i.e., nuclease-dead).

A nuclease, such as a nucleic acid-guided nuclease, that functions as a nickase only comprises a single functioning nuclease domain. In some of these embodiments, additional nuclease domains have been mutated such that the nuclease activity of that particular domain is reduced or eliminated.

In other embodiments, the nuclease (e.g., RNA-guided nuclease) lacks nuclease activity completely and is referred to herein as nuclease-dead. In some of these embodiments, all nuclease domains within the nuclease have been mutated such that all nuclease activity of the polypeptide has been eliminated. Any method known in the art can be used to introduce mutations into one or more nuclease domains of a site-directed nuclease, including those set forth in U.S. Publ. Nos. 2014/0068797 and U.S. Pat. No. 9,790,490, each of which is incorporated by reference in its entirety.

Any mutation within a nuclease domain that reduces or eliminates the nuclease activity can be used to generate a nucleic acid-guided nuclease having nickase activity or a nuclease-dead nucleic acid-guided nuclease. Such mutations are known in the art and include, but are not limited to the D10A mutation within the RuvC domain or H840A mutation within the HNH domain of the S. pyogenes Cas9 or at similar position(s) within another nucleic acid-guided nuclease when aligned for maximal homology with the S. pyogenes Cas9. Other positions within the nuclease domains of S. pyogenes Cas9 that can be mutated to generate a nickase or nuclease-dead protein include G12, G17, E762, N854, N863, H982, H983, and D986. Other mutations within a nuclease domain of a nucleic acid-guided nuclease that can lead to nickase or nuclease-dead proteins include a D917A, E1006A, E1028A, D1227A, D1255A, N1257A, D917A, E1006A, E1028A, D1227A, D1255A, and N1257A of the Francisella novicida Cpf1 protein or at similar position(s) within another nucleic acid-guided nuclease when aligned for maximal homology with the F. novicida Cpf1 protein (U.S. Pat. No. 9,790,490, which is incorporated by reference in its entirety).

Site-directed modifying polypeptides comprising a nuclease-dead domain can further comprise a domain capable of modifying a polynucleotide. Non-limiting examples of modifying domains that may be fused to a nuclease-dead domain include but are not limited to, a transcriptional activation or repression domain, a base editing domain, and an epigenetic modification domain. In other embodiments, the site-directed modifying polypeptide comprising a nuclease-dead domain further comprises a detectable label that can aid in detecting the presence of the target sequence.

The epigenetic modification domain that can be fused to a nuclease-dead domain serves to covalently modify DNA or histone proteins to alter histone structure and/or chromosomal structure without altering the DNA sequence itself, leading to changes in gene expression (upregulation or downregulation). Non-limiting examples of epigenetic modifications that can be induced by site-directed modifying polypeptides include the following alterations in histone residues and the reverse reactions thereof: sumoylation, methylation of arginine or lysine residues, acetylation or ubiquitination of lysine residues, phosphorylation of serine and/or threonine residues; and the following alterations of DNA and the reverse reactions thereof: methylation or hydroxymethylation of cystosine residues. Non-limiting examples of epigenetic modification domains thus include histone acetyltransferase domains, histone deacetylation domains, histone methyltransferase domains, histone demethylase domains, DNA methyltransferase domains, and DNA demethylase domains.

In some embodiments, the site-directed polypeptide comprises a transcriptional activation domain that activates the transcription of at least one adjacent gene through the interaction with transcriptional control elements and/or transcriptional regulatory proteins, such as transcription factors or RNA polymerases. Suitable transcriptional activation domains are known in the art and include, but are not limited to, VP16 activation domains.

In other embodiments, the site-directed polypeptide comprises a transcriptional repressor domain, which can also interact with transcriptional control elements and/or transcriptional regulatory proteins, such as transcription factors or RNA polymerases, to reduce or terminate transcription of at least one adjacent gene. Suitable transcriptional repression domains are known in the art and include, but are not limited to, IκB and KRAB domains.

In still other embodiments, the site-directed modifying polypeptide comprising a nuclease-dead domain further comprises a detectable label that can aid in detecting the presence of the target sequence, which may be a disease-associated sequence. A detectable label is a molecule that can be visualized or otherwise observed. The detectable label may be fused to the nucleic-acid guided nuclease as a fusion protein (e.g., fluorescent protein) or may be a small molecule conjugated to the nuclease polypeptide that can be detected visually or by other means. Detectable labels that can be fused to the presently disclosed nucleic-acid guided nucleases as a fusion protein include any detectable protein domain, including but not limited to, a fluorescent protein or a protein domain that can be detected with a specific antibody. Non-limiting examples of fluorescent proteins include green fluorescent proteins (e.g., GFP, EGFP, ZsGreen1) and yellow fluorescent proteins (e.g., YFP, EYFP, ZsYellow1). Non-limiting examples of small molecule detectable labels include radioactive labels, such as ³H and ³⁵S.

The nucleic acid-guided nuclease can be delivered as part of a delivery system into a cell as a nucleoprotein complex comprising the nucleic acid-guided nuclease bound to its guide nucleic acid. Alternatively, the nucleic acid-guided nuclease and the guide nucleic acid are provided separately. In certain embodiments, a guide RNA can be introduced into a target cell as an RNA molecule. The guide RNA can be transcribed in vitro or chemically synthesized. In other embodiments, a nucleotide sequence encoding the guide RNA is introduced into the cell. In some of these embodiments, the nucleotide sequence encoding the guide RNA is operably linked to a promoter (e.g., an RNA polymerase Ill promoter), which can be a native promoter or heterologous to the guide RNA-encoding nucleotide sequence.

In certain embodiments, the site-directed polypeptide can comprise additional amino acid sequences, such as at least one nuclear localization sequence (NLS). Nuclear localization sequences enhance transport of the site-directed polypeptide into the nucleus of a cell. Proteins that are imported into the nucleus bind to one or more of the proteins within the nuclear pore complex, such as importin/karypherin proteins, which generally bind best to lysine and arginine residues. The best characterized pathway for nuclear localization involves short peptide sequence which binds to the importin-a protein. These nuclear localization sequences often comprise stretches of basic amino acids and given that there are two such binding sites on importin-a, two basic sequences separated by at least 10 amino acids can make up a bipartite NLS. The second most characterized pathway of nuclear import involves proteins that bind to the importin-β1 protein, such as the HIV-TAT and HIV-REV proteins, which use the sequences RKKRRQRRR (SEQ ID NO: 322) and RQARRNRRRRWR (SEQ ID NO: 323), respectively to bind to importin-β1. Other nuclear localization sequences are known in the art (see, e.g., Lange et al., J. Biol. Chem. (2007) 282:5101-5105). The NLS can be the naturally-occurring NLS of the site-directed polypeptide or a heterologous NLS. As used herein, “heterologous” in reference to a sequence is a sequence that originates from a foreign species, or, if from the same species, is substantially modified from its native form in composition and/or genomic locus by deliberate human intervention. Non-limiting examples of NLS sequences that can be used to enhance the nuclear localization of the site-directed polypeptides include the NLS of the SV40 Large T-antigen and c-Myc. In certain embodiments, the NLS comprises the amino acid sequence PKKKRKV (SEQ ID NO: 324).

The site-directed polypeptide can comprise more than one NLS, such as two, three, four, five, six, or more NLS sequences. Each of the multiple NLSs can be unique in sequence or there can be more than one of the same NLS sequence used. The NLS can be on the amino-terminal (N-terminal) end of the site-directed polypeptide, the carboxy-terminal (C-terminal) end, or both the N-terminal and C-terminal ends of the polypeptide. In certain embodiments, the site-directed polypeptide comprises four NLS sequences on its N-terminal end. In other embodiments, the site-directed polypeptide comprises two NLS sequences on the C-terminal end of the site-directed polypeptide. In still other embodiments, the site-directed polypeptide comprises four NLS sequences on its N-terminal end and two NLS sequences on its C-terminal end.

In certain embodiments, the site-directed polypeptide comprises a cell penetrating peptide (CPP), which induces the absorption of a linked protein or peptide through the plasma membrane of a cell. Generally, CPPs induce entry into the cell because of their general shape and tendency to either self-assemble into a membrane-spanning pore, or to have several positively charged residues, which interact with the negatively charged phospholipid outer membrane inducing curvature of the membrane, which in turn activates internalization. Exemplary permeable peptides include, but are not limited to, transportan, PEP1, MPG, p-VEC, MAP, CADY, polyR, HIV-TAT, HIV-REV, Penetratin, R6W3, P22N, DPV3, DPV6, K-FGF, and C105Y, and are reviewed in van den Berg and Dowdy (2011) Current Opinion in Biotechnology 22:888-893 and Farkhani et al. (2014) Peptides 57:78-94, each of which is herein incorporated by reference in its entirety.

Along with or as an alternative to an NLS, the site-directed polypeptide can comprise additional heterologous amino acid sequences, such as a detectable label (e.g., fluorescent protein) described elsewhere herein, or a purification tag, to form a fusion protein. A purification tag is any molecule that can be utilized to isolate a protein or fused protein from a mixture (e.g., biological sample, culture medium). Non-limiting examples of purification tags include biotin, myc, maltose binding protein (MBP), and glutathione-S-transferase (GST).

The presently disclosed compositions and methods can be used to edit genomes through the introduction of a sequence-specific, double-stranded break that is repaired (via e.g., error-prone non-homologous end-joining (NHEJ), microhomology-mediated end joining (MMEJ), or alternative end-joining (alt-EJ) pathway) to introduce a mutation at a specific genomic location. Due to the error-prone nature of repair processes, repair of the double-stranded break can result in a modification to the target sequence. Alternatively, a donor template polynucleotide may be integrated into or exchanged with the target sequence during the course of repair of the introduced double-stranded break, resulting in the introduction of the exogenous donor sequence. Accordingly, the compositions and methods can further comprise a donor template polynucleotide that may comprise flanking homologous ends. In some of these embodiments, the donor template polynucleotide is tethered to the site-directed polypeptide via a linker as described elsewhere herein (e.g., the donor template polynucleotide is bound to the site-directed polypeptide via a cleavable linker).

In some embodiments, the donor sequence alters the original target sequence such that the newly integrated donor sequence will not be recognized and cleaved by the nucleic acid-guided nuclease. The donor sequence may comprise flanking sequences that have substantial sequence identity with the sequences flanking the target sequence to enhance the integration of the donor sequence via homology-directed repair. In particular embodiments wherein the nucleic acid-guided nuclease generates double-stranded staggered breaks, the donor polynucleotide can be flanked by compatible overhangs, allowing for incorporation of the donor sequence via a non-homologous repair process during repair of the double-stranded break.

The nucleic acid manipulation reagents of the present disclosure can be introduced into stem cells using any suitable delivery method. Delivery can be via in vitro, ex vivo, or in vivo administration. Exemplary methods for introducing the nucleic acid manipulation reagents include, but are not limited to, transfection, electroporation and viral-based methods. In some embodiments, the stem cell is isolated from the subject prior to introduction of gene editing components. Stem cells can be genetically altered ex vivo and returned (e.g., transplanted) to a subject. In one embodiment, the subject is the same subject from whom the cell is isolated. In another embodiment, the subject is different from the subject from whom the cell is isolated. In particular embodiments, an autologous stem/progenitor cell is altered ex vivo and returned to the subject. In another embodiment, a heterologous stem/progenitor cell is altered ex vivo and returned into the subject.

Genetic modification of a stem cell can include delivery of a gRNA molecule, a Cas9 molecule, and optionally, a donor template nucleic acid, to a stem cell described herein. In one embodiment, the gRNA molecule, the Cas9 molecule, or both, and optionally the template nucleic acid, are delivered by a viral vector, e.g., an AAV vector or lentivirus vector, e.g., integration deficient lentivirus (IDLV). In another embodiment, the gRNA molecule and the Cas9 molecule are delivered as a gRNA molecule/Cas9molecule ribonucleoprotein complex. In another embodiment, the gRNAmolecule and the Cas9 molecule are delivered as RNA. A template nucleic acid can include at least one exon of a target gene for gene replacement therapy. In certain embodiments, the template nucleic acid does not contain the mutation associated with a disease or risk of disease. The template nucleic acid can include a promoter sequence functional in the target stem cell. In particular embodiments, the template nucleic acid comprises a splice donor or acceptor. In another embodiment, the template nucleic acid comprises a polyadenylation signal.

In some embodiments, the one or more cell uptake reagents are transfection reagents. Transfection reagents include, for example, polymer based (e.g., DEAE dextran) transfection reagents and cationic liposome-mediated transfection reagents. Electroporation methods may also be used to facilitate uptake of the nucleic acid manipulation reagents. By applying an external field, an altered transmembrane potential in a cell is induced, and when the transmembrane potential net value (the sum of the applied and the resting potential difference) is larger than a threshold, transient permeation structures are generated in the membrane and electroporation is achieved. See, e.g., Gehl et al., Acta Physiol. Scand. 177:437-447 (2003).

Nucleic acid manipulation reagents may also be delivered through viral transduction into the stem cells. Suitable viral delivery systems include, but are not limited to, adeno-associated virus (AAV) retroviral and lentivirus delivery systems. Such viral delivery systems are particularly useful in instances where the stem cell is resistant to transfection. Methods that use a viral-mediated delivery system may further include a step of preparing viral vectors encoding the nucleic acid manipulation reagents and packaging of the vectors into viral particles.

Other methods of delivery of nucleic acid reagents include, but are not limited to, lipofection, nucleofection, microinjection, biolistics, virosomes, liposomes, immunoliposomes, polycation or lipid:nucleic acid conjugates, naked DNA, artificial virions, nanoparticles, and agent-enhanced uptake of nucleic acids. See, also Neiwoehner et al., Nucleic Acids Res. 42:1341-1353 (2014), which is herein incorporated by reference in its entirety for all purposes, and particularly for all teachings relating to reagent delivery systems. In some embodiments, the introduction is performed by non-viral vector delivery systems include DNA plasmids, RNA (e.g. a transcript of a vector described herein), naked nucleic acid, and nucleic acid complexed with a delivery vehicle, such as a liposome.

Genetically modifying a stem cell can alter a target position (e.g., a target mutant position) in a gene of interest. Altering the target position can be achieved, for example, by repairing (e.g., correcting or altering) one or more mutations in the gene. In specific embodiments, mutations can be repaired by homology directed repair. Using homology directed repair, mutant allele(s) are corrected and restored to wild type state. In one embodiment, correction of a mutation in a gene restores wild type gene activity. Stem cells can also be modified by knocking in a polynucleotide into a target gene. In one embodiment, knocking in a polynucleotide restores wild type gene activity.

In particular embodiments, stem cells can be modified to knock out or knock down activity of a target gene. Altering the target position can be achieved, by: (1) knocking out the gene: (a) insertion or deletion (e.g., NHEJ-mediated insertion or deletion) of one or more nucleotides in close proximity to or within the early coding region of the gene, or (b) deletion (e.g., NHEJ-mediated deletion) of a genomic sequence including at least a portion of the gene, or (2) knocking down the gene mediated by enzymatically inactive Cas9 (eiCas9) molecule or an eiCas9-fusion protein (e.g., fused to a transcriptional repressor) by targeting the promoter region of the gene.

2. Indications for Treatment

As described herein, genetically modified HSC transplant therapy can be administered to a subject in need of treatment so as to populate one or more blood cell types with an alteration of a target gene. Hematopoietic stem cells generally exhibit multi-potency, and can thus differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Hematopoietic stem cells are additionally capable of self-renewal, and can thus give rise to daughter cells that have equivalent potential as the mother cell, and also feature the capacity to be reintroduced into a transplant recipient whereupon they home to the hematopoietic stem cell niche and re-establish productive and sustained hematopoiesis.

The compositions and methods described herein can thus be used to treat a non-malignant hemoglobinopathy (e.g., a hemoglobinopathy selected from the group consisting of sickle cell anemia, thalassemia, Fanconi anemia, aplastic anemia, and Wiskott-Aldrich syndrome). Additionally, or alternatively, the compositions and methods described herein can be used to treat an immunodeficiency, such as a congenital immunodeficiency. Additionally, or alternatively, the compositions and methods described herein can be used to treat an acquired immunodeficiency (e.g., an acquired immunodeficiency selected from the group consisting of HIV and AIDS). The compositions and methods described herein can be used to treat a metabolic disorder (e.g., a metabolic disorder selected from the group consisting of glycogen storage diseases, mucopolysaccharidosis, Gaucher's Disease, Hurlers Disease, sphingolipidoses, and metachromatic leukodystrophy).

In some embodiments, the present methods can be used to treat sickle cell disease, a group of disorders that affects hemoglobin. Subjects with this disorder have atypical hemoglobin molecules (hemoglobin S), which can distort red blood cells into a sickle, or crescent, shape. Characteristic features of this disorder include a low number of red blood cells (anemia), repeated infections, and periodic episodes of pain. Mutations in the HBB gene cause sickle cell disease. The HBB gene provides instructions for making beta-globin. Various versions of beta-globin result from different mutations in the HBB gene. One particular HBB gene mutation produces an abnormal version of beta-globin known as hemoglobin S (HbS). Other mutations in the HBB gene lead to additional abnormal versions of beta-globin such as hemoglobin C (HbC) and hemoglobin E (HbE). HBB gene mutations can also result in an unusually low level of beta-globin, i.e., beta thalassemia. In people with sickle cell disease, at least one of the beta-globin subunits in hemoglobin is replaced with hemoglobin S. In sickle cell anemia, which is a common form of sickle cell disease, hemoglobin S replaces both beta-globin subunits in hemoglobin. In other types of sickle cell disease, just one beta-globin subunit in hemoglobin is replaced with hemoglobin S. The other beta-globin subunit is replaced with a different abnormal variant, such as hemoglobin C. For example, people with sickle-hemoglobin C (HbSC) disease have hemoglobin molecules with hemoglobin S and hemoglobin C instead of beta-globin. If mutations that produce hemoglobin S and beta thalassemia occur together, individuals have hemoglobin S-beta thalassemia (HbSBetaThal) disease. Using known gene editing methods, any one of more of the mutations that cause sickle cell disease can be altered in the stem cell for use in the present methods. Additionally, or alternatively, a functional HBB gene can be introduced into the stem cell for transplantation.

In some embodiments, the present methods can be used to treat beta thalassemia (also called Beta Thal), a blood disorder that reduces the production of hemoglobin. In subjects with beta thalassemia, low levels of hemoglobin lead to a lack of oxygen in many parts of the body. Affected individuals also have a shortage of red blood cells (anemia), which can cause pale skin, weakness, fatigue, and more serious complications. People with beta thalassemia are at an increased risk of developing abnormal blood clots. Beta thalassemia is classified into two types depending on the severity of symptoms: thalassemia major (also known as Cooley's anemia) and thalassemia intermedia. Of the two types, thalassemia major is more severe. Mutations in the HBB gene cause beta thalassemia. The HBB gene provides instructions for making beta-globin. Some mutations in the HBB gene prevent the production of any beta-globin. The absence of beta-globin is referred to as beta-zero (B°) thalassemia. Other HBB gene mutations allow some beta-globin to be produced but in reduced amounts, i.e., beta-plus (B⁺) thalassemia. People with both types have been diagnosed with thalassemia major and thalassemia intermedia. In some embodiments, certain rare forms of beta thalassemias are caused by defective production of delta- or gamma-globin (HBG1 and HBG2, UniProt P69891 and P69892, respectively). Using known gene editing methods, any one or more of the mutations that cause beta thalassemia can be altered in the stem cell for use in the present methods. Additionally, or alternatively, any one or more functional gene (e.g., HBB, HBG1, HBG2) can be introduced into the stem cell for transplantation. See, e.g., http://dx.doi.org/10.5772/61441; Pondarre and Badens, Ann. Biol. Clin (Paris) 72(6):639-668, 2014.

In some embodiments, the present methods can be used to treat adenosine deaminase deficiency (also called ADA deficiency or ADA-SCID (severe combined immunodeficiency)), a metabolic disorder that causes immunodeficiency due to a lack of the enzyme adenosine deaminase (ADA). People with SCID lack virtually all immune protection from bacteria, viruses, and fungi. They are prone to repeated and persistent infections that can be very serious or life-threatening. These infections are often caused by “opportunistic” organisms that ordinarily do not cause illness in people with a normal immune system. Most individuals with ADA deficiency are diagnosed with SCID in the first 6 months of life. Without treatment, these babies usually do not survive past age 2. In about 10 percent to 15 percent of cases, onset of immune deficiency is delayed to between 6 and 24 months of age (delayed onset) or even until adulthood (late onset). Immune deficiency in these later-onset cases tends to be less severe, causing primarily recurrent upper respiratory and ear infections. Over time, affected individuals may develop chronic lung damage, malnutrition, and other health problems. Using known gene editing methods, any one of more of the mutations in the adenosine deaminase gene that causes ADA can be altered in the stem cell for use in the present methods. Additionally, or alternatively, a functional adenosine deaminase gene can be introduced into the stem cell for transplantation. In a yet further embodiment, the genetically modified stem cell is Strimvelis™ (autologous CD34+ enriched cell fraction that contains CD34+ cells transduced with retroviral vector that encodes for the human ADA cDNA sequence).

In some embodiments, the present methods can be used to treat metachromatic leukodystrophy (also called MLD or Arylsulfatase A deficiency), a lysosomal storage disease caused by a deficiency of the enzyme arylsulfatase A (ARSA), and is characterized by the accumulation of fats called sulfatides in cells. This accumulation especially affects cells in the nervous system that produce myelin, the substance that insulates and protects nerves. Nerve cells covered by myelin make up a tissue called white matter. Sulfatide accumulation in myelin-producing cells causes progressive destruction of white matter (leukodystrophy) throughout the nervous system, including in the brain and spinal cord (the central nervous system) and the nerves connecting the brain and spinal cord to muscles and sensory cells that detect sensations such as touch, pain, heat, and sound (the peripheral nervous system). In people with metachromatic leukodystrophy, white matter damage causes progressive deterioration of intellectual functions and motor skills, such as the ability to walk. Affected individuals also develop loss of sensation in the extremities (peripheral neuropathy), incontinence, seizures, paralysis, an inability to speak, blindness, and hearing loss. Eventually they lose awareness of their surroundings and become unresponsive. Using known gene editing methods, any one of more of the mutations in the ARSA gene that cause MLD can be altered in the stem cell for use in the present methods. Additionally, or alternatively, a functional ARSA gene can be introduced into the stem cell for transplantation.

In some embodiments, the present methods can be used to treat Wiskott-Aldrich syndrome (also called WAS or eczema-thrombocytopenia-immunodeficiency syndrome), an X-linked recessive disease caused by mutations in the WASp gene, and is characterized by abnormal immune system function (immune deficiency) and a reduced ability to form blood clots. Individuals with Wiskott-Aldrich syndrome have microthrombocytopenia, which is a decrease in the number and size of blood cell fragments involved in clotting (platelets). This platelet abnormality, which is typically present from birth, can lead to easy bruising, bloody diarrhea, or episodes of prolonged bleeding following minor trauma. Microthrombocytopenia can also lead to small areas of bleeding just under the surface of the skin, resulting in purplish spots called purpura or rashes of tiny red spots called petechiae. In some cases, the bleeding episodes can be life-threatening. Wiskott-Aldrich syndrome is also characterized by abnormal or nonfunctional immune system cells, e.g., shite blood cells. Changes in white blood cells lead to an increased risk of several immune and inflammatory disorders in people with Wiskott-Aldrich syndrome. These immune problems vary in severity and include an increased susceptibility to infection and eczema (an inflammatory skin disorder characterized by abnormal patches of red, irritated skin). People with Wiskott-Aldrich syndrome are at greater risk of developing autoimmune disorders, such as rheumatoid arthritis or hemolytic anemia, which occur when the immune system malfunctions and attacks the body's own tissues and organs. The chance of developing certain types of cancer, such as cancer of the immune system cells (lymphoma), is also increased in people with Wiskott-Aldrich syndrome. Using known gene editing methods, any one of more of the mutations in the WASp gene that cause Wiskott-Aldrich syndrome can be altered in the stem cell for use in the present methods. Additionally, or alternatively, a functional WASp gene can be introduced into the stem cell for transplantation.

In some embodiments, the present methods can be used to treat chronic granulomatous disease (also called CGD), caused by mutations in any one of five different genes which leads to a defect in the enzyme phagocyte NADPH oxidase, causing immunodeficiency. Individuals with chronic granulomatous disease may have recurrent bacterial and fungal infections. People with this condition may also have areas of inflammation (granulomas) in various tissues that can result in damage to those tissues. In these patients, the lungs are the most frequent area of infection; pneumonia is a common feature of this condition. Individuals with chronic granulomatous disease may develop a type of fungal pneumonia, called mulch pneumonitis, which causes fever and shortness of breath after exposure to decaying organic materials such as mulch, hay, or dead leaves. Exposure to these organic materials and the numerous fungi involved in their decomposition causes people with chronic granulomatous disease to develop fungal infections in their lungs. Other common areas of infection in people with chronic granulomatous disease include the skin, liver, and lymph nodes. Further, inflammation can occur in many different areas of the body in CGD patients. Most commonly, granulomas occur in the gastrointestinal tract and the genitourinary tract. Using known gene editing methods, any one of more of the mutations in the phagocyte NADPH oxidase gene that cause CGD can be altered in the stem cell for use in the present methods. Additionally, or alternatively, a functional phagocyte NADPH oxidase gene can be introduced into the stem cell for transplantation.

In certain embodiments, the present methods can be used to treat globoid cell leukodystrophy (also called GCL, galactosylceramide lipidosis or Krabbe disease), caused by mutations in the GALC gene. The GALC gene provides instructions for making galactosylceramidase, which breaks down certain fats (e.g., galactolipids). One galactolipid broken down by galactosylceramidase, called galactosylceramide, is an important component of myelin. Breakdown of galactosylceramide is part of the normal turnover of myelin that occurs throughout life. Another galactolipid, called psychosine, which is formed during the production of myelin, is toxic if not broken down by galactosylceramidase. Generally, GCL affects the growth of the nerve's protective myelin sheath and causes severe degeneration of motor skills. GCL is also characterized by abnormal cells in the brain called globoid cells, which are large cells that usually have more than one nucleus. Using known gene editing methods, any one of more of the mutations in the GALC gene that cause GCL can be altered in the stem cell for use in the present methods. Additionally, or alternatively, a functional GALC gene can be introduced into the stem cell for transplantation.

In some embodiments, the present methods can be used to treat mucopolysaccharidosis Type I (also called MPS Type I), a form of MPS (i.e. an inability to metabolize complex carbohydrates known as mucopolysaccharides into simpler molecules) caused by mutations in the iduronidase alpha-L gene (IDUA gene), leading to a deficiency of the enzyme alpha-L-iduronidase. The lack of IDUA enzyme activity leads to the accumulation of glycosaminoglycans (GAGs) within cells, specifically inside the lysosomes. Individuals with MPS1 may have a large head (macrocephaly), a buildup of fluid in the brain (hydrocephalus), heart valve abnormalities, an enlarged liver and spleen (hepatosplenomegaly), and a large tongue (macroglossia). Vocal cords can also enlarge, resulting in a deep, hoarse voice. The airway may become narrow in some people with MPS I, causing frequent upper respiratory infections and short pauses in breathing during sleep (sleep apnea). People with MPS I often develop clouding of the cornea, which can cause significant vision loss. Affected individuals may also have hearing loss and recurrent ear infections. Some individuals with MPS I have short stature and joint deformities (contractures) that affect mobility. Most people with the severe form of the disorder also have dysostosis multiplex, which refers to multiple skeletal abnormalities. Narrowing of the spinal canal (spinal stenosis) the neck can compress and damage the spinal cord. Using known gene editing methods, any one of more of the mutations in the IDUA gene that cause MPS Type I can be altered in the stem cell for use in the present methods. Additionally, or alternatively, a functional IDUA gene can be introduced into the stem cell for transplantation.

Additionally, or alternatively, the compositions and methods described herein can be used to treat a malignancy or proliferative disorder, such as a hematologic cancer, myeloproliferative disease, in particular the conjugates described herein. In the case of cancer treatment, the compositions and methods described herein may be administered to a patient so as to deplete a population of endogenous hematopoietic stem cells prior to hematopoietic stem cell transplantation therapy, in which case the transplanted cells can home to a niche created by the endogenous cell depletion step and establish productive hematopoiesis. This, in turn, can re-constitute a population of cells depleted during cancer cell eradication, such as during systemic chemotherapy. Exemplary hematological cancers that can be treated using the compositions and methods described herein include, without limitation, acute myeloid leukemia, acute lymphoid leukemia, chronic myeloid leukemia, chronic lymphoid leukemia, multiple myeloma, diffuse large B-cell lymphoma, and non-Hodgkin's lymphoma, as well as other cancerous conditions, including neuroblastoma.

Additional diseases that can be treated with the compositions and methods described herein include, without limitation, adenosine deaminase deficiency and severe combined immunodeficiency, hyper immunoglobulin M syndrome, Chediak-Higashi disease, hereditary lymphohistiocytosis, osteopetrosis, osteogenesis imperfecta, storage diseases, thalassemia major, systemic sclerosis, systemic lupus erythematosus, multiple sclerosis, and juvenile rheumatoid arthritis.

The compositions and methods described herein can be used to treat an autoimmune disease by depleting a population of endogenous hematopoietic stem cells prior to hematopoietic stem cell transplantation therapy, in which case the transplanted cells can home to a niche created by the endogenous cell depletion step and establish productive hematopoiesis. This, in turn, can re-constitute a population of cells depleted during autoimmune cell eradication.

Autoimmune diseases that can be treated using the compositions and methods described herein include, without limitation, psoriasis, psoriatic arthritis, Type 1 diabetes mellitus (Type 1 diabetes), rheumatoid arthritis (RA), human systemic lupus (SLE), multiple sclerosis (MS), inflammatory bowel disease (IBD), lymphocytic colitis, acute disseminated encephalomyelitis (ADEM), Addison's disease, alopecia universalis, ankylosing spondylitisis, antiphospholipid antibody syndrome (APS), aplastic anemia, autoimmune hemolytic anemia, autoimmune hepatitis, autoimmune inner ear disease (AIED), autoimmune lymphoproliferative syndrome (ALPS), autoimmune oophoritis, Balo disease, Behcet's disease, bullous pemphigoid, cardiomyopathy, Chagas' disease, chronic fatigue immune dysfunction syndrome (CFIDS), chronic inflammatory demyelinating polyneuropathy, Crohn's disease, cicatrical pemphigoid, coeliac sprue-dermatitis herpetiformis, cold agglutinin disease, CREST syndrome, Degos disease, discoid lupus, dysautonomia, endometriosis, essential mixed cryoglobulinemia, fibromyalgia-fibromyositis, Goodpasture's syndrome, Grave's disease, Guillain-Barre syndrome (GBS), Hashimoto's thyroiditis, Hidradenitis suppurativa, idiopathic and/or acute thrombocytopenic purpura, idiopathic pulmonary fibrosis, IgA neuropathy, interstitial cystitis, juvenile arthritis, Kawasaki's disease, lichen planus, Lyme disease, Meniere disease, mixed connective tissue disease (MCTD), myasthenia gravis, neuromyotonia, opsoclonus myoclonus syndrome (OMS), optic neuritis, Ord's thyroiditis, pemphigus vulgaris, pernicious anemia, polychondritis, polymyositis and dermatomyositis, primary biliary cirrhosis, polyarteritis nodosa, polyglandular syndromes, polymyalgia rheumatica, primary agammaglobulinemia, Raynaud phenomenon, Reiter's syndrome, rheumatic fever, sarcoidosis, scleroderma, Sjögren's syndrome, stiff person syndrome, Takayasu's arteritis, temporal arteritis (also known as “giant cell arteritis”), ulcerative colitis, collagenous colitis, uveitis, vasculitis, vitiligo, vulvodynia (“vulvar vestibulitis”), and Wegener's granulomatosis.

### C. Antibody Drug Conjugates (ADCs)

1. Antibodies

As described herein, the present methods include the use of ADCs that target specific molecules on hemoatopoietic stem cells and/or immune cells, including, e.g., CD2, CD5, CD7, CDwl2, CD13, CD15, CD19, CD21, CD22, CD29, CD30, CD33, CD34, CD36, CD38, CD40, CD41, CD42a, CD42b, CD42c, CD42d, CD43, CD45, CD45RA, CD45RB, CD45RC, CD45RO, CD47, CD48, CD49b, CD49d, CD49e, CD49f, CD50, CD53, CD55, CD64a, CD68, CD71, CD72, CD73, CD81, CD82, CD85A, CD85K, CD90, CD99, CD104, CD105, CD109, CD110, CD111, CD112, CD114, CD115, CD117, CD123, CD124, CD126, CD127, CD130, CD131, CD133, CD134, CD135, CD137, CD138, CD151, CD157, CD162, CD164, CD168, CD172a, CD173, CD174, CD175, CD175s, CD176, CD183, CD191, CD200, CD201, CD205, CD217, CD220, CD221, CD222, CD223, CD224, CD225, CD226, CD227, CD228, CD229, CD230, CD235a, CD235b, CD236, CD236R, CD238, CD240, CD242, CD243, CD252, CD277, CD292, CDw293, CD295, CD298, CD309, CD318, CD324, CD325, CD338, CD344, CD349, or CD350. In some embodiments, the ADC comprises an antibody or antigen-binding fragment thereof that specifically binds to one or more of the specific molecules on HSCs and/or immune cells. Methods of generating suitable antibodies for use in the present methods are readily available to those of skill in the art.

a. Anti-CD117 Antibodies

Antibodies, or antigen-binding fragments thereof, capable of binding CD117, such as GNNK+CD117, can be used as therapeutic agents alone or as conjugates (ADCs) to, for example, (i) treat cancers and autoimmune diseases characterized by CD117+ cells and (ii) promote the engraftment of transplanted genetically modified hematopoietic stem cells in a patient in need of transplant therapy. These therapeutic activities can be caused, for instance, by the binding of isolated anti-CD117 antibodies, antigen-binding fragments thereof, that bind to CD117 (e.g., GNNK+CD117) expressed on the surface of a cell, such as a cancer cell, autoimmune cell, or hematopoietic stem cell and subsequently inducing cell death. The depletion of endogenous hematopoietic stem cells can provide a niche toward which transplanted hematopoietic stem cells can home, and subsequently establish productive hematopoiesis. In this way, transplanted hematopoietic stem cells may successfully engraft in a patient, such as human patient suffering from a stem cell disorder described herein.

Antibodies and antigen-binding fragments capable of binding human CD117 (also referred to as c-Kit, mRNA NCBI Reference Sequence: NM_000222.2, Protein NCBI Reference Sequence: NP_000213.1), including those capable of binding GNNK+CD117, can be used in conjunction with the compositions and methods described herein in order to condition a patient for hematopoietic stem cell transplant therapy. Polymorphisms affecting the coding region or extracellular domain of CD117 in a significant percentage of the population are not currently well-known in non-oncology indications. There are at least four isoforms of CD117 that have been identified, with the potential of additional isoforms expressed in tumor cells. Two of the CD117 isoforms are located on the intracellular domain of the protein, and two are present in the external juxtamembrane region. The two extracellular isoforms, GNNK+ and GNNK−, differ in the presence (GNNK+) or absence (GNNK−) of a 4 amino acid sequence. These isoforms are reported to have the same affinity for the ligand (SCF), but ligand binding to the GNNK− isoform was reported to increase internalization and degradation. The GNNK+ isoform can be used as an immunogen in order to generate antibodies capable of binding CD117, as antibodies generated against this isoform will be inclusive of the GNNK+ and GNNK− proteins. The amino acid sequences of human CD117 isoforms 1 and 2 are described in SEQ ID Nos: 145 and 146, respectively. In certain embodiments, anti-human CD117 (hCD117) antibodies disclosed herein are able to bind to both isoform 1 and isoform 2 of human CD117.

As described below, a yeast library screen of human antibodies was performed to identify novel anti-CD117 antibodies, and fragments thereof, having diagnostic and therapeutic use. Antibody 54 (Ab54), Antibody 55 (Ab55), Antibody 56 (Ab56), Antibody 57 (Ab57), Antibody 58 (Ab58), Antibody 61 (Ab61), Antibody 66 (Ab66), Antibody 67 (Ab67), Antibody 68 (Ab68), and Antibody 69 (Ab69) were human antibodies that were identified in this screen. These antibodies cross react with human CD117 and rhesus CD117. Further, these antibodies disclosed herein are able to bind to both isoforms of human CD117, i.e., isoform 1 (SEQ ID NO: 145) and isoform 2 (SEQ ID NO: 146).

The amino acid sequences for the various binding regions of anti-CD117 antibodies Ab54, Ab55, Ab56, Ab57, Ab58, Ab61, Ab66, Ab67, Ab68, and Ab69 are described in Table 9. Included in the present disclosure are human anti-CD117 antibodies comprising the CDRs as set forth in Table 9, as well as human anti-CD117 antibodies comprising the variable regions set forth in Table 9.

In one embodiment, the present disclosure provides an anti-CD117 antibody, or antigen-binding fragment thereof, comprising binding regions, e.g., CDRs, variable regions, corresponding to those of Antibody 55. The heavy chain variable region (VH) amino acid sequence of Antibody 55 (i.e., Ab55) is set forth in SEQ ID NO: 19 (see Table 9). The VH CDR domain amino acid sequences of Antibody 55 are set forth in SEQ ID NO: 21 (VH CDR1); SEQ ID NO: 22 (VH CDR2), and SEQ ID NO: 23 (VH CDR3). The light chain variable region (VL) amino acid sequence of Antibody 55 is described in SEQ ID NO: 20 (see Table 9). The VL CDR domain amino acid sequences of Antibody 55 are set forth in SEQ ID NO: 24 (VL CDR1); SEQ ID NO: 25 (VL CDR2), and SEQ ID NO: 26 (VL CDR3). The heavy chain constant region of Antibody 55 is set forth in SEQ ID NO: 122. The light chain constant region of Antibody 55 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 21, 22, and 23, and a light chain variable region CDR set as set forth in SEQ ID Nos: 24, 25, and 26. In other embodiments, an anti-CD117 antibody, or antigen-binding portion thereof, comprises a variable light chain comprising the amino acid residues set forth in SEQ ID NO: 20, and a heavy chain variable region as set forth in SEQ ID NO: 19.

In one embodiment, the present disclosure provides an anti-CD117 antibody, or antigen-binding fragment thereof, comprising binding regions, e.g., CDRs, variable regions, corresponding to those of Antibody 54. The heavy chain variable region (VH) amino acid sequence of Antibody 54 (i.e., Ab54) is set forth in SEQ ID NO: 29 (see Table 9). The VH CDR domain amino acid sequences of Antibody 54 are set forth in SEQ ID NO: 31 (VH CDR1); SEQ ID NO: 32 (VH CDR2), and SEQ ID NO: 33 (VH CDR3). The light chain variable region (VL) amino acid sequence of Antibody 54 is described in SEQ ID NO: 30 (see Table 9). The VL CDR domain amino acid sequences of Antibody 54 are set forth in SEQ ID NO: 34 (VL CDR1); SEQ ID NO: 35 (VL CDR2), and SEQ ID NO: 36 (VL CDR3). The heavy chain constant region of Antibody 54 is set forth in SEQ ID NO: 122. The light chain constant region of Antibody 54 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 31, 32, and 33, and a light chain variable region CDR set as set forth in SEQ ID Nos: 34, 35, and 36. In other embodiments, an anti-CD117 antibody, or antigen-binding portion thereof, comprises a variable light chain comprising the amino acid residues set forth in SEQ ID NO: 30, and a heavy chain variable region as set forth in SEQ ID NO: 29.

In one embodiment, the present disclosure provides an anti-CD117 antibody, or antigen-binding fragment thereof, comprising binding regions, e.g., CDRs, variable regions, corresponding to those of Antibody 56. The heavy chain variable region (VH) amino acid sequence of Antibody 56 (i.e., Ab56) is set forth in SEQ ID NO: 39 (see Table 9). The VH CDR domain amino acid sequences of Antibody 56 are set forth in SEQ ID NO: 41 (VH CDR1); SEQ ID NO: 42 (VH CDR2), and SEQ ID NO: 43 (VH CDR3). The light chain variable region (VL) amino acid sequence of Antibody 56 is described in SEQ ID NO: 40 (see Table 9). The VL CDR domain amino acid sequences of Antibody 56 are set forth in SEQ ID NO: 44 (VL CDR1); SEQ ID NO: 45 (VL CDR2), and SEQ ID NO: 46 (VL CDR3). The heavy chain constant region of Antibody 56 is set forth in SEQ ID NO: 122. The light chain constant region of Antibody 56 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 41, 42, and 43, and a light chain variable region CDR set as set forth in SEQ ID Nos: 44, 45, and 46. In other embodiments, an anti-CD117 antibody, or antigen-binding portion thereof, comprises a variable light chain comprising the amino acid residues set forth in SEQ ID NO: 40, and a heavy chain variable region as set forth in SEQ ID NO: 39.

In one embodiment, the present disclosure provides an anti-CD117 antibody, or antigen-binding fragment thereof, comprising binding regions, e.g., CDRs, variable regions, corresponding to those of Antibody 57. The heavy chain variable region (VH) amino acid sequence of Antibody 57 (i.e., Ab57) is set forth in SEQ ID NO: 49 (see Table 9). The VH CDR domain amino acid sequences of Antibody 57 are set forth in SEQ ID NO: 51 (VH CDR1); SEQ ID NO: 52 (VH CDR2), and SEQ ID NO: 53 (VH CDR3). The light chain variable region (VL) amino acid sequence of Antibody 57 is described in SEQ ID NO: 50 (see Table 9). The VL CDR domain amino acid sequences of Antibody 57 are set forth in SEQ ID NO: 54 (VL CDR1); SEQ ID NO: 55 (VL CDR2), and SEQ ID NO: 56 (VL CDR3). The heavy chain constant region of Antibody 57 is set forth in SEQ ID NO: 122. The light chain constant region of Antibody 57 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 51, 52, and 53, and a light chain variable region CDR set as set forth in SEQ ID Nos: 54, 55, and 56. In other embodiments, an anti-CD117 antibody, or antigen-binding portion thereof, comprises a variable light chain comprising the amino acid residues set forth in SEQ ID NO: 50, and a heavy chain variable region as set forth in SEQ ID NO: 49.

In one embodiment, the present disclosure provides an anti-CD117 antibody, or antigen-binding fragment thereof, comprising binding regions, e.g., CDRs, variable regions, corresponding to those of Antibody 58. The heavy chain variable region (VH) amino acid sequence of Antibody 58 (i.e., Ab58) is set forth in SEQ ID NO: 59 (see Table 9). The VH CDR domain amino acid sequences of Antibody 58 are set forth in SEQ ID NO: 61 (VH CDR1); SEQ ID NO: 62 (VH CDR2), and SEQ ID NO: 63 (VH CDR3). The light chain variable region (VL) amino acid sequence of Antibody 58 is described in SEQ ID NO: 60 (see Table 9). The VL CDR domain amino acid sequences of Antibody 58 are set forth in SEQ ID NO: 64 (VL CDR1); SEQ ID NO: 65 (VL CDR2), and SEQ ID NO: 66 (VL CDR3). The heavy chain constant region of Antibody 58 is set forth in SEQ ID NO: 122. The light chain constant region of Antibody 58 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 61, 62, and 63, and a light chain variable region CDR set as set forth in SEQ ID Nos: 64, 65, and 66. In other embodiments, an anti-CD117 antibody, or antigen-binding portion thereof, comprises a variable light chain comprising the amino acid residues set forth in SEQ ID NO: 60, and a heavy chain variable region as set forth in SEQ ID NO: 59.

In one embodiment, the present disclosure provides an anti-CD117 antibody, or antigen-binding fragment thereof, comprising binding regions, e.g., CDRs, variable regions, corresponding to those of Antibody 61. The heavy chain variable region (VH) amino acid sequence of Antibody 61 (i.e., Ab61) is set forth in SEQ ID NO: 69 (see Table 9). The VH CDR domain amino acid sequences of Antibody 61 are set forth in SEQ ID NO: 71 (VH CDR1); SEQ ID NO: 72 (VH CDR2), and SEQ ID NO: 73 (VH CDR3). The light chain variable region (VL) amino acid sequence of Antibody 61 is described in SEQ ID NO: 70 (see Table 9). The VL CDR domain amino acid sequences of Antibody 61 are set forth in SEQ ID NO: 74 (VL CDR1); SEQ ID NO: 75 (VL CDR2), and SEQ ID NO: 76 (VL CDR3). The heavy chain constant region of Antibody 61 is set forth in SEQ ID NO: 122. The light chain constant region of Antibody 61 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 71, 72, and 73, and a light chain variable region CDR set as set forth in SEQ ID Nos: 74, 75, and 76. In other embodiments, an anti-CD117 antibody, or antigen-binding portion thereof, comprises a variable light chain comprising the amino acid residues set forth in SEQ ID NO: 70, and a heavy chain variable region as set forth in SEQ ID NO: 69.

In one embodiment, the present disclosure provides an anti-CD117 antibody, or antigen-binding fragment thereof, comprising binding regions, e.g., CDRs, variable regions, corresponding to those of Antibody 66. The heavy chain variable region (VH) amino acid sequence of Antibody 66 (i.e., Ab66) is set forth in SEQ ID NO: 79 (see Table 9). The VH CDR domain amino acid sequences of Antibody 66 are set forth in SEQ ID NO: 81 (VH CDR1); SEQ ID NO: 82 (VH CDR2), and SEQ ID NO: 83 (VH CDR3). The light chain variable region (VL) amino acid sequence of Antibody 66 is described in SEQ ID NO: 80 (see Table 9). The VL CDR domain amino acid sequences of Antibody 66 are set forth in SEQ ID NO: 84 (VL CDR1); SEQ ID NO: 85 (VL CDR2), and SEQ ID NO: 86 (VL CDR3). The heavy chain constant region of Antibody 66 is set forth in SEQ ID NO: 122. The light chain constant region of Antibody 66 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 81, 82, and 83, and a light chain variable region CDR set as set forth in SEQ ID Nos: 84, 85, and 86. In other embodiments, an anti-CD117 antibody, or antigen-binding portion thereof, comprises a variable light chain comprising the amino acid residues set forth in SEQ ID NO: 80, and a heavy chain variable region as set forth in SEQ ID NO: 79.

In one embodiment, the present disclosure provides an anti-CD117 antibody, or antigen-binding fragment thereof, comprising binding regions, e.g., CDRs, variable regions, corresponding to those of Antibody 67. The heavy chain variable region (VH) amino acid sequence of Antibody 67 is set forth in SEQ ID NO: 9 (see Table 9). The VH CDR domain amino acid sequences of Antibody 67 are set forth in SEQ ID NO 11 (VH CDR1); SEQ ID NO: 12 (VH CDR2), and SEQ ID NO: 13 (VH CDR3). The light chain variable region (VL) amino acid sequence of Antibody 67 is described in SEQ ID NO: 10 (see Table 9). The VL CDR domain amino acid sequences of Antibody 67 are set forth in SEQ ID NO 14 (VL CDR1); SEQ ID NO: 15 (VL CDR2), and SEQ ID NO: 16 (VL CDR3). The full length heavy chain (HC) of Antibody 67 is set forth in SEQ ID NO: 110, and the full length heavy chain constant region of Antibody 67 is set forth in SEQ ID NO: 122. The light chain (LC) of Antibody 67 is set forth in SEQ ID NO: 109. The light chain constant region of Antibody 67 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 11, 12, and 13, and a light chain variable region CDR set as set forth in SEQ ID Nos: 14, 15, and 16. In other embodiments, an anti-CD117 antibody, or antigen-binding portion thereof, comprises a variable heavy chain comprising the amino acid residues set forth in SEQ ID NO: 9, and a heavy chain variable region as set forth in SEQ ID NO: 10. In further embodiments, an anti-CD117 antibody comprises a heavy chain comprising SEQ ID NO: 110 and a light chain comprising SEQ ID NO: 109.

In one embodiment, the present disclosure provides an anti-CD117 antibody, or antigen-binding fragment thereof, comprising binding regions, e.g., CDRs, variable regions, corresponding to those of Antibody 68. The heavy chain variable region (VH) amino acid sequence of Antibody 68 (i.e., Ab68) is set forth in SEQ ID NO: 89 (see Table 9). The VH CDR domain amino acid sequences of Antibody 68 are set forth in SEQ ID NO: 91 (VH CDR1); SEQ ID NO: 92 (VH CDR2), and SEQ ID NO: 93 (VH CDR3). The light chain variable region (VL) amino acid sequence of Antibody 68 is described in SEQ ID NO: 90 (see Table 9). The VL CDR domain amino acid sequences of Antibody 68 are set forth in SEQ ID NO: 94 (VL CDR1); SEQ ID NO: 95 (VL CDR2), and SEQ ID NO: 96 (VL CDR3). The heavy chain constant region of Antibody 68 is set forth in SEQ ID NO: 122. The light chain constant region of Antibody 68 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 91, 92, and 93, and a light chain variable region CDR set as set forth in SEQ ID Nos: 94, 95, and 96. In other embodiments, an anti-CD117 antibody, or antigen-binding portion thereof, comprises a variable light chain comprising the amino acid residues set forth in SEQ ID NO: 90, and a heavy chain variable region as set forth in SEQ ID NO: 89.

In one embodiment, the present disclosure provides an anti-CD117 antibody, or antigen-binding fragment thereof, comprising binding regions, e.g., CDRs, variable regions, corresponding to those of Antibody 69. The heavy chain variable region (VH) amino acid sequence of Antibody 69 (i.e., Ab69) is set forth in SEQ ID NO: 99 (see Table 9). The VH CDR domain amino acid sequences of Antibody 69 are set forth in SEQ ID NO: 101 (VH CDR1); SEQ ID NO: 102 (VH CDR2), and SEQ ID NO: 103 (VH CDR3). The light chain variable region (VL) amino acid sequence of Antibody 69 is described in SEQ ID NO: 100 (see Table 9). The VL CDR domain amino acid sequences of Antibody 69 are set forth in SEQ ID NO: 104 (VL CDR1); SEQ ID NO: 105 (VL CDR2), and SEQ ID NO: 106 (VL CDR3). The heavy chain constant region of Antibody 69 is set forth in SEQ ID NO: 122. The light chain constant region of Antibody 69 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 101, 102, and 103, and a light chain variable region CDR set as set forth in SEQ ID Nos: 104, 105, and 106. In other embodiments, an anti-CD117 antibody, or antigen-binding portion thereof, comprises a variable light chain comprising the amino acid residues set forth in SEQ ID NO: 100, and a heavy chain variable region as set forth in SEQ ID NO: 99.

Further, the amino acid sequences for the various binding regions of the anti-CD117 antibodies Ab77, Ab79, Ab81, Ab85, Ab86, Ab87, Ab88, and Ab89 are described in Table 9.

In one embodiment, the present disclosure provides an anti-CD117 antibody, or antigen-binding fragment thereof, comprising binding regions, e.g., CDRs, variable regions, corresponding to those of Antibody 77. The heavy chain variable region (VH) amino acid sequence of Antibody 77 (i.e., Ab77) is set forth in SEQ ID NO: 147 (see Table 9). The VH CDR domain amino acid sequences of Antibody 77 are set forth in SEQ ID NO: 263 (VH CDR1); SEQ ID NO: 2 (VH CDR2), and SEQ ID NO: 3 (VH CDR3). The light chain variable region (VL) amino acid sequence of Antibody 77 is described in SEQ ID NO: 231 (see Table 9). The VL CDR domain amino acid sequences of Antibody 77 are set forth in SEQ ID NO: 264 (VL CDR1); SEQ ID NO: 265 (VL CDR2), and SEQ ID NO: 266 (VL CDR3). The heavy chain constant region of Antibody 77 is set forth in SEQ ID NO: 269. The light chain constant region of Antibody 77 is set forth in SEQ ID NO: 283. Thus, in certain embodiments, an anti-CD117 antibody, or antigen-binding portion thereof, comprises a variable heavy chain CDR set (CDR1, CDR2, and CDR3) as set forth in SEQ ID Nos: 263, 2, and 3, and a light chain variable region CDR set as set forth in SEQ ID Nos: 264, 265, and 266. In other embodiments, an anti-CD117 antibody, or antigen-binding portion thereof, comprises a variable light chain comprising the amino acid residues set forth in SEQ ID NO: 231, and a heavy chain variable region as set forth in SEQ ID NO: 147.

In one embodiment, the present disclosure provides an anti-CD117 antibody, or antigen-binding fragment thereof, comprising binding regions, e.g., CDRs, variable regions, corresponding to those of Antibody 79. The heavy chain variable region (VH) amino acid sequence of Antibody 79 (i.e., Ab79) is set forth in SEQ ID NO: 147 (see Table 9). The VH CDR domain amino acid sequences of Antibody 79 are set forth in SEQ ID NO: 263 (VH CDR1); SEQ ID NO: 2 (VH CDR2), and SEQ ID NO: 3 (VH CDR3). The light chain variable region (VL) amino acid sequence of Antibody 79 is described in SEQ ID NO: 233 (see Table 9). The VL CDR domain amino acid sequences of Antibody 79 are set forth in SEQ ID NO: 267 (VL CDR1); SEQ ID NO: 265 (VL CDR2), and SEQ ID NO: 266 (VL CDR3). The heavy chain constant region of Antibody 79 is set forth in SEQ ID NO: 269. The light chain constant region of Antibody 79 is set forth in SEQ ID NO: 283. Thus, in certain embodiments, an anti-CD117 antibody, or antigen-binding portion thereof, comprises a variable heavy chain CDR set (CDR1, CDR2, and CDR3) as set forth in SEQ ID Nos: 263, 2, and 3, and a light chain variable region CDR set as set forth in SEQ ID Nos: 267, 265, and 266. In other embodiments, an anti-CD117 antibody, or antigen-binding portion thereof, comprises a variable light chain comprising the amino acid residues set forth in SEQ ID NO: 233, and a heavy chain variable region as set forth in SEQ ID NO: 147.

In one embodiment, the present disclosure provides an anti-CD117 antibody, or antigen-binding fragment thereof, comprising binding regions, e.g., CDRs, variable regions, corresponding to those of Antibody 81. The heavy chain variable region (VH) amino acid sequence of Antibody 81 (i.e., Ab81) is set forth in SEQ ID NO: 147 (see Table 9). The VH CDR domain amino acid sequences of Antibody 81 are set forth in SEQ ID NO: 263 (VH CDR1); SEQ ID NO: 2 (VH CDR2), and SEQ ID NO: 3 (VH CDR3). The light chain variable region (VL) amino acid sequence of Antibody 81 is described in SEQ ID NO: 235 (see Table 9). The VL CDR domain amino acid sequences of Antibody 81 are set forth in SEQ ID NO: 264 (VL CDR1); SEQ ID NO: 268 (VL CDR2), and SEQ ID NO: 266 (VL CDR3). The heavy chain constant region of Antibody 81 is set forth in SEQ ID NO: 269. The light chain constant region of Antibody 81 is set forth in SEQ ID NO: 283. Thus, in certain embodiments, an anti-CD117 antibody, or antigen-binding portion thereof, comprises a variable heavy chain CDR set (CDR1, CDR2, and CDR3) as set forth in SEQ ID Nos: 263, 2, and 3, and a light chain variable region CDR set as set forth in SEQ ID Nos: 264, 268, and 266. In other embodiments, an anti-CD117 antibody, or antigen-binding portion thereof, comprises a variable light chain comprising the amino acid residues set forth in SEQ ID NO: 235, and a heavy chain variable region as set forth in SEQ ID NO: 147.

In one embodiment, the present disclosure provides an anti-CD117 antibody, or antigen-binding fragment thereof, comprising binding regions, e.g., CDRs, variable regions, corresponding to those of Antibody 85. The heavy chain variable region (VH) amino acid sequence of Antibody 85 (i.e., Ab86) is set forth in SEQ ID NO: 243 (see Table 9). The VH CDR domain amino acid sequences of Antibody 85 are set forth in SEQ ID NO: 245 (VH CDR1); SEQ ID NO: 246 (VH CDR2), and SEQ ID NO: 247 (VH CDR3). The light chain variable region (VL) amino acid sequence of Antibody 85 is described in SEQ ID NO: 242 (see Table 9). The VL CDR domain amino acid sequences of Antibody 85 are set forth in SEQ ID NO: 248 (VL CDR1); SEQ ID NO: 249 (VL CDR2), and SEQ ID NO: 250 (VL CDR3). The heavy chain constant region of Antibody 85 is set forth in SEQ ID NO: 269. The light chain constant region of Antibody 85 is set forth in SEQ ID NO: 283. Thus, in certain embodiments, an anti-CD117 antibody, or antigen-binding portion thereof, comprises a variable heavy chain CDR set (CDR1, CDR2, and CDR3) as set forth in SEQ ID Nos: 245, 246, and 247, and a light chain variable region CDR set as set forth in SEQ ID Nos: 248, 249, and 250. In other embodiments, an anti-CD117 antibody, or antigen-binding portion thereof, comprises a variable light chain comprising the amino acid residues set forth in SEQ ID NO: 244, and a heavy chain variable region as set forth in SEQ ID NO: 243.

In one embodiment, the present disclosure provides an anti-CD117 antibody, or antigen-binding fragment thereof, comprising binding regions, e.g., CDRs, variable regions, corresponding to those of Antibody 86. The heavy chain variable region (VH) amino acid sequence of Antibody 86 (i.e., Ab86) is set forth in SEQ ID NO: 251 (see Table 9). The VH CDR domain amino acid sequences of Antibody 86 are set forth in SEQ ID NO: 245 (VH CDR1); SEQ ID NO: 253 (VH CDR2), and SEQ ID NO: 3 (VH CDR3). The light chain variable region (VL) amino acid sequence of Antibody 86 is described in SEQ ID NO: 252 (see Table 9). The VL CDR domain amino acid sequences of Antibody 86 are set forth in SEQ ID NO: 254 (VL CDR1); SEQ ID NO: 249 (VL CDR2), and SEQ ID NO: 255 (VL CDR3). The heavy chain constant region of Antibody 86 is set forth in SEQ ID NO: 269. The light chain constant region of Antibody 86 is set forth in SEQ ID NO: 283. Thus, in certain embodiments, an anti-CD117 antibody, or antigen-binding portion thereof, comprises a variable heavy chain CDR set (CDR1, CDR2, and CDR3) as set forth in SEQ ID Nos: 245, 253, and 3, and a light chain variable region CDR set as set forth in SEQ ID Nos: 254, 249, and 255. In other embodiments, an anti-CD117 antibody, or antigen-binding portion thereof, comprises a variable light chain comprising the amino acid residues set forth in SEQ ID NO: 252, and a heavy chain variable region as set forth in SEQ ID NO: 251.

In one embodiment, the present disclosure provides an anti-CD117 antibody, or antigen-binding fragment thereof, comprising binding regions, e.g., CDRs, variable regions, corresponding to those of Antibody 87. The heavy chain variable region (VH) amino acid sequence of Antibody 87 (i.e., Ab87) is set forth in SEQ ID NO: 243 (see Table 9). The VH CDR domain amino acid sequences of Antibody 87 are set forth in SEQ ID NO: 245 (VH CDR1); SEQ ID NO: 246 (VH CDR2), and SEQ ID NO: 247 (VH CDR3). The light chain variable region (VL) amino acid sequence of Antibody 87 is described in SEQ ID NO: 256 (see Table 9). The VL CDR domain amino acid sequences of Antibody 87 are set forth in SEQ ID NO: 257 (VL CDR1); SEQ ID NO: 5 (VL CDR2), and SEQ ID NO: 255 (VL CDR3). The heavy chain constant region of Antibody 87 is set forth in SEQ ID NO: 269. The light chain constant region of Antibody 87 is set forth in SEQ ID NO: 283. Thus, in certain embodiments, an anti-CD117 antibody, or antigen-binding portion thereof, comprises a variable heavy chain CDR set (CDR1, CDR2, and CDR3) as set forth in SEQ ID Nos: 245, 246, and 247, and a light chain variable region CDR set as set forth in SEQ ID Nos: 257, 5, and 255. In other embodiments, an anti-CD117 antibody, or antigen-binding portion thereof, comprises a variable light chain comprising the amino acid residues set forth in SEQ ID NO: 256, and a heavy chain variable region as set forth in SEQ ID NO: 243.

In one embodiment, the present disclosure provides an anti-CD117 antibody, or antigen-binding fragment thereof, comprising binding regions, e.g., CDRs, variable regions, corresponding to those of Antibody 88. The heavy chain variable region (VH) amino acid sequence of Antibody 88 (i.e., Ab88) is set forth in SEQ ID NO: 258 (see Table 9). The VH CDR domain amino acid sequences of Antibody 88 are set forth in SEQ ID NO: 245 (VH CDR1); SEQ ID NO: 259 (VH CDR2), and SEQ ID NO: 3 (VH CDR3). The light chain variable region (VL) amino acid sequence of Antibody 88 is described in SEQ ID NO: 256 (see Table 9). The VL CDR domain amino acid sequences of Antibody 88 are set forth in SEQ ID NO: 257 (VL CDR1); SEQ ID NO: 5 (VL CDR2), and SEQ ID NO: 255 (VL CDR3). The heavy chain constant region of Antibody 88 is set forth in SEQ ID NO: 269. The light chain constant region of Antibody 88 is set forth in SEQ ID NO: 283. Thus, in certain embodiments, an anti-CD117 antibody, or antigen-binding portion thereof, comprises a variable heavy chain CDR set (CDR1, CDR2, and CDR3) as set forth in SEQ ID Nos: 245, 259, and 3, and a light chain variable region CDR set as set forth in SEQ ID Nos: 257, 5, and 255. In other embodiments, an anti-CD117 antibody, or antigen-binding portion thereof, comprises a variable light chain comprising the amino acid residues set forth in SEQ ID NO: 256, and a heavy chain variable region as set forth in SEQ ID NO: 258.

In one embodiment, the present disclosure provides an anti-CD117 antibody, or antigen-binding fragment thereof, comprising binding regions, e.g., CDRs, variable regions, corresponding to those of Antibody 89. The heavy chain variable region (VH) amino acid sequence of Antibody 89 (i.e., Ab89) is set forth in SEQ ID NO: 260 (see Table 9). The VH CDR domain amino acid sequences of Antibody 89 are set forth in SEQ ID NO: 245 (VH CDR1); SEQ ID NO: 2 (VH CDR2), and SEQ ID NO: 3 (VH CDR3). The light chain variable region (VL) amino acid sequence of Antibody 89 is described in SEQ ID NO: 252 (see Table 9). The VL CDR domain amino acid sequences of Antibody 89 are set forth in SEQ ID NO: 254 (VL CDR1); SEQ ID NO: 249 (VL CDR2), and SEQ ID NO: 255 (VL CDR3). The heavy chain constant region of Antibody 89 is set forth in SEQ ID NO: 269. The light chain constant region of Antibody 89 is set forth in SEQ ID NO: 283. Thus, in certain embodiments, an anti-CD117 antibody, or antigen-binding portion thereof, comprises a variable heavy chain CDR set (CDR1, CDR2, and CDR3) as set forth in SEQ ID Nos: 245, 2, and 3, and a light chain variable region CDR set as set forth in SEQ ID Nos: 254, 249, and 255. In other embodiments, an anti-CD117 antibody, or antigen-binding portion thereof, comprises a variable light chain comprising the amino acid residues set forth in SEQ ID NO: 252, and a heavy chain variable region as set forth in SEQ ID NO: 260.

In one embodiment, the present disclosure provides an anti-CD117 antibody, or antigen-binding fragment thereof, comprising binding regions, e.g., CDRs, variable regions, corresponding to those of Antibody 249. The heavy chain variable region (VH) amino acid sequence of Antibody 249 (i.e., Ab249) is set forth in SEQ ID NO: 238 (see Table 9). The VH CDR domain amino acid sequences of Antibody 249 are set forth in SEQ ID NO: 286 (VH CDR1); SEQ ID NO: 2 (VH CDR2), and SEQ ID NO: 287 (VH CDR3). The light chain variable region (VL) amino acid sequence of Antibody 249 is described in SEQ ID NO: 242 (see Table 9). The VL CDR domain amino acid sequences of Antibody 249 are set forth in SEQ ID NO: 288 (VL CDR1); SEQ ID NO: 249 (VL CDR2), and SEQ ID NO: 289 (VL CDR3). The heavy chain constant region of Antibody 249 is set forth in SEQ ID NO: 269. The light chain constant region of Antibody 249 is set forth in SEQ ID NO: 283. Thus, in certain embodiments, an anti-CD117 antibody, or antigen-binding portion thereof, comprises a variable heavy chain CDR set (CDR1, CDR2, and CDR3) as set forth in SEQ ID Nos: 286, 2, and 287, and a light chain variable region CDR set as set forth in SEQ ID Nos: 288, 249, and 289. In other embodiments, an anti-CD117 antibody, or antigen-binding portion thereof, comprises a variable light chain comprising the amino acid residues set forth in SEQ ID NO: 242, and a heavy chain variable region as set forth in SEQ ID NO: 238.

Further, included in the disclosure is anti-CD117 antibody drug conjugates comprising binding regions (heavy and light chain CDRs or variable regions) as set forth in SEQ ID Nos: 147 to 168. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 148. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 149. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 150. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 151. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 152. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 153. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 154. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 155. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 156. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 157. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 158. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 159. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 160. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 161. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 162. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 163. In one embodiment, the anti-CD117 antibody, or antigen binding portion thereof, comprises a heavy chain variable region as set forth in the amino acid sequence of SEQ ID NO: 164, and a light chain variable region as set forth in the amino acid sequence of SEQ ID NO: 165. In one embodiment, the anti-CD117 antibody, or antigen binding portion thereof, comprises a heavy chain variable region as set forth in the amino acid sequence of SEQ ID NO: 166, and a light chain variable region as set forth in the amino acid sequence of SEQ ID NO: 167. In one embodiment, the anti-CD117 antibody, or antigen binding portion thereof, comprises a heavy chain variable region as set forth in the amino acid sequence of SEQ ID NO: 168, and a light chain variable region as set forth in the amino acid sequence of SEQ ID NO: 169. In one embodiment, the anti-CD117 antibody, or antigen binding portion thereof, comprises a heavy chain variable region as set forth in the amino acid sequence of SEQ ID NO: 170, and a light chain variable region as set forth in the amino acid sequence of SEQ ID NO: 171. In one embodiment, the anti-CD117 antibody, or antigen binding portion thereof, comprises a heavy chain variable region as set forth in the amino acid sequence of SEQ ID NO: 172, and a light chain variable region as set forth in the amino acid sequence of SEQ ID NO: 173. In one embodiment, the anti-CD117 antibody, or antigen binding portion thereof, comprises a heavy chain variable region as set forth in the amino acid sequence of SEQ ID NO: 174, and a light chain variable region as set forth in the amino acid sequence of SEQ ID NO: 175. In one embodiment, the anti-CD117 antibody, or antigen binding portion thereof, comprises a heavy chain variable region as set forth in the amino acid sequence of SEQ ID NO: 176, and a light chain variable region as set forth in the amino acid sequence of SEQ ID NO: 177. In one embodiment, the anti-CD117 antibody, or antigen binding portion thereof, comprises a heavy chain variable region as set forth in the amino acid sequence of SEQ ID NO: 178, and a light chain variable region as set forth in the amino acid sequence of SEQ ID NO: 179. In one embodiment, the anti-CD117 antibody, or antigen binding portion thereof, comprises a heavy chain variable region as set forth in the amino acid sequence of SEQ ID NO: 180, and a light chain variable region as set forth in the amino acid sequence of SEQ ID NO: 181. In one embodiment, the anti-CD117 antibody, or antigen binding portion thereof, comprises a heavy chain variable region as set forth in the amino acid sequence of SEQ ID NO: 172, and a light chain variable region as set forth in the amino acid sequence of SEQ ID NO: 182. In one embodiment, the anti-CD117 antibody, or antigen binding portion thereof, comprises a heavy chain variable region as set forth in the amino acid sequence of SEQ ID NO: 183, and a light chain variable region as set forth in the amino acid sequence of SEQ ID NO: 184. In one embodiment, the anti-CD117 antibody, or antigen binding portion thereof, comprises a heavy chain variable region as set forth in the amino acid sequence of SEQ ID NO: 185, and a light chain variable region as set forth in the amino acid sequence of SEQ ID NO: 186. In one embodiment, the anti-CD117 antibody, or antigen binding portion thereof, comprises a heavy chain variable region as set forth in the amino acid sequence of SEQ ID NO: 187, and a light chain variable region as set forth in the amino acid sequence of SEQ ID NO: 188. In one embodiment, the anti-CD117 antibody, or antigen binding portion thereof, comprises a heavy chain variable region as set forth in the amino acid sequence of SEQ ID NO: 189, and a light chain variable region as set forth in the amino acid sequence of SEQ ID NO: 190. In one embodiment, the anti-CD117 antibody, or antigen binding portion thereof, comprises a heavy chain variable region as set forth in the amino acid sequence of SEQ ID NO: 191, and a light chain variable region as set forth in the amino acid sequence of SEQ ID NO: 192. In one embodiment, the anti-CD117 antibody, or antigen binding portion thereof, comprises a heavy chain variable region as set forth in the amino acid sequence of SEQ ID NO: 193, and a light chain variable region as set forth in the amino acid sequence of SEQ ID NO: 194. In one embodiment, the anti-CD117 antibody, or antigen binding portion thereof, comprises a heavy chain variable region as set forth in the amino acid sequence of SEQ ID NO: 195, and a light chain variable region as set forth in the amino acid sequence of SEQ ID NO: 196. In one embodiment, the anti-CD117 antibody, or antigen binding portion thereof, comprises a heavy chain variable region as set forth in the amino acid sequence of SEQ ID NO: 197, and a light chain variable region as set forth in the amino acid sequence of SEQ ID NO: 198. In one embodiment, the anti-CD117 antibody, or antigen binding portion thereof, comprises a heavy chain variable region as set forth in the amino acid sequence of SEQ ID NO: 199, and a light chain variable region as set forth in the amino acid sequence of SEQ ID NO: 200. In one embodiment, the anti-CD117 antibody, or antigen binding portion thereof, comprises a heavy chain variable region as set forth in the amino acid sequence of SEQ ID NO: 201, and a light chain variable region as set forth in the amino acid sequence of SEQ ID NO: 190. In one embodiment, the anti-CD117 antibody, or antigen binding portion thereof, comprises a heavy chain variable region as set forth in the amino acid sequence of SEQ ID NO: 202, and a light chain variable region as set forth in the amino acid sequence of SEQ ID NO: 203. In one embodiment, the anti-CD117 antibody, or antigen binding portion thereof, comprises a heavy chain variable region as set forth in the amino acid sequence of SEQ ID NO: 204, and a light chain variable region as set forth in the amino acid sequence of SEQ ID NO: 205. In one embodiment, the anti-CD117 antibody, or antigen binding portion thereof, comprises a heavy chain variable region as set forth in the amino acid sequence of SEQ ID NO: 206, and a light chain variable region as set forth in the amino acid sequence of SEQ ID NO: 207. In one embodiment, the anti-CD117 antibody, or antigen binding portion thereof, comprises a heavy chain variable region as set forth in the amino acid sequence of SEQ ID NO: 208, and a light chain variable region as set forth in the amino acid sequence of SEQ ID NO: 209. In one embodiment, the anti-CD117 antibody, or antigen binding portion thereof, comprises a heavy chain variable region as set forth in the amino acid sequence of SEQ ID NO: 210, and a light chain variable region as set forth in the amino acid sequence of SEQ ID NO: 211. In one embodiment, the anti-CD117 antibody, or antigen binding portion thereof, comprises a heavy chain variable region as set forth in the amino acid sequence of SEQ ID NO: 212, and a light chain variable region as set forth in the amino acid sequence of SEQ ID NO: 213. In one embodiment, the anti-CD117 antibody, or antigen binding portion thereof, comprises a heavy chain variable region as set forth in the amino acid sequence of SEQ ID NO: 214, and a light chain variable region as set forth in the amino acid sequence of SEQ ID NO: 215. In one embodiment, the anti-CD117 antibody, or antigen binding portion thereof, comprises a heavy chain variable region as set forth in the amino acid sequence of SEQ ID NO: 216, and a light chain variable region as set forth in the amino acid sequence of SEQ ID NO: 217. In one embodiment, the anti-CD117 antibody, or antigen binding portion thereof, comprises a heavy chain variable region as set forth in the amino acid sequence of SEQ ID NO: 218, and a light chain variable region as set forth in the amino acid sequence of SEQ ID NO: 219. In one embodiment, the anti-CD117 antibody, or antigen binding portion thereof, comprises a heavy chain variable region as set forth in the amino acid sequence of SEQ ID NO: 220, and a light chain variable region as set forth in the amino acid sequence of SEQ ID NO: 221. In one embodiment, the anti-CD117 antibody, or antigen binding portion thereof, comprises a heavy chain variable region as set forth in the amino acid sequence of SEQ ID NO: 222, and a light chain variable region as set forth in the amino acid sequence of SEQ ID NO: 223. In one embodiment, the anti-CD117 antibody, or antigen binding portion thereof, comprises a heavy chain variable region as set forth in the amino acid sequence of SEQ ID NO: 224, and a light chain variable region as set forth in the amino acid sequence of SEQ ID NO: 225. In one embodiment, the anti-CD117 antibody, or antigen binding portion thereof, comprises a heavy chain variable region as set forth in the amino acid sequence of SEQ ID NO: 226, and a light chain variable region as set forth in the amino acid sequence of SEQ ID NO: 227. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 228. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 229. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 230. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 231. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 232. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 233. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 234. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 235. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 236.

In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 237. In one embodiment, the anti-CD117 antibody, or antigen binding portion thereof, comprises a heavy chain variable region as set forth in the amino acid sequence of SEQ ID NO: 243, and a light chain variable region as set forth in the amino acid sequence of SEQ ID NO: 244. In one embodiment, the anti-CD117 antibody, or antigen binding portion thereof, comprises a heavy chain variable region as set forth in the amino acid sequence of SEQ ID NO: 251, and a light chain variable region as set forth in the amino acid sequence of SEQ ID NO: 252. In one embodiment, the anti-CD117 antibody, or antigen binding portion thereof, comprises a heavy chain variable region as set forth in the amino acid sequence of SEQ ID NO: 243, and a light chain variable region as set forth in the amino acid sequence of SEQ ID NO: 256. In one embodiment, the anti-CD117 antibody, or antigen binding portion thereof, comprises a heavy chain variable region as set forth in the amino acid sequence of SEQ ID NO: 258, and a light chain variable region as set forth in the amino acid sequence of SEQ ID NO: 256. In one embodiment, the anti-CD117 antibody, or antigen binding portion thereof, comprises a heavy chain variable region as set forth in the amino acid sequence of SEQ ID NO: 260, and a light chain variable region as set forth in the amino acid sequence of SEQ ID NO: 252. In one embodiment, the anti-CD117 antibody, or antigen binding portion thereof, comprises a heavy chain variable region as set forth in the amino acid sequence of SEQ ID NO: 238, and a light chain variable region as set forth in the amino acid sequence of SEQ ID NO: 239. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 239. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 240. In one embodiment, the anti-CD117 antibody, or antigen binding portion thereof, comprises a heavy chain variable region as set forth in the amino acid sequence of SEQ ID NO: 238, and a light chain variable region as set forth in the amino acid sequence of SEQ ID NO: 241. In one embodiment, the anti-CD117 antibody, or antigen binding portion thereof, comprises a heavy chain variable region as set forth in the amino acid sequence of SEQ ID NO: 238, and a light chain variable region as set forth in the amino acid sequence of SEQ ID NO: 242.

Certain of the anti-CD117 antibodies described herein are neutral antibodies, in that the antibodies do not substantially inhibit CD117 activity on a CD117 expressing cell. Neutral antibodies can be identified using, for example, an in in vitro stem cell factor (SCF)-dependent cell proliferation assay (see, e.g., Example 11 described herein). In an SCF dependent cell proliferation assay, a neutral CD117 antibody will not kill CD34+ cells that are dependent on SCF to divide, as a neutral antibody will not block SCF from binding to CD117 such as to inhibit CD117 activity.

Neutral antibodies can be used for diagnostic purposes, given their ability to specifically bind to human CD117, but are also effective for killing CD117 expressing cells when conjugated to a cytotoxin, such as those described herein. Typically, antibodies used in conjugates have agonistic or antagonistic activity that is unique to the antibody. Described herein, however, is a unique approach to conjugates, especially in the context wherein the conjugate is being used as a conditioning agent prior to a stem cell transplantation. While antagonistic antibodies alone or in combination with a cytotoxin as a conjugate can be effective given the killing ability of the antibody alone in addition to the cytotoxin, conditioning with a conjugate comprising a neutral anti-CD117 antibody presents an alternative strategy where the activity of the antibody is secondary to the effect of the cytotoxin, but the internalizing and affinity characteristics, e.g., dissociation rate, of the antibody are important for effective delivery of the cytotoxin.

Examples of neutral anti-CD117 antibodies include Ab58, Ab61, Ab66, Ab67, Ab68, and Ab69. A comparison of the amino acid sequences of the CDRs of neutral, anti-CD117 antibody CDRs reveals consensus sequences among two groups of neutral antibodies identified. A comparison of the heavy and light chain variable regions of Ab58 and Ab61 is described in PCT/US2018/057172, incorporated by reference in its entirety. Ab58 and Ab61 share the same light chain CDRs and HC CDR3, with slight variations in the HC CDR1 and HC CDR2. Consensus sequences for the HC CDR1 and CDR2 are described in SEQ ID Nos: 133 and 134. Ab66, Ab67, Ab68, and Ab69 are also neutral antibodies. The heavy and light chain variable regions of these antibodies are described in PCT/US2018/057172, incorporated by reference in its entirety. While Ab66, Ab67, Ab68, and Ab69 share the same light chain CDRs and the same HC CDR3, these antibodies have variability within their HC CDR1 and HC CDR2 regions. Consensus sequences for these antibodies in the HC CDR1 and HC CDR2 regions are provided in SEQ ID Nos: 139 and 140, respectively.

Antagonist antibodies are also provided herein, including Ab54, Ab55, Ab56, and Ab57. A comparison of the variable heavy and light chain amino acid sequences for these antibodies is provided in PCT/US2018057172, incorporated by reference in its entirety. While Ab54, Ab55, Ab56, and Ab57 share the same light chain CDRs and the same HC CDR3, these antibodies have variability within their HC CDR1 and HC CDR2 regions. Consensus sequences for these antibodies in the HC CDR1 and HC CDR2 regions are provided in SEQ ID Nos: 127 and 128, respectively.

The anti-CD117 antibodies described herein can be in the form of full-length antibodies, bispecific antibodies, dual variable domain antibodies, multiple chain or single chain antibodies, and/or binding fragments that specifically bind human CD117, including but not limited to Fab, Fab′, (Fab′)2, Fv), scFv (single chain Fv), surrobodies (including surrogate light chain construct), single domain antibodies, camelized antibodies and the like. They also can be of, or derived from, any isotype, including, for example, IgA (e.g., IgA1 or IgA2), IgD, IgE, IgG (e.g. IgG1, IgG2, IgG3 or IgG4), or IgM. In some embodiments, the anti-CD117 antibody is an IgG (e.g. IgG1, IgG2, IgG3 or IgG4).

Antibodies for use in conjunction with the methods described herein include variants of those antibodies described above, such as antibody fragments that contain or lack an Fc domain, as well as humanized variants of non-human antibodies described herein and antibody-like protein scaffolds (e.g., ¹⁰Fn3 domains) containing one or more, or all, of the CDRs or equivalent regions thereof of an antibody, or antibody fragment, described herein. Exemplary antigen-binding fragments of the foregoing antibodies include a dual-variable immunoglobulin domain, a single-chain Fv molecule (scFv), a diabody, a triabody, a nanobody, an antibody-like protein scaffold, a Fv fragment, a Fab fragment, a F(ab′)₂ molecule, and a tandem di-scFv, among others.

In one embodiment, anti-CD117 antibodies comprising one or more radiolabeled amino acids are provided. A radiolabeled anti-CD117 antibody may be used for both diagnostic and therapeutic purposes (conjugation to radiolabeled molecules is another possible feature). Nonlimiting examples of labels for polypeptides include, but are not limited to 3H, 14C, 15N, 35S, 90Y, 99Tc, and 1251, 1311, and 186Re. Methods for preparing radiolabeled amino acids and related peptide derivatives are known in the art (see for instance Junghans et al., in Cancer Chemotherapy and Biotherapy 655-686 (2d edition, Chafner and Longo, eds., Lippincott Raven (1996)) and U.S. Pat. Nos. 4,681,581, 4,735,210, 5,101,827, 5,102,990 (U.S. RE35,500), 5,648,471 and 5,697,902. For example, a radioisotope may be conjugated by a chloramine T method.

The anti-CD117 antibodies or binding fragments described herein may also include modifications and/or mutations that alter the properties of the antibodies and/or fragments, such as those that increase half-life, increase or decrease ADCC, etc., as is known in the art.

In one embodiment, the anti-CD117 antibody, or binding fragment thereof, comprises a variant (or modified) Fc region, wherein said variant Fc region comprises at least one amino acid modification relative to a wild-type Fc region, such that said molecule has an altered affinity for an FcgammaR. Certain amino acid positions within the Fc region are known through crystallography studies to make a direct contact with FcγR. Specifically, amino acids 234-239 (hinge region), amino acids 265-269 (B/C loop), amino acids 297-299 (C′/E loop), and amino acids 327-332 (F/G) loop. (see Sondermann et al., 2000 Nature, 406: 267-273). In some embodiments, the anti-CD117 antibodies described herein may comprise variant Fc regions comprising modification of at least one residue that makes a direct contact with an Fcγ R based on structural and crystallographic analysis. In one embodiment, the Fc region of the anti-CD117 antibody (or fragment thereof) comprises an amino acid substitution at amino acid 265 according to the EU index as in Kabat et al., Sequences of Proteins of Immunological Interest, 5th Ed. Public Health Service, NH1, MD (1991), expressly incorporated herein by references. The “EU index as in Kabat” refers to the numbering of the human IgG1 EU antibody. The EU index or EU index as in Kabat or EU numbering scheme refers to the numbering of the EU antibody (Edelman et al., 1969, Proc Natl Acad Sci USA 63:78-85, hereby entirely incorporated by reference). In one embodiment, the Fc region comprises a D265A mutation. In one embodiment, the Fc region comprises a D265C mutation. In some embodiments, the Fc region of the anti-CD117 antibody (or fragment thereof) comprises an amino acid substitution at amino acid 234 according to the EU index as in Kabat. In one embodiment, the Fc region comprises a L234A mutation. In some embodiments, the Fc region of the anti-CD117 antibody (or fragment thereof) comprises an amino acid substitution at amino acid 235 according to the EU index as in Kabat. In one embodiment, the Fc region comprises a L235A mutation. In yet another embodiment, the Fc region comprises a L234A and L235A mutation. In a further embodiment, the Fc region comprises a D265C, L234A, and L235A mutation.

In certain aspects a variant IgG Fc domain comprises one or more amino acid substitutions resulting in decreased or ablated binding affinity for an Fc.gamma.R and/or C1q as compared to the wild type Fc domain not comprising the one or more amino acid substitutions. Fc binding interactions are essential for a variety of effector functions and downstream signaling events including, but not limited to, antibody dependent cell-mediated cytotoxicity (ADCC) and complement dependent cytotoxicity (CDC). Accordingly, in certain aspects, an antibody comprising a modified Fc region (e.g., comprising a L234A, L235A, and a D265C mutation) has substantially reduced or abolished effector functions.

Affinity to an Fc region can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE™. analysis or Octet™ analysis (forteBIO)), and other methods such as indirect binding assays, competitive binding assays fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions. One example of a competitive binding assay is a radioimmunoassay comprising the incubation of labeled antigen with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound in the presence of increasing amounts of an unlabeled second antibody.

The antibodies of the present disclosure may be further engineered to further modulate antibody (e.g., relative to an antibody having an unmodified Fc region) half-life by introducing additional Fc mutations, such as those described for example in (Dall'Acqua et al. (2006) J Biol Chem 281: 23514-24), (Zalevsky et al. (2010) Nat Biotechnol 28: 157-9), (Hinton et al. (2004) J Biol Chem 279: 6213-6), (Hinton et al. (2006) J Immunol 176: 346-56), (Shields et al. (2001) J Biol Chem 276: 6591-604), (Petkova et al. (2006) Int Immunol 18: 1759-69), (Datta-Mannan et al. (2007) Drug Metab Dispos 35: 86-94), (Vaccaro et al. (2005) Nat Biotechnol 23: 1283-8), (Yeung et al. (2010) Cancer Res 70: 3269-77) and (Kim et al. (1999) Eur J Immunol 29: 2819-25), and include positions 250, 252, 253, 254, 256, 257, 307, 376, 380, 428, 434 and 435. Exemplary mutations that may be made singularly or in combination are T250Q, M252Y, I253A, S254T, T256E, P2571, T307A, D376V, E380A, M428L, H433K, N434S, N434A, N434H, N434F, H435A and H435R mutations.

Thus, in one embodiment, the Fc region comprises a mutation resulting in a decrease in half life. An antibody having a short half life may be advantageous in certain instances where the antibody is expected to function as a short-lived therapeutic, e.g., the conditioning step described herein where the antibody is administered followed by HSCs. Ideally, the antibody would be substantially cleared prior to delivery of the HSCs, which also generally express CD117 but are not the target of the anti-CD117 antibody, unlike the endogenous stem cells. In one embodiment, the Fc regions comprise a mutation at position 435 (EU index according to Kabat). In one embodiment, the mutation is an H435A mutation.

In one embodiment, the anti-CD117 antibody described herein has a half life of equal to or less than about 24 hours, a half life of equal to or less than about 22 hours, a half life of equal to or less than about 21 hours, a half life of equal to or less than about 20 hours, a half life of equal to or less than about 19 hours, a half life of equal to or less than about 18 hours, a half life of equal to or less than about 17 hours, a half life of equal to or less than about 16 hours, a half life of equal to or less than about 15 hours, a half life of equal to or less than about 14 hours, equal to or less than about 13 hours, equal to or less than about 12 hours, equal to or less than about 11 hours, or equal to or less than about 10 hours. In one embodiment, the half life of the antibody is about 11 hours to about 24 hours; about 12 hours to about 22 hours; about 10 hours to about 20 hours; about 8 hours to about 18 hours; or about 14 hours to about 24 hours. In another embodiment, the anti-CD117 antibody described herein has a half-life (e.g., in humans) about 1-5 hours, about 5-10 hours, about 10-15 hours, about 15-20 hours, or about 20 to 25 hours.

In some aspects, the Fc region comprises two or more mutations that confer reduced half-life and greatly diminish or completely abolish an effector function of the antibody. In some embodiments, the Fc region comprises a mutation resulting in a decrease in half-life and a mutation of at least one residue that can make direct contact with an FcγR (e.g., as based on structural and crystallographic analysis). In one embodiment, the Fc region comprises a H435A mutation, a L234A mutation, and a L235A mutation. In one embodiment, the Fc region comprises a H435A mutation and a D265C mutation. In one embodiment, the Fc region comprises a H435A mutation, a L234A mutation, a L235A mutation, and a D265C mutation.

In some embodiments, the anti-CD117 antibody or antigen-binding fragment thereof is conjugated to a cytotoxin (e.g., amatoxin) by way of a cysteine residue in the Fc domain of the antibody or antigen-binding fragment thereof. In some embodiments, the cysteine residue is introduced by way of a mutation in the Fc domain of the antibody or antigen-binding fragment thereof. For instance, the cysteine residue may be selected from the group consisting of Cys118, Cys239, and Cys265. In one embodiment, the Fc region of the anti-CD117 antibody (or fragment thereof) comprises an amino acid substitution at amino acid 265 according to the EU index as in Kabat. In one embodiment, the Fc region comprises a D265C mutation. In one embodiment, the Fc region comprises a D265C and H435A mutation. In one embodiment, the Fc region comprises a D265C, a L234A, and a L235A mutation. In one embodiment, the Fc region comprises a D265C, a L234A, a L235A, and a H435A mutation. In one embodiment, the Fc region of the anti-CD117 antibody, or antigen-binding fragment thereof, comprises an amino acid substitution at amino acid 239 according to the EU index as in Kabat. In one embodiment, the Fc region comprises a S239C mutation. In one embodiment, the Fc region comprises a L234A mutation, a L235A mutation, a S239C mutation and a D265A mutation. In another embodiment, the Fc region comprises a S239C and H435A mutation. In another embodiment, the Fc region comprises a L234A mutation, a L235A mutation, and S239C mutation. In yet another embodiment, the Fc region comprises a H435A mutation, a L234A mutation, a L235A mutation, and S239C mutation. In yet another embodiment, the Fc region comprises a H435A mutation, a L234A mutation, a L235A mutation, a S239C mutation and D265A mutation.

Notably, Fc amino acid positions are in reference to the EU numbering index unless otherwise indicated.

In some embodiments of these aspects, the cysteine residue is naturally occurring in the Fc domain of the anti-CD117 antibody or antigen-binding fragment thereof. For instance, the Fc domain may be an IgG Fc domain, such as a human IgG1 Fc domain, and the cysteine residue may be selected from the group consisting of Cys261, Csy321, Cys367, and Cys425.

For example, in one embodiment, the Fc region of Antibody 67 is modified to comprise a D265C mutation (e.g., SEQ ID NO: 111). In another embodiment, the Fc region of Antibody 67 is modified to comprise a D265C, L234A, and L235A mutation (e.g., SEQ ID NO: 112). In yet another embodiment, the Fc region of Antibody 67 is modified to comprise a D265C and H435A mutation (e.g., SEQ ID NO: 113). In a further embodiment, the Fc region of Antibody 67 is modified to comprise a D265C, L234A, L235A, and H435A mutation (e.g., SEQ ID NO: 114).

In regard to Antibody 55, in one embodiment, the Fc region of Antibody 55 is modified to comprise a D265C mutation (e.g., SEQ ID NO: 117). In another embodiment, the Fc region of Antibody 55 is modified to comprise a D265C, L234A, and L235A mutation (e.g., SEQ ID NO: 118). In yet another embodiment, the Fc region of Antibody 55 is modified to comprise a D265C and H435A mutation (e.g., SEQ ID NO: 119). In a further embodiment, the Fc region of Antibody 55 is modified to comprise a D265C, L234A, L235A, and H435A mutation (e.g., SEQ ID NO: 120).

The Fc regions of any one of Antibody 54, Antibody 55, Antibody 56, Antibody 57, Antibody 58, Antibody 61, Antibody 66, Antibody 67, Antibody 68, or Antibody 69 can be modified to comprise a D265C mutation (e.g., as in SEQ ID NO: 123); a D265C, L234A, and L235A mutation (e.g., as in SEQ ID NO: 124); a D265C and H435A mutation (e.g., as in SEQ ID NO: 125); or a D265C, L234A, L235A, and H435A mutation (e.g., as in SEQ ID NO: 126).

The variant Fc domains described herein are defined according to the amino acid modifications that compose them. For all amino acid substitutions discussed herein in regard to the Fc region, numbering is always according to the EU index. Thus, for example, D265C is an Fc variant with the aspartic acid (D) at EU position 265 substituted with cysteine (C) relative to the parent Fc domain. Likewise, e.g., D265C/L234A/L235A defines a variant Fc variant with substitutions at EU positions 265 (D to C), 234 (L to A), and 235 (L to A) relative to the parent Fc domain. A variant can also be designated according to its final amino acid composition in the mutated EU amino acid positions. For example, the L234A/L235A mutant can be referred to as LALA. It is noted that the order in which substitutions are provided is arbitrary.

In one embodiment, the anti-CD117 antibody, or antigen binding fragment thereof, comprises variable regions having an amino acid sequence that is at least about 90%, about 95%, about 96%, about 97%, about 98% or about 99% identical to the SEQ ID Nos disclosed herein. Alternatively, the anti-CD117 antibody, or antigen binding fragment thereof, comprises CDRs comprising the SEQ ID Nos disclosed herein with framework regions of the variable regions described herein having an amino acid sequence that is at least about 90%, about 95%, about 96%, about 97%, about 98% or about 99% identical to the SEQ ID NOs disclosed herein.

In certain embodiments, an anti-CD117 antibody, or antigen binding fragment thereof, has a certain dissociation rate which is particularly advantageous when used as a part of a conjugate. For example, an anti-CD117 antibody has, in certain embodiments, an off rate constant (Koff) for human CD117 and/or rhesus CD117 of 1×10⁻² to 1×10⁻³, 1×10⁻³ to 1×10⁻⁴, 1×10⁻⁵ to 1×10⁻⁶, 1×10⁻⁶ to 1×10⁻⁷ or 1×10⁻⁷ to 1×10⁻⁸, as measured by bio-layer interferometry (BLI). In some embodiments, the antibody or antigen-binding fragment thereof binds CD117 (e.g., human CD117 and/or rhesus CD117) with a K_{D} of about 100 nM or less, about 90 nM or less, about 80 nM or less, about 70 nM or less, about 60 nM or less, about 50 nM or less, about 40 nM or less, about 30 nM or less, about 20 nM or less, about 10 nM or less, about 8 nM or less, about 6 nM or less, about 4 nM or less, about 2 nM or less, about 1 nM or less as determined by a Bio-Layer Interferometry (BLI) assay.

The antibodies, and binding fragments thereof, disclosed herein can be used in conjugates, as described in more detail below.

Antibodies may be produced using recombinant methods and compositions, e.g., as described in U.S. Pat. No. 4,816,567. In one embodiment, isolated nucleic acid encoding an anti-CD117 antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-CLL-1 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-CD117 antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

In one embodiment, the anti-CD117 antibody, or antigen binding fragment thereof, comprises variable regions having an amino acid sequence that is at least about 90%, about 95%, about 96%, about 97%, about 98% or about 99% identical to the SEQ ID Nos disclosed herein. Alternatively, the anti-CD117 antibody, or antigen binding fragment thereof, comprises CDRs comprising the SEQ ID Nos disclosed herein with framework regions of the variable regions described herein having an amino acid sequence that is at least about 90%, about 95%, about 96%, about 97%, about 98% or about 99% identical to the SEQ ID Nos disclosed herein.

In one embodiment, the anti-CD117 antibody, or antigen binding fragment thereof, comprises a heavy chain variable region and a heavy chain constant region having an amino acid sequence that is disclosed herein. In another embodiment, the anti-CD117 antibody, or antigen binding fragment thereof, comprises a light chain variable region and a light chain constant region having an amino acid sequence that is disclosed herein. In yet another embodiment, the anti-CD117 antibody, or antigen binding fragment thereof, comprises a heavy chain variable region, a light chain variable region, a heavy chain constant region and a light chain constant region having an amino acid sequence that is disclosed herein.

b. Anti-CD45 Antibodies

The present methods also include the use of antibodies, and antigen-binding fragments thereof, that specifically bind to a CD45 polypeptide, e.g., a human CD45 polypeptide, and uses thereof. In an exemplary embodiment, the antibody, or antigen-binding fragment thereof, that specifically binds to a CD45 polypeptide comprises a heavy chain variable region and a light chain variable region. Anti-CD45 antibodies may be used in ADCs described herein.

CD45 is a hematopoietic cell-specific transmembrane protein tyrosine phosphatase essential for T and B cell antigen receptor-mediated signaling. CD45 includes a large extracellular domain, and a phosphatase containing cytosolic domain. CD45 may act as both a positive and negative regulator depending on the nature of the stimulus and the cell type involved. Although there are a large number of permutations possible in the CD45 gene, only six isoforms are traditionally identified in humans. The isoforms are RA, RO, RB, RAB, RBC and RABC (Hermiston et al. 2003 “CD45: a critical regulator of signaling thresholds in immune cells.” Annu Rev Immunol. 2:107-137.). CD45RA is expressed on naïve T cells, and CD45RO is expressed on activated and memory T cells, some B cell subsets, activated monocytes/macrophages, and granulocytes. CD45RB is expressed on peripheral B cells, naïve T cells, thymocytes, weakly on macrophages, and dendritic cells. Antibodies and antigen-binding fragments capable of binding human CD45 (mRNA NCBI Reference Sequence: NM_080921.3, Protein NCBI Reference Sequence: NP_563578.2), including those capable of binding the isoform CD45RO, can be used in conjunction with the compositions and methods disclosed herein, such as to promote engraftment of hematopoietic stem cell grafts in a patient in need of hematopoietic stem cell transplant therapy. Multiple isoforms of CD45 arise from the alternative splicing of 34 exons in the primary transcript. Splicing of exons 4, 5, 6, and potentially 7 give rise to multiple CD45 variations. Selective exon expression is observed in the CD45 isoforms described in Table 1, below.

TABLE 1
Exon expression in various CD45 isoforms

| CD45 Isoform | Exon Expression Pattern |
|---|---|
| CD45RA | Expresses exon 4 only |
| CD45RB | Expresses exon 5 only |
| CD45RC | Expresses exon 6 only |
| CD45RO | Does not express exons 4-6 |

Alternative splicing can result in individual exons or combinations of exons expressed in various isoforms of the CD45 protein (for example, CD45RA, CD45RAB, CD45RABC). In contrast, CD45RO lacks expression of exons 4-6 and is generated from a combination of exons 1-3 and 7-34. There is evidence that exon 7 can also be excluded from the protein, resulting in splicing together of exons 1-3 and 8-34. This protein, designated E3-8, has been detected at the mRNA level but has not been currently identified by flow cytometry.

CD45RO is currently the only known CD45 isoform expressed on hematopoietic stem cells. CD45RA and CD45RABC have not been detected or are excluded from the phenotype of hematopoietic stem cells. There is evidence from studies conducted in mice that CD45RB is expressed on fetal hematopoietic stem cells, but it is not present on adult bone marrow hematopoietic stem cells. Notably, CD45RC has a high rate of polymorphism in exon 6 found within Asian populations (a polymorphism at exon 6 in CD45RC is found in approximately 25% of the Japanese population). This polymorphism leads to high expression of CD45RO and decreased levels of CD45RA, CD45RB, and CD45RC. Additionally, CD45RA variants (such as CD45RAB and CD45RAC) exhibit a polymorphism in exon 4 that has been associated with autoimmune disease.

The presence of CD45RO on hematopoietic stem cells and its comparatively limited expression on other immune cells (such as T and B lymphocyte subsets and various myeloid cells) renders CD45RO a particularly well-suited target for conditioning therapy for patients in need of a hematopoietic stem cell transplant. As CD45RO only lacks expression of exons 4, 5, and 6, its use as an immunogen enables the screening of pan CD45 Abs and CD45RO-specific antibodies.

Anti-CD45 antibodies that can be used in conjunction with the patient conditioning methods described herein include, for example, the anti-CD45 antibody clone H130, which is commercially available from BIOLEGEND® (San Diego, Calif.), as well as humanized variants thereof. Humanization of antibodies can be performed by replacing framework residues and constant region residues of a non-human antibody with those of a germline human antibody according to procedures known in the art. Additional anti-CD45 antibodies that can be used in conjunction with the methods described herein include the anti-CD45 antibodies ab10558, EP322Y, MEM-28, ab10559, 0.N.125, F10-89-4, Hle-1, 2B111, YTH24.5, PD7/26/16, F10-89-4, 1 B7, ab154885, B-A11, phosphor S1007, ab170444, EP350, Y321, GA90, D3/9, X1 6/99, and LT45, which are commercially available from ABCAM® (Cambridge, Mass.), as well as humanized variants thereof.

Further anti-CD45 antibodies that may be used in conjunction with the patient conditioning procedures described herein include anti-CD45 antibody HPA000440, which is commercially available from SIGMA-ALDRICH® (St. Louis, Mo.), and humanized variants thereof. Additional anti-CD45 antibodies that can be used in conjunction with the patient conditioning methods described herein include murine monoclonal antibody BC8, which is described, for instance, in Matthews et al., Blood 78:1864-1874, 1991, the disclosure of which is incorporated herein by reference as it pertains to anti-CD45 antibodies, as well as humanized variants thereof. Further anti-CD45 antibodies that can be used in conjunction with the methods described herein include monoclonal antibody YAML568, which is described, for instance, in Glatting et al., J. Nucl. Med. 8:1335-1341, 2006, the disclosure of which is incorporated herein by reference as it pertains to anti-CD45 antibodies, as well as humanized variants thereof. Additional anti-CD45 antibodies that can be used in conjunction with the patient conditioning procedures described herein include monoclonal antibodies YTH54.12 and YTH25.4, which are described, for instance, in Brenner et al., Ann. N.Y. Acad. Sci. 996:80-88, 2003, the disclosure of which is incorporated herein by reference as it pertains to anti-CD45 antibodies, as well as humanized variants thereof. Additional anti-CD45 antibodies for use with the patient conditioning methods described herein include UCHL1, 2H4, SN130, MD4.3, MBI, and MT2, which are described, for instance, in Brown et al., Immunology 64:331-336, 1998, the disclosure of which is incorporated herein by reference as it pertains to anti-CD45 antibodies, as well as humanized variants thereof. Additional anti-CD45 antibodies that can be used in conjunction with the methods described herein include those produced and released from American Type Culture Collection (ATCC) Accession Nos. RA3-6132, RA3-2C2, and TIB122, as well as monoclonal antibodies C363.16A, and 13/2, which are described, for instance, in Johnson et al., J. Exp. Med. 169:1179-1184, 1989, the disclosure of which is incorporated herein by reference as it pertains to anti-CD45 antibodies, as well as humanized variants thereof. Further anti-CD45 antibodies that can be used in conjunction with the patient conditioning methods described herein include the monoclonal antibodies AHN-12.1, AHN-12, AHN-12.2, AHN-12.3, AHN-12.4, HLe-1, and KC56(T200), which are described, for instance, in Harvath et al., J. Immunol. 146:949-957, 1991, the disclosure of which is incorporated herein by reference as it pertains to anti-CD45 antibodies, as well as humanized variants thereof.

Additional anti-CD45 antibodies that can be used in conjunction with the patient conditioning methods described herein include those described, for example, in U.S. Pat. No. 7,265,212 (which describes, e.g., anti-CD45 antibodies 39E11, 16C9, and 1G10, among other clones); U.S. Pat. No. 7,160,987 (which describe, e.g., anti-CD45 antibodies produced and released by ATCC Accession No. HB-11873, such as monoclonal antibody 6G3); and U.S. Pat. No. 6,099,838 (which describes, e.g., anti-CD45 antibody MT3, as well as antibodies produced and released by ATCC Accession Nos. HB220 (also designated MB23G2) and HB223), as well as US 2004/0096901 and US 2008/0003224 (which describes, e.g., anti-CD45 antibodies produced and released by ATCC Accession No. PTA-7339, such as monoclonal antibody 17.1), the disclosures of each of which are incorporated herein by reference as they pertain to anti-CD45 antibodies.

Further anti-CD45 antibodies that can be used in conjunction with the patient conditioning methods described herein include antibodies produced and released from ATCC Accession Nos. MB4B4, MB23G2, 14.8, GAP 8.3, 74-9-3, 1/24.D6, 9.4, 4B2, M1/9.3.4.HL.2, as well as humanized and/or affinity-matured variants thereof. Affinity maturation can be performed, for instance, using in vitro display techniques described herein or known in the art, such as phage display.

Additional anti-CD45 antibodies that can be used in conjunction with the patient conditioning methods described herein include anti-CD45 antibody T29/33, which is described, for instance, in Morikawa et al., Int. J. Hematol. 54:495-504, 1991, the disclosure of which is incorporated herein by reference as it pertains to anti-CD45 antibodies.

In certain embodiments, the anti-CD45 antibody is selected from apamistamab (also known 90Y-BC8, lomab-B, BC8; as described in, e.g., US20170326259, WO2017155937, and Orozco et al. Blood. 127.3 (2016): 352-359.) or BC8-B10 (as described, e.g., in Li et al. PloS one 13.10 (2018): e0205135.), each of which is incorporated by reference. Other anti-CD45 antibodies have been described, for example, in WO2003048327, WO2016016442, US20170226209, US20160152733, U.S. Pat. No. 9,701,756; US20110076270, or U.S. Pat. No. 7,825,222, each of which is incorporated by reference as it pertains to anti-CD45 antibodies.

In one embodiment, the anti-CD45 antibody comprises a heavy chain of an anti-CD45 antibody described herein, and a light chain variable region of anti-CD45 antibody described herein. In one embodiment, the anti-CD45 antibody comprises a heavy chain comprising a CDR1, CDR2 and CDR3 of an anti-CD45 antibody described herein, and a light chain variable region comprising a CDR1, CDR2 and CDR3 of an anti-CD45 antibody described herein.

In another embodiment, the antibody, or antigen-binding fragment thereof, comprises a heavy chain variable region that comprises an amino acid sequence having at least about 90% identity to an anti-CD45 antibody herein, e.g., at least about 95%, at least about 96%, at least about 97%, at least about 98%, at least about 99%, or 100% identity to an anti-CD45 antibody herein. In certain embodiments, an antibody comprises a modified heavy chain (HC) variable region comprising an HC variable domain of an anti-CD45 antibody herein, or a variant thereof, which variant (i) differs from the anti-CD45 antibody in 1, 2, 3, 4 or 5 amino acids substitutions, additions or deletions; (ii) differs from the anti-CD45 antibody in at most 5, 4, 3, 2, or 1 amino acids substitutions, additions or deletions; (iii) differs from the anti-CD45 antibody in 1-5, 1-3, 1-2, 2-5 or 3-5 amino acids substitutions, additions or deletions and/or (iv) comprises an amino acid sequence that is at least about 75%, at least about 80%, at least about 85%, at least about 90%, at least about 95%, at least about 96%, at least about 97%, at least about 98% or at least about 99% or more identical to the anti-CD45 antibody, wherein in any of (i)-(iv), an amino acid substitution may be a conservative amino acid substitution or a non-conservative amino acid substitution; and wherein the modified heavy chain variable region can have an enhanced biological activity relative to the heavy chain variable region of the anti-CD45 antibody, while retaining the CD45 binding specificity of the antibody.

The disclosures of each of the foregoing publications are incorporated herein by reference as they pertain to anti-CD45 antibodies. Antibodies and antigen-binding fragments that may be used in conjunction with the compositions and methods described herein include the above-described antibodies and antigen-binding fragments thereof, as well as humanized variants of those non-human antibodies and antigen-binding fragments described above and antibodies or antigen-binding fragments that bind the same epitope as those described above, as assessed, for instance, by way of a competitive CD45 binding assay.

c. Anti-CD2 Antibodies

Human CD2 is also referred to as T-cell Surface Antigen T11/Leu-5, T11, CD2 antigen (p50), and Sheep Red Blood Cell Receptor (SRBC). CD2 is expressed on T cells. Two isoforms of human CD2 have been identified. Isoform 1 contains 351 amino acids is described in Seed, B. et al. (1987) 84: 3365-69 (see also Sewell et al. (1986) 83: 8718-22) and below (NCBI Reference Sequence: NP_001758.2):

(SEQ ID NO: 293)
msfpckfvas fllifnvssk gayskeitna
letwgalgqd inldipsfqm sddiddikwe
ktsdkkkiaq frkeketfke kdtyklfkng
tlkikhlktd dqdiykvsiy dtkgknvlek
ifdlkiqery skpkiswtci nttltcevmn
gtdpelnlyq dgkhlklsqr vithkwttsl
sakfkctagn kvskessvep vscpekgldi
yliigicggg sllmvfvall vfyitkrkkq
rsrrndeele trahrvatee rgrkphqipa
stpqnpatsq hpppppghrs qapshrpppp
ghrvqhqpqk rppapsgtqv hqqkgpplpr
prvqpkpphg aaenslspss n

A second isoform of CD2 is 377 amino acids and is identified herein as NCBI Reference Sequence: NP_001315538.1.

In one embodiment, an anti-CD2 antibody that may be used in conjunction with the compositions and methods described herein include those that have one or more, or all, of the following CDRs:
a CDR-H1 having the amino acid sequence EYYMY (SEQ ID NO: 294);
a CDR-H2 having the amino acid sequence RIDPEDGSIDYVEKFKK (SEQ ID NO: 295);
a CDR-H3 having the amino acid sequence GKFNYRFAY (SEQ ID NO: 296);
a CDR-L1 having the amino acid sequence RSSQSLLHSSGNTYLN (SEQ ID NO: 297);
a CDR-L2 having the amino acid sequence LVSKLES (SEQ ID NO: 298); and
a CDR-L3 having the amino acid sequence MQFTHYPYT (SEQ ID NO: 299).

In one embodiment, an anti-CD2 antibody, or antigen binding portion thereof, comprises a heavy chain variable region having the amino acid sequence QVQLVQSGAEVKKPGASVKVSCKASGYTFTEYYMYWVRQAPGQGLELMGRIDPEDGSI DYVEKFKKKVTLTADTSSSTAYMELSSLTSDDTAVYYCARGKFNYRFAYWGQGTLVTVSS SEQ ID NO: 300), and a light chain variable region having the amino acid sequence

(SEQ ID NO: 301)
DVVMTQSPPSLLVTLGQPASISCRSSQSLLHSSGNTYLN
WLLQRPGQSPQPLIYLVSKLESGVPDRFSGSGSGTDFTL
KISGVEAEDVGVYYCMQFTHYPYTFGQGTKLEIK.

In one embodiment, an anti-CD2 antibody that may be used in conjunction with the compositions and methods described herein include those that have one or more, or all, of the following CDRs:
a CDR-H1 having the amino acid sequence GFTFSSY (SEQ ID NO: 302);
a CDR-H2 having the amino acid sequence SGGGF (SEQ ID NO: 303);
a CDR-H3 having the amino acid sequence SSYGEIMDY (SEQ ID NO: 304);
a CDR-L1 having the amino acid sequence RASQRIGTSIH (SEQ ID NO: 305);
a CDR-L2 having the amino acid sequence YASESIS (SEQ ID NO: 306); and
a CDR-L3 having the amino acid sequence QQSHGWPFTF (SEQ ID NO: 307).

In one embodiment, an anti-CD2 antibody, or antigen binding portion thereof, comprises a heavy chain variable region having the amino acid sequence EVKLVESGGGLVKPGGSLKLSCAASGFTFSSYDMSWVRQTPEKRLEWVASISGGGFLY YLDSVKGRFTISRDNARNILYLHMTSLRSEDTAMYYCARSSYGEIMDYWGQGTSVTVSS (SEQ ID NO: 308), and a light chain variable region having the amino acid sequence

(SEQ ID NO: 309)
DILLTQSPAILSVSPGERVSFSCRASQRIGTSIHWYQQR
TTGSPRLLIKYASESISGIPSRFSGSGSGTDFTLSINSV
ESEDVADYYCQQSHGWPFTFGGGTKLEIE.

In another embodiment, an anti-CD2 antibody that may be used in conjunction with the compositions and methods described herein include those that have one or more, or all, of the following CDRs:
a CDR-H1 having the amino acid sequence GFTFSSY (SEQ ID NO: 302);
a CDR-H2 having the amino acid sequence SGGGF (SEQ ID NO: 303);
a CDR-H3 having the amino acid sequence SSYGELMDY (SEQ ID NO: 310);
a CDR-L1 having the amino acid sequence RASQRIGTSIH (SEQ ID NO: 305);
a CDR-L2 having the amino acid sequence YASESIS (SEQ ID NO: 306); and
a CDR-L3 having the amino acid sequence QQSHGWPFTF (SEQ ID NO: 307).

In one embodiment, an anti-CD2 antibody, or antigen binding portion thereof, comprises a heavy chain variable region having the amino acid sequence EVKLVESGGGLVKPGGSLKLSCAASGFTFSSYDMSWVRQTPEKRLEWVASISGGGFLY YLDSVKGRFTISRDNARNILYLHMTSLRSEDTAMYYCARSSYGELMDYWGQGTSVTVSS (SEQ ID NO: 311), and a light chain variable region having the amino acid sequence

(SEQ ID NO: 309)
DILLTQSPAILSVSPGERVSFSCRASQRIGTSIHWYQQR
TTGSPRLLIKYASESISGIPSRFSGSGSGTDFTLSINSV
ESEDVADYYCQQSHGWPFTFGGGTKLEIE.

Antibodies and antigen-binding fragments thereof containing the foregoing CDR sequences are described, e.g., in U.S. Pat. No. 6,849,258, the disclosure of which is incorporated herein by reference as it pertains to anti-CD2 antibodies and antigen-binding fragments thereof.

d. Anti-CD5 Antibodies

Human CD5 is also referred to as Lymphocyte Antigen T1, T1, Leu-1, and LEU1. CD5 is expressed on human T cells. Two isoforms of human CD5 have been identified. Isoform 1 contains 495 amino acids and is described in Gladkikh et al (2017) Cancer Med. 6(12):2984 and Jones et al. (1986) Nature 323 (6086): 346). The amino acid sequence of CD5 (isoform 1) is provided below (NCBI Reference Sequence: NP_055022.2):

(SEQ ID NO: 312)
mpmgslqpla tlyllgmlva sclgrlswyd
pdfqarltrs nskcqgqlev ylkdgwhmvc
sqswgrsskq wedpsqaskv cqrlncgvpl
slgpflvtyt pqssiicygq lgsfsncshs
rndmchslgl tclepqkttp pttrpppttt
peptapprlq lvaqsggghc agvvefysgs
lggtisyeag dktqdlenfl cnnlqcgsfl
khlpeteagr agdpgepreh qplpigwkiq
nssctslehc frkikpqksg rvlallcsgf
qpkvqsrlvg gssicegtve vrqgaqwaal
cdsssarssl rweevcreqq cgsvnsyrvl
dagdptsrgl fcphqklsqc helwernsyc
kkvfvtcadp npaglaagtv asiilalvll
vvllvvcapl aykklvkkfr akkarawigp
tgmnqnmsfh rnhtatvrsh aenptashvd
nevsqpprns hlsaypaleg alhrssmgpd
nssdsdydlh gaqrl

A second isoform of human CD5 is 438 amino acids (see underlined portion above) and is identified as NCBI Reference Sequence: NP_001333385.1. Unlike isoform 1, CD5 isoform 2 is an intracellular protein. Isoform 2 contains a distinct 5′ UTR and lacks an in-frame portion of the 5′ coding region, compared to isoform 1. The resulting isoform 2 has a shorter N-terminus, compared to isoform 1. The CD5 isoform 2 lacks the leader peptide, compared to isoform 1 and represents an intracellular isoform found in a subset of B lymphocytes. The ADCs described herein are specific for human CD5 isoform 1 which represents the extracellular version of human CD5.

In one embodiment, an anti-CD5 antibody that may be used in the methods and compositions described herein is Antibody 5D7 (Ab5D7). The heavy chain variable region (VH) amino acid sequence of Ab5D7 is provided below as SEQ ID NO: 313.

(SEQ ID NO: 313)
QVTLKESGPVLVKPTETLTLTCTFSGFSLSTSGMGVGWIRQ
APGKGLEWVAHIWWDDDVYYNPSLKSRLTITKDASKDQVSL
KLSSVTAADTAVYYCVRRRATGTGFDYWGQGTLVTVSS

The VH CDR amino acid sequences of Ab5D7 are underlined above and are as follows: FSLSTSGMG (VH CDR1; SEQ ID NO: 315); WWDDD (VH CDR2; SEQ ID NO: 316); and RRATGTGFDY (VH CDR3; SEQ ID NO: 317).

The light chain variable region (VL) amino acid sequence of Ab5D7 is provided below as SEQ ID NO 314.

(SEQ ID NO: 314)
NIVMTQSPSSLSASVGDRVTITCQASQDVGTAVAWYQQKPDQSP
KLLIYWTSTRHTGVPDRFTGSGSGTDFTLTISSLQPEDIATYFC
HQYNSYNTFGSGTKLEIK

The VL CDR amino acid sequences of Ab5D7 are underlined above and are as follows: QDVGTA (VL CDR1; SEQ ID NO: 318); WTSTRHT (VL CDR2; SEQ ID NO: 319); and YNSYNT (VL CDR3; SEQ ID NO: 320).

In one embodiment, an anti-CD5 ADC comprises an anti-CD5 antibody comprising a heavy chain comprising a CDR1 domain comprising the amino acid sequence set forth in SEQ ID NO: 315, a CDR2 domain comprising the amino acid sequence set forth in SEQ ID NO: 316, and a CDR3 domain comprising the amino acid sequence set forth in SEQ ID NO: 317, and comprises a light chain comprising a CDR1 domain comprising the amino acid sequence set forth in SEQ ID NO: 318, a CDR2 domain comprising the amino acid sequence set forth in SEQ ID NO: 319, and a CDR3 domain comprising the amino acid sequence set forth in SEQ ID NO: 320, wherein the antibody is conjugated to a cytotoxin via a linker.

In one embodiment, an anti-CD5 ADC comprises an anti-CD5 antibody comprising a heavy chain comprising a variable region comprising an amino acid sequence as set forth in SEQ ID NO:313, and a light chain comprising a variable region comprising an amino acid sequence as set forth in SEQ ID NO: 314, wherein the antibody is conjugated to a cytotoxin via a linker.

In another embodiment, an anti-CD5 antibody used in the ADCs described herein is the 5D7 antibody (see, e.g., US 20080254027, the disclosure of which is incorporated herein by reference). In another embodiment, an anti-CD5 antibody that may be used in the methods and compositions (including ADCs) described herein is a variant of the 5D7 antibody (see, e.g., US 20080254027, the disclosure of which is incorporated herein by reference).

Additional sequence for anti-CD5 antibodies or binding fragments, described herein, are known in the art, including sequences set forth in WO 2019/108863, the contents of which are incorporated herein.

Additional anti-CD5 antibodies that can be used in the ADCs described herein can be identified using techniques known in the art, such as hybridoma production. Hybridomas can be prepared using a murine system. Protocols for immunization and subsequent isolation of splenocytes for fusion are known in the art. Fusion partners and procedures for hybridoma generation are also known. Alternatively, anti-CD5 antibodies can be generated using the HuMAb-Mouse® or XenoMouse™. In making additional anti-CD5 antibodies, the CD5 antigen is isolated and/or purified. The CD5 antigen may be a fragment of CD5 from the extracellular domain of CD5. Immunization of animals can be performed by any method known in the art. See, e.g., Harlow and Lane, Antibodies: A Laboratory Manual, New York: Cold Spring Harbor Press, 1990. Methods for immunizing animals such as mice, rats, sheep, goats, pigs, cattle and horses are well known in the art. See, e.g., Harlow and Lane, supra, and U.S. Pat. No. 5,994,619. The CD5 antigen may be administered with an adjuvant to stimulate the immune response. Adjuvants known in the art include complete or incomplete Freund's adjuvant, RIBI (muramyl dipeptides) or ISCOM (immunostimulating complexes). After immunization of an animal with a CD5 antigen, antibody-producing immortalized cell lines are prepared from cells isolated from the immunized animal. After immunization, the animal is sacrificed and lymph node and/or splenic B cells are immortalized by methods known in the art (e.g., oncogene transfer, oncogenic virus transduction, exposure to carcinogenic or mutating compounds, fusion with an immortalized cell, e.g., a myeloma cell, and inactivating a tumor suppressor gene. See, e.g., Harlow and Lane, supra. Hybridomas can be selected, cloned and further screened for desirable characteristics, including robust growth, high antibody production and desirable antibody characteristics.

Anti-CD5 antibodies for use in the anti-CD5 ADCs described herein can also be identified using high throughput screening of libraries of antibodies or antibody fragments for molecules capable of binding CD5. Such methods include in vitro display techniques known in the art, such as phage display, bacterial display, yeast display, mammalian cell display, ribosome display, mRNA display, and cDNA display, among others. The use of phage display to isolate antibodies, antigen-binding fragments, or ligands that bind biologically relevant molecules has been reviewed, for example, in Felici et al., Biotechnol. Annual Rev. 1:149-183, 1995; Katz, Annual Rev. Biophys. Biomol. Struct. 26:27-45, 1997; and Hoogenboom et al., Immunotechnology 4:1-20, 1998, the disclosures of each of which are incorporated herein by reference as they pertain to in vitro display techniques. Randomized combinatorial peptide libraries have been constructed to select for polypeptides that bind cell surface antigens as described in Kay, Perspect. Drug Discovery Des. 2:251-268, 1995 and Kay et al., Mol. Divers. 1:139-140, 1996, the disclosures of each of which are incorporated herein by reference as they pertain to the discovery of antigen-binding molecules. Proteins, such as multimeric proteins, have been successfully phage-displayed as functional molecules (see, for example, EP 0349578; EP 4527839; and EP 0589877, as well as Chiswell and McCafferty, Trends Biotechnol. 10:80-84 1992, the disclosures of each of which are incorporated herein by reference as they pertain to the use of in vitro display techniques for the discovery of antigen-binding molecules. In addition, functional antibody fragments, such as Fab and scFv fragments, have been expressed in in vitro display formats (see, for example, McCafferty et al., Nature 348:552-554, 1990; Barbas et al., Proc. Natl. Acad. Sci. USA 88:7978-7982, 1991; and Clackson et al., Nature 352:624-628, 1991, the disclosures of each of which are incorporated herein by reference as they pertain to in vitro display platforms for the discovery of antigen-binding molecules).

In addition to in vitro display techniques, computational modeling techniques can be used to design and identify anti-CD5 antibodies or antibody fragments in silico, for instance, using the procedures described in US 2013/0288373, the disclosure of which is incorporated herein as it pertains to molecular modeling methods for identifying anti-CD5 antibodies. For example, using computational modeling techniques, one of skill in the art can screen libraries of antibodies or antibody fragments in silico for molecules capable of binding specific epitopes on CD5, such as extracellular epitopes of CD5.

In one embodiment, the anti-CD5 antibody used in the ADCs described herein are able to internalize into the cell. In identifying an anti-CD5 antibody (or fragment thereof) additional techniques can be used to identify antibodies or antigen-binding fragments that bind CD5 on the surface of a cell (e.g., a T cell) and further are able to be internalized by the cell, for instance, by receptor-mediated endocytosis. For example, the in vitro display techniques described above can be adapted to screen for antibodies or antigen-binding fragments thereof that bind CD5 on the surface of a hematopoietic stem cell and that are subsequently internalized. Phage display represents one such technique that can be used in conjunction with this screening paradigm. To identify anti-CD5 antibodies or fragments thereof that bind CD5 and are subsequently internalized a CD5+ cell, one of skill in the art can use the phage display techniques described in Williams et al., Leukemia 19:1432-1438, 2005, the disclosure of which is incorporated herein by reference in its entirety. Such techniques may be applied to antibodies targeting other antigens as well.

The internalizing capacity of an anti-CD5 antibody or fragment thereof can be assessed, for instance, using radionuclide internalization assays known in the art. For example, an anti-CD5 antibody or fragment thereof, identified using in vitro display techniques described herein or known in the art can be functionalized by incorporation of a radioactive isotope, such as ¹⁸F, ⁷⁵Br, ⁷⁷Br, ¹²²I, ¹²³I, ¹²⁴I, ¹²⁵I, ¹²⁹I, ¹³¹I, ²¹¹At, ⁶⁷Ga, ¹¹¹In, ⁹⁹Tc, ¹⁶⁹Yb, ¹⁸⁶Re, ⁶⁴Cu, ⁶⁷Cu, ¹⁷⁷Lu, ⁷⁷As, ⁷²As, ⁸⁶Y, ⁹⁰Y, ⁸⁹Zr, ²¹²Bi, ²¹³Bi, or ²²⁵Ac. For instance, radioactive halogens, such as ¹⁸F, ⁷⁵Br, ⁷⁷Br, ¹²²I, ¹²³I, ¹²⁴I, ¹²⁵I, ¹²⁹I, ¹³¹I, ²¹¹At, can be incorporated into antibodies, fragments thereof, or ligands using beads, such as polystyrene beads, containing electrophilic halogen reagents (e.g., Iodination Beads, Thermo Fisher Scientific, Inc., Cambridge, Mass.). Radiolabeled antibodies, or fragments thereof, can be incubated with hematopoietic stem cells for a time sufficient to permit internalization. Internalized antibodies, or fragments thereof, can be identified by detecting the emitted radiation (e.g., γ-radiation) of the resulting hematopoietic stem cells in comparison with the emitted radiation (e.g., γ-radiation) of the recovered wash buffer. The foregoing internalization assays can also be used to characterize ADCs, as well as ADCs targeting HSC antigens other than CD5.

e. Methods of Identifying and Producing Antibodies

Provided herein are specific anti-CD117 and anti-CD45 antibodies that may be used, for example, in conditioning methods prior to genetically modified HSC transplantation. In view of the disclosure herein, other anti-CD117 antibodies, e.g., neutral antibodies, or other anti-CD45 antibodies can be identified.

Methods for high throughput screening of antibody, or antibody fragment libraries for molecules capable of binding CD117 (e.g., GNNK+CD117) or CD45 expressed by HSCs and/or immune cells can be used to identify and affinity mature antibodies useful for treating cancers, autoimmune diseases, and conditioning a patient (e.g., a human patient) in need of stem cell gene therapy as described herein. Such methods can be used to identify equivalent or even improved versions of the antibodies described herein. Such methods include in vitro display techniques known in the art, such as phage display, bacterial display, yeast display, mammalian cell display, ribosome display, mRNA display, and cDNA display, among others.

The use of phage display to isolate ligands or antibodies or antibody fragments (e.g., scFv) that bind biologically relevant molecules has been reviewed, for example, in Felici et al., Biotechnol. Annual Rev. 1:149-183, 1995; Katz, Annual Rev. Biophys. Biomol. Struct. 26:27-45, 1997; and Hoogenboom et al., Immunotechnology 4:1-20, 1998, the disclosures of each of which are incorporated herein by reference as they pertain to in vitro display techniques. Randomized combinatorial peptide libraries have been constructed to select for polypeptides that bind cell surface antigens as described in Kay, Perspect. Drug Discovery Des. 2:251-268, 1995 and Kay et al., Mol. Divers. 1:139-140, 1996, the disclosures of each of which are incorporated herein by reference as they pertain to the discovery of antigen-binding molecules. Proteins, such as multimeric proteins, have been successfully phage-displayed as functional molecules (see, for example, EP 0349578; EP 4527839; and EP 0589877, as well as Chiswell and McCafferty, Trends Biotechnol. 10:80-84 1992, the disclosures of each of which are incorporated herein by reference as they pertain to the use of in vitro display techniques for the discovery of antigen-binding molecules). In addition, functional antibody fragments, such as Fab and scFv fragments, have been expressed in in vitro display formats (see, for example, McCafferty et al., Nature 348:552-554, 1990; Barbas et al., Proc. Natl. Acad. Sci. USA 88:7978-7982, 1991; and Clackson et al., Nature 352:624-628, 1991, the disclosures of each of which are incorporated herein by reference as they pertain to in vitro display platforms for the discovery of antigen-binding molecules). Human antibodies can also be generated, for example, in the HuMAb-Mouse® or XenoMouse™. These techniques, among others, can be used to identify and improve the affinity of antibodies that bind CD117 (e.g., GNNK+CD117) that can in turn be used to deplete endogenous hematopoietic stem cells in a patient (e.g., a human patient) in need of hematopoietic stem cell transplant therapy.

In addition to in vitro display techniques, computational modeling techniques can be used to design and identify antibodies, and antibody fragments, capable of binding an antigen such as CD117 (e.g., GNNK+CD117) or CD45. For example, using computational modeling techniques, one of skill in the art can screen libraries of antibodies, and antibody fragments, in silico for molecules capable of binding specific epitopes, such as extracellular epitopes of this antigen. The antibodies, and antigen-binding fragments thereof, identified by these computational techniques can be used in conjunction with the therapeutic methods described herein, such as the cancer and autoimmune disease treatment methods described herein and the patient conditioning procedures described herein.

Additional techniques can be used to identify antibodies, and antigen-binding fragments thereof, capable of binding, e.g., CD117 (e.g., GNNK+CD117) or CD45 on the surface of a cell (e.g., a cancer cell, autoimmune cell, or hematopoietic stem cell) and that are internalized by the cell, for instance, by receptor-mediated endocytosis. For example, the in vitro display techniques described above can be adapted to screen for antibodies, and antigen-binding fragments thereof, that bind CD117 (e.g., GNNK+CD117) or CD45 and that are subsequently internalized. Phage display represents one such technique that can be used in conjunction with this screening paradigm. To identify antibodies, and fragments thereof, that bind, e.g., CD117 (e.g., GNNK+CD117) or CD45 and are subsequently internalized by hematopoietic stem cells, one of skill in the art can adapt the phage display techniques described, for example, in Williams et al., Leukemia 19:1432-1438, 2005, the disclosure of which is incorporated herein by reference in its entirety. For example, using mutagenesis methods known in the art, recombinant phage libraries can be produced that encode antibodies, antibody fragments, such as scFv fragments, Fab fragments, diabodies, triabodies, and ¹⁰Fn3 domains, among others, or ligands that contain randomized amino acid cassettes (e.g., in one or more, or all, of the CDRs or equivalent regions thereof or an antibody or antibody fragment). The framework regions, hinge, Fc domain, and other regions of the antibodies or antibody fragments may be designed such that they are non-immunogenic in humans, for instance, by virtue of having human germline antibody sequences or sequences that exhibit only minor variations relative to human germline antibodies.

Using phage display techniques described herein or known in the art, phage libraries containing randomized antibodies, or antibody fragments, covalently bound to the phage particles can be incubated with, e.g., CD117 (e.g., GNNK+CD117) or CD45 antigen, for instance, by first incubating the phage library with blocking agents (such as, for instance, milk protein, bovine serum albumin, and/or IgG so as to remove phage encoding antibodies, or fragments thereof, that exhibit non-specific protein binding and phage that encode antibodies or fragments thereof that bind Fc domains, and then incubating the phage library with a population of hematopoietic stem cells. The phage library can be incubated with the target cells, such as cancer cells, autoimmune cells, or hematopoietic stem cells for a time sufficient to allow CD45- or CD117-specific antibodies, or antigen-binding fragments thereof, (e.g., GNNK+CD117-specific antibodies, or antigen-binding fragments thereof; CD45-specific antibodies, or antigen-binding fragments thereof) to bind cell-surface CD117 (e.g., sell-surface GNNK+CD117) or CD45 antigen and to subsequently be internalized by the hematopoietic stem cells (e.g., from 30 minutes to 6 hours at 4° C., such as 1 hour at 4° C.). Phage containing antibodies, or fragments thereof, that do not exhibit sufficient affinity for one or more of these antigens so as to permit binding to, and internalization by, cancer cells, autoimmune cells, or hematopoietic stem cells can subsequently be removed by washing the cells, for instance, with cold (4° C.) 0.1 M glycine buffer at pH 2.8. Phage bound to antibodies, or fragments thereof, that have been internalized by the cancer cells, autoimmune cells, or hematopoietic stem cells can be identified, for instance, by lysing the cells and recovering internalized phage from the cell culture medium. The phage can then be amplified in bacterial cells, for example, by incubating bacterial cells with recovered phage in 2×YT medium using methods known in the art. Phage recovered from this medium can then be characterized, for instance, by determining the nucleic acid sequence of the gene(s) encoding the antibodies, or fragments thereof, inserted within the phage genome. The encoded antibodies, or fragments thereof, can subsequently be prepared de novo by chemical synthesis (for instance, of antibody fragments, such as scFv fragments) or by recombinant expression (for instance, of full-length antibodies).

An exemplary method for in vitro evolution of anti-CD117 (e.g., anti-GNNK+CD117) or anti-CD45 antibodies for use with the compositions and methods described herein is phage display. Phage display libraries can be created by making a designed series of mutations or variations within a coding sequence for the CDRs of an antibody or the analogous regions of an antibody-like scaffold (e.g., the BC, CD, and DE loops of ¹⁰Fn3 domains). The template antibody-encoding sequence into which these mutations are introduced may be, for example, a naïve human germline sequence. These mutations can be performed using standard mutagenesis techniques known in the art. Each mutant sequence thus encodes an antibody corresponding to the template save for one or more amino acid variations. Retroviral and phage display vectors can be engineered using standard vector construction techniques known in the art. P3 phage display vectors along with compatible protein expression vectors can be used to generate phage display vectors for antibody diversification.

The mutated DNA provides sequence diversity, and each transformant phage displays one variant of the initial template amino acid sequence encoded by the DNA, leading to a phage population (library) displaying a vast number of different but structurally related amino acid sequences. Due to the well-defined structure of antibody hypervariable regions, the amino acid variations introduced in a phage display screen are expected to alter the binding properties of the binding peptide or domain without significantly altering its overall molecular structure.

In a typical screen, a phage library may be contacted with and allowed to bind one of the foregoing antigens or an epitope thereof. To facilitate separation of binders and non-binders, it is convenient to immobilize the target on a solid support. Phage bearing a CD117-binding or CD45-binding moiety can form a complex with the target on the solid support, whereas non-binding phage remain in solution and can be washed away with excess buffer. Bound phage can then be liberated from the target by changing the buffer to an extreme pH (pH 2 or pH 10), changing the ionic strength of the buffer, adding denaturants, or other known means.

The recovered phage can then be amplified through infection of bacterial cells, and the screening process can be repeated with the new pool that is now depleted in non-binding antibodies and enriched for antibodies that bind, e.g., CD117 (e.g., GNNK+CD117) or CD45. The recovery of even a few binding phage is sufficient to amplify the phage for a subsequent iteration of screening. After a few rounds of selection, the gene sequences encoding the antibodies or antigen-binding fragments thereof derived from selected phage clones in the binding pool are determined by conventional methods, thus revealing the peptide sequence that imparts binding affinity of the phage to the target. During the panning process, the sequence diversity of the population diminishes with each round of selection until desirable peptide-binding antibodies remain. The sequences may converge on a small number of related antibodies or antigen-binding fragments thereof. An increase in the number of phage recovered at each round of selection is an indication that convergence of the library has occurred in a screen.

Another method for identifying, e.g., anti-CD117 or anti-CD45 antibodies includes using humanizing non-human antibodies that bind CD117 (e.g., GNNK+CD117) or CD45, for instance, according to the following procedure. Consensus human antibody heavy chain and light chain sequences are known in the art (see e.g., the “VBASE” human germline sequence database; Kabat et al. Sequences of Proteins of Immunological Interest, Fifth Edition, U.S. Department of Health and Human Services, NIH Publication No. 91-3242, 1991; Tomlinson et al., J. Mol. Biol. 227:776-798, 1992; and Cox et al. Eur. J. Immunol. 24:827-836, 1994, the disclosures of each of which are incorporated herein by reference as they pertain to consensus human antibody heavy chain and light chain sequences. Using established procedures, one of skill in the art can identify the variable domain framework residues and CDRs of a consensus antibody sequence (e.g., by sequence alignment). One can substitute one or more CDRs of the heavy chain and/or light chain variable domains of consensus human antibody with one or more corresponding CDRs of a non-human antibody that binds CD117 (e.g., GNNK+CD117) or CD45 as described herein in order to produce a humanized antibody. This CDR exchange can be performed using gene editing techniques described herein or known in the art.

One example of a consensus human antibody that may be used in the preparation of a humanized antibody comprises a heavy chain variable domain set forth in SEQ ID NO: 7: EVQLVESGGGLVQPGGSLRLSCAASGFTFSDYAMSWVRQAPGKGLEWVAVISENGSD TYYADSVKGRFTISRDDSKNTLYLQMNSLRAEDTAVYYCARDRGGAVSYFDVWGQGTLVTVSS (SEQ ID NO: 7) and a light chain variable domain set forth in SEQ ID NO: 8: DIQMTQSPSSLSASVGDRVTITCRASQDVSSYLAWYQQKPGKAPKLLIYAASSLESGVP SRFSGSGSGTDFTLTISSLQPEDFATYYCQQYNSLPYTFGQGTKVEIKRT (SEQ ID NO: 8), identified in U.S. Pat. No. 6,054,297 (Genentech), the disclosure of which is incorporated herein by reference as it pertains to human antibody consensus sequences. The CDRs in the above sequences are shown in bold.

To produce humanized antibodies, one can recombinantly express a polynucleotide encoding the above consensus sequence in which one or more variable region CDRs have been replaced with one or more variable region CDR sequences of a non-human antibody that binds, e.g., CD117 (e.g., GNNK+CD117) or CD45. As the affinity of the antibody for the hematopoietic stem cell antigen is determined primarily by the CDR sequences, the resulting humanized antibody is expected to exhibit an affinity for the hematopoietic stem cell antigen that is about the same as that of the non-human antibody from which the humanized antibody was derived. Methods of determining the affinity of an antibody for a target antigen include, for instance, ELISA-based techniques described herein and known in the art, as well as surface plasmon resonance, fluorescence anisotropy, and isothermal titration calorimetry, among others.

The internalizing capacity of the prepared antibodies, or antibody fragments, can be assessed, for instance, using radionuclide internalization assays known in the art. For example, antibodies, or fragments thereof, identified using in vitro display techniques described herein or known in the art can be functionalized by incorporation of a radioactive isotope, such as ¹⁸F, ⁷⁵Br, ⁷⁷Br, ¹²²I, ¹²³I, ¹²⁴I, ¹²⁵I, ¹²⁹I, ¹³¹I, ²¹¹At, ⁶⁷Ga, ¹¹¹In, ⁹⁹Tc, ¹⁶⁹Yb, ¹⁸⁶Re, ⁶⁴CU, ⁶⁷Cu, ¹⁷⁷Lu, ⁷⁷As, ⁷²As, ⁸⁶Y, ⁹⁰Y, ⁸⁹Zr, ²¹²Bi, ²¹³Bi, or ²²⁵Ac. For instance, radioactive halogens, such as ¹⁸F, ⁷⁵Br, ⁷⁷Br, ¹²²I, ¹²³I, ¹²⁴I, ¹²⁵I, ¹²⁹I, ¹³¹I, ²¹¹At, can be incorporated into antibodies, or fragments thereof, using beads, such as polystyrene beads, containing electrophilic halogen reagents (e.g., Iodination Beads, Thermo Fisher Scientific, Inc., Cambridge, Mass.). Radiolabeled antibodies, or fragments thereof, can be incubated with cancer cells, autoimmune cells, or hematopoietic stem cells for a time sufficient to permit internalization (e.g., from 30 minutes to 6 hours at 4° C., such as 1 hour at 4° C.). The cells can then be washed to remove non-internalized antibodies, or fragments thereof, (e.g., using cold (4° C.) 0.1 M glycine buffer at pH 2.8). Internalized antibodies, or fragments thereof, can be identified by detecting the emitted radiation (e.g., γ-radiation) of the resulting cancer cells, autoimmune cells, or hematopoietic stem cells in comparison with the emitted radiation (e.g., γ-radiation) of the recovered wash buffer. The foregoing internalization assays can also be used to characterize ADCs.

Antibodies may be produced using recombinant methods and compositions, e.g., as described in U.S. Pat. No. 4,816,567. In one embodiment, isolated nucleic acid encoding an anti-CD117 or anti-CD45 antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-CLL-1 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-CD117 or anti-CD45 antibody, a nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B.K.C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003). In one embodiment, the host cell is eukaryotic, e.g. a Chinese Hamster Ovary (CHO) cell or lymphoid cell (e.g., Y0, NS0, Sp20 cell).

f. Fc-Modified Antibodies

In some embodiments, the antibodies, or antigen-binding fragments thereof, disclosed herein include Fc modifications that allow Fc silencing. Such Fc-modified antibodies are capable of binding an antigen expressed by hematopoietic stem cells, such as CD45 or CD117, can be conjugated to a drug as described herein to promote the engraftment of transplanted genetically modified hematopoietic stem cells as described herein. These therapeutic activities can be caused, for instance, by the binding of an antibody, e.g., an anti-CD45 antibody, or antigen-binding fragment thereof, or an anti-CD117 antibody, or antigen-binding fragment thereof, to CD45 or CD117, respectively, expressed by a hematopoietic cell (e.g., hematopoietic stem cell or mature immune cell (e.g., T cell)), such as a cancer cell, autoimmune cell, or hematopoietic stem cell and subsequently inducing cell death. The depletion of endogenous hematopoietic stem cells can provide a niche toward which transplanted genetically modified HSCs can home, and subsequently establish productive hematopoiesis. In this way, transplanted genetically modified HSCs may successfully engraft in a patient, such as human patient suffering from a stem cell disorder described herein. The Fc-modified antibodies and ADCs herein not only allow for selective depletion of endogenous hematopoietic stem cells, but also have reduced cytotoxic effects on the transplanted genetically modified HSC, thereby further promoting engraftment of the HSC graft.

The antibodies, or antigen-binding fragments thereof, described herein may also include modifications and/or mutations that alter the properties of the antibodies and/or fragments, such as those that increase half-life, or increase or decrease ADCC.

In one embodiment, antibodies comprising one or more radiolabeled amino acids are provided. A radiolabeled antibody may be used for both diagnostic and therapeutic purposes (conjugation to radiolabeled molecules is another possible feature). Non-limiting examples of labels for polypeptides include, but are not limited to ³H, ¹⁴C, ¹⁵N, ³⁵S, ⁹⁰Y, ⁹⁹Tc, and ¹²⁵I, ¹³¹I, and ¹⁸⁶Re. Methods for preparing radiolabeled amino acids and related peptide derivatives are known in the art (see for instance Junghans et al., in Cancer Chemotherapy and Biotherapy 655-686 (2d edition, Chafner and Longo, eds., Lippincott Raven (1996)) and U.S. Pat. Nos. 4,681,581, 4,735,210, 5,101,827, 5,102,990 (U.S. RE35,500), 5,648,471 and 5,697,902. For example, a radioisotope may be conjugated by a chloramine T method.

In one embodiment, the anti-CD45 antibody, or antigen-binding fragment thereof, or the anti-CD117 antibody, or antigen-binding fragment thereof, comprises a modified Fc region, wherein said modified Fc region comprises at least one amino acid modification relative to a wild-type Fc region, such that said molecule has an altered affinity for or binding to an FcgammaR (FcγR). Certain amino acid positions within the Fc region are known through crystallography studies to make a direct contact with FcγR. Specifically, amino acids 234-239 (hinge region), amino acids 265-269 (B/C loop), amino acids 297-299 (C′/E loop), and amino acids 327-332 (F/G) loop. (see, e.g., Sondermann et al., 2000 Nature, 406: 267-273). In some embodiments, the antibodies described herein may comprise variant Fc regions comprising modification of at least one residue that makes a direct contact with an FcγR based on structural and crystallographic analysis. In one embodiment, the Fc region of the anti-CD45 antibody, or antigen-binding fragment thereof, or the anti-CD117 antibody, or antigen-binding fragment thereof, comprises an amino acid substitution at amino acid 265 according to the EU index as in Kabat et al., Sequences of Proteins of Immunological Interest, 5th Ed. Public Health Service, NH1, MD (1991), expressly incorporated herein by reference. The “EU index as in Kabat” refers to the numbering of the human IgG1 EU antibody. In one embodiment, the Fc region comprises a D265A mutation. In one embodiment, the Fc region comprises a D265C mutation. In some embodiments, the Fc region of the antibody (or fragment thereof) comprises an amino acid substitution at amino acid 234 according to the EU index as in Kabat.

In one embodiment, the Fc region comprises a mutation at an amino acid position of D265, V205, H435, I253, and/or H310. For example, specific mutations at these positions include D265C, V205C, H435A, I253A, and/or H310A.

In one embodiment, the Fc region comprises a L234A mutation. In some embodiments, the Fc region of the anti-CD45 antibody, or antigen-binding fragment thereof, or the anti-CD117 antibody, or antigen-binding fragment thereof, comprises an amino acid substitution at amino acid 235 according to the EU index as in Kabat. In one embodiment, the Fc region comprises a L235A mutation. In yet another embodiment, the Fc region comprises a L234A and L235A mutation. In a further embodiment, the Fc region comprises a D265C, L234A, and L235A mutation. In yet a further embodiment, the Fc region comprises a D265C, L234A, L235A, and H435A mutation. In a further embodiment, the Fc region comprises a D265C and H435A mutation. In a further embodiment, the Fc region comprises an S239C.

In yet another embodiment, the Fc region comprises a L234A and L235A mutation (also referred to herein as “L234A.L235A” or as “LALA”). In another embodiment, the Fc region comprises a L234A and L235A mutation, wherein the Fc region does not include a P329G mutation. In a further embodiment, the Fc region comprises a D265C, L234A, and L235A mutation (also referred to herein as “D265C.L234A.L235A”). In another embodiment, the Fc region comprises a D265C, L234A, and L235A mutation, wherein the Fc region does not include a P329G mutation. In yet a further embodiment, the Fc region comprises a D265C, L234A, L235A, and H435A mutation (also referred to herein as “D265C.L234A.L235A.H435A”). In another embodiment, the Fc region comprises a D265C, L234A, L235A, and H435A mutation, wherein the Fc region does not include a P329G mutation. In a further embodiment, the Fc region comprises a D265C and H435A mutation (also referred to herein as “D265C.H435A”). In yet another embodiment, the Fc region comprises a D265A, S239C, L234A, and L235A mutation (also referred to herein as “D265A.S239C.L234A.L235A”). In yet another embodiment, the Fc region comprises a D265A, S239C, L234A, and L235A mutation, wherein the Fc region does not include a P329G mutation. In another embodiment, the Fc region comprises a D265C, N297G, and H435A mutation (also referred to herein as “D265C.N297G.H435A”). In another embodiment, the Fc region comprises a D265C, N297Q, and H435A mutation (also referred to herein as “D265C.N297Q.H435A”). In another embodiment, the Fc region comprises a E233P, L234V, L235A and delG236 (deletion of 236) mutation (also referred to herein as “E233P.L234V.L235A.delG236” or as “EPLVLAdelG”). In another embodiment, the Fc region comprises a E233P, L234V, L235A and delG236 (deletion of 236) mutation, wherein the Fc region does not include a P329G mutation. In another embodiment, the Fc region comprises a E233P, L234V, L235A, delG236 (deletion of 236) and H435A mutation (also referred to herein as “E233P.L234V.L235A.delG236.H435A” or as “EPLVLAdelG.H435A”). In another embodiment, the Fc region comprises a E233P, L234V, L235A, delG236 (deletion of 236) and H435A mutation, wherein the Fc region does not include a P329G mutation. In another embodiment, the Fc region comprises a L234A, L235A, S239C and D265A mutation. In another embodiment, the Fc region comprises a L234A, L235A, S239C and D265A mutation, wherein the Fc region does not include a P329G mutation. In another embodiment, the Fc region comprises a H435A, L234A, L235A, and D265C mutation. In another embodiment, the Fc region comprises a H435A, L234A, L235A, and D265C mutation, wherein the Fc region does not include a P329G mutation.

In some embodiments, the antibody has a modified Fc region such that, the antibody decreases an effector function in an in vitro effector function assay with a decrease in binding to an Fc receptor (Fc R) relative to binding of an identical antibody comprising an unmodified Fc region to the FcR. In some embodiments, the antibody has a modified Fc region such that, the antibody decreases an effector function in an in vitro effector function assay with a decrease in binding to an Fc gamma receptor (FcγR) relative to binding of an identical antibody comprising an unmodified Fc region to the FcγR. In some embodiments, the FcγR is FcγR1. In some embodiments, the FcγR is FcγR2A. In some embodiments, the FcγR is FcγR2B. In other embodiments, the FcγR is FcγR2C. In some embodiments, the FcγR is FcγR3A. In some embodiments, the FcγR is FcγR3B. In other embodiments, the decrease in binding is at least about a 70% decrease, at least about a 80% decrease, at least about a 90% decrease, at least about a 95% decrease, at least about a 98% decrease, at least about a 99% decrease, or a 100% decrease in antibody binding to a FcγR relative to binding of the identical antibody comprising an unmodified Fc region to the FcγR. In other embodiments, the decrease in binding is at least about a 70% to a 100% decrease, at least about a 80% to a 100% decrease, at least about a 90% to a 100% decrease, at least about a 95% to a 100% decrease, or at least about a 98% to a 100% decrease, in antibody binding to a FcγR relative to binding of the identical antibody comprising an unmodified Fc region to the FcγR.

In some embodiments, the antibody has a modified Fc region such that, the antibody decreases cytokine release in an in vitro cytokine release assay with a decrease in cytokine release of at least about 50% relative to cytokine release of an identical antibody comprising an unmodified Fc region. In some embodiments, the decrease in cytokine release is at least about a 70% decrease, at least about a 80% decrease, at least about a 90% decrease, at least about a 95% decrease, at least about a 98% decrease, at least about a 99% decrease, or a 100% decrease in cytokine release relative to cytokine release of the identical antibody comprising an unmodified Fc region. In some embodiments, the decrease in cytokine release is at least about a 70% to a 100% decrease, at least about a 80% to a 100% decrease, at least about a 90% to a 100% decrease, at least about a 95% to a 100% decrease in cytokine release relative to cytokine release of the identical antibody comprising an unmodified Fc region. In certain embodiments, cytokine release is by immune cells.

In some embodiments, the antibody has a modified Fc region such that, the antibody decreases mast cell degranulation in an in vitro mast cell degranulation assay with a decrease in mast cell degranulation of at least about 50% relative to mast cell degranulation of an identical antibody comprising an unmodified Fc region. In some embodiments, the decrease in mast cell degranulation is at least about a 70% decrease, at least about a 80% decrease, at least about a 90% decrease, at least about a 95% decrease, at least about a 98% decrease, at least about a 99% decrease, or a 100% decrease in mast cell degranulation relative to mast cell degranulation of the identical antibody comprising an unmodified Fc region. In some embodiments, the decrease in mast cell degranulation is at least about a 70% to a 100% decrease, at least about a 80% to a 100% decrease, at least about a 90% to a 100% decrease, or at least about a 95% to a 100% decrease, in mast cell degranulation relative to mast cell degranulation of the identical antibody comprising an unmodified Fc region.

In some embodiments, the antibody has a modified Fc region such that, the antibody decreases or prevents antibody dependent cell phagocytosis (ADCP) in an in vitro antibody dependent cell phagocytosis assay, with a decrease in ADCP of at least about 50% relative to ADCP of an identical antibody comprising an unmodified Fc region. In some embodiments, the decrease in ADCP is at least about a 70% decrease, at least about a 80% decrease, at least about a 90% decrease, at least about a 95% decrease, at least about a 98% decrease, at least about a 99% decrease, or a 100% decrease in antibody dependent cell phagocytosis to antibody dependent cell phagocytosis of the identical antibody comprising an unmodified Fc region.

In some embodiments, the anti-CD45 antibody, or antigen-binding fragment thereof, or the anti-CD117 antibody, or antigen-binding fragment thereof, described herein comprises an Fc region comprising one of the following modifications or combinations of modifications: D265A, D265C, D265C/H435A, D265C/LALA, D265C/LALA/H435A, D265A/S239C/L234A/L235A/H435A, D265A/S239C/L234A/L235A, D265C/N297G, D265C/N297G/H435A, D265C (EPLVLAdelG), D265C (EPLVLAdelG)/H435A, D265C/N297Q/H435A, D265C/N297Q, EPLVLAdelG/H435A, EPLVLAdelG/D265C, EPLVLAdelG/D265A, N297A, N297G, or N297Q. In some embodiments, the anti-CD45 antibody, or antigen-binding fragment thereof, or the anti-CD117 antibody, or antigen-binding fragment thereof, described herein comprises an Fc region comprising one of the following modifications or combinations of modifications: D265A, D265C, D265C/H435A, D265C/LALA, D265C/LALA/H435A, D265C/N297G, D265C/N297G/H435A, D265C (IgG2*), D265C (IgG2)/H435A, D265C/N297Q/H435A, D265C/N297Q, EPLVLAdelG/H435A, N297A, N297G, or N297Q.

Binding or affinity between a modified Fc region and a Fc gamma receptor can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE® analysis or Octet™ analysis (forteBIO)), and other methods such as indirect binding assays, competitive binding assays fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions. One example of a competitive binding assay is a radioimmunoassay comprising the incubation of labeled antigen with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound in the presence of increasing amounts of an unlabeled second antibody.

In one embodiment, an antibody having the Fc modifications described herein (e.g., D265C, L234A, L235A, and/or H435A) has at least about a 70% decrease, at least about a 75% decrease, at least about a 80% decrease, at least about a 85% decrease, at least about a 90% decrease, at least about a 95% decrease, at least about a 98% decrease, at least about a 99% decrease, or a 100% decrease in binding to a Fc gamma receptor relative to binding of the identical antibody comprising an unmodified Fc region to the Fc gamma receptor (e.g., as assessed by biolayer interferometry (BLI)).

Without wishing to be bound by any theory, it is believed that Fc region binding interactions with a Fc gamma receptor are essential for a variety of effector functions and downstream signaling events including, but not limited to, antibody dependent cell-mediated cytotoxicity (ADCC) and complement dependent cytotoxicity (CDC). Accordingly, in certain aspects, an antibody comprising a modified Fc region (e.g., comprising a L234A, L235A, and/or a D265C mutation) has substantially reduced or abolished effector functions. Effector functions can be assayed using a variety of methods known in the art, e.g., by measuring cellular responses (e.g., mast cell degranulation or cytokine release) in response to the antibody of interest. For example, using standard methods in the art, the Fc-modified antibodies can be assayed for their ability to trigger mast cell degranulation in vitro or for their ability to trigger cytokine release, e.g. by human peripheral blood mononuclear cells.

Thus, in one embodiment, the Fc region comprises a mutation resulting in a decrease in half life (e.g., relative to an antibody having an unmodified Fc region). An antibody having a short half life may be advantageous in certain instances where the antibody is expected to function as a short-lived therapeutic, e.g., the conditioning step described herein where the antibody is administered followed by transplant of genetically modified HSCs. Ideally, the antibody would be substantially cleared prior to delivery of the genetically modified HSCs, which also generally express a target antigen (e.g., CD45 or CD117) but are not the target of the anti-CD45 or anti-CD117 antibody unlike the endogenous stem cells. In one embodiment, the Fc regions comprise a mutation at position 435 (EU index according to Kabat). In one embodiment, the mutation is an H435A mutation.

In one embodiment, the anti-CD45 or anti-CD117 antibody described herein has a half-life (e.g., in humans) equal to or less than about 24 hours, equal to or less than about 23 hours, equal to or less than about 22 hours, equal to or less than about 21 hours, equal to or less than about 20 hours, equal to or less than about 19 hours, equal to or less than about 18 hours, equal to or less than about 17 hours, equal to or less than about 16 hours, equal to or less than about 15 hours, equal to or less than about 14 hours, equal to or less than about 13 hours, equal to or less than about 12 hours, or equal to or less than about 11 hours.

In one embodiment, the anti-CD45 or anti-CD117 antibody described herein has a half-life (e.g., in humans) about 1-5 hours, about 5-10 hours, about 10-15 hours, about 15-20 hours, or about 20 to 25 hours. In another embodiment, the half-life of the anti-CD45 or anti-CD117 antibody described herein is about 5-7 hours; about 5-9 hours; about 5-11 hours; about 5-13 hours; about 5-15 hours; about 5-20 hours; about 5-24 hours; about 7-24 hours; about 9-24 hours; about 11-24 hours; about 12-22 hours; about 10-20 hours; about 8-18 hours; or about 14-24 hours.

In some aspects, the Fc region comprises two or more mutations that confer reduced half-life and reduce an effector function of the antibody. In some embodiments, the Fc region comprises a mutation resulting in a decrease in half-life and a mutation of at least one residue that can make direct contact with an FcγR (e.g., as based on structural and crystallographic analysis). In one embodiment, the Fc region comprises a H435A mutation, a L234A mutation, and a L235A mutation. In one embodiment, the Fc region comprises a H435A mutation and a D265C mutation. In one embodiment, the Fc region comprises a H435A mutation, a L234A mutation, a L235A mutation, and a D265C mutation.

In some embodiments, the antibody or antigen-binding fragment thereof as described herein is conjugated to a cytotoxin (e.g., amatoxin) by way of a cysteine residue in the Fc domain of the antibody or antigen-binding fragment thereof. In some embodiments, the cysteine residue is introduced by way of a mutation in the Fc domain of the antibody or antigen-binding fragment thereof. For instance, the cysteine residue may be selected from the group consisting of Cys118, Cys239, and Cys265. In one embodiment, the Fc region of the anti-CD45 antibody, or antigen-binding fragment thereof, of the anti-CD117 antibody, or antigen-binding fragment thereof, described herein comprises an amino acid substitution at amino acid 265 according to the EU index as in Kabat. In one embodiment, the Fc region comprises a D265C mutation. In one embodiment, the Fc region comprises a D265C and H435A mutation. In one embodiment, the Fc region comprises a D265C, a L234A, and a L235A mutation. In one embodiment, the Fc region comprises a D265C, a L234A, a L235A, and a H435A mutation. In one embodiment, the Fc region of the anti-CD117 antibody or antigen-binding fragment thereof, or the anti-CD45 antibody or antigen-binding fragment thereof, (or, e.g., an anti-CD2 antibody, an anti-CD5 antibody, an anti-CD137 antibody, or an anti-CD252 antibody), comprises an amino acid substitution at amino acid 239 according to the EU index as in Kabat. In one embodiment, the Fc region comprises a S239C mutation. In one embodiment, the Fc region comprises a L234A mutation, a L235A mutation, a S239C mutation and a D265A mutation. In another embodiment, the Fc region comprises a S239C and H435A mutation. In another embodiment, the Fc region comprises a L234A mutation, a L235A mutation, and S239C mutation. In yet another embodiment, the Fc region comprises a H435A mutation, a L234A mutation, a L235A mutation, and S239C mutation. In yet another embodiment, the Fc region comprises a H435A mutation, a L234A mutation, a L235A mutation, a S239C mutation and D265A mutation.

Notably, Fc amino acid positions are in reference to the EU numbering index unless otherwise indicated.

The disclosures of each of the foregoing publications are incorporated herein by reference as they pertain to an anti-CD45 antibody or an anti-CD117 antibody. Antibodies and antigen-binding fragments that may be used in conjunction with the compositions and methods described herein include the above-described antibodies and antigen-binding fragments thereof, as well as humanized variants of those non-human antibodies and antigen-binding fragments described above and antibodies or antigen-binding fragments that bind the same epitope as those described above, as assessed, for instance, by way of a competitive antigen binding assay.

The antibodies of the present disclosure may be further engineered to further modulate antibody half-life by introducing additional Fc mutations, such as those described for example in (Dall'Acqua et al. (2006) J Biol Chem 281: 23514-24), (Zalevsky et al. (2010) Nat Biotechnol 28: 157-9), (Hinton et al. (2004) J Biol Chem 279: 6213-6), (Hinton et al. (2006) J Immunol 176: 346-56), (Shields et al. (2001) J Biol Chem 276: 6591-604), (Petkova et al. (2006) Int Immunol 18: 1759-69), (Datta-Mannan et al. (2007) Drug Metab Dispos 35: 86-94), (Vaccaro et al. (2005) Nat Biotechnol 23: 1283-8), (Yeung et al. (2010) Cancer Res 70: 3269-77) and (Kim et al. (1999) Eur J Immunol 29: 2819-25), and include positions 250, 252, 253, 254, 256, 257, 307, 376, 380, 428, 434 and 435. Exemplary mutations that may be made singularly or in combination are T250Q, M252Y, I253A, S254T, T256E, P2571, T307A, D376V, E380A, M428L, H433K, N434S, N434A, N434H, N434F, H435A and H435R mutations.

Methods of engineering antibodies to include any of the Fc modifications herein are well known in the art. These methods include, but are not limited to, preparation by site-directed (or oligonucleotide-mediated) mutagenesis, PCR mutagenesis, and cassette mutagenesis of a prepared DNA molecule encoding the antibody or at least the constant region of the antibody. Site-directed mutagenesis is well known in the art (see, e.g., Carter et al., Nucleic Acids Res., 13:4431-4443 (1985) and Kunkel et al., Proc. Natl. Acad. Sci. USA, 82:488 (1987)). PCR mutagenesis is also suitable for making amino acid sequence variants of the starting polypeptide. See Higuchi, in PCR Protocols, pp. 177-183 (Academic Press, 1990); and Vallette et al., Nuc. Acids Res. 17:723-733 (1989). Another method for preparing sequence variants, cassette mutagenesis, is based on the technique described by Wells et al., Gene, 34:315-323 (1985).

g. Other Antigen Binding Proteins

In certain embodiments an antigen binding protein (the antigen targeting moiety), such as a ligand or functionally active fragment thereof, is used in a conjugate or fusion protein described herein. For example, stem cell factor (SCF) is a ligand for CD117, where SCF can be conjugated or fused to a toxin to achieve the conditioning methods disclosed herein.

In certain embodiments, an antibody mimetic is used as the antigen targeting moiety in the compositions and methods disclosed herein. Examples of antibody mimetics include, but are not limited to, an adnectins, an affibody, an afflins, an affimer, an affitin, and alphabody, and anticalin, an aptamer, an armadillo repeat protein-based scaffold, an atrimer, an avimer, a DARpin, a fynomer, a knottin, a Kunitz domain peptide, a monobody, and a nanofitin.

2. Cytotoxins

Antibodies and antigen-binding fragments thereof described herein can be conjugated (linked) to a cytotoxin via a linker. In some embodiments, the cytotoxic molecule is conjugated to a cell internalizing antibody, or antigen-binding fragment thereof as disclosed herein such that following the cellular uptake of the antibody, or fragment thereof, the cytotoxin may access its intracellular target and mediate hematopoietic cell death. Any number of cytotoxins, e.g., 1, 2, 3, 4, 5, 6, 7, or 8 can be conjugated to the antibody, e.g., anti-CD117 or anti-CD45.

In some embodiments, the ADCs described herein include an antibody (or an antigen-binding fragment thereof) conjugated (i.e., covalently attached by a linker) to a cytotoxic moiety (or cytotoxin). In various embodiments, the cytotoxic moiety exhibits reduced or no cytotoxicity when bound in a conjugate, but resumes cytotoxicity after cleavage from the linker. In various embodiments, the cytotoxic moiety maintains cytotoxicity without cleavage from the linker. In some embodiments, the cytotoxic molecule is conjugated to a cell internalizing antibody, or antigen-binding fragment thereof as disclosed herein, such that following the cellular uptake of the antibody, or fragment thereof, the cytotoxin may access its intracellular target and, e.g., mediate T cell death.

Antibodies, and antigen-binding fragments thereof, described herein (e.g., antibodies, and antigen-binding fragments thereof, that recognize and bind CD117 or CD45) can be conjugated (or linked) to a cytotoxin.

ADCs of the present disclosure therefore may be of the general formula Ab-(Z-L-D)ₙ wherein an antibody or antigen-binding fragment thereof (Ab) is conjugated (covalently linked) to linker (L), through a chemical moiety (Z), to a cytotoxic moiety (“drug,” D). “n” represents the number of drugs linked to the antibody, and generally ranges from 1 to 8.

Accordingly, the antibody or antigen-binding fragment thereof described herein may be conjugated to a number of drug moieties as indicated by integer n, which represents the average number of cytotoxins per antibody, which may range, e.g., from about 1 to about 20. In some embodiments, n is from 1 to 4. In some embodiments, n is 1. In some embodiments, n is 2. In some embodiments, n is 3. In some embodiments, n is 4. The average number of drug moieties per antibody in preparations of ADC from conjugation reactions may be characterized by conventional means such as mass spectroscopy, ELISA assay, and HPLC. The quantitative distribution of ADC in terms of n may also be determined. In some instances, separation, purification, and characterization of homogeneous ADC where n is a certain value from ADC with other drug loadings may be achieved by means such as reverse phase HPLC or electrophoresis.

For some anti-CD117 ADCs or anti-CD45 ADCs described herein, the average number of cytotoxins per antibody may be limited by the number of attachment sites on the antibody. For example, where the attachment is a cysteine thiol, an antibody may have only one or several cysteine thiol groups, or may have only one or several sufficiently reactive thiol groups through which a linker and chemical moiety may be attached. Generally, antibodies do not contain many free and reactive cysteine thiol groups which may be linked to a drug moiety; primarily, cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups.

In certain embodiments, fewer than the theoretical maximum of drug moieties are conjugated to an antibody during a conjugation reaction. An antibody may contain, for example, lysine residues that do not react with the drug-linker intermediate or linker reagent, as discussed below. Only the most reactive lysine groups may react with an amine-reactive linker reagent. In certain embodiments, an antibody is subjected to denaturing conditions to reveal reactive nucleophilic groups such as lysine or cysteine.

The loading (drug/antibody ratio) of an ADC may be controlled in different ways, e.g., by: (i) limiting the molar excess of drug-linker intermediate or linker reagent relative to antibody, (ii) limiting the conjugation reaction time or temperature, (iii) partial or limiting reductive conditions for cysteine thiol modification, (iv) engineering by recombinant techniques the amino acid sequence of the antibody such that the number and position of cysteine residues is modified for control of the number and/or position of linker-drug attachments.

Cytotoxins suitable for use in the ADCs described herein include DNA-intercalating agents, (e.g., anthracyclines), agents capable of disrupting the mitotic spindle apparatus (e.g., vinca alkaloids, maytansine, maytansinoids, and derivatives thereof), RNA polymerase inhibitors (e.g., an amatoxin, such as a-amanitin, and derivatives thereof), and agents capable of disrupting protein biosynthesis (e.g., agents that exhibit rRNA N-glycosidase activity, such as saporin and ricin A-chain), among others known in the art.

Cytotoxins suitable for use with the compositions and methods described herein include, without limitation, 5-ethynyluracil, abiraterone, acylfulvene, adecypenol, adozelesin, aldesleukin, altretamine, ambamustine, amidox, amifostine, aminolevulinic acid, amrubicin, amsacrine, anagrelide, anastrozole, andrographolide, angiogenesis inhibitors, antarelix, anti-dorsalizing morphogenetic protein-1, antiandrogen, prostatic carcinoma, antiestrogen, antineoplaston, antisense oligonucleotides, aphidicolin glycinate, apoptosis gene modulators, apoptosis regulators, apurinic acid, asulacrine, atamestane, atrimustine, axinastatin 1, axinastatin 2, axinastatin 3, azasetron, azatoxin, azatyrosine, baccatin Ill derivatives, balanol, batimastat, BCR/ABL antagonists, benzochlorins, benzoylstaurosporine, beta lactam derivatives, beta-alethine, betaclamycin B, betulinic acid, bFGF inhibitors, bicalutamide, bisantrene, bisaziridinylspermine, bisnafide, bistratene A, bizelesin, breflate, bleomycin A2, bleomycin B2, bropirimine, budotitane, buthionine sulfoximine, calcipotriol, calphostin C, camptothecin derivatives (e.g., 10-hydroxy-camptothecin), capecitabine, carboxamide-amino-triazole, carboxyamidotriazole, carzelesin, casein kinase inhibitors, castanospermine, cecropin B, cetrorelix, chlorins, chloroquinoxaline sulfonamide, cicaprost, cis-porphyrin, cladribine, clomifene and analogues thereof, clotrimazole, collismycin A, collismycin B, combretastatin A4, combretastatin analogues, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, decitabine, dehydrodidemnin B, 2′deoxycoformycin (DCF), deslorelin, dexifosfamide, dexrazoxane, dexverapamil, diaziquone, didemnin B, didox, diethylnorspermine, dihydro-5-azacytidine, dihydrotaxol, dioxamycin, diphenyl spiromustine, discodermolide, docosanol, dolasetron, doxifluridine, droloxifene, dronabinol, duocarmycin SA, ebselen, ecomustine, edelfosine, edrecolomab, eflornithine, elemene, emitefur, epothilones, epithilones, epristeride, estramustine and analogues thereof, etoposide, etoposide 4′-phosphate (also referred to as etopofos), exemestane, fadrozole, fazarabine, fenretinide, filgrastim, finasteride, flavopiridol, flezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors, gemcitabine, glutathione inhibitors, hepsulfam, homoharringtonine (HHT), hypericin, ibandronic acid, idoxifene, idramantone, ilmofosine, ilomastat, imidazoacridones, imiquimod, immunostimulant peptides, iobenguane, iododoxorubicin, ipomeanol, irinotecan, iroplact, irsogladine, isobengazole, jasplakinolide, kahalalide F, lamellarin-N triacetate, lanreotide, leinamycin, lenograstim, lentinan sulfate, leptolstatin, letrozole, lipophilic platinum compounds, lissoclinamide 7, lobaplatin, lometrexol, lonidamine, losoxantrone, loxoribine, lurtotecan, lutetium texaphyrin, lysofylline, masoprocol, maspin, matrix metalloproteinase inhibitors, menogaril, rnerbarone, meterelin, methioninase, metoclopramide, MIF inhibitor, ifepristone, miltefosine, mirimostim, mithracin, mitoguazone, mitolactol, mitomycin and analogues thereof, mitonafide, mitoxantrone, mofarotene, molgramostim, mycaperoxide B, myriaporone, N-acetyldinaline, N-substituted benzamides, nafarelin, nagrestip, napavin, naphterpin, nartograstim, nedaplatin, nemorubicin, neridronic acid, nilutamide, nisamycin, nitrullyn, octreotide, okicenone, onapristone, ondansetron, oracin, ormaplatin, oxaliplatin, oxaunomycin, paclitaxel and analogues thereof, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, phenazinomycin, picibanil, pirarubicin, piritrexim, podophyllotoxin, porfiromycin, purine nucleoside phosphorylase inhibitors, raltitrexed, rhizoxin, rogletimide, rohitukine, rubiginone B1, ruboxyl, safingol, saintopin, sarcophytol A, sargramostim, sobuzoxane, sonermin, sparfosic acid, spicamycin D, spiromustine, stipiamide, sulfinosine, tallimustine, tegafur, temozolomide, teniposide, thaliblastine, thiocoraline, tirapazamine, topotecan, topsentin, triciribine, trimetrexate, veramine, vinorelbine, vinxaltine, vorozole, zeniplatin, and zilascorb, among others.

In some embodiments, the cytotoxin is a microtubule-binding agent (for instance, maytansine or a maytansinoid), an amatoxin, pseudomonas exotoxin A, deBouganin, diphtheria toxin, saporin, an auristatin, an anthracycline, a calicheamicin, irinotecan, SN-38, a duocarmycin, a pyrrolobenzodiazepine, a pyrrolobenzodiazepine dimer, an indolinobenzodiazepine, an indolinobenzodiazepine dimer, an indolinobenzodiazepine pseudodimer, or a variant thereof, or another cytotoxic compound described herein or known in the art.

In some embodiments, the cytotoxin is a maytansinoid selected from the group consisting of DM1 and DM4. In some embodiments, the cytotoxin is an auristatin selected from the group consisting of monomethyl auristatin E and monomethyl auristatin F.

In some embodiments, the cytotoxin is an anthracycline selected from the group consisting of daunorubicin, doxorubicin, epirubicin, and idarubicin.

In some embodiments, the cytotoxin of the antibody-drug conjugate is an RNA polymerase inhibitor. In some embodiments, the RNA polymerase inhibitor is an amatoxin or derivative thereof. Amatoxins are potent and selective inhibitors of RNA polymerase II and thereby also inhibit the transcription and protein biosynthesis of the affected cells. As used herein, the term “amatoxin” refers to a member of the amatoxin family of peptides produced by Amanita phalloides mushrooms, or a variant or derivative thereof, such as a variant or derivative thereof capable of inhibiting RNA polymerase II activity. Amatoxins are rigid bicyclic octapeptides having the basic sequence Ile-Trp-Gly-Ile-Gly-Cys-Asn (or Asp)-Pro (SEQ ID NO: 325), crosslinked by an attachment between the Cys sulfur and position 2 of the Trp indole ring, forming a tryptathionine.

In some embodiments, the cytotoxin to which the antibody, antibody fragment, or other antigen binding agent, e.g., a ligand such as stem cell factor, is attached is a protein-based toxin. An example of a protein based toxin is a shiga toxin. Thus, in some embodiments, the cytotoxin to which the antibody, antibody fragment, or other antigen binding agent, e.g., a ligand such as stem cell factor, is attached is a Shiga toxin, or a mutant, fragment or derivative thereof, for example Shiga-like toxin A subunit, and mutants, fragments, and derivatives thereof. In some embodiments, the cytotoxin to which the antibody, antibody fragment, or other antigen binding agent is conjugated is a Shiga-like toxin such as SLT I, SLT II, SLT IIV, LT toxin, or C3 toxin.

In certain embodiments, the cytotoxin is part of a fusion protein comprising a protein-based toxin and an antigen binding protein. For example, a fusion protein in certain embodiments is an engineered toxin body comprising an antibody fragment, such as an scFv, and a protein-based toxin, e.g., a protein synthesis inhibitor, e.g., a ribosome inactivating protein, e.g., Shiga toxin, Shiga-like toxin A subunit, saporin, ricin, and mutants, fragments, and derivatives thereof, etc.

### Maytansinoids

In some embodiments, the antibodies and antigen-binding fragments thereof described herein can be conjugated to a cytotoxin that is a microtubule binding agent. In some embodiments, the microtubule binding agent is a maytansine, a maytansinoid or a maytansinoid analog. Maytansinoids are mitototic inhibitors which bind microtubules and act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533. Maytansinoid drug moieties are attractive drug moieties in antibody drug conjugates because they are: (i) relatively accessible to prepare by fermentation or chemical modification, derivatization of fermentation products, (ii) amenable to derivatization with functional groups suitable for conjugation through the non-disulfide linkers to antibodies, (iii) stable in plasma, and (iv) effective against a variety of tumor cell lines.

Examples of suitable maytansinoids include esters of maytansinol, synthetic maytansinol, and maytansinol analogs and derivatives. Included herein are any cytotoxins that inhibit microtubule formation and that are highly toxic to mammalian cells, as are maytansinoids, maytansinol, and maytansinol analogs, and derivatives.

Examples of suitable maytansinol esters include those having a modified aromatic ring and those having modifications at other positions. Such suitable maytansinoids are disclosed in U.S. Pat. Nos. 4,137,230; 4,151,042; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,362,663; 4,364,866; 4,424,219; 4,450,254; 4,322,348; 4,362,663; 4,371,533; 5,208,020; 5,416,064; 5,475,092; 5,585,499; 5,846,545; 6,333,410; 7,276,497; and 7,473,796, the disclosures of each of which are incorporated herein by reference as they pertain to maytansinoids and derivatives thereof.

In some embodiments, the antibody-drug conjugates (ADCs) of the present disclosure utilize the thiol-containing maytansinoid (DM1), formally termed N²′-deacetyl-N²′-(3-mercapto-1-oxopropyl)-maytansine, as the cytotoxic agent. DM1 is represented by the structural formula (IV):

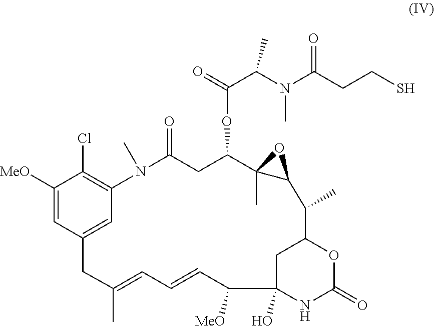 [SMILES: COc1cc2cc(c1Cl)N(C)C(=O)C[C@H](OC(=O)[C@H](C)N(C)C(=O)CCS)[C@]1(C)O[C@H]1[C@H](C)C1C[C@@](O)(NC(=O)O1)[C@H](OC)/C=C/C=C(\C)C2]

In another embodiment, the conjugates of the present disclosure utilize the thiol-containing maytansinoid N²′-deacetyl-N²′(4-methyl-4-mercapto-1-oxopentyl)-maytansine (e.g., DM4) as the cytotoxic agent. DM4 is represented by the structural formula (V):

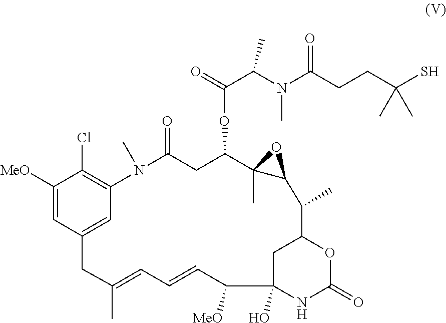 [SMILES: COc1cc2cc(c1Cl)N(C)C(=O)C[C@H](OC(=O)[C@H](C)N(C)C(=O)CCC(C)(C)S)[C@]1(C)O[C@H]1[C@H](C)C1C[C@@](O)(NC(=O)O1)[C@H](OC)/C=C/C=C(\C)C2]

Another maytansinoid comprising a side chain that contains a sterically hindered thiol bond is N²′-deacetyl-N-²′(4-mercapto-1-oxopentyl)-maytansine (termed DM3), represented by the structural formula (VI):

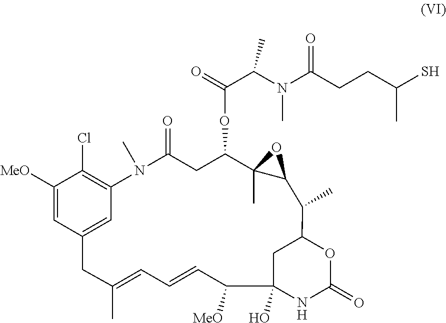 [SMILES: COc1cc2cc(c1Cl)N(C)C(=O)C[C@H](OC(=O)[C@H](C)N(C)C(=O)CCC(C)S)[C@]1(C)O[C@H]1[C@H](C)C1C[C@@](O)(NC(=O)O1)[C@H](OC)/C=C/C=C(\C)C2]

Each of the maytansinoids taught in U.S. Pat. Nos. 5,208,020 and 7,276,497, can also be used in the conjugate of the present disclosure. In this regard, the entire disclosure of U.S. Pat. Nos. 5,208,020 and 7,276,697 is incorporated herein by reference.

Many positions on maytansinoids can serve as the position to chemically link the linking moiety. For example, the C-3 position having a hydroxyl group, the C-14 position modified with hydroxymethyl, the C-15 position modified with hydroxy and the C-20 position having a hydroxy group are all expected to be useful. In some embodiments, the C-3 position serves as the position to chemically link the linking moiety, and in some particular embodiments, the C-3 position of maytansinol serves as the position to chemically link the linking moiety. There are many linking groups known in the art for making antibody-maytansinoid conjugates, including, for example, those disclosed in U.S. Pat. Nos. 5,208,020, 6,441,163, and EP Patent No. 0425235 B1; Chari et al., Cancer Research 52:127-131 (1992); and U.S. 2005/0169933 A1, the disclosures of which are hereby expressly incorporated by reference. Additional linking groups are described and exemplified herein.

The present disclosure also includes various isomers and mixtures of maytansinoids and conjugates. Certain compounds and conjugates of the present disclosure may exist in various stereoisomeric, enantiomeric, and diastereomeric forms. Several descriptions for producing such antibody-maytansinoid conjugates are provided in U.S. Pat. Nos. 5,208,020, 5,416,064 6,333,410, 6,441,163, 6,716,821, and 7,368,565, each of which is incorporated herein in its entirety.

A therapeutically effective number of maytansinoid molecules bound per antibody molecule can be determined by measuring spectrophotometrically the ratio of the absorbance at 252 nm and 280 nm. In certain embodiments, an average of 3 to 4 maytansinoid molecules conjugated per antibody molecule may enhance the cytotoxicity of target cells without negatively affecting the function or solubility of the antibody, although one molecule of toxin/antibody can enhance cytotoxicity over antibody alone. The average number of maytansinoid molecules/antibody or antigen binding fragment thereof can be, for example, 1-10 or 2-5.

### Anthracyclines

In other embodiments, the antibodies and antigen-binding fragments thereof described herein can be conjugated to a cytotoxin that is an anthracycline molecule. Anthracyclines are antibiotic compounds that exhibit cytotoxic activity. Studies have indicated that anthracyclines may operate to kill cells by a number of different mechanisms including: 1) intercalation of the drug molecules into the DNA of the cell thereby inhibiting DNA-dependent nucleic acid synthesis; 2) production by the drug of free radicals which then react with cellular macromolecules to cause damage to the cells or 3) interactions of the drug molecules with the cell membrane [see, e.g., C. Peterson et al.,” Transport And Storage Of Anthracycline In Experimental Systems And Human Leukemia” in Anthracycline Antibiotics In Cancer Therapy; N. R. Bachur, “Free Radical Damage” id. at pp. 97-102]. Because of their cytotoxic potential, anthracyclines have been used in the treatment of numerous cancers such as leukemia, breast carcinoma, lung carcinoma, ovarian adenocarcinoma and sarcomas [see e.g., P.H-Wiernik, in Anthracycline: Current Status And New Developments p 11]. Commonly used anthracyclines include doxorubicin, epirubicin, idarubicin and daunomycin.

In some embodiments, the cytotoxin is an anthracycline selected from the group consisting of daunorubicin, doxorubicin, epirubicin, and idarubicin. Representative examples of anthracyclines include, but are not limited to daunorubicin (Cerubidine; Bedford Laboratories), doxorubicin (Adriamycin; Bedford Laboratories; also referred to as doxorubicin hydrochloride, hydroxy-daunorubicin, and Rubex), epirubicin (Ellence; Pfizer), and idarubicin (Idamycin; Pfizer Inc.).

The anthracycline analog, doxorubicin (ADRIAMYCINO) is thought to interact with DNA by intercalation and inhibition of the progression of the enzyme topoisomerase II, which unwinds DNA for transcription. Doxorubicin stabilizes the topoisomerase II complex after it has broken the DNA chain for replication, preventing the DNA double helix from being resealed and thereby stopping the process of replication. Doxorubicin and daunorubicin (DAUNOMYCIN) are prototype cytotoxic natural product anthracycline chemotherapeutics (Sessa et al., (2007) Cardiovasc. Toxicol. 7:75-79).

One non-limiting example of a suitable anthracycline for use herein is PNU-159682 (“PNU”). PNU exhibits greater than 3000-fold cytotoxicity relative to the parent nemorubicin (Quintieri et al., Clinical Cancer Research 2005, 11, 1608-1617). PNU is represented by structural formula:

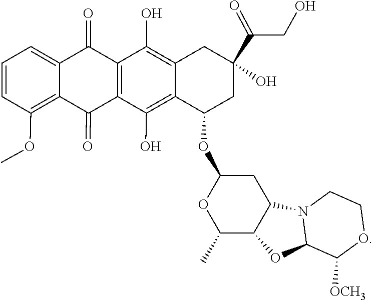

Multiple positions on anthracyclines such as PNU can serve as the position to covalently bond the linking moiety and, hence the antibodies and antigen-binding fragments thereof described herein. For example, linkers may be introduced through modifications to the hydroxymethyl ketone side chain.

In some embodiments, the cytotoxin is a PNU derivative represented by structural formula:

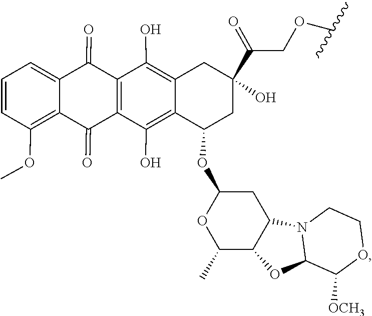

wherein the wavy line indicates the point of covalent attachment to the linker of the ADC as described herein.

In some embodiments, the cytotoxin is a PNU derivative represented by structural formula:

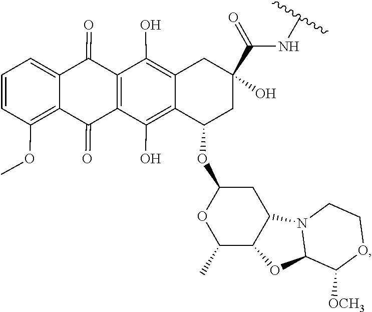

wherein the wavy line indicates the point of covalent attachment to the linker of the ADC as described herein.

### Pyrrolobenzodiazepines (PBDs)

In other embodiments, the antibodies and antigen-binding fragments thereof described herein can be conjugated to a cytotoxin that is a pyrrolobenzodiazepine (PBD) or a cytotoxin that comprises a PBD. PBDs are known to be sequence selective DNA alkylating compounds. PBD cytotoxins include, but are not limited to, anthramycin, dimeric PBDs, and those disclosed in, for example, Hartley, J.A. (2011). “The development of pyrrolobenzodiazepines as antitumour agents.” Expert Opin. Inv. Drug, 20(6), 733-744; and Antonow, D.; Thurston, D. E. (2011) “Synthesis of DNA-interactive pyrrolo[2,1-c][1,4]benzodiazepines (PBDs).” Chem. Rev. 111: 2815-2864.

In some embodiments, the cytotoxin may be a pyrrolobenzodiazepine dimer represented by the formula:

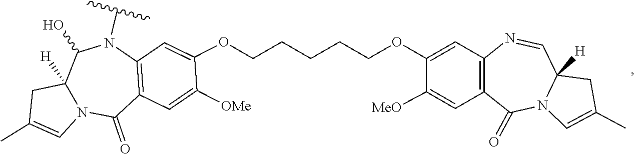

wherein the wavy line indicates the attachment point of the linker.

In some embodiments, the cytotoxin is conjugated to the antibody, or the antigen-binding fragment thereof, by way of a maleimidocaproyl linker.

In some embodiments, the linker comprises one or more of a peptide, oligosaccharide, —(CH₂)ₚ—, —(CH₂CH₂O)_{q}—, —(C═O)(CH₂)ᵣ—, —(C═O)(CH₂CH₂O)—, —(NHCH₂CH₂)ᵤ—, -PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB, wherein each of p, q, r, t, and u are integers from 1-12, selected independently for each occurrence.

In some embodiments, the linker has the structure of formula:

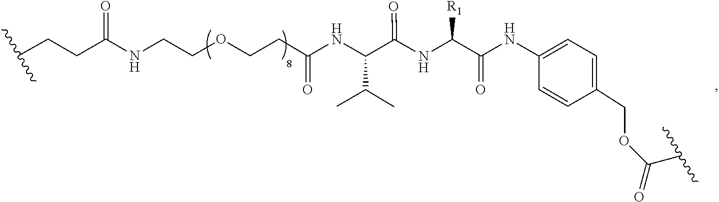

wherein R₁ is CH₃ (Ala) or (CH₂)₃NH(CO)NH₂ (Cit).

In some embodiments, the linker, prior to conjugation to the antibody and including the reactive substituent Z′, taken together as L-Z′, has the structure:

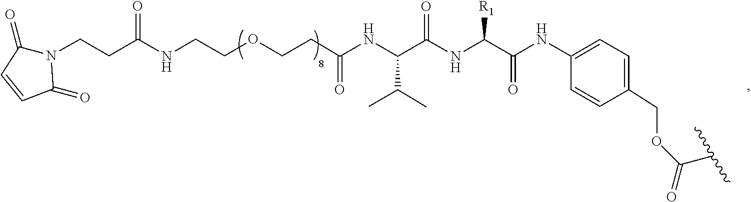

wherein the wavy line indicates the attachment point to the cytotoxin (e.g., a PBD). In certain embodiments, R₁ is CH₃.

In some embodiments, the cytotoxin-linker conjugate, prior to conjugation to the antibody and including the reactive substituent Z′, taken together as Cy-L-Z′, has the structure of formula:

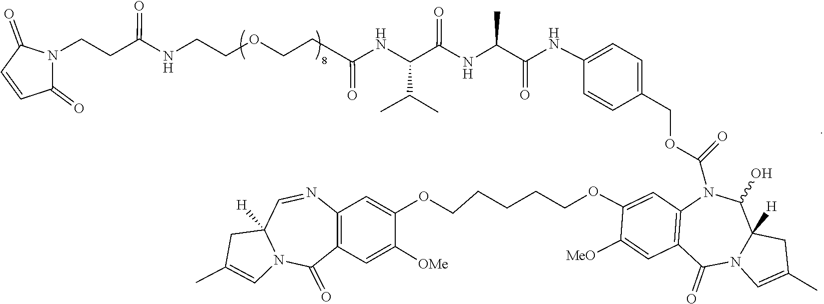

This particular cytotoxin-linker conjugate is known as tesirine (SG3249), and has been described in, for example, Howard et al., ACS Med. Chem. Lett. 2016, 7(11), 983-987, the disclosure of which is incorporated by reference herein in its entirety.

In some embodiments, the cytotoxin may be a pyrrolobenzodiazepine dimer represented by formula:

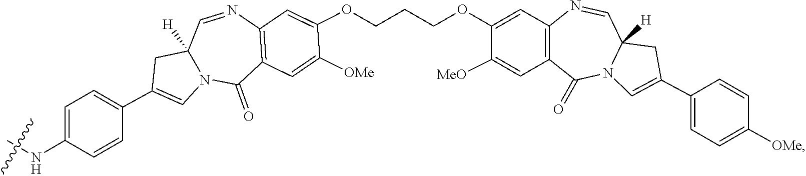

wherein the wavy line indicates the attachment point of the linker.

In some embodiments, the cytotoxin-linker conjugate, prior to conjugation to the antibody and including the reactive substituent Z′, taken together as Cy-L-Z′, has the structure of formula:

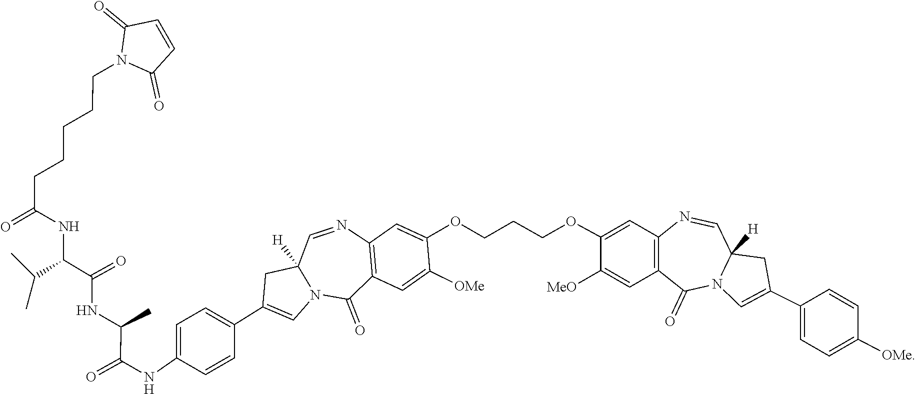 [SMILES: COc1ccc(C2=CN3C(=O)c4cc(OC)c(OCCCOc5cc6c(cc5OC)C(=O)N5C=C(c7ccc(NC(=O)[C@H](C)NC(=O)[C@@H](NC(=O)CCCCCN8C(=O)C=CC8=O)C(C)C)cc7)C[C@H]5C=N6)cc4N=C[C@@H]3C2)cc1]

This particular cytotoxin-linker conjugate is known as talirine, and has been described, for example, in connection with the ADC Vadastuximab talirine (SGN-CD33A), Mantaj et al., Angewandte Chemie International Edition English 2017, 56, 462-488, the disclosure of which is incorporated by reference herein in its entirety.

In some embodiments, the cytotoxin may be an indolinobenzodiazepine pseudodimer having the structure of formula:

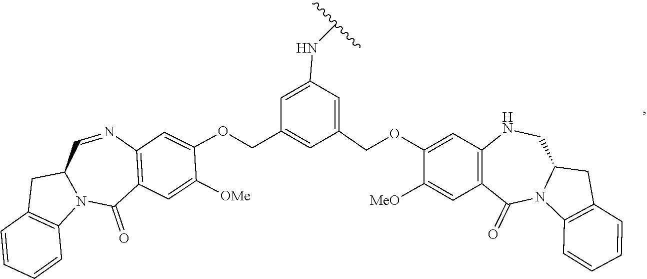 [SMILES: *Nc1cc(COc2cc3c(cc2OC)C(=O)N2c4ccccc4C[C@H]2C=N3)cc(COc2cc3c(cc2OC)C(=O)N2c4ccccc4C[C@H]2CN3)c1]

wherein the wavy line indicates the attachment point of the linker.

In some embodiments, the cytotoxin-linker conjugate, prior to conjugation to the antibody and including the reactive substituent Z′, taken together as Cy-L-Z′, has the structure of formula:

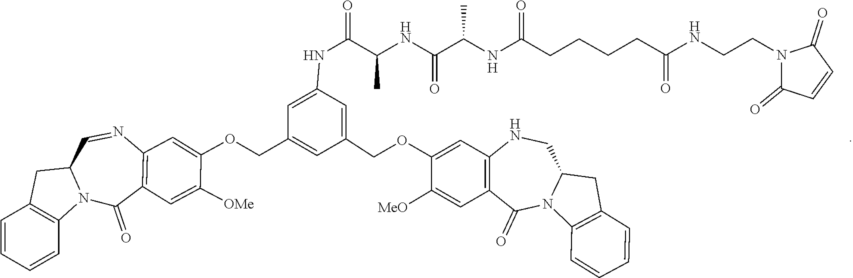 [SMILES: COc1cc2c(cc1OCc1cc(COc3cc4c(cc3OC)C(=O)N3c5ccccc5C[C@H]3CN4)cc(NC(=O)[C@H](C)NC(=O)[C@H](C)NC(=O)CCCCC(=O)NCCN3C(=O)C=CC3=O)c1)N=C[C@@H]1Cc3ccccc3N1C2=O]

which comprises the ADC IMGN632, disclosed in, for example, International Patent Application Publication No. WO2017004026, which is incorporated by reference herein.

### Calicheamicin

In other embodiments, the antibodies and antigen-binding fragments thereof described herein can be conjugated to a cytotoxin that is an enediyne antitumor antibiotic (e.g., calicheamicins, ozogamicin). The calicheamicin family of antibiotics are capable of producing double-stranded DNA breaks at sub-picomolar concentrations. For the preparation of conjugates of the calicheamicin family, see U.S. Pat. Nos. 5,712,374; 5,714,586; 5,739,116; 5,767,285; 5,770,701; 5,770,710; 5,773,001; and 5,877,296 (all to American Cyanamid Company). Structural analogues of calicheamicin which may be used include, but are not limited to, those disclosed in, for example, Hinman et al., Cancer Research 53:3336-3342 (1993), Lode et al., Cancer Research 58:2925-2928 (1998), and the aforementioned U.S. patents to American Cyanamid.

An exemplary calicheamicin is designated γ₁, which is herein referenced simply as gamma, and has the structural formula:

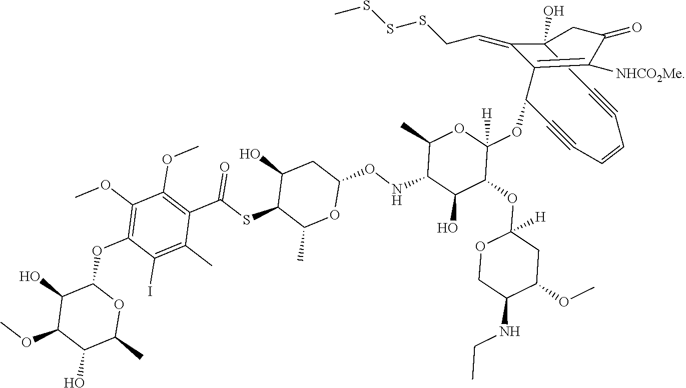

In some embodiments, the calicheamicin may be a gamma-calicheamicin derivative or an N-acetyl gamma-calicheamicin derivative. Structural analogues of calicheamicin which may be used include, but are not limited to, those disclosed in, for example, Hinman et al., Cancer Research 53:3336-3342 (1993), Lode et al., Cancer Research 58:2925-2928 (1998), and the aforementioned U.S. patents. Calicheamicins contain a methyltrisulfide moiety that can be reacted with appropriate thiols to form disulfides, at the same time introducing a functional group that is useful in attaching a calicheamicin derivative to an anti-CD117 antibody or antigen-binding fragment thereof as described herein, via a linker. For the preparation of conjugates of the calicheamicin family, see U.S. Pat. Nos. 5,712,374; 5,714,586; 5,739,116; 5,767,285; 5,770,701; 5,770,710; 5,773,001; and 5,877,296 (all to American Cyanamid Company). Structural analogues of calicheamicin which may be used include, but are not limited to, those disclosed in, for example, Hinman et al., Cancer Research 53:3336-3342 (1993), Lode et al., Cancer Research 58:2925-2928 (1998), and the aforementioned U.S. patents to American Cyanamid.

In one embodiment, the cytotoxin of the ADC as disclosed herein may be a calicheamicin disulfide derivative represented by the formula:

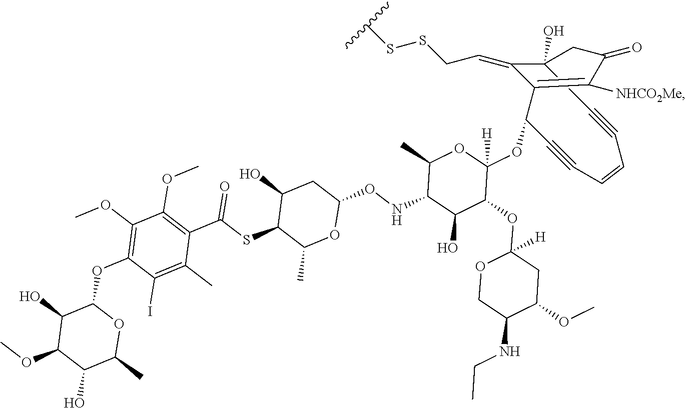

wherein the wavy line indicates the attachment point of the linker.

### Ribosome Inactivating Proteins (RIPs)

In some embodiments, the cytotoxin conjugated to the antibodies and antigen-binding fragments thereof described herein is a ribosome-inactivating protein (RIP). Ribosome inactivating proteins are protein synthesis inhibitors that act on ribosomes, usually irreversibly. RIPs are found in plants, as well as bacteria. Examples of RIPs include, but are not limited to, saporin, ricin, abrin, gelonin, Pseudomonas exotoxin (or exotoxin A), trichosanthin, luffin, agglutinin and the diphtheria toxin.

Another example of an RIP that may be used in the ADCs and methods disclosed herein are a Shiga toxin (Stx) or a Shiga-like toxins (SLT). Shiga toxin (Stx) is a bacterial toxin found in Shigella dysenteriae 1 and in some serogroups (including serotypes O157:H7, and 0104:H4) of Escherichia coli (called Stx1 in E. coli). In addition to Stx1, some E. coli strains produce a second type of Stx (Stx2) that has the same mode of action as Stx/Stx1 but is antigenically distinct. The toxins are named after Kiyoshi Shiga, who first described the bacterial origin of dysentery caused by Shigella dysenteriae. SLT is a historical term for similar or identical toxins produced by Escherichia coli. Because subtypes of each toxin have been identified, the prototype toxin for each group is now designated Stx1a or Stx2a. Stx1a and Stx2a exhibit differences in cytotoxicity to various cell types, bind dissimilarly to receptor analogs or mimics, induce differential chemokine responses, and have several distinctive structural characteristics.

A member of the Shiga toxin family refers to any member of a family of naturally occurring protein toxins which are structurally and functionally related, notably, toxins isolated from S. dysenteriae and E. coli (Johannes L, Romer W, Nat Rev Microbiol 8: 105-16 (2010)). For example, the Shiga toxin family encompasses true Shiga toxin (Stx) isolated from S. dysenteriae serotype 1, Shiga-like toxin 1 variants (SLT1 or Stx1 or SLT-1 or Slt-1) isolated from serotypes of enterohemorrhagic E. coli, and Shiga-like toxin 2 variants (SLT2 or Stx2 or SLT-2) isolated from serotypes of enterohemorrhagic E. coli. SLT1 differs by only one residue from Stx, and both have been referred to as Verocytotoxins or Verotoxins (VTs) (O'Brien A et al., Curr Top Microbiol Immunol 180: 65-94 (1992)). Although SLT1 and SLT2 variants are only about 53-60% similar to each other at the amino acid sequence level, they share mechanisms of enzymatic activity and cytotoxicity common to the members of the Shiga toxin family (Johannes, Nat Rev Microbiol 8: 105-16 (2010)).

Members of the Shiga toxin family have two subunits; A subunit and a B subunit. The B subunit of the toxin binds to a component of the cell membrane known as glycolipid globotriaosylceramide (Gb3). Binding of the subunit B to Gb3 causes induction of narrow tubular membrane invaginations, which drives formation of inward membrane tubules for the bacterial uptake into the cell. The Shiga toxin (a non-pore forming toxin) is transferred to the cytosol via Golgi network and ER. From the Golgi toxin is trafficked to the ER. Shiga toxins act to inhibit protein synthesis within target cells by a mechanism similar to that of ricin (Sandvig and van Deurs (2000) EMBO J 19(220:5943). After entering a cell, the A subunit of the toxin cleaves a specific adenine nucleobase from the 28S RNA of the 60S subunit of the ribosome, thereby halting protein synthesis (Donohue-Rolfe et al. (2010) Reviews of Infectious Diseases 13 Suppl. 4(7): S293-297).

As used herein, reference to Shiga family toxin refers to any member of the Shiga toxin family of naturally occurring protein toxins (e.g., toxins isolated from S. dysenteriae and E. coli) which are structurally and functionally related. For example, the Shiga toxin family encompasses true Shiga toxin (Stx) isolated from S. dysenteriae serotype 1, Shiga-like toxin 1 variants (SLT1 or Stx1 or SLT-1 or Slt-1) isolated from serotypes of enterohemorrhagic E. coli, and Shiga-like toxin 2 variants (SLT2 or Stx2 or SLT-2) isolated from serotypes of enterohemorrhagic E. coli. As used herein, “subunit A from a Shiga family toxin” or “Shiga family toxin subunit A” refers to a subunit A from any member of the Shiga toxin family, including Shiga toxins or Shiga-like toxins.

In one embodiment, the ADC comprises any one of the antibodies and antigen-binding fragments thereof described herein conjugated to a Shiga family toxin subunit A, or a portion of a Shiga family toxin subunit A having cytotoxic activity, i.e., ribosome inhibiting activity. Shiga toxin subunit A cytotoxic activities include, for example, ribosome inactivation, protein synthesis inhibition, N-glycosidase activity, polynucleotide:adenosine glycosidase activity, RNAase activity, and DNAase activity. Non-limiting examples of assays for Shiga toxin effector activity measure protein synthesis inhibitory activity, depurination activity, inhibition of cell growth, cytotoxicity, supercoiled DNA relaxation activity, and nuclease activity.

In certain embodiments, the antibodies and antigen-binding fragments thereof described herein, are conjugated to Shiga family toxin A subunit, or a fragment thereof having ribosome inhibiting activity. An example of a Shiga family toxin subunit A is Shiga-like toxin 1 subunit A (SLT-1A), the amino acid sequence of which is provided below

(SEQ ID NO: 290)
KEFTLDFSTAKTYVDSLNVIRSAIGTPLQTISSGG
TSLLMIDSGSGDNLFAVDVRGIDPEEGRFQNNLRL
IVERNNLYVTGFVNRTNNVFYRFADFSHVTFPGTT
AVTLSGDSSYTTLQRVAGISRTQGMQINRHSLTTS
YLDLMSHSGTSLTQSVARAMLRFVTVTAEALRFRQ
IQRGFRTTLDDLSGRQSYVMTAEDVDLTLNWGRLS
SVLPDYHGQDSVRVGRISFGSINAILGSVALILNC
HHHASRVAQRMASDEFPSMCPADGRVRGITHNKIL
WDSSTLGAILMRRTISS.

Another example of a Shiga family toxin subunit A is Shiga toxin subunit A (StxA), the amino acid sequence of which is provided below

(SEQ ID NO: 291)
KEFTLDFSTAKTYVDSLNVIRSAIGTPLQTISSGG
TSLLMIDSGTGDNLFAVDVRGIDPEEGRFQNNLRL
IVERNNLYVTGFVNRTNNVFYRFADFSHVTFPGTT
AVTLSGDSSYTTLQRVAGISRTQGMQINRHSLTTS
YLDLMSHSGTSLTQSVARAMLRFVTVTAEALRFRQ
IQRGFRTTLDDLSGRQSYVMTAEDVDLTLNWGRLS
SVLPDYHGQDSVRVGRISFGSINAILGSVALILNC
HHHASRVAQRMASDEFPSMCPADGRVRGITHNKIL
WDSSTLGAILMRRTISS.

Another example of a Shiga family toxin subunit A is Shiga-like toxin 2 subunit A (SLT-2A), the amino acid sequence of which is provided below

(SEQ ID NO: 292)
DEFTVDFSSQKSYVDSLNSIRSAISTPLGNISQGG
VSVSVINHVLGGNYISLNVRGLDPYSERQFNHLRL
IMERNNLYVAGFINTETNIFYRFSDFSHISVPDVI
TVSMTTDSSYSSLQRIADLERIGQMQIGRHSLVGS
YLDLMEFRGRSMTRASSRAMLRFVTVIAEALRFRQ
IQRGFRPALSEASPLYQTMTAQDVDLTLNWGRISN
VLPEYRGEEGVRIGRISFNSLSAILGSVAVILNCH
STGSYSVRSVQSQKQKTECQIVGDRAAIKVNNVLW
EANTIAALLNRKPQDLTEPNQ.

In certain circumstances, naturally occurring Shiga family toxin subunits A may comprise precursor forms containing signal sequences of about 22 amino acids at their amino-terminals which are removed to produce mature Shiga family toxin A subunits and are recognizable to the skilled worker. Cytotoxic fragments or truncated versions of Shiga family toxin subunit A may also be used in the ADCs and methods disclosed herein.

In certain embodiments, a Shiga family toxin subunit A differs from a naturally occurring Shiga toxin A subunit by up to 1, 2, 3, 4, 5, 6, 7, 8, 9, 10, 15, 20, 25, 30, 35, 40 or more amino acid residues (but by no more than that which retains at least about 85%, at least about 90%, at least about 95%, at least about 96%, at least about 97%, at least about 98%, at least about 99%, or more amino acid sequence identity). In some embodiments, the Shiga family toxin subunit A differs from a naturally occurring Shiga family toxin A subunit by up to 1, 2, 3, 4, 5, 6, 7, 8, 9, 10, 15, 20, 25, 30, 35, 40 or more amino acid residues (but by no more than that which retains at least about 85%, at least about 90%, at least about 95%, at least about 96%, at least about 97%, at least about 98%, at least about 99% or more amino acid sequence identity). Thus, a polypeptide region derived from an A Subunit of a member of the Shiga toxin family may comprise additions, deletions, truncations, or other alterations from the original sequence as long as at least about 85%, at least about 90%, at least about 95%, at least about 96%, at least about 97%, at least about 98%, at least about 99% or more amino acid sequence identity is maintained to a naturally occurring Shiga family toxin subunit A.

Accordingly, in certain embodiments, the Shiga family toxin subunit A comprises or consists essentially of amino acid sequences having at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, at least about 99.5%, at least about 99.6%, at least about 99.7%, at least about 99.8%, at least about 99.9% or more overall sequence identity to a naturally occurring Shiga family toxin subunit A, such as SLT-1A (SEQ ID NO: 290), StxA (SEQ ID NO:291), and/or SLT-2A (SEQ ID NO:292).

Suitable Shiga toxins and RIPs suitable as cytotoxins are disclosed in, for example, US20180057544, which is incorporated by reference herein in its entirety.

### Auristatins

The antibodies and antigen-binding fragments thereof described herein can be conjugated to a cytotoxin that is an auristatin (U.S. Pat. Nos. 5,635,483; 5,780,588). Auristatins are anti-mitotic agents that interfere with microtubule dynamics, GTP hydrolysis, and nuclear and cellular division (Woyke et al (2001) Antimicrob. Agents and Chemother. 45(12):3580-3584) and have anticancer (U.S. Pat. No. 5,663,149) and antifungal activity (Pettit et al (1998) Antimicrob. Agents Chemother. 42:2961-2965). (U.S. Pat. Nos. 5,635,483; 5,780,588). The auristatin drug moiety may be attached to the antibody through the N-(amino) terminus or the C-(carboxyl) terminus of the peptidic drug moiety (WO 02/088172).

Exemplary auristatin embodiments include the N-terminus linked monomethylauristatin drug moieties DE and DF, disclosed in Senter et al, Proceedings of the American Association for Cancer Research, Volume 45, Abstract Number 623, presented Mar. 28, 2004, the disclosure of which is expressly incorporated by reference in its entirety.

An exemplary auristatin embodiment is MMAE:

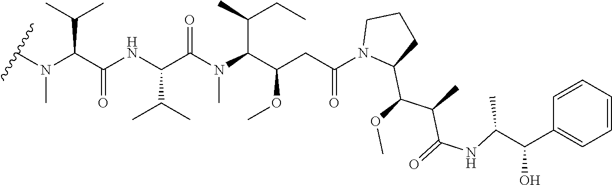 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

wherein the wavy line indicates the point of covalent attachment to the linker of an antibody-linker conjugate (-L-Z-Ab, as described herein).

Another exemplary auristatin embodiment is MMAF:

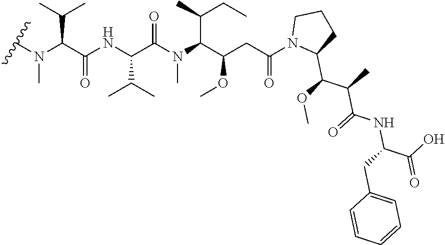 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)O)OC)C(C)C)C(C)C]

wherein the wavy line indicates the point of covalent attachment to the linker of an antibody-linker conjugate (-L-Z-Ab, as described herein), as disclosed in US 2005/0238649.

Auristatins may be prepared according to the methods of: U.S. Pat. Nos. 5,635,483; 5,780,588; Pettit et al (1989) J. Am. Chem. Soc. 111:5463-5465; Pettit et al (1998) Anti-Cancer Drug Design 13:243-277; Pettit, G. R., et al. Synthesis, 1996, 719-725; Pettit et al (1996) J. Chem. Soc. Perkin Trans. 15:859-863; and Doronina (2003) Nat. Biotechnol. 21(7):778-784.

### Amatoxins

In some embodiments, the cytotoxin of the ADC is an amatoxin or derivative thereof. In some embodiments, the cytotoxin is an amatoxin or derivative thereof, such as a-amanitin, p-amanitin, y-amanitin, E-amanitin, amanin, amaninamide, amanullin, amanullinic acid, and proamanullin. Structures of the various amatoxins are represented by formula (II) and accompanying Table 2, and are disclosed in, e.g., Zanotti et al., Int. J. Peptide Protein Res. 30, 1987, 450-459, which is incorporated by reference herein in its entirety.

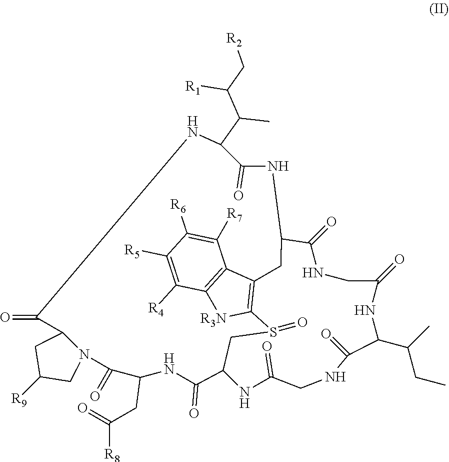

TABLE 2
Amatoxin structures

| Name | R₁ | R₂ | R₃, R₄ | R₅ | R₆, R₇ | R₈ | R₉ |
|---|---|---|---|---|---|---|---|
| α-amanitin | OH | OH | H | OH | H | NH₂ | OH |
| β-amanitin | OH | OH | H | OH | H | OH | OH |
| γ-amanitin | OH | H | H | OH | H | NH₂ | OH |
| ε-amanitin | OH | H | H | OH | H | OH | OH |
| Amanin | OH | OH | H | H | H | OH | OH |
| Amaninamide | OH | OH | H | H | H | NH₂ | OH |
| Amanullin | H | H | H | OH | H | NH₂ | OH |
| Amanullinic acid | H | H | H | OH | H | OH | OH |
| Proamanullin | H | H | H | OH | H | NH₂ | H |

Amatoxins and derivatives thereof useful in conjunction with the compositions and methods described herein include, but are not limited to, α-amanitins, β-amanitins, γ-amanitins, ε-amanitins, amanins, amaninamides, amanullins, amanullinic acids, or proamanullins and derivatives thereof. In one embodiment, the cytotoxin is an amanitin or derivative thereof. In one embodiment, the cytotoxin is an a-amanitin or derivative thereof. For instance, antibodies, or antigen-binding fragments thereof, that recognize and bind to an antigen expressed on the cell surface of a human stem cell or a T cell can be conjugated to an amatoxin, such as an a-amanitin or a derivative thereof, as described in, for example, U.S. Pat. Nos. 9,233,173 and 9,399,681 and US Patent Application Publication Nos. 2016/0089450, 2016/0002298, 2015/0218220, 2014/0294865, the disclosure of each of which is incorporated herein by reference as it pertains, for example, to amatoxins, such as α-amanitin, as well as covalent linkers that can be used for covalent conjugation. Exemplary methods of amatoxin conjugation and linkers useful for such processes are described herein. Exemplary linker-containing amatoxins useful for conjugation to an antibody, or antigen-binding fragment, in accordance with the compositions and methods are also described herein.

As used herein, the term “amatoxin derivative” or “amanitin derivative” refers to an amatoxin that has been chemically modified at one or more positions relative to a naturally occurring amatoxin, such as α-amanitin, β-amanitin, γ-amanitin, ε-amanitin, amanin, amaninamide, amanullin, amanullinic acid, or proamanullin. In each instance, the derivative may be obtained by chemical modification of a naturally occurring compound (“semi-synthetic”), or may be obtained from an entirely synthetic source. Synthetic routes to various amatoxin derivatives are disclosed in, for example, U.S. Pat. No. 9,676,702 and in Perrin et al., J. Am. Chem. Soc. 2018, 140, p. 6513-6517, each of which is incorporated by reference herein in their entirety with respect to synthetic methods for preparing and derivatizing amatoxins.

In some embodiments, the amatoxin or derivative thereof is represented by formula (III):

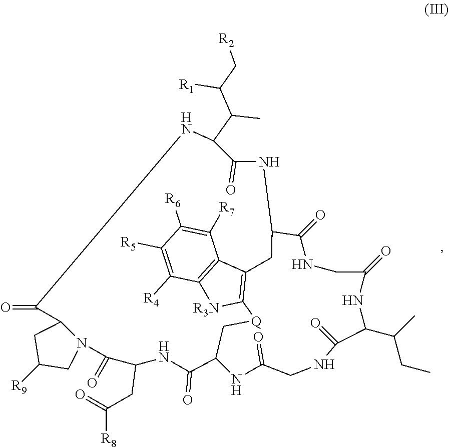

or is an enantiomer or diastereomer thereof, wherein:

Q is —S—, —S(O)—, or —SO₂—;

R₁ is H, OH, or OR_{A};

R₂ is H, OH, or OR_{B};

R_{A} and R_{B}, when present, together with the oxygen atoms to which they are bound, combine to form a 5-membered heterocycloalkyl group;

R₃ is H or R_{C};

each of R₄, R₅, R₆, and R₇ is independently H, OH, or R_{C};

R₈ is OH, NH₂, OR_{C}, or NHR_{C};

R₉ is H or OH or OR_{C}; and

R_{C} is C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, cycloalkyl, heterocycloalkyl, aryl, heteroaryl, or a combination thereof, wherein each C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, cycloalkyl, heterocycloalkyl, aryl, or heteroaryl may optionally be substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

In some embodiments, R_{A} and R_{B}, when present, together with the oxygen atoms to which they are bound, combine to form a 5-membered heterocycloalkyl group of formula:

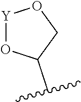

wherein Y is —(C═O)—, —(C═S)—, —(C═NH)—, or —(CH)₂—.

In some embodiments, the amatoxin or derivative thereof is represented by formula (IIIa):

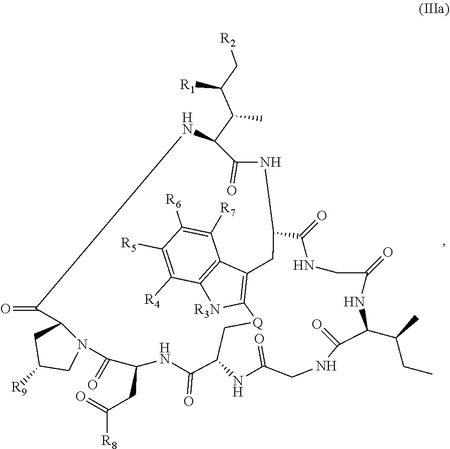

wherein each of Q, R₁-R₉, R_{A}, R_{B}, and R_{C} are as previously defined for formula (III).

In some embodiments, the amatoxin or derivative thereof of formula (III) is represented by formula (IIIb):

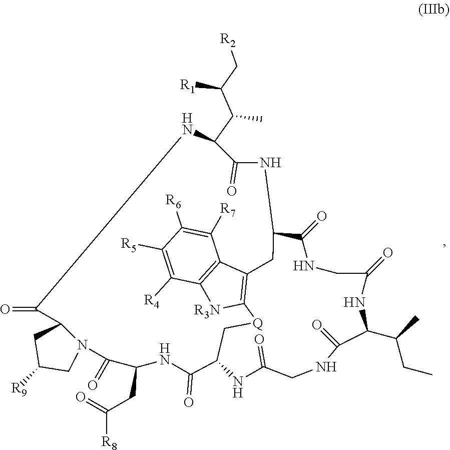

wherein each of Q, R₁-R₉, R_{A}, R_{B}, and R_{C} are as previously defined for formula (III).

In some embodiments, the amatoxin or derivative thereof of formula (III) is represented by formula (IIIc):

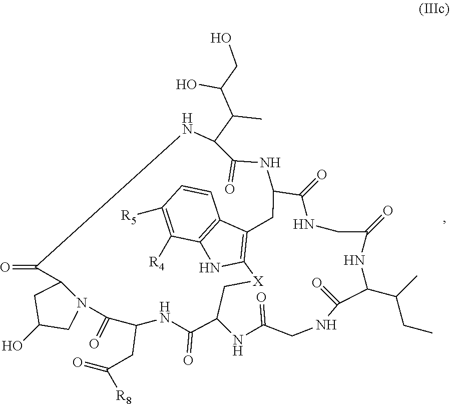

wherein R₄, R₅, X, and Rs are each as defined above.

Additional amatoxins that may be used for conjugation to an antibody, or antigen-binding fragment thereof, in accordance with the compositions and methods described herein are described, for example, in WO 2016/142049; WO 2016/071856; WO 2017/149077; WO 2018/115466; and WO 2017/046658, the disclosures of which are incorporated herein by reference in their entirety.

3. Linkers

The term “Linker” as used herein means a divalent chemical moiety comprising a covalent bond or a chain of atoms that covalently attaches an antibody or fragment thereof (Ab) to a cytotoxin (e.g., an amatoxin) to form an antibody-drug conjugate (ADC).

Covalent attachment of the antibody and the drug moiety requires the linker to have two reactive functional groups, i.e. bivalency in a reactive sense. Bivalent linker reagents which are useful to attach two or more functional or biologically active moieties, such as peptides, nucleic acids, drugs, toxins, antibodies, haptens, and reporter groups are known, and methods have been described their resulting conjugates (Hermanson, G. T. (1996) Bioconjugate Techniques; Academic Press: New York, p. 234-242).

Accordingly, present linkers have two reactive termini, one for conjugation to an antibody and the other for conjugation to a cytotoxin. The antibody conjugation reactive terminus of the linker (reactive moiety, defined herein as Z′) is typically a chemical moiety that is capable of conjugation to the antibody through, e.g., a cysteine thiol or lysine amine group on the antibody, and so is typically a thiol-reactive group such as a Michael acceptor (as in maleimide), a leaving group, such as a chloro, bromo, iodo, or an R-sulfanyl group, or an amine-reactive group such as a carboxyl group. Conjugation of the linker to the antibody is described more fully herein below.

The cytotoxin (e.g., amatoxin) conjugation reactive terminus of the linker is typically a chemical moiety that is capable of conjugation to the cytotoxin through formation of a bond with a reactive substituent within the cytotoxin molecule. Non-limiting examples include, for example, formation of an amide bond with a basic amine or carboxyl group on the cytotoxin via a carboxyl or basic amine group on the linker, respectively, or formation of an ether, an amide, or the like, via alkylation of an OH or NH group, respectively, on the cytotoxin.

When the term “linker” is used in describing the linker in conjugated form, one or both of the reactive termini will be absent (such as reactive moiety Z′, having been converted to chemical moiety Z, as described herein below) or incomplete (such as being only the carbonyl of the carboxylic acid) because of the formation of the bonds between the linker and/or the cytotoxin, and between the linker and/or the antibody or antigen-binding fragment thereof. Such conjugation reactions are described further herein below.

A variety of linkers can be used to conjugate the antibodies, antigen-binding fragments, and ligands described to a cytotoxic molecule. Generally, linkers suitable for the present disclosure may be substantially stable in circulation, but allow for release of the cytotoxin within or in close proximity to the target cells. In some embodiments, certain linkers suitable for the present disclosure may be categorized as cleavable or non-cleavable. Generally, cleavable linkers contain one or more functional groups that is cleaved in response to a physiological environment. For example, a cleavable linker may contain an enzymatic substrate (e.g., valine-alanine) that degrades in the presence of an intracellular enzyme (e.g., cathepsin B), an acid-cleavable group (e.g., a hydrozone) that degrades in the acidic environment of a cellular compartment, or a reducible group (e.g., a disulfide) that degrades in an intracellular reducing environment. By contrast, generally, non-cleavable linkers are released from the ADC during degradation (e.g., lysosomal degradation) of the antibody moiety of the ADC inside the target cell.

a. Non-Cleavable Linkers

Non-cleavable linkers suitable for use herein further may include one or more groups selected from a bond, —(C═O)—, C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, heteroarylene, and combinations thereof, each of which may be optionally substituted, and/or may include one or more heteroatoms (e.g., S, N, or O) in place of one or more carbon atoms. Non-limiting examples of such groups include alkylene (CH₂)ₚ, (C═O)(CH₂)ₚ, and polyethyleneglycol (PEG; (CH₂CH₂O)ₚ), units, wherein p is an integer from 1-6, independently selected for each occasion.

In some embodiments, the linker L comprises one or more of a bond, —(C═O)—, a —C(O)NH— group, an —OC(O)NH— group, C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, heteroarylene, a —(CH₂CH₂O)ₚ— group where p is an integer from 1-6, or a solubility enhancing group;

wherein each C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, or heteroarylene may optionally be substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro;

In some embodiments, each C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, or heteroarylene may optionally be interrupted by one or more heteroatoms selected from O, S and N.

In some embodiments, each C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, or heteroarylene may optionally be interrupted by one or more heteroatoms selected from O, S and N and may be optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

In some embodiments, the linker comprises a —(CH₂)ₙ— unit, where n is an integer from, 2-12, e.g., 2-6. In some embodiments, the linker comprises a —(CH₂)ₙ— where n is 6.

In some embodiments, the linker is —(CH₂)ₙ— where n is 1, 2, 3, 4, 5, or 6, represented by the formula:

b. Cleavable Linkers

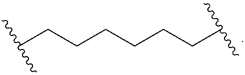 [SMILES: *CCCCCC*]

In some embodiments, the linker conjugating the antibody or antigen binding fragment thereof to the cytotoxin is cleavable under intracellular conditions, such that cleavage of the linker releases the drug unit from the antibody in the intracellular environment. Cleavable linkers are designed to exploit the differences in local environments, e.g., extracellular and intracellular environments, including, for example, pH, reduction potential or enzyme concentration, to trigger the release of the cytotoxin in the target cell. Generally, cleavable linkers are relatively stable in circulation, but are particularly susceptible to cleavage in the intracellular environment through one or more mechanisms (e.g., including, but not limited to, activity of proteases, peptidases, and glucuronidases). Cleavable linkers used herein are substantially stable in circulating plasma and/or outside the target cell and may be cleaved at some efficacious rate inside the target cell or in close proximity to the target cell.

Suitable cleavable linkers include those that may be cleaved, for instance, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012, the disclosure of which is incorporated herein by reference as it pertains to linkers suitable for covalent conjugation). Suitable cleavable linkers may include, for example, chemical moieties such as a hydrazine, a disulfide, a thioether or a dipeptide.

Linkers hydrolyzable under acidic conditions include, for example, hydrazones, semicarbazones, thiosemicarbazones, cis-aconitic amides, orthoesters, acetals, ketals, or the like. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661, the disclosure of each of which is incorporated herein by reference in its entirety as it pertains to linkers suitable for covalent conjugation. Such linkers are relatively stable under neutral pH conditions, such as those in the blood, but are unstable at below pH 5.5 or 5.0, the approximate pH of the lysosome.

Linkers cleavable under reducing conditions include, for example, a disulfide. A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935, the disclosure of each of which is incorporated herein by reference in its entirety as it pertains to linkers suitable for covalent conjugation.

Linkers susceptible to enzymatic hydrolysis can be, e.g., a peptide-containing linker that is cleaved by an intracellular peptidase or protease enzyme, including, but not limited to, a lysosomal or endosomal protease. One advantage of using intracellular proteolytic release of the therapeutic agent is that the agent is typically attenuated when conjugated and the serum stabilities of the conjugates are typically high. In some embodiments, the peptidyl linker is at least two amino acids long or at least three amino acids long. Exemplary amino acid linkers include a dipeptide, a tripeptide, a tetrapeptide or a pentapeptide. Examples of suitable peptides include those containing amino acids such as Valine, Alanine, Citrulline (Cit), Phenylalanine, Lysine, Leucine, and Glycine. Amino acid residues which comprise an amino acid linker component include those occurring naturally, as well as minor amino acids and non-naturally occurring amino acid analogs, such as citrulline. Exemplary dipeptides include valine-citrulline (vc or val-cit) and alanine-phenylalanine (af or ala-phe). Exemplary tripeptides include glycine-valine-citrulline (gly-val-cit) and glycine-glycine-glycine (gly-gly-gly). In some embodiments, the linker includes a dipeptide such as Val-Cit, Ala-Val, or Phe-Lys, Val-Lys, Ala-Lys, Phe-Cit, Leu-Cit, Ile-Cit, Phe-Arg, or Trp-Cit. Linkers containing dipeptides such as Val-Cit or Phe-Lys are disclosed in, for example, U.S. Pat. No. 6,214,345, the disclosure of which is incorporated herein by reference in its entirety as it pertains to linkers suitable for covalent conjugation. In some embodiments, the linker comprises a dipeptide selected from Val-Ala and Val-Cit.

Linkers suitable for conjugating the antibodies, antigen-binding fragments, and ligands described herein to a cytotoxic molecule include those capable of releasing a cytotoxin by a 1,6-elimination process. Chemical moieties capable of this elimination process include the p-aminobenzyl (PAB) group, 6-maleimidohexanoic acid, pH-sensitive carbonates, and other reagents as described in Jain et al., Pharm. Res. 32:3526-3540, 2015, the disclosure of which is incorporated herein by reference in its entirety as it pertains to linkers suitable for covalent conjugation.

In some embodiments, the linker includes a “self-immolative” group such as the afore-mentioned PAB or PABC (para-aminobenzyloxycarbonyl), which are disclosed in, for example, Carl et al., J. Med. Chem. (1981) 24:479-480; Chakravarty et al (1983) J. Med. Chem. 26:638-644; U.S. Pat. No. 6,214,345; US20030130189; US20030096743; U.S. Pat. No. 6,759,509; US20040052793; U.S. Pat. Nos. 6,218,519; 6,835,807; 6,268,488; US20040018194; WO98/13059; US20040052793; U.S. Pat. Nos. 6,677,435; 5,621,002; US20040121940; WO2004/032828). Other such chemical moieties capable of this process (“self-immolative linkers”) include methylene carbamates and heteroaryl groups such as aminothiazoles, aminoimidazoles, aminopyrimidines, and the like. Linkers containing such heterocyclic self-immolative groups are disclosed in, for example, U.S. Patent Publication Nos. 20160303254 and 20150079114, and U.S. Pat. No. 7,754,681; Hay et al. (1999) Bioorg. Med. Chem. Lett. 9:2237; US 2005/0256030; de Groot et al (2001) J. Org. Chem. 66:8815-8830; and U.S. Pat. No. 7,223,837. In some embodiments, a dipeptide is used in combination with a self-immolative linker.

Suitable linkers may contain groups having solubility enhancing properties. Linkers including the (CH₂CH₂O)ₚ unit (polyethylene glycol, PEG), for example, can enhance solubility, as can alkyl chains substituted with amino, sulfonic acid, phosphonic acid or phosphoric acid residues. Linkers including such moieties are disclosed in, for example, U.S. Pat. Nos. 8,236,319 and 9,504,756, the disclosure of each of which is incorporated herein by reference in its entirety as it pertains to linkers suitable for covalent conjugation. Further solubility enhancing groups include, for example, acyl and carbamoyl sulfamide groups, having the structure:

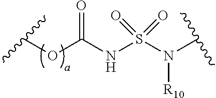

wherein a is 0 or 1; and

R¹⁰ is selected from the group consisting of hydrogen, C₁-C₂₄ alkyl groups, C₃-C₂₄ cycloalkyl groups, C₁-C₂₄ (hetero)aryl groups, C₁-C₂₄ alkyl(hetero)aryl groups and C₁-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, each of which may be optionally substituted and/or optionally interrupted by one or more heteroatoms selected from O, S and NR¹¹R¹², wherein R¹¹ and R¹² are independently selected from the group consisting of hydrogen and C₁-C₄ alkyl groups; or R¹⁰ is a cytotoxin, wherein the cytotoxin is optionally connected to N via a spacer moiety. Linkers containing such groups are described, for example, in U.S. Pat. No. 9,636,421 and U.S. Patent Application Publication No. 2017/0298145, the disclosures of which are incorporated herein by reference in their entirety as they pertain to linkers suitable for covalent conjugation to cytotoxins and antibodies or antigen-binding fragments thereof.

In some embodiments, the linker L comprises one or more of a hydrazine, a disulfide, a thioether, an amino acid, a peptide consisting of up to 10 amino acids, a p-aminobenzyl (PAB) group, a heterocyclic self-immolative group, C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, C₃-C₆ cycloalkyl, heterocycloalkyl, aryl, heteroaryl, a —(C═O)— group, a —C(O)NH— group, an —OC(O)NH— group, or a —(CH₂CH₂O)ₚ— group where p is an integer from 1-6;

wherein each C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, C₃-C₆ cycloalkyl, heterocycloalkyl, aryl, or heteroaryl group may be optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

In some embodiments, each C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, C₃-C₆ cycloalkyl, heterocycloalkyl, aryl, or heteroaryl group may optionally be interrupted by one or more heteroatoms selected from O, S and N.

In some embodiments, each C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, C₃-C₆ cycloalkyl, heterocycloalkyl, aryl, or heteroaryl group may optionally be interrupted by one or more heteroatoms selected from O, S and N and may be optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

One of skill in the art will recognize that one or more of the groups listed may be present in the form of a bivalent (diradical) species, e.g., C₁-C₆ alkylene and the like.

In some embodiments, the linker L comprises the moiety *-L₁L₂-**, wherein: L₁ is absent or is —(CH₂)ₘNR¹³C(═O)—, —(CH₂)ₘNR¹³—, —(CH₂)ₘX₃(CH₂)ₘ—,

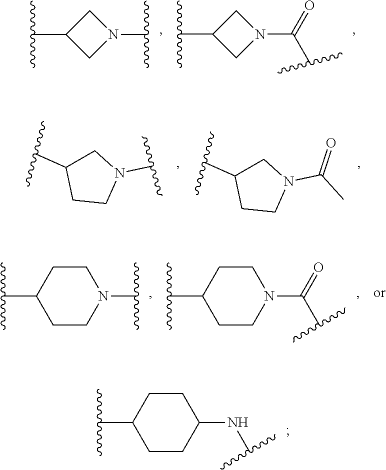

L₂ is absent or is —(CH₂)ₘ—, —NR¹³(CH₂)ₘ—, —(CH₂)ₘNR¹³C(═O)(CH₂)ₘ—, —X₄, —(CH₂)ₘNR¹³C(═O)X₄, —(CH₂)ₘNR¹³C(═O)—, —((CH₂)ₘO)ₙ(CH₂)ₘ—, —((CH₂)ₘO)ₙ(CH₂)ₘX₃(CH₂)ₘ—, —NR¹³((CH₂)ₘO)ₙX₃(CH₂)ₘ—, —NR¹³((CH₂)ₘO)ₙ(CH₂)ₘX₃(CH₂)ₘ—, —X₁X₂C(═O)(CH₂)ₘ—, —(CH₂)ₘ(O(CH₂)ₘ)ₙ—, —(CH₂)ₘNR¹³(CH₂)ₘ—, —(CH₂)ₘNR¹³C(═O)(CH₂)ₘX₃(CH₂)ₘ—, —(CH₂)ₘC(═O)NR¹³(CH₂)ₘNR¹³C(═O)(CH₂)ₘ—, —(CH₂)ₘC(═O)—, —(CH₂)ₘNR¹³(CH₂)ₘC(═O)X₂X₁C(═O)—, —(CH₂)ₘX₃(CH₂)ₘC(═O)X₂X₁C(═O)—, —(CH₂)ₘC(═O)NR¹³(CH₂)ₘ—, —(CH₂)ₘC(═O)NR¹³(CH₂)ₘX₃(CH₂)ₘ—, —(CH₂)ₘX₃(CH₂)ₘNR¹³C(═O)(CH₂)ₘ—, —(CH₂)ₘX₃(CH₂)ₘC(═O)NR¹³(CH₂)ₘ—, —(CH₂)ₘO)ₙ(CH₂)ₘNR¹³C(═O)(CH₂)ₘ—, —(CH₂)ₘC(═O)NR¹³(CH₂)ₘ(O(CH₂)ₘ)ₙ—, —(CH₂)ₘ(O(CH₂)ₘ)ₙC(═O)—, —(CH₂)ₘNR¹³(CH₂)ₘC(═O)—, —(CH₂)ₘC(═O)NR¹³(CH₂)ₘNR¹³C(═O)—, —(CH₂)ₘ(O(CH₂)ₘ)ₙX₃(CH₂)ₘ—, —(CH₂)ₘX₃((CH₂)ₘO)ₙ(CH₂)ₘ—, —(CH₂)ₘX₃(CH₂)ₘC(═O)—, —(CH₂)ₘC(═O)NR¹³(CH₂)ₘO)ₙ(CH₂)ₘX₃(CH₂)ₘ—, —(CH₂)ₘX₃(CH₂)ₘ(O(CH₂)ₘ)ₙNR¹³C(═O)(CH₂)ₘ—, —(CH₂)ₘX₃(CH₂)ₘ(O(CH₂)ₘ)ₙC(═O)—, —(CH₂)ₘX₃(CH₂)ₘ(O(CH₂)ₘ)ₙ—, —(CH₂)ₘC(═O)NR¹³(CH₂)ₘC(═O)—, —(CH₂)ₘC(═O)NR¹³(CH₂)ₘ(O(CH₂)ₘ)ₙC(═O)—, —((CH₂)ₘO)ₙ(CH₂)ₘNR¹³C(═O)(CH₂)ₘ—, —(CH₂)ₘC(═O)NR¹³(CH₂)ₘC(═O)NR¹³(CH₂)ₘ—, —(CH₂)ₘNR¹³C(═O)(CH₂)ₘNR¹³C(═O)(CH₂)—(CH₂)ₘX₃(CH₂)ₘC(═O)NR¹³—, —(CH₂)ₘC(═O)NR¹³—, —(CH₂)ₘX₃-, —C(R¹³)2(CH₂)ₘ—, —(CH₂)ₘC(R¹³)2NR¹³—, —(CH₂)ₘC(═O)NR¹³(CH₂)ₘNR¹³—, —(CH₂)ₘC(═O)NR¹³(CH₂)ₘNR¹³C(═O)NR¹³—, —(CH₂)ₘC(═O)X₂X₁C(═O)—, —C(R¹³)2(CH₂)ₘNR¹³C(═O)(CH₂)ₘ—, —(CH₂)ₘC(═O)NR¹³(CH₂)ₘC(R¹³)2NR¹³—, —C(R¹³)₂(CH₂)ₘX₃(CH₂)ₘ—, —(CH₂)ₘX₃(CH₂)ₘC(R¹³)₂NR¹³—, —C(R¹³)₂(CH₂)ₘOC(═O)NR¹³(CH₂)ₘ—, —(CH₂)ₘNR¹³C(═O)O(CH₂)ₘC(R¹³)2NR¹³—, —(CH₂)ₘX₃(CH₂)ₘNR¹³—, —(CH₂)ₘX₃(CH₂)ₘ(O(CH₂)ₘ)ₙNR¹³—, —(CH₂)ₘNR¹³—, —(CH₂)ₘC(═O)NR¹³(CH₂)ₘ(O(CH₂)ₘ)ₙNR¹³—, —(CH₂)ₘ(O(CH₂)ₘ)ₙNR¹³—, —(CH₂CH₂O)ₙ(CH₂)ₘ—, —(CH₂)ₘ(OCH₂CH₂)ₙ; —(CH₂)ₘO(CH₂)ₘ—, —(CH₂)ₘS(═O)₂—, —(CH₂)ₘC(═O)NR¹³(CH₂)ₘS(═O)₂—, —(CH₂)ₘX₃(CH₂)ₘS(═O)₂—, —(CH₂)ₘX₂XC(═O)—, —(CH₂)ₘ(O(CH₂)ₘ)ₙC(═O)X₂XC(═O)—, —(CH₂)ₘ(O(CH₂)ₘ)ₙX₂X₁C(═O)—, —(CH₂)ₘX₃(CH₂)ₘX₂X₁C(═O)—, —(CH₂)ₘX₃(CH₂)ₘ(O(CH₂)ₘ)ₙX₂X, C(═O)—, —(CH₂)ₘX₃(CH₂)ₘC(═O)NR¹³(CH₂)ₘNR¹³C(═O)—, —(CH₂)ₘX₃(CH₂)ₘC(═O)NR¹³(CH₂)ₘC(═O)—, —(CH₂)ₘX₃(CH₂)ₘC(═O)NR¹³(CH₂)ₘ(O(CH₂)ₘ)ₙC(═O)—, —(CH₂)ₘC(═O)X₂X₁C(═O)NR¹³(CH₂)ₘ—, —(CH₂)ₘX₃(O(CH₂)ₘ)ₙC(═O)—, —(CH₂)ₘNR¹³C(═O)((CH₂)ₘO)ₙ(CH₂)ₘ—, —(CH₂)ₘ(O(CH₂)ₘ)ₙC(═O)NR¹³(CH₂)ₘ—, —(CH₂)ₘNR¹³C(═O)NR¹³(CH₂)ₘ— or —(CH₂)ₘX₃(CH₂)ₘNR¹³C(═O)—;

wherein

X₁ is

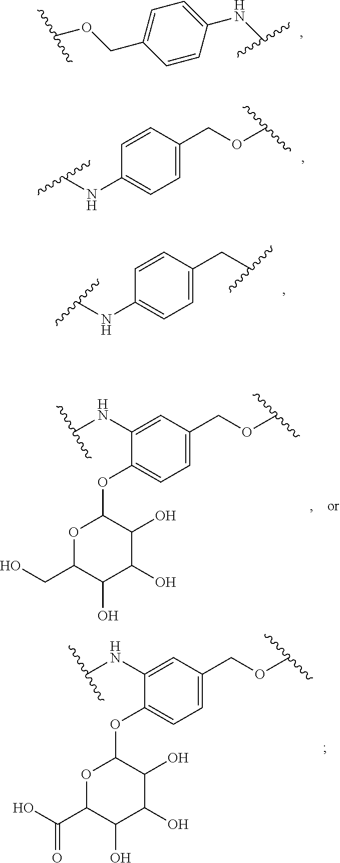

X₂ is

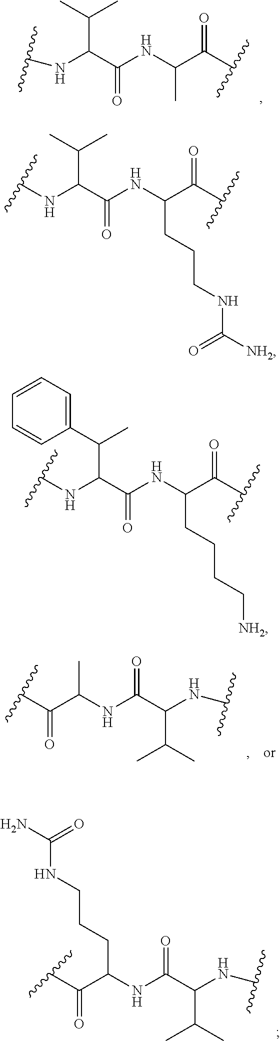

X₃ is

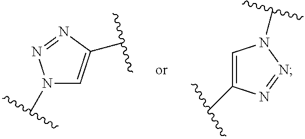 [SMILES: *c1cn(*)nn1 ; *c1cn(*)nn1]

and

X₄ is

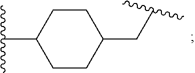

wherein

R¹³ is independently selected for each occasion from H and C₁-C₆ alkyl;

m is independently selected for each occasion from 1, 2, 3, 4, 5, 6, 7, 8, 9 and 10;

n is independently selected for each occasion from 1, 2, 3, 4, 5, 6, 7, 8, 9, 10, 11, 12, 13 and 14; and

wherein the single asterisk (*) indicates the attachment point to the cytotoxin (e.g., an amatoxin), and the double asterisk (**) indicates the attachment point to the reactive substituent Z′ or chemical moiety Z, with the proviso that L₁ and L₂ are not both absent.

In some embodiments, the linker includes a p-aminobenzyl group (PAB). In one embodiment, the p-aminobenzyl group is disposed between the cytotoxic drug and a protease cleavage site in the linker. In one embodiment, the p-aminobenzyl group is part of a p-aminobenzyloxycarbonyl unit. In one embodiment, the p-aminobenzyl group is part of a p-aminobenzylamido unit.

In some embodiments, the linker comprises a peptide selected from the group consisting of Phe-Lys, Val-Lys, Phe-Ala, Phe-Cit, Val-Ala, Val-Cit, and Val-Arg.

In some embodiments, the linker comprises one or more of PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB.

In some embodiments, the linker comprises one or more of a peptide, oligosaccharide, —(CH₂)ₚ—, —(CH₂CH₂O)ₚ—, —(C═O)(CH₂)ₚ—, PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB, wherein p is an integer from 1-6.

In some embodiments, the linker comprises PAB-Ala-Val-propionyl, represented by the formula:

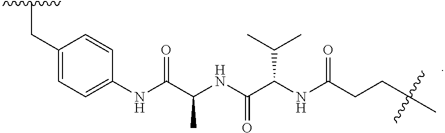 [SMILES: *CCC(=O)N[C@H](C(=O)N[C@@H](C)C(=O)Nc1ccc(C*)cc1)C(C)C]

In some embodiments, the linker comprises PAB-Cit-Val-propionyl, represented by the formula:

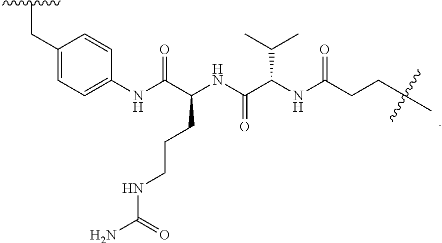 [SMILES: *CCC(=O)N[C@H](C(=O)N[C@@H](CCCNC(N)=O)C(=O)Nc1ccc(C*)cc1)C(C)C]

Such PAB-dipeptide-propionyl linkers are disclosed in, e.g., International Patent Application Publication No. WO2017/149077, which is incorporated by reference herein in its entirety.

In certain embodiments, the linker of the ADC is maleimidocaproyl-Val-Ala-para-aminobenzyl (mc-Val-Ala-PAB).

In certain embodiments, the linker of the ADC is maleimidocaproyl-Val-Cit-para-aminobenzyl (mc-vc-PAB).

In some embodiments, the linker comprises

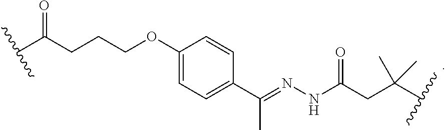 [SMILES: *C(=O)CCCOc1ccc(/C(C)=N/NC(=O)CC(*)(C)C)cc1]

In some embodiments, the linker comprises MCC (4-[N-maleimidomethyl]cyclohexane-1-carboxylate).

It will be recognized by one of skill in the art that any one or more of the chemical groups, moieties and features disclosed herein may be combined in multiple ways to form linkers useful for conjugation of the antibodies and cytotoxins as disclosed herein. Further linkers useful in conjunction with the compositions and methods described herein, are described, for example, in U.S. Patent Application Publication No. 2015/0218220, the disclosure of which is incorporated herein by reference in its entirety.

4. Linker-Cytotoxin and Linker-Antibody Conjugation

In certain embodiments, the linker is reacted with the cytotoxin under appropriate conditions to form a linker-cytotoxin conjugate. In certain embodiments, reactive groups are used on the cytotoxin or linker to form a covalent attachment. In some embodiments, the cytotoxin is an amatoxin or derivative thereof according to any of formulae (III), (IIIa), (IIIb), or (IIIc). The cytotoxin-linker conjugate is subsequently reacted with the antibody, derivatized antibody, or antigen-binding fragment thereof, under appropriate conditions to form the ADC.

Alternatively, the linker may first be reacted with the antibody, derivatized antibody or antigen-binding fragment thereof, to form a linker-antibody conjugate, and then reacted with the cytotoxin to form the ADC. Such conjugation reactions will now be described more fully.

A number of different reactions are available for covalent attachment of linkers or cytotoxin-linker conjugates to the antibody or antigen-binding fragment thereof. Suitable attachment points on the antibody molecule include, but are not limited to, the amine groups of lysine, the free carboxylic acid groups of glutamic acid and aspartic acid, the sulfhydryl groups of cysteine, and the various moieties of aromatic amino acids. For instance, non-specific covalent attachment may be undertaken using a carbodiimide reaction to link a carboxy (or amino) group on a linker to an amino (or carboxy) group on an antibody moiety. Additionally, bifunctional agents such as dialdehydes or imidoesters may also be used to link the amino group on a linker to an amino group on an antibody moiety. Also available for attachment of cytotoxins to antibody moieties is the Schiff base reaction. This method involves the periodate oxidation of a glycol or hydroxy group on either the antibody or linker, thus forming an aldehyde which is then reacted with the linker or antibody, respectively. Covalent bond formation occurs via formation of a Schiff base between the aldehyde and an amino group. Isothiocyanates may also be used as coupling agents for covalently attaching cytotoxins or antibody moieties to linkers. Other techniques are known to the skilled artisan and within the scope of the present disclosure.

Linkers useful in for conjugation to the antibodies or antigen-binding fragments as described herein include, without limitation, linkers containing a chemical moiety Z formed by a coupling reaction between the antibody and a reactive chemical moiety (referred to herein as a reactive substituent, Z′) on the linker as depicted in Table 3, below. Wavy lines designate points of attachment to the antibody or antigen-binding fragment, and the cytotoxic molecule, respectively.

TABLE 3
Exemplary chemical moieties Z formed by coupling reactions in the formation of
antibody-drug conjugates.

| Exemplary Coupling | |
|---|---|
| Reactions | Chemical Moiety Z Formed by Coupling Reactions |
| [3 + 2] Cycloaddition | 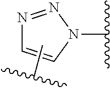 |
| [3 + 2] Cycloaddition | 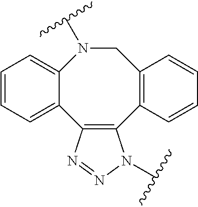 [SMILES: *N1Cc2ccccc2-c2c(nnn2*)-c2ccccc21] |
| [3 + 2] Cycloaddition, Esterification | 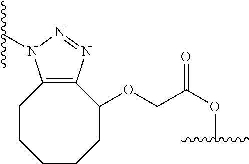 [SMILES: *OC(=O)COC1CCCCCc2c1nnn2*] |
| [3 + 2] Cycloaddition, Esterification | 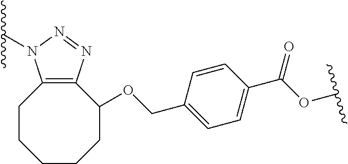 [SMILES: *OC(=O)c1ccc(COC2CCCCCc3c2nnn3*)cc1] |
| [3 + 2] Cycloaddition, Esterification | 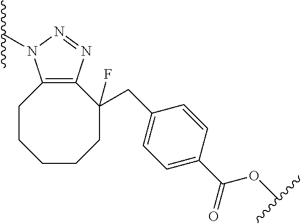 [SMILES: *OC(=O)c1ccc(CC2(F)CCCCCc3c2nnn3*)cc1] |
| [3 + 2] Cycloaddition, Esterification | 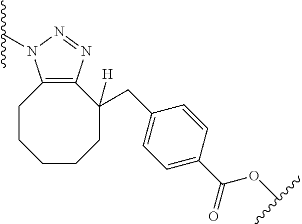 [SMILES: *OC(=O)c1ccc(CC2CCCCCc3c2nnn3*)cc1] |
| [3 + 2] Cycloaddition, Esterification | 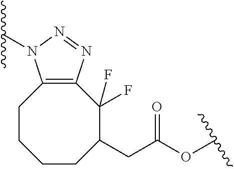 [SMILES: *OC(=O)CC1CCCCc2c(nnn2*)C1(F)F] |
| [3 + 2] Cycloaddition, Esterification | 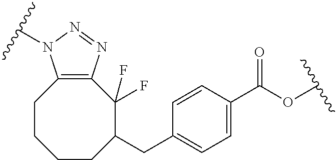 [SMILES: *OC(=O)c1ccc(CC2CCCCc3c(nnn3*)C2(F)F)cc1] |
| [3 + 2] Cycloaddition, Esterification | 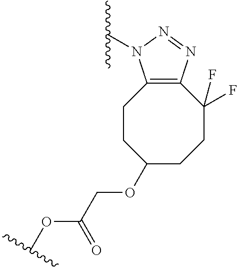 [SMILES: *OC(=O)COC1CCc2c(nnn2*)C(F)(F)CC1] |
| [3 + 2] Cycloaddition, Esterification | 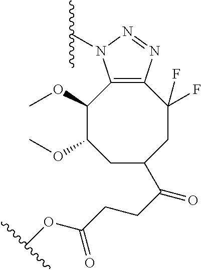 [SMILES: *OC(=O)CCC(=O)C1C[C@H](OC)[C@@H](OC)c2c(nnn2*)C(F)(F)C1] |
| [3 + 2] Cycloaddition, Esterification | 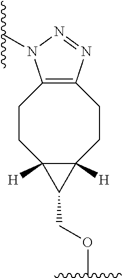 [SMILES: *OC[C@@H]1[C@@H]2CCc3c(nnn3*)CC[C@@H]21] |
| [3 + 2] Cycloaddition, Esterification | 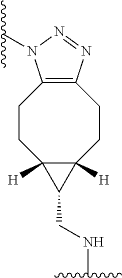 [SMILES: *NC[C@@H]1[C@@H]2CCc3c(nnn3*)CC[C@@H]21] |
| [3 + 2] Cycloaddition, Esterification | 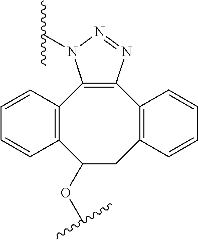 [SMILES: *OC1Cc2ccccc2-c2nnn(*)c2-c2ccccc21] |
| [3 + 2] Cycloaddition, Esterification | 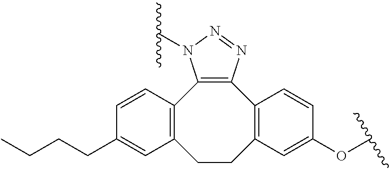 [SMILES: *Oc1ccc2c(c1)CCc1cc(CCCC)ccc1-c1c-2nnn1*] |
| [3 + 2] Cycloaddition | 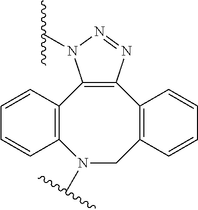 [SMILES: *N1Cc2ccccc2-c2nnn(*)c2-c2ccccc21] |
| Michael addition | 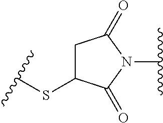 [SMILES: *SC1CC(=O)N(*)C1=O] |
| Michael addition | 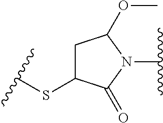 [SMILES: *SC1CC(OC)N(*)C1=O] |
| Imine condensation, Amidation | 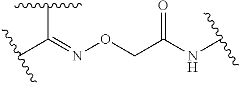 [SMILES: *NC(=O)CON=C(*)*] |
| Imine condensation | 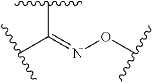 [SMILES: *ON=C(*)*] |
| Disulfide formation | 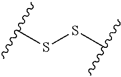 [SMILES: *SS*] |
| Thiol alkylation | 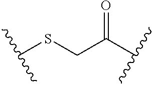 [SMILES: *SCC(*)=O] |
| Condensation, Michael addition | 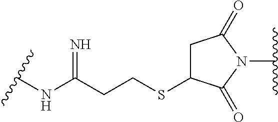 [SMILES: *NC(=N)CCSC1CC(=O)N(*)C1=O] |

One of skill in the art will recognize that a reactive substituent Z′ attached to the linker and a reactive substituent on the antibody or antigen-binding fragment thereof, are engaged in the covalent coupling reaction to produce the chemical moiety Z, and will recognize the reactive substituent Z′. Therefore, antibody-drug conjugates useful in conjunction with the methods described herein may be formed by the reaction of an antibody, or antigen-binding fragment thereof, with a linker or cytotoxin-linker conjugate, as described herein, the linker or cytotoxin-linker conjugate including a reactive substituent Z′, suitable for reaction with a reactive substituent on the antibody, or antigen-binding fragment thereof, to form the chemical moiety Z.

In some embodiments, Z′ is —NR¹³C(═O)CH═CH₂, —N₃, —SH, —S(═O)₂(CH═CH₂), —(CH₂)₂S(═O)₂(CH═CH₂), —NR¹³S(═O)₂(CH═CH₂), —NR¹³C(═O)CH₂R¹⁴, —NR¹³C(═O)CH₂Br, —NR¹³C(═O)CH₂I, —NHC(═O)CH₂Br, —NHC(═O)CH₂I, —ONH₂, —C(O)NHNH₂, —CO₂H, —NH₂, —NH(C═O), —NC(═S),

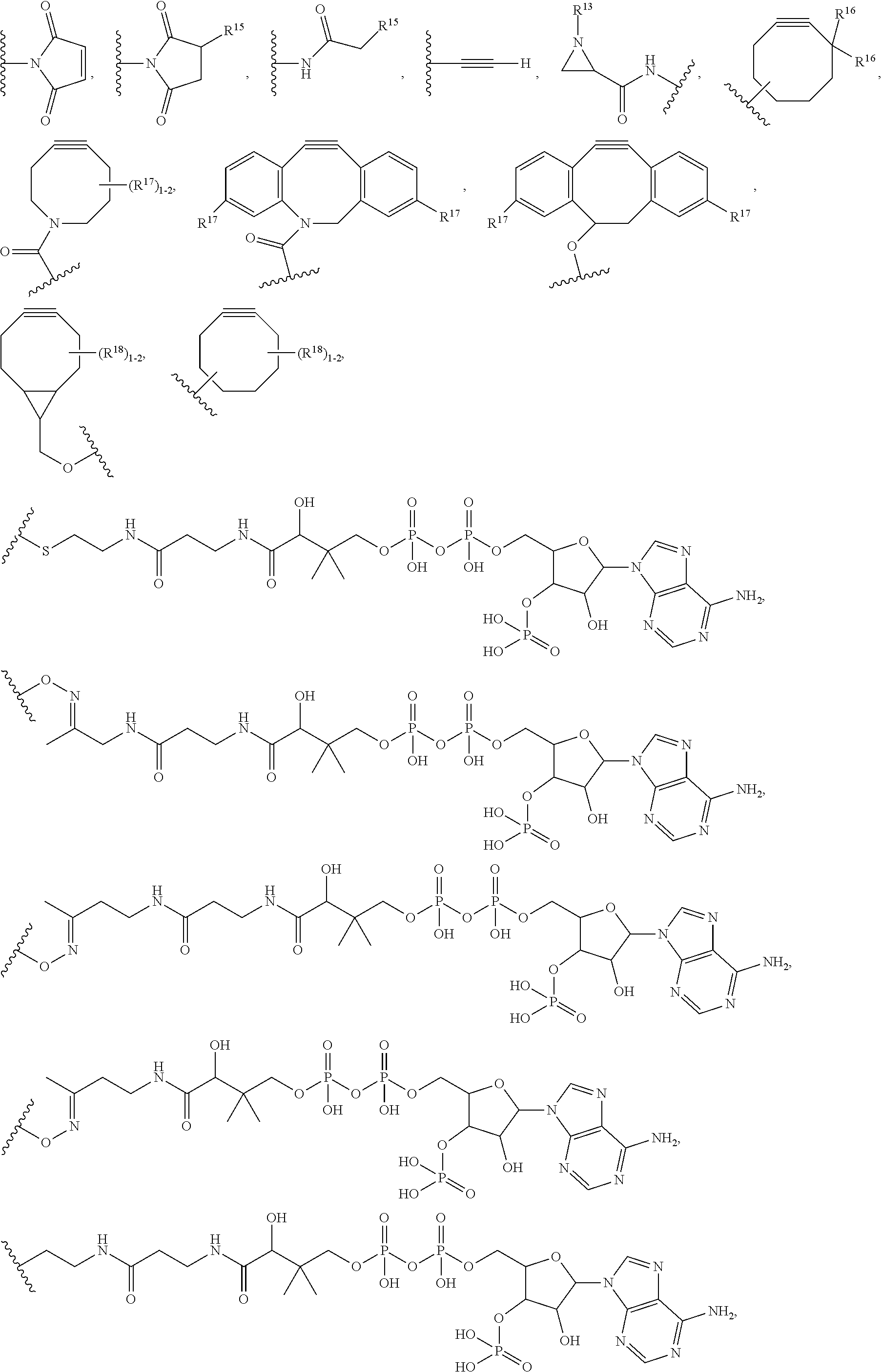

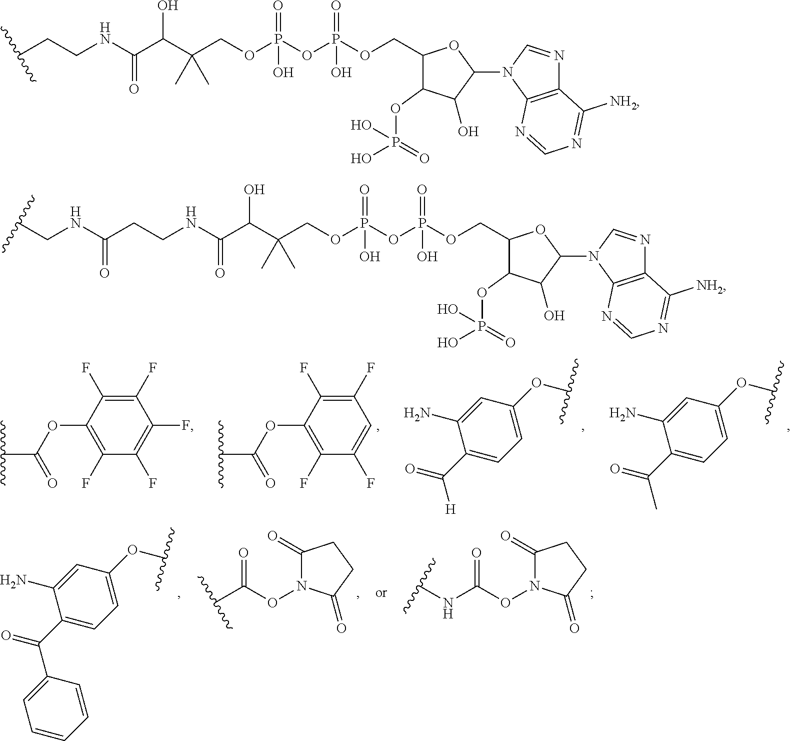

wherein

R¹³ is independently selected for each occasion from H and C₁-C₆ alkyl;

R¹⁴ is —S(CH₂)ₙCHR¹⁵NHC(═O)R¹³;

R¹⁵ is R¹³ or —C(═O)OR¹³;

R¹⁶ is independently selected for each occasion from H, C₁-C₆ alkyl, F, Cl, and —OH;

R¹⁷ is independently selected for each occasion from H, C₁-C₆ alkyl, F, Cl, —NH₂, —OCH₃, —OCH₂CH₃, —N(CH₃)2, —CN, —NO₂ and —OH; and

R¹⁸ is independently selected for each occasion from H, C₁-C₆ alkyl, F, benzyloxy substituted with —C(═O)OH, benzyl substituted with —C(═O)OH, C₁-C₄ alkoxy substituted with —C(═O)OH, and C₁-C₄ alkyl substituted with —C(═O)OH.

As depicted in Table 3, examples of suitably reactive substituents Z′ on the linker and reactive substituents on the antibody or antigen-binding fragment thereof include a nucleophile/electrophile pair (e.g., a thiol/haloalkyl pair, an amine/carbonyl pair, or a thiol/α,β-unsaturated carbonyl pair, and the like), a diene/dienophile pair (e.g., an azide/alkyne pair, or a diene/α,β-unsaturated carbonyl pair, among others), and the like. Coupling reactions between the reactive substitutents to form the chemical moiety Z include, without limitation, thiol alkylation, hydroxyl alkylation, amine alkylation, amine or hydroxylamine condensation, hydrazine formation, amidation, esterification, disulfide formation, cycloaddition (e.g., [4+2] Diels-Alder cycloaddition, [3+2] Huisgen cycloaddition, among others), nucleophilic aromatic substitution, electrophilic aromatic substitution, and other reactive modalities known in the art or described herein. In some embodiments, the reactive substituent Z′ is an electrophilic functional group suitable for reaction with a nucleophilic functional group on the antibody, or antigen-binding fragment thereof.

Reactive substituents that may be present within an antibody, or antigen-binding fragment thereof, as disclosed herein include, without limitation, nucleophilic groups such as (i) N-terminal amine groups, (ii) side chain amine groups, e.g. lysine, (iii) side chain thiol groups, e.g. cysteine, and (iv) sugar hydroxyl or amino groups where the antibody is glycosylated. Reactive substituents that may be present within an antibody, or antigen-binding fragment thereof, as disclosed herein include, without limitation, hydroxyl moieties of serine, threonine, and tyrosine residues; amino moieties of lysine residues; carboxyl moieties of aspartic acid and glutamic acid residues; and thiol moieties of cysteine residues, as well as propargyl, azido, haloaryl (e.g., fluoroaryl), haloheteroaryl (e.g., fluoroheteroaryl), haloalkyl, and haloheteroalkyl moieties of non-naturally occurring amino acids. In some embodiments, the reactive substituents present within an antibody, or antigen-binding fragment thereof as disclosed herein include, are amine or thiol moieties. Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by introducing one, two, three, four, or more cysteine residues (e.g., preparing mutant antibodies comprising one or more non-native cysteine amino acid residues). U.S. Pat. No. 7,521,541 teaches engineering antibodies by introduction of reactive cysteine amino acids.

In some embodiments, the reactive substituent Z′ attached to the linker is a nucleophilic group which is reactive with an electrophilic group present on an antibody. Useful electrophilic groups on an antibody include, but are not limited to, aldehyde and ketone carbonyl groups. A nucleophilic group (e.g., a) heteroatom of can react with an electrophilic group on an antibody and form a covalent bond to the antibody. Useful nucleophilic groups include, but are not limited to, hydrazide, oxime, amino, hydroxyl, hydrazine, thiosemicarbazone, hydrazine carboxylate, and arylhydrazide.

In some embodiments, chemical moiety Z is the product of a reaction between reactive nucleophilic substituents present within the antibodies, or antigen-binding fragments thereof, such as amine and thiol moieties, and a reactive electrophilic substituent Z′ attached to the linker. For instance, Z′ may be a Michael acceptor (e.g., maleimide), activated ester, electron-deficient carbonyl compound, or an aldehyde, among others.

Several representative and non-limiting examples of reactive substituents and the resulting chemical moieties are provided in Table 4.

TABLE 4
Complementary reactive substituents and chemical moieties

| | Functional Group on | | |
|---|---|---|---|
| | Antibody | Z′ group | Z group |
| Naturally Occurring | 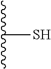 [SMILES: *S] | 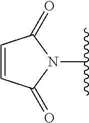 [SMILES: *N1C(=O)C=CC1=O] | 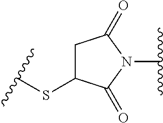 [SMILES: *SC1CC(=O)N(*)C1=O] |
| | | 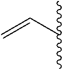 [SMILES: *C=C] | 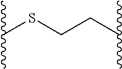 [SMILES: *CCS*] |
| | 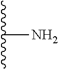 [SMILES: *N] | 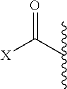 | 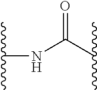 [SMILES: *NC(*)=O] |
| Synthetically Introduced | 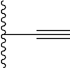 [SMILES: *C#C] | 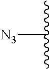 [SMILES: *N=[N+]=[N-]] | 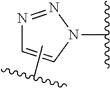 |
| | 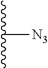 [SMILES: *N=[N+]=[N-]] | 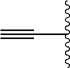 [SMILES: *C#C] | 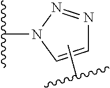 |
| | | 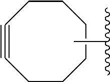 | 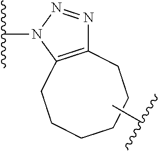 |
| | 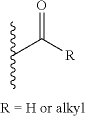 | 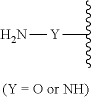 | 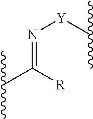 |

For instance, linkers suitable for the synthesis of linker-antibody conjugates and ADCs include, without limitation, reactive substituents Z′ attached to the linker, such as a maleimide or haloalkyl group. These may be attached to the linker by, for example, reagents such as succinimidyl 4-(N-maleimidomethyl)-cyclohexane-L-carboxylate (SMCC), N-succinimidyl iodoacetate (SIA), sulfo-SMCC, m-maleimidobenzoyl-N-hydroxysuccinimidyl ester (MBS), sulfo-MBS, and succinimidyl iodoacetate, among others described, in for instance, Liu et al., 18:690-697, 1979, the disclosure of which is incorporated herein by reference as it pertains to linkers for chemical conjugation.

In some embodiments, the reactive substituent Z′ attached to linker L is a maleimide, azide, or alkyne. An example of a maleimide-containing linker is the non-cleavable maleimidocaproyl-based linker, which is particularly useful for the conjugation of microtubule-disrupting agents such as auristatins. Such linkers are described by Doronina et al., Bioconjugate Chem. 17:14-24, 2006, the disclosure of which is incorporated herein by reference as it pertains to linkers for chemical conjugation.

In some embodiments, the reactive substituent Z′ is —(C═O)— or —NH(C═O)—, such that the linker may be joined to the antibody, or antigen-binding fragment thereof, by an amide or urea moiety, respectively, resulting from reaction of the —(C═O)— or —NH(C═O)— group with an amino group of the antibody or antigen-binding fragment thereof.

In some embodiments, the reactive substituent Z′ is an N-maleimidyl group, halogenated N-alkylamido group, sulfonyloxy N-alkylamido group, carbonate group, sulfonyl halide group, thiol group or derivative thereof, alkynyl group comprising an internal carbon-carbon triple bond, (hetero)cycloalkynyl group, bicyclo[6.1.0]non-4-yn-9-yl group, alkenyl group comprising an internal carbon-carbon double bond, cycloalkenyl group, tetrazinyl group, azido group, phosphine group, nitrile oxide group, nitrone group, nitrile imine group, diazo group, ketone group, (O-alkyl)hydroxylamino group, hydrazine group, halogenated N-maleimidyl group, 1,1-bis (sulfonylmethyl)methylcarbonyl group or elimination derivatives thereof, carbonyl halide group, or an allenamide group, each of which may be optionally substituted. In some embodiments, the reactive substituent comprises a cycloalkene group, a cycloalkyne group, or an optionally substituted (hetero)cycloalkynyl group.

In some embodiments, the chemical moiety Z is selected from Table 3 or Table 4. In some embodiments, the chemical moiety Z is:

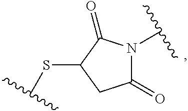

where S is a sulfur atom which represents the reactive substituent present within an antibody, or an antigen-binding fragment thereof, that specifically binds to an antigen expressed on the cell surface of a human stem cell or a T cell (e.g., from the —SH group of a cysteine residue).

In some embodiments, the linker-reactive substituent group, taken together as L-Z′, prior to conjugation with the antibody or antigen binding fragment thereof, has the structure:

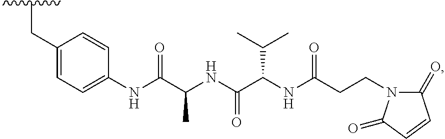 [SMILES: *Cc1ccc(NC(=O)[C@H](C)NC(=O)[C@@H](NC(=O)CCN2C(=O)C=CC2=O)C(C)C)cc1]

where the wavy line indicates the point of attachment to the cytotoxin (e.g., an amatoxin or derivative thereof). This linker-reactive substituent group L-Z′ may alternatively be referred to as N-beta-maleimidopropyl-Val-Ala-para-aminobenzyl (BMP-Val-Ala-PAB).

In some embodiments, the linker L and the chemical moiety Z, after conjugation to the antibody, taken together as L-Z-Ab, has the structure:

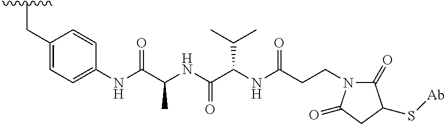

where S is a sulfur atom which represents the reactive substituent present within an antibody, or an antigen-binding fragment thereof, that specifically binds to an antigen expressed on the cell surface of a human stem cell or a T cell (e.g., from the —SH group of a cysteine residue. The wavy line at the linker terminus indicates the point of attachment to the cytotoxin, e.g., an amatoxin or derivative thereof.

In some embodiments, the linker-reactive substituent group, taken together as L-Z′, prior to conjugation with the antibody or antigen binding fragment thereof, has the structure:

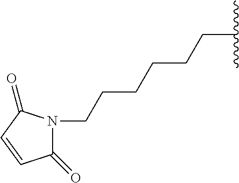 [SMILES: *CCCCCCN1C(=O)C=CC1=O]

where the wavy line indicates the point of attachment to the cytotoxin (e.g., an amatoxin or derivative thereof).

In some embodiments, the linker L and the chemical moiety Z, after conjugation to the antibody, taken together as L-Z-Ab, has the structure:

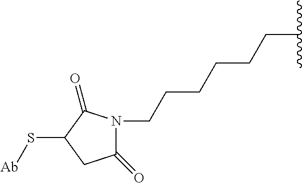

where S is a sulfur atom which represents the reactive substituent present within an antibody, or an antigen-binding fragment thereof, that specifically binds to an antigen expressed on the cell surface of a human stem cell or a T cell (e.g., from the —SH group of a cysteine residue. The wavy line at the linker terminus indicates the point of attachment to the cytotoxin, e.g., an amatoxin or derivative thereof.

In some embodiments, an amatoxin as disclosed herein is conjugated to a linker-reactive moiety -L-Z′ having the following formula:

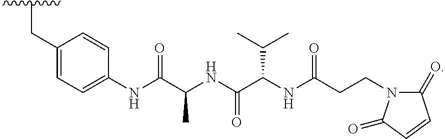 [SMILES: *Cc1ccc(NC(=O)[C@H](C)NC(=O)[C@@H](NC(=O)CCN2C(=O)C=CC2=O)C(C)C)cc1]

In some embodiments, an amatoxin as disclosed herein is conjugated to a linker-reactive moiety -L-Z′ having the following formula:

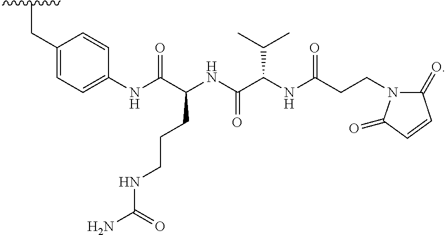 [SMILES: *Cc1ccc(NC(=O)[C@H](CCCNC(N)=O)NC(=O)[C@@H](NC(=O)CCN2C(=O)C=CC2=O)C(C)C)cc1]

The foregoing linker moieties and amatoxin-linker conjugates, among others useful in conjunction with the compositions and methods described herein, are described, for example, in U.S. Patent Application Publication No. 2015/0218220 and Patent Application Publication No. WO2017/149077, the disclosure of each of which is incorporated herein by reference in its entirety.

In one aspect, the cytotoxin of the ADC as disclosed herein as an amatoxin or derivative thereof. In some embodiments, the amatoxin is represented by any of formulae (III), (IIIa), (IIIb), or (IIIc). One of skill in the art will recognize that such amatoxins present multiple possibilities for attachment points to the linker (e.g., at any of positions denoted by variables R₁ through R₉).

For instance, the antibodies, or antigen-binding fragments, described herein may be bound to an amatoxin so as to form a conjugate represented by the formula Ab-Z-L-Am, wherein Ab is the antibody, or antigen-binding fragment thereof, L is a linker, Z is a chemical moiety and Am is an amatoxin. In some embodiments, Ab-Z-L-Am is represented by the structural formula (I):

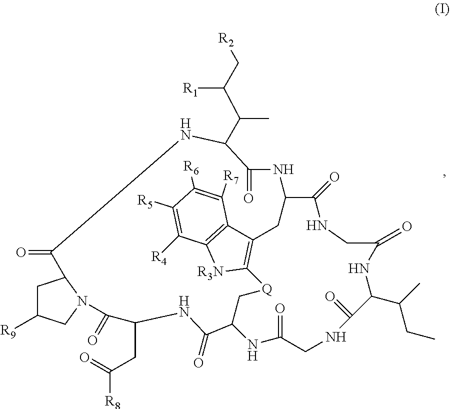

wherein:

Q is —S—, —S(O)—, or —SO₂—;

R₁ is H, OH, OR_{A}, or OR_{D};

R₂ is H, OH, OR_{B}, or OR_{D};

R_{A} and R_{B}, when present, together with the oxygen atoms to which they are bound, combine to form an optionally substituted 5-membered heterocycloalkyl group;

R₃ is H, R_{C}, or R_{D};

R₄ is H, OH, OR_{C}, OR_{D}, R_{C}, or R_{D};

R₅ is H, OH, OR_{C}, OR_{D}, R_{C}, or R_{D};

R₆ is H, OH, OR_{C}, OR_{D}, R_{C}, or R_{D};

R₇ is H, OH, OR_{C}, OR_{D}, R_{C}, or R_{D};

R₈ is OH, NH₂, OR_{C}, OR_{D}, NHR_{D}, or NR_{C}R_{D};

R₉ is H, OH, OR_{C}, or OR_{D};

R_{C} is C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, cycloalkyl, heterocycloalkyl, aryl, heteroaryl, or a combination thereof, wherein each C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, cycloalkyl, heterocycloalkyl, aryl, or heteroaryl may optionally be substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro; and

R_{D} is -L-Z-Ab, where each of L, Z, and Ab are as disclosed herein.

In some embodiments, the ADC of formula (I) contains exactly one R_{D} substituent.

In some embodiments, the ADC of formula (I) is represented by formula (Ia):

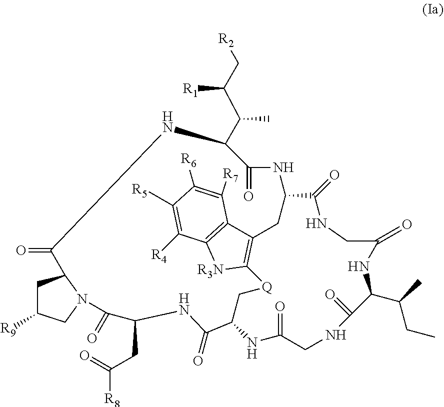

wherein each of Q, R₁-R₉, R_{A}, R_{B}, R_{C}, and R_{D} are as previously defined for formula (I).

In some embodiments, the ADC of formula (Ia) contains exactly one R_{D} substituent.

In some embodiments, the ADC of formula (I) is represented by formula (Ib)

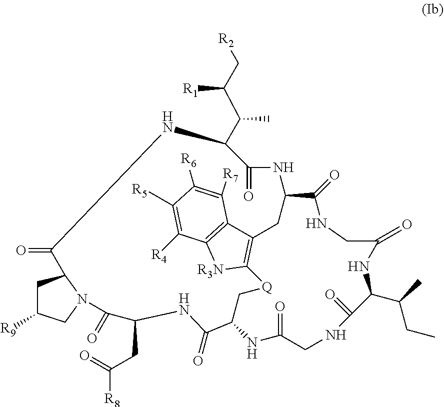

wherein each of Q, R₁-R₉, R_{A}, R_{B}, R_{C}, and R_{D} are as previously defined for formula (I).

In some embodiments, the ADC of formula (Ib) contains exactly one R_{D} substituent.

In some embodiments, the ADC is represented by formula (I), (Ia), or (Ib), wherein:
R_{A} and R_{B}, when present, together with the oxygen atoms to which they are bound, combine to form:

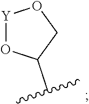

wherein:

Y is —(C═O)—, —(C═S)—, —(C═NR_{E})—, or CR_{E}R_{E}′; and

R_{E} and R_{E}′ are each independently selected from H, C₁-C₆ alkylene-R_{D}, C₁-C₆ heteroalkylene-R_{D}, C₂-C₆ alkenylene-R_{D}, C₂-C₆ heteroalkenylene-R_{D}, C₂-C₆ alkynylene-R_{D}, C₂-C₆ heteroalkynylene-R_{D}, cycloalkylene-R_{D}, heterocycloalkylene-R_{D}, arylene-R_{D}, heteroarylene-R_{D};

wherein each of said C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, cycloalkylene, heterocycloalkylene, arylene, or heteroarylene may optionally be substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

In some embodiments, the ADC is represented by formula (I), (Ia), or (Ib), wherein:
R₁ is H, OH, OR_{A}, or OR_{D};
R₂ is H, OH, OR_{B}, or OR_{D};
R_{A} and R_{B}, when present, together with the oxygen atoms to which they are bound, combine to form:

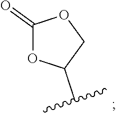 [SMILES: *C1COC(=O)O1]

R₃ is H, R_{C}, or R_{D};
R₄ is H, OH, OR₀, OR_{D}, RP, or R_{D};
R₅ is H, OH, OR_{C}, OR_{D}, R_{C}, or R_{D};
R₆ is H, OH, OR_{C}, OR_{D}, R_{C}, or R_{D};
R₇ is H, OH, OR_{C}, OR_{D}, R_{C}, or R_{D};
R₈ is OH, NH₂, OR_{D}, or NHR_{D}; and
R₉ is H or OH.

In some embodiments, the ADC is represented by formula (I), (Ia), or (Ib), wherein:
R₁ is H, OH, OR_{A}, or OR_{D};
R₂ is H, OH, OR_{B}, or OR_{D};
R_{A} and R_{B}, when present, together with the oxygen atoms to which they are bound, combine to form:

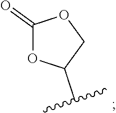 [SMILES: *C1COC(=O)O1]

R₃ is H or R_{D};
R₄ and R₅ are each independently H, OH, OR_{C}, R_{D}, or OR_{D};
R₆ and R₇ are each H;
R₈ is OH, NH₂, OR_{D}, or NHR_{D}; and
R₉ is H or OH.

In some embodiments, the ADC is represented by formula (I), (Ia), or (Ib), wherein
R₁ is H, OH, or OR_{A};
R₂ is H, OH, or OR_{B};
R_{A} and R_{B}, when present, together with the oxygen atoms to which they are bound, combine to form:
R₃, R₄, R₆, and R₇ are each H;

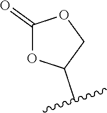 [SMILES: *C1COC(=O)O1]

R₅ is ORD;
R₈ is OH or NH₂; and
R₉ is H or OH.

Such amatoxin conjugates are described, for example, in US Patent Application Publication No. 2016/0002298, the disclosure of which is incorporated herein by reference in its entirety.

In some embodiments, the ADC is represented by formula (I), (Ia), or (Ib), wherein
R₁ and R₂ are each independently H or OH;
R₃ is R_{D};
R₄, R₆, and R₇ are each H;
R₅ is H, OH, or OC₁-C₆ alkyl;
R₈ is OH or NH₂; and
R₉ is H or OH.

Such amatoxin conjugates are described, for example, in US Patent Application Publication No. 2014/0294865, the disclosure of which is incorporated herein by reference in its entirety.

In some embodiments, the ADC is represented by formula (I), (Ia), or (Ib), wherein
R₁ and R₂ are each independently H or OH;
R₃, R₆, and R₇ are each H;
R₄ and R₅ are each independently H, OH, OR_{D}, or R_{D};
R₈ is OH or NH₂; and
R₉ is H or OH.

Such amatoxin conjugates are described, for example, in US Patent Application Publication No. 2015/0218220, the disclosure of which is incorporated herein by reference in its entirety.

In some embodiments, the ADC is represented by formula (I), (Ia), or (Ib), wherein
R₁ and R₂ are each independently H or OH;
R₃, R₆, and R₇ are each H;
R₄ and R₅ are each independently H or OH;
R₈ is OH, NH₂, OR_{D}, or NHR_{D}; and
R₉ is H or OH.

Such amatoxin conjugates are described, for example, in U.S. Pat. Nos. 9,233,173 and 9,399,681, as well as in US 2016/0089450, the disclosures of each of which are incorporated herein by reference in their entirety.

In some embodiments, the ADC is represented by formula (I), (Ia), or (Ib), wherein
R₁ and R₂ are each OH;
R₃, R₄, R₆, and R₇ are each H;
R₅ is ORD;
R₈ is NH₂; and
R₉ is OH.

In some embodiments, the ADC is represented by formula (I), (Ia), or (Ib), wherein
R₁ and R₂ are each independently H or OH;
R₃, R₆, and R₇ are each H;
R₄ and R₅ are each independently H or OH;
R₈ is OR_{D}, or NHR_{D}; and
R₉ is H or OH.

In some embodiments, the linker L comprises one or more of a hydrazine, a disulfide, a thioether, an amino acid, a peptide consisting of up to 10 amino acids, a p-aminobenzyl (PAB) group, a heterocyclic self-immolative group, C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, C₃-C₆ cycloalkyl, heterocycloalkyl, aryl, heteroaryl, a —(C═O)— group, a —C(O)NH— group, an —OC(O)NH— group, a —(CH₂CH₂O)ₚ— group where p is an integer from 1-6, or a solubility enhancing group;

wherein each C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, C₃-C₆ cycloalkyl, heterocycloalkyl, aryl, or heteroaryl group may optionally be substituted with from 1 to 5 substituents, independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

In some embodiments, each C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, C₃-C₆ cycloalkyl, heterocycloalkyl, aryl, or heteroaryl group may optionally be interrupted by one or more heteroatoms selected from O, S and N.

In some embodiments, each C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, C₃-C₆ cycloalkyl, heterocycloalkyl, aryl, or heteroaryl group may optionally be interrupted by one or more heteroatoms selected from O, S and N and may be optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

In some embodiments, the linker includes a p-aminobenzyl group (PAB). In one embodiment, the p-aminobenzyl group is disposed between the cytotoxic drug and a protease cleavage site in the linker. In one embodiment, the p-aminobenzyl group is part of a p-aminobenzyloxycarbonyl unit. In one embodiment, the p-aminobenzyl group is part of a p-aminobenzylamido unit.

In some embodiments, the linker comprises a peptide selected from the group consisting of Phe-Lys, Val-Lys, Phe-Ala, Phe-Cit, Val-Ala, Val-Cit, and Val-Arg. In some embodiments, the linker comprises one or more of PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB.

In some embodiments, the linker comprises one or more of a peptide, oligosaccharide, —(CH₂)ₚ—, —(CH₂CH₂O)ₚ—, —(C═O)(CH₂)ₚ—, PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB, wherein p is an integer from 1-6.

In some embodiments, the linker comprises PAB-Ala-Val-propionyl, represented by the formula:

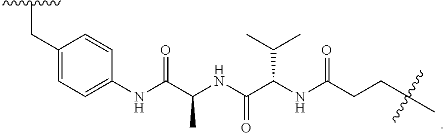 [SMILES: *CCC(=O)N[C@H](C(=O)N[C@@H](C)C(=O)Nc1ccc(C*)cc1)C(C)C]

In some embodiments, the linker comprises PAB-Cit-Val-propionyl, represented by the formula:

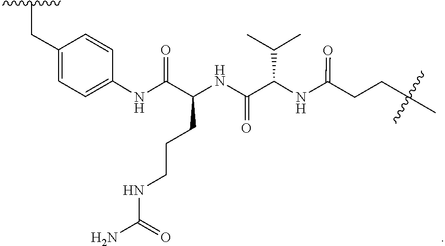 [SMILES: *CCC(=O)N[C@H](C(=O)N[C@@H](CCCNC(N)=O)C(=O)Nc1ccc(C*)cc1)C(C)C]

In some embodiments, the chemical moiety Z is selected from Table 3 or Table 4. In some embodiments, the chemical moiety Z is:

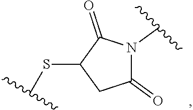

where S is a sulfur atom which represents the reactive substituent present within an antibody, or antigen-binding fragment thereof, that specifically binds to an antigen expressed on the cell surface of a human stem cell or a T cell (e.g., from the —SH group of a cysteine residue).

In some embodiments, the linker-reactive substituent group, taken together as L-Z′, prior to conjugation with the antibody or antigen binding fragment thereof, has the structure:

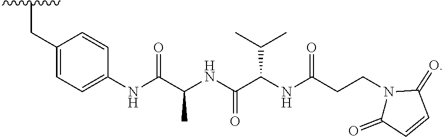 [SMILES: *Cc1ccc(NC(=O)[C@H](C)NC(=O)[C@@H](NC(=O)CCN2C(=O)C=CC2=O)C(C)C)cc1]

In some embodiments, the linker L and the chemical moiety Z, after conjugation to the antibody, taken together as L-Z-Ab, has the structure:

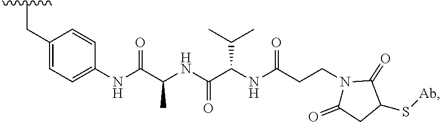

where S is a sulfur atom which represents the reactive substituent present within an antibody, or antigen-binding fragment thereof, that specifically binds to an antigen expressed on the cell surface of a human stem cell or a T cell (e.g., from the —SH group of a cysteine residue.

In some embodiments, the linker comprises a —(CH₂)ₙ— unit, where n is an integer from 2-6. In some embodiments, the linker comprises a —(CH₂)ₙ— where n is 6. In some embodiments, the linker is —(CH₂)ₙ— where n is 6, represented by the formula:

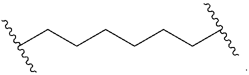 [SMILES: *CCCCCC*]

In some embodiments, the linker-reactive substituent group, taken together as L-Z′, prior to conjugation with the antibody or antigen binding fragment thereof, has the structure:

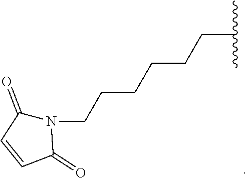 [SMILES: *CCCCCCN1C(=O)C=CC1=O]

In some embodiments, the linker L and the chemical moiety Z, after conjugation to the antibody, taken together as L-Z-Ab, has the structure:

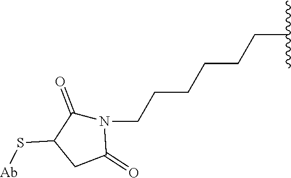

where S is a sulfur atom which represents the reactive substituent present within an antibody, or antigen-binding fragment thereof, that specifically binds to an antigen expressed on the cell surface of a human stem cell or a T cell (e.g., from the —SH group of a cysteine residue.

In particular embodiments, the ADC of formula (I) has one of the following structures:

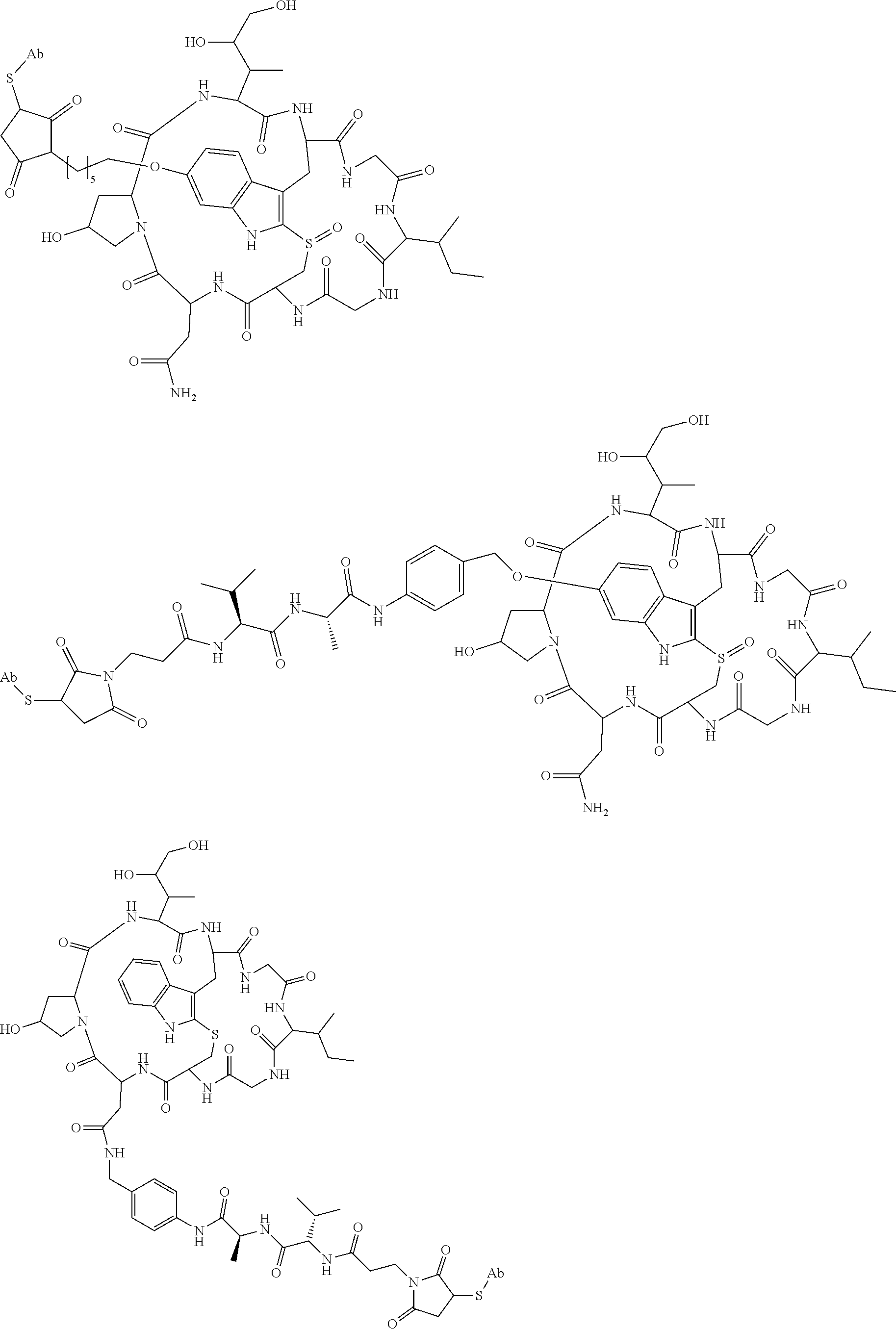

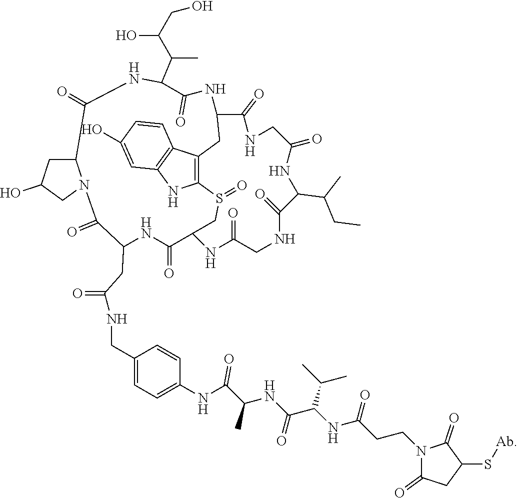

In particular embodiments, the ADC of formula (Ia) has one of the following structures:

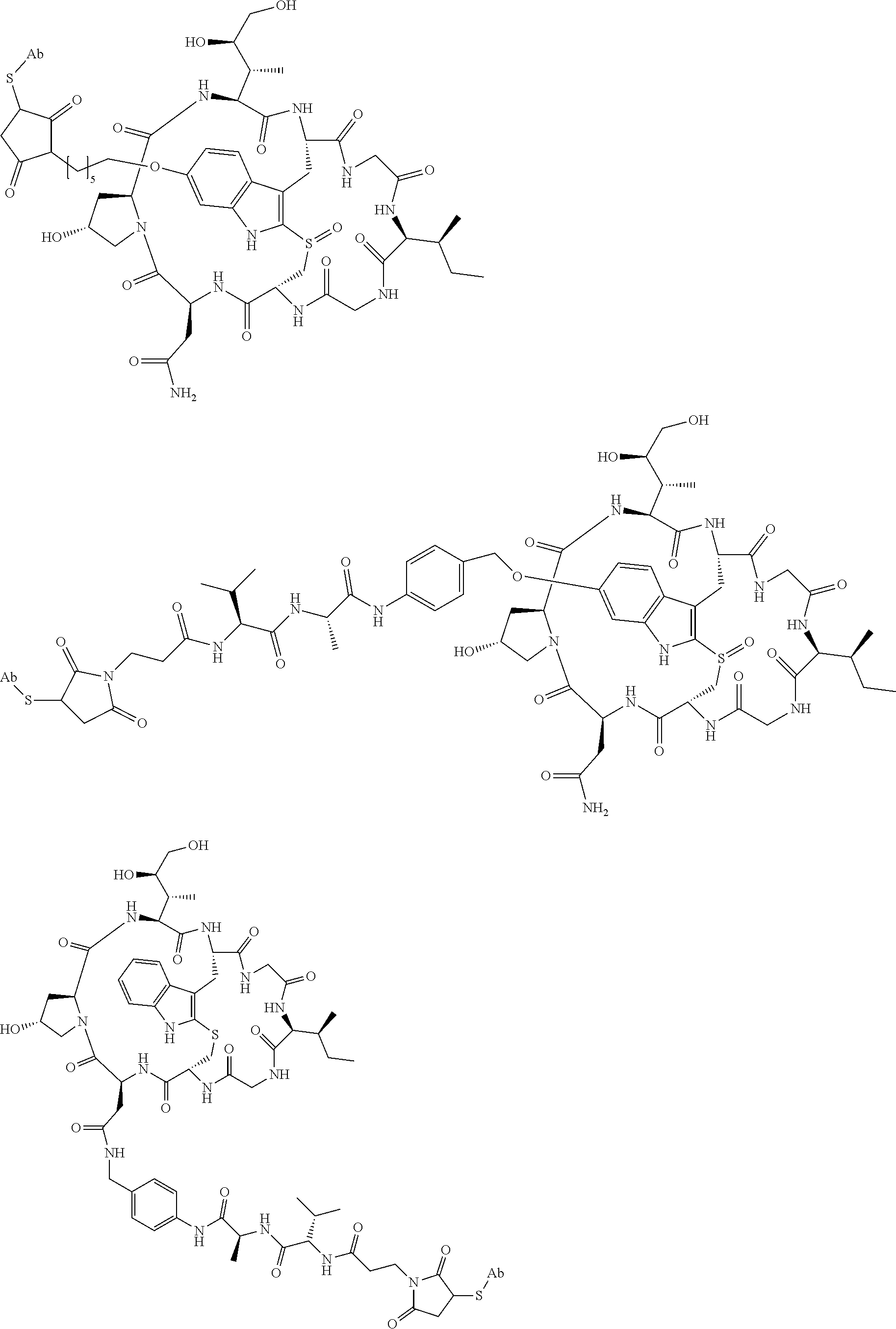

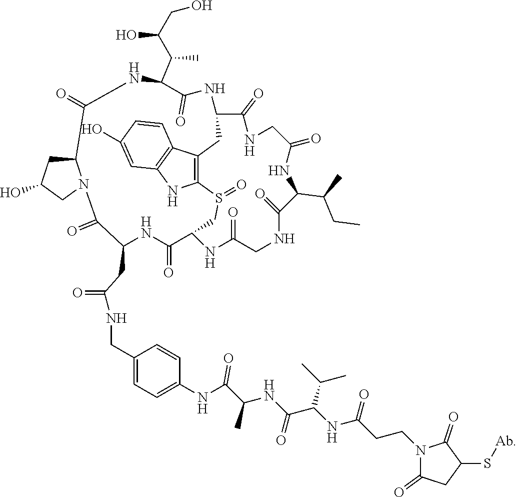

In particular embodiments, the ADC of formula (Ib) has one of the following structures:

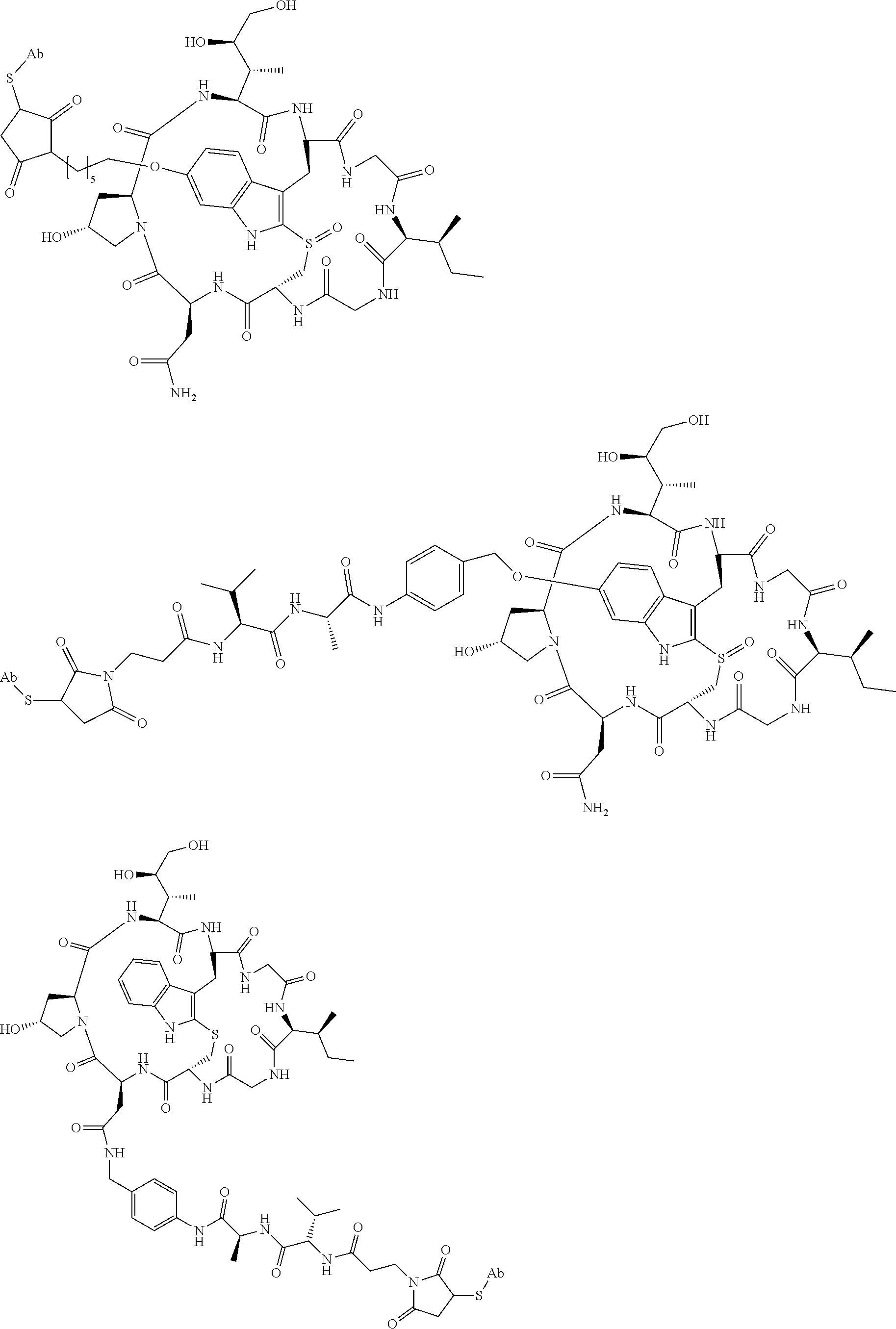

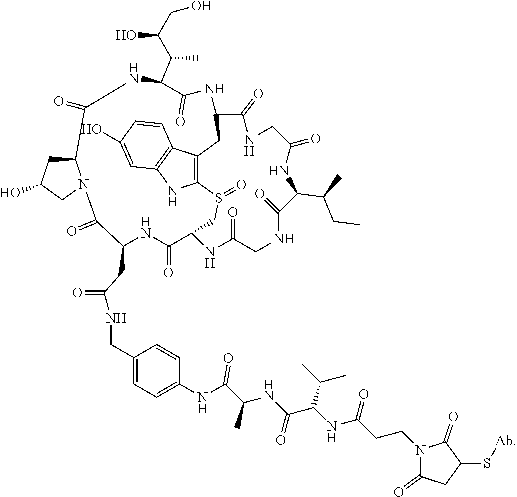

5. Preparation of Antibody-Drug Conjugates

In the ADCs of formula Ab-(Z-L-Cy)ₙ as disclosed herein, such as an ADO of any of formulae (I), (Ia), or (Ib), an antibody or antigen binding fragment thereof (Ab) is conjugated to one or more cytotoxic drug moieties (Cy; e.g., an amatoxin), for example, from about 1 to about 20 cytotoxic moieties per antibody, through a linker L and a chemical moiety Z as disclosed herein. In some embodiments, n is 1.

The ADCs of the present disclosure may be prepared by several routes, employing organic chemistry reactions, conditions, and reagents known to those skilled in the art, including: (1) reaction of a reactive substituent of an antibody or antigen binding fragment thereof with a bivalent linker reagent to form Ab-Z-L as described herein above, followed by reaction with a cytotoxic moiety Cy; or (2) reaction of a reactive substituent of a cytotoxic moiety with a bivalent linker reagent to form Cy-L-Z′, followed by reaction with a reactive substituent of an antibody or antigen binding fragment thereof as described herein above, to form an ADC of formula Ab-(Z-L-Cy)ₙ. Additional methods for preparing ADC are described herein.

In one embodiment, the antibody or antigen binding fragment thereof can have one or more carbohydrate groups that can be chemically modified to have one or more sulfhydryl groups. The ADC is then formed by conjugation through the sulfhydryl group's sulfur atom as described herein above.

In another embodiment, the antibody can have one or more carbohydrate groups that can be oxidized to provide an aldehyde (-CHO) group (see, for e.g., Laguzza, et al., J. Med. Chem. 1989, 32(3), 548-55). The ADC is then formed by conjugation through the corresponding aldehyde as described herein above. Other protocols for the modification of proteins for the attachment or association of cytotoxins are described in Coligan et al., Current Protocols in Protein Science, vol. 2, John Wiley & Sons (2002), incorporated herein by reference.

Methods for the conjugation of linker-drug moieties to cell-targeted proteins such as antibodies, immunoglobulins or fragments thereof are found, for example, in U.S. Pat. Nos. 5,208,020; 6,441,163; WO2005037992; WO2005081711; and WO2006/034488, all of which are hereby expressly incorporated by reference in their entirety.

Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

6. Pharmaceutical Compositions

ADCs described herein can be administered to a patient (e.g., a human patient suffering from an immune disease or cancer) in a variety of dosage forms. For instance, ADCs described herein can be administered to a patient suffering from an immune disease or cancer in the form of an aqueous solution, such as an aqueous solution containing one or more pharmaceutically acceptable excipients. Suitable pharmaceutically acceptable excipients for use with the compositions and methods described herein include viscosity-modifying agents. The aqueous solution may be sterilized using techniques known in the art.

Pharmaceutical formulations comprising ADCs as described herein are prepared by mixing such ADC with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

D. Routes of Administration and Dosing

1. Antibody Drug Conjugates (ADC) Administration

Antibodies, or antigen-binding fragments thereof, described herein can be administered to a patient (e.g., a human patient suffering from cancer, an autoimmune disease, or in need of hematopoietic stem cell transplant therapy) in a variety of dosage forms. For instance, antibodies, or antigen-binding fragments thereof, described herein can be administered to a patient suffering from cancer, an autoimmune disease, or in need of hematopoietic stem cell transplant therapy in the form of an aqueous solution, such as an aqueous solution containing one or more pharmaceutically acceptable excipients. Pharmaceutically acceptable excipients for use with the compositions and methods described herein include viscosity-modifying agents. The aqueous solution may be sterilized using techniques known in the art.

Pharmaceutical formulations comprising anti-CD117 or anti-CD45 antibodies and ADCs as described herein are prepared by mixing such antibody or ADC with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

The antibodies, and antigen-binding fragments, described herein may be administered by a variety of routes, such as orally, transdermally, subcutaneously, intranasally, intravenously, intramuscularly, intraocularly, or parenterally. The most suitable route for administration in any given case will depend on the particular antibody, or antigen-binding fragment, administered, the patient, pharmaceutical formulation methods, administration methods (e.g., administration time and administration route), the patient's age, body weight, sex, severity of the diseases being treated, the patient's diet, and the patient's excretion rate.

The effective dose of an anti-CD117 or anti-CD45 conjugate, antibody, or antigen-binding fragment thereof, described herein can range, for example from about 0.001 to about 100 mg/kg of body weight per single (e.g., bolus) administration, multiple administrations, or continuous administration, or to achieve an optimal serum concentration (e.g., a serum concentration of 0.0001-5000 pg/mL) of the antibody, or antigen-binding fragment thereof. The dose may be administered one or more times (e.g., 2-10 times) per day, week, or month to a subject (e.g., a human) suffering from cancer, an autoimmune disease, or undergoing conditioning therapy in preparation for receipt of a hematopoietic stem cell transplant. In the case of a conditioning procedure prior to hematopoietic stem cell transplantation, the antibody, or antigen-binding fragment thereof can be administered to the patient at a time that optimally promotes engraftment of the exogenous (genetically modified) hematopoietic stem cells, for instance, from about 1 hour to about 1 week (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, or about 7 days) or more prior to administration of the exogenous (genetically modified) hematopoietic stem cell transplant.

Using the methods disclosed herein, a physician of skill in the art can administer to a human patient in need of genetically modified hematopoietic stem cell transplant therapy an anti-CD117 or anti-CD45 ADC, an antibody or an antigen-binding fragment thereof capable of binding an antigen expressed by hematopoietic stem cells, such as an antibody or antigen-binding fragment thereof that binds, e.g., CD117 (for example, an antibody or antigen-binding fragment thereof that binds GNNK+CD117) or CD45. In this fashion, a population of endogenous hematopoietic stem cells can be depleted prior to administration of a genetically modified hematopoietic stem cell graft so as to promote engraftment of the hematopoietic stem cell graft.

As described above, the antibody may be covalently conjugated to a toxin, such as a cytotoxic molecule described herein or known in the art. For instance, an anti-CD117 antibody or antigen-binding fragment thereof (such as an anti-GNNK+CD117 antibody or antigen-binding fragment thereof) or anti-CD45 antibody or antigen-binding fragment thereof can be covalently conjugated to a cytotoxin, such as pseudomonas exotoxin A, deBouganin, diphtheria toxin, an amatoxin, such as γ-amanitin, α-amanitin, saporin, maytansine, a maytansinoid, an auristatin, an anthracycline, a calicheamicin, irinotecan, SN-38, a duocarmycin, a pyrrolobenzodiazepine, a pyrrolobenzodiazepine dimer, an indolinobenzodiazepine, an indolinobenzodiazepine dimer, or a variant thereof. This conjugation can be performed using covalent bond-forming techniques described herein or known in the art. The antibody, antigen-binding fragment thereof, or drug-antibody conjugate can subsequently be administered to the patient, for example, by intravenous administration, prior to transplantation of genetically modified hematopoietic stem cells (such as autologous, syngeneic, or allogeneic hematopoietic stem cells) to the patient.

The anti-CD117 antibody (e.g., anti-GNNK+CD117) or anti-CD45 antibody, or antigen-binding fragments thereof, or antibody drug conjugates thereof can be administered in an amount sufficient to reduce the quantity of endogenous hematopoietic stem cells, for example, by about 10%, about 20%, about 30%, about 40%, about 50%, about 60%, about 70%, about 80%, about 90%, about 95%, or more prior to hematopoietic stem cell transplant therapy. The reduction in hematopoietic stem cell count can be monitored using conventional techniques known in the art, such as by FACS analysis of cells expressing characteristic hematopoietic stem cell surface antigens in a blood sample withdrawn from the patient at varying intervals during conditioning therapy. For instance, a physician of skill in the art can withdraw a blood sample from the patient at various time points during conditioning therapy and determine the extent of endogenous hematopoietic stem cell reduction by conducting a FACS analysis to elucidate the relative concentrations of hematopoietic stem cells in the sample using antibodies that bind to hematopoietic stem cell marker antigens. According to some embodiments, when the concentration of hematopoietic stem cells has reached a minimum value in response to conditioning therapy with an anti-CD117 antibody (e.g., anti-GNNK+CD117) or anti-CD45 antibody, or antigen-binding fragments thereof, or antibody drug conjugates thereof, the physician may conclude the conditioning therapy, and may begin preparing the patient for hematopoietic stem cell transplant therapy.

The anti-CD117 antibody (e.g., anti-GNNK+CD117) or anti-CD45 antibody, or antigen-binding fragments thereof, or antibody drug conjugates thereof can be administered to the patient in an aqueous solution containing one or more pharmaceutically acceptable excipients, such as a viscosity-modifying agent. The aqueous solution may be sterilized using techniques described herein or known in the art. The antibody, antigen-binding fragment thereof, or antibody drug conjugate thereof can be administered to the patient at a dosage of, for example, from 0.001 mg/kg to 100 mg/kg prior to administration of a hematopoietic stem cell graft to the patient. The antibody, antigen-binding fragment thereof, or drug-antibody conjugate can be administered to the patient at a time that optimally promotes engraftment of the genetically modified hematopoietic stem cells, for instance, from about 1 hour to about 1 week (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, or about 7 days) or more prior to administration of the genetically modified hematopoietic stem cell transplant.

2. Genetically Modified Stem Cell Administration

Following the conclusion of conditioning therapy, the patient may then receive an infusion (e.g., an intravenous infusion) of genetically modified hematopoietic stem cells, such as from the same physician that performed the conditioning therapy or from a different physician. The physician may administer the patient an infusion of autologous, syngeneic, or allogeneic genetically modified hematopoietic stem cells, for instance, at a dosage of from 1×10³ to 1×10⁹ hematopoietic stem cells/kg. In some embodiments, the physician may administer the patient an infusion of the geneticially modified HSCs at a dosage of about 1×10³, about 2×10³, about 3×10³, about 4×10³, about 5×10³, about 6×10³, about 7×10³, about 8×10³, about 9×10³, about 1×10⁴, about 2×10⁴, about 3×10⁴, about 4×10⁴, about 5×10⁴, about 6×10⁴, about 7×10⁴, about 8×10⁴, about 9×10⁴, about 1×10⁵, about 2×10⁵, about 3×10⁵, about 4×10⁵, about 5×10⁵, about 6×10⁵, about 7×10⁵, about 8×10⁵, about 9×10⁵, about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, about 1×10⁸, about 2×10⁸, about 3×10⁸, about 4×10⁸, about 5×10⁸, about 6×10⁸, about 7×10⁸, about 8×10⁸, about 9×10⁸, about 1×10⁹, about 2×10⁹, about 3×10⁹, about 4×10⁹, about 5×10⁹, about 6×10⁹, about 7×10⁹, about 8×10⁹, or about 9×10⁹ HSC/kg. In some embodiments, the physician may administer the patient an infusion of the geneticially modified HSCs at a dosage of about 1×10³ to about 1×10⁸ HSCs/kg. In some embodiments, the physician may administer the patient an infusion of the geneticially modified HSCs at a dosage of about 1×10³ to about 1×10⁷ HSCs/kg. In some embodiments, the physician may administer the patient an infusion of the geneticially modified HSCs at a dosage of about 1×10³ to about 1×10⁶ HSCs/kg. In some embodiments, the physician may administer the patient an infusion of the geneticially modified HSCs at a dosage of about 1×10³ to about 1×10⁵ HSCs/kg. In some embodiments, the physician may administer the patient an infusion of the geneticially modified HSCs at a dosage of about 1×10⁴ to about 1×10⁸ HSCs/kg. In some embodiments, the physician may administer the patient an infusion of the geneticially modified HSCs at a dosage of about 1×10⁵ to about 1×10⁸ HSCs/kg. In some embodiments, the physician may administer the patient an infusion of the geneticially modified HSCs at a dosage of about 1×10⁶ to about 1×10⁸ HSCs/kg.

The physician may monitor the engraftment of the genetically modified hematopoietic stem cell transplant, for example, by withdrawing a blood sample from the patient and determining the increase in concentration of hematopoietic stem cells or cells of the hematopoietic lineage (such as megakaryocytes, thrombocytes, platelets, erythrocytes, mast cells, myeloblasts, basophils, neutrophils, eosinophils, microglia, granulocytes, monocytes, osteoclasts, antigen-presenting cells, macrophages, dendritic cells, natural killer cells, T-lymphocytes, and B-lymphocytes) following administration of the transplant. This analysis may be conducted, for example, from 1 hour to 6 months, or more, following genetically modified hematopoietic stem cell transplant therapy (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, or more; about 1 hour to about 24 weeks, about 1 week to about 10 weeks, about 1 day to about 24 weeks, about 1 day to about 6 months, about 1 day to about 5 months, about 1 day to about 4 months, about 1 day to about 3 months, about 1 day to about 2 months, about 1 day to about 1 month, about). Ranges encompassing the aforementioned times, e.g., about 4 weeks, are also contemplated herein. A finding that the concentration of hematopoietic stem cells or cells of the hematopoietic lineage has increased (e.g., by about 1%, about 2%, about 3%, about 4%, about 5%, about 6%, about 7%, about 8%, about 9%, about 10%, about 20%, about 30%, about 40%, about 50%, about 60%, about 70%, about 80%, about 90%, about 100%, about 200%, about 500%, or more; about 1% to about 500%, about 5% to about 250%, about 10% to about 100%, about 15% to about 200%, about 20% to about 200%, about 30% to about 300%, about 1% to about 100%) following the transplant therapy relative to the concentration of the corresponding cell type prior to transplant therapy provides one indication that treatment with the anti-CD117 (e.g., anti-GNNK+CD117) or anti-CD45 antibody, antigen-binding fragment thereof, or drug-antibody conjugate (ADC) has successfully promoted engraftment of the transplanted genetically modified hematopoietic stem cell graft. Ranges encompassing the aforementioned percentages, e.g., about 10%, are also contemplated herein. In some embodiments, successful engraftment can be determined by detecting the presence of an altered gene sequence. For example, in a treatment for sickle cell disease, engraftment can be determined by detecting the presence of the corrected HBB gene sequence.

### EXAMPLES

The following examples are put forth so as to provide those of ordinary skill in the art with a description of how the compositions and methods described herein may be used, made, and evaluated, and are intended to be purely exemplary of the present disclosure and are not intended to limit the scope of what the inventors regard as their invention.

### Example 1. Analysis of Anti-CD117 Antibody Conjugates Using an In Vitro Cell Killing Assay

Various antibodies described herein have been further characterized as described in PCT/US2018/057172, US 2019/0144558, US 2019/0153114, and PCT/US2018/057185, incorporated by reference in their entireties.

Whole IgG of the 10 anti-CD117 human IgG1 antibodies were pre-incubated with an anti-human Fab conjugated to a toxin (saporin) to test the ability of the various antibodies to kill Kasumi-1 cells (ATCC No. CRL-2724) in vitro. Following the cell killing assay, the level of cytotoxicity was quantified.

For in vitro killing assays using Kasumi-1 cells, Kasumi-1 cells were grown according to ATCC guidelines. More specifically, Kasumi-1 cells were cultured for three days in the presence of CD117-ADC or the positive control antibody (CK6; an antagonist antibody). Cell viability was measured by Celltiter Glo.

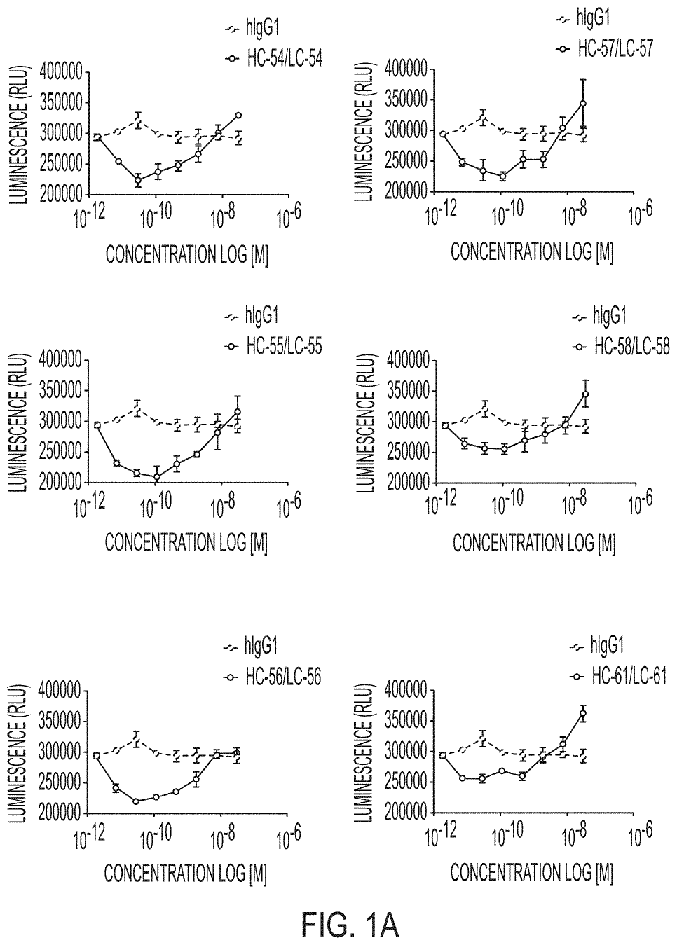

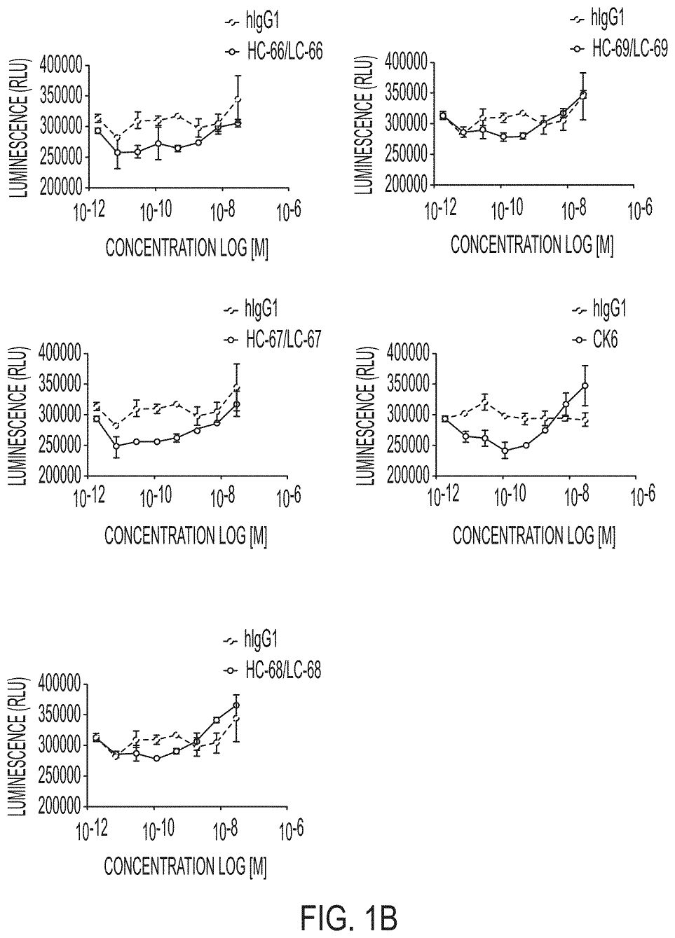

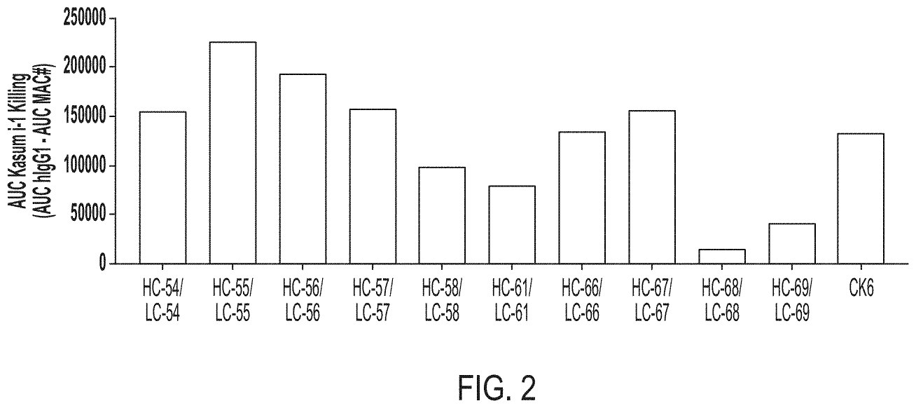

The results described in FIGS. 1A and 1B indicates that each of the various IgG:Fab-saporin complexes were effective at killing CD117 expressing cells (i.e., Kasumi-1 cells) in vitro and indicates that the complexes were internalized. FIG. 2 describes a quantification of the Kasumi-1 cell killing assay. Table 5 below provides additional data relating to this quantification of the Kasumi-1 cell killing assay. The Kasumi-1 cell killing assay was performed in the absence of SCF (as Kasumi-1 cells are not SCF dependent). As such, the antagonist/neutral characteristic of the tested antibodies was not apparent as in SCF-dependent cell killing assays (described in PCT/US2018/057172, incorporated by reference in its entirety). Notably, the antibodies identified as antagonist in the SCF-dependent cell killing assay exhibited a higher level of killing in the Kasumi-1 assay, e.g., compare AUC value of Ab55 (antagonist) to Ab67 (neutral).

TABLE 5
Quantification of IgG:Fab-saporin complex cell killing

| | AUC Kasumi-1 Killing |
|---|---|
| HC/LC | (AUC hIgG1 - AUC MAC ID#) |
| HC-54; LC-54 | 154918 |
| HC-55; LC-55 | 225764 |
| HC-56; LC-56 | 193260 |
| HC-57; LC-57 | 157277 |
| HC-58; LC-58 | 97423 |
| HC-61; LC-61 | 78558 |
| HC-66; LC-66 | 134742 |
| HC-67; LC-67 | 155931 |
| HC-68; LC-68 | 14411 |
| HC-69; LC-69 | 39988 |
| CK6 | 132193 |

### Example 2: A Single Dose of an Anti-CD117 ADC is Potent on Primary Human and NHP CD34+ Cells and Selective for HSCs

In vitro killing assays were performed using human and non-human primate (NHP) HSCs (i.e., isolated primary human and NHP CD34+ selected Bone Marrow Cells (BMCs)). Human CD34+ BMCs and NHP CD34+ BMCs were cultured for six days with an anti-CD117-ADC or a control (i.e., human isotype or NHP isotype). Viable CD34+ cells were evaluated by flow cytometry (data not shown).

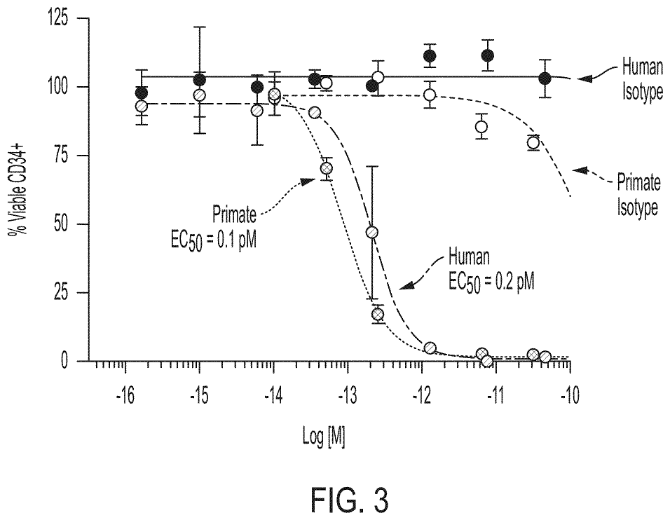

The anti-CD117 ADC is potent on both primary human and NHP CD34+ cells in vitro with EC₅₀ of 0.2 and 0.1 pM, respectively (FIG. 3), demonstrating that hematopoietic stem cell and progenitor cell depletion that was comparable to HSPC depletion observed following busufulan conditioning (6 mg/kg/day×4); data not shown) by targeting CD117, is sufficient to enable gene therapy.

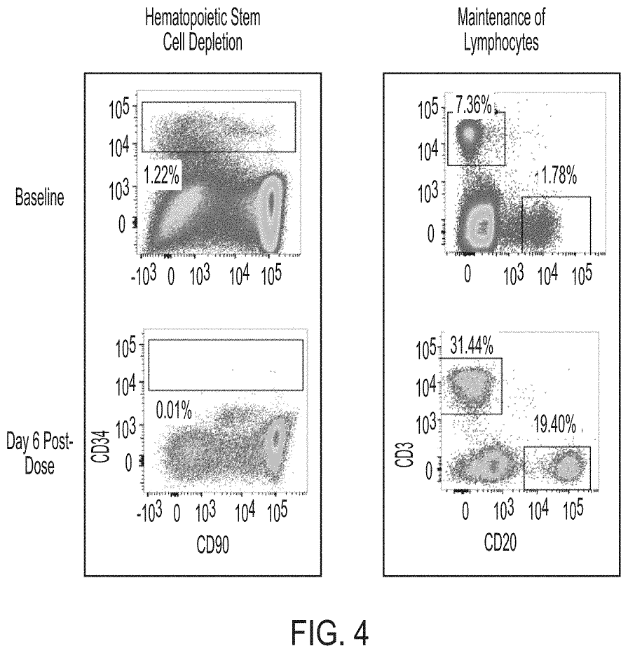

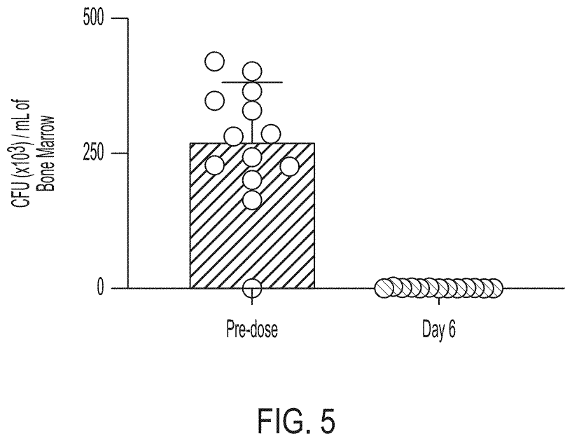

The efficacy and tolerability of the anti-CD117 ADC was evaluated in Rhesus primates. A single dose of the CD117-ADC was administered to Rhesus primates with the results analyzed using flow cytotmetry (FIG. 4). The data demonstrated that a single dose of the CD117-ADC led to significant CD34+ cell and colony forming cell depletion in the bone marrow 7 days after dosing (FIG. 5). In addition, the lymphocyte compartment was maintained (FIG. 4), demonstrating the specificity of the anti-CD117 ADC.

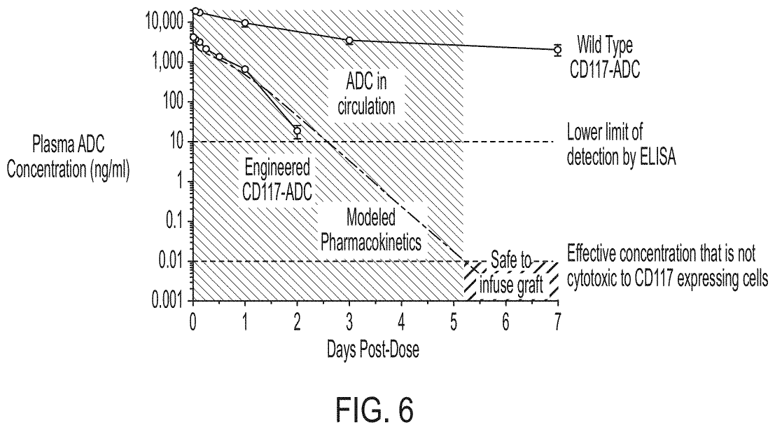

To facilitate the use in HSCT, an anti-CD117 ADC was engineered to have a fast clearance (t_{1/2}=about 10 hours). As described in FIG. 6, the ADC level decreased below the lower ELISA detection limit 48 hours post-administration, with modeled pharmacokinetics indicating that the ADC was below cytotoxic concentrations after 5 days post-adminstration (FIG. 6).

Taken together, these data indicated that the anti-CD117 ADC is fully myeloablative and robustly depletes Rhesus HSCs in vivo. No substantial effect of the anti-CD117 ADC on the lymphocytes was observed. These data indicated that the anti-CD117 ADC has a favorable safety profile, spares the immune system and is cleared rapidly to allow for optimal timing of graft infusion.

### Example 3: A Single Dose of an Anti-CD117 ADC Enables Autologous Gene-Modified Hematopoietic Stem Cell Transplant (Gene Therapy) in Nonhuman Primates

To determine whether the anti-CD117 ADC is sufficient to enable autologous HSC-based gene therapy (without the need for chemotherapy or radiation), engraftment of beta-globin transduced CD34+ cells was evaluated in two Rhesus macaque animals following a single dose of an anti-CD117 ADC. The anti-CD117 antibody used in the ADC of this Example is a Ab85 LALA/S239C/H435A fast half-life variant, which is conjugated to a PBD via the S239C mutation.

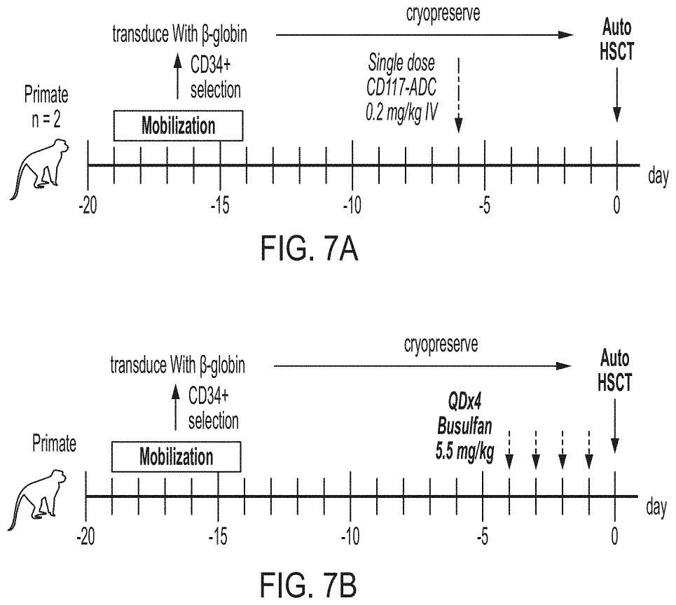

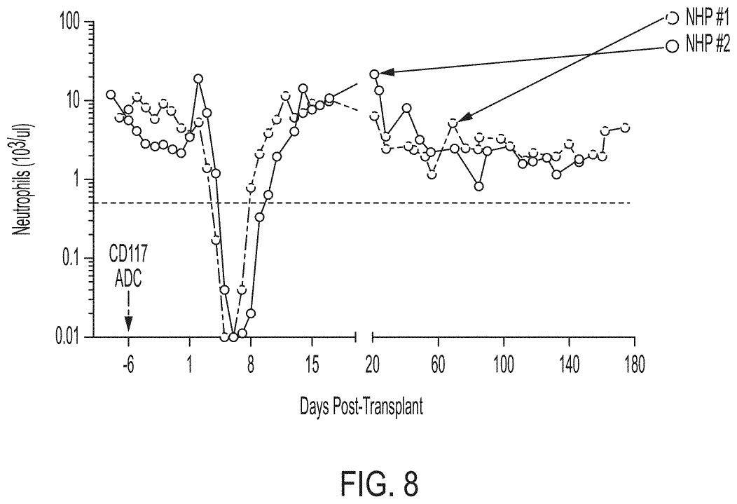

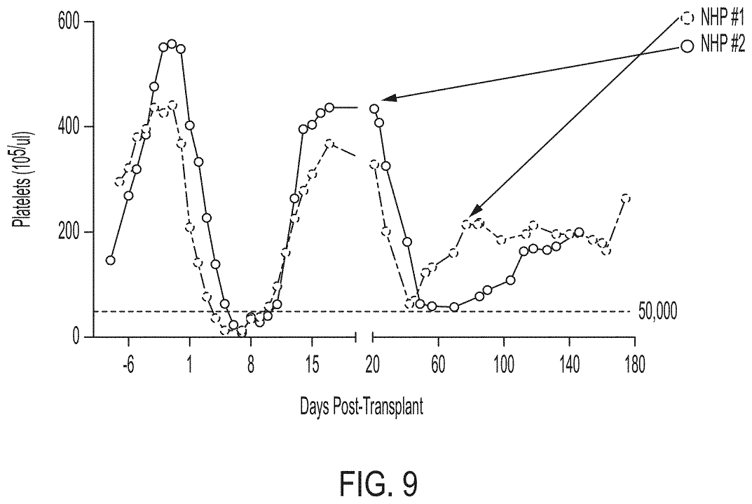

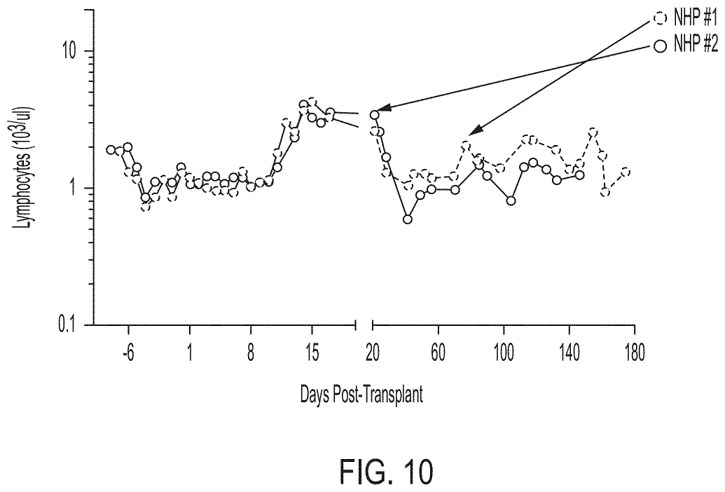

Two rhesus macaque were mobilized with granulocyte-colony stimulating factor (G-CSF, 20 mcg/kg/day×5) and plerixafor (1 mg/kg on day 5 of G-CSF) prior to apheresis (FIG. 7). The isolated CD34+ cells were transduced with a lentiviral vector encoding the p-globin gene and cryopreserved (FIG. 7). The anti-CD117 ADC was dosed on day −6 (0.2 mg/kg IV) and the cryopreserved gene modified cells were thawed and infused (3.3×10⁶ CD34+ cells/kg) on day 0 (i.e., the cells were transplanted back into the same animal 6 days after a single dose of the anti-CD117 ADC; see FIG. 7A). The anti-CD117 ADC conditioned animals became neutropenic 10 days following ADC dosing (FIG. 8). The animal recovered neutrophils on day 8 and 10 (FIG. 8) and platelets on day 10 and 11 (FIG. 9), while the lymphocytes were maintained throughout the transplant (FIG. 10). These data are comparable to busulfan conditioned animals, which recovered neutrophils on day 8 and 10 and platelets on day 10 and 15. Busulfan is similarly non-lympho ablative. Results are also provided below in Table 6.

TABLE 6

| | | Neutrophil | Platelet | Lymphocyte |
|---|---|---|---|---|
| Conditioning | | Recovery | Recovery | Counts |
| Regimen | Animal Number | Days post Transplant | | Range (10³/uL) |
| Anti-CD117 | NHP #1 | 8 | 10 | 0.9-4.2 |
| ADC | NHP #2 | 10 | 11 | 0.9-4.0 |
| Busulfan | Historical Cohort | 8-10 | 10-15 | 0.6-20 |
| | * Uchida et al. | | | |
| | Mol Ther 2019 | | | |

The peripheral granulocyte vector copy number (VCN) of the transduced CD34+ cells used for the anti-CD117-ADC conditioned animals was determined and found to be lower when compared to the VCN of the cells used in the busulfan conditioned animals (Table 7).

TABLE 7

| | | CD34 dose | VCN of | Peripheral |
|---|---|---|---|---|
| Conditioning | | (×10⁶ | infused | VCN |
| Regimen | Animal Number | cells/kg) | cells | @ 1-6 months |
| Anti-CD117 | NHP #1 | 3.3 | 5 | 0.01-0.04 |
| ADC | NHP #2 | 1.1 | 4 | 0.01-0.05 |
| Busulfan | Historical Cohort | 4.1-4.2 | 8-10 | 0.004-0.08 |
| | * Uchida et al. | | | |
| | Mol Ther 2019 | | | |

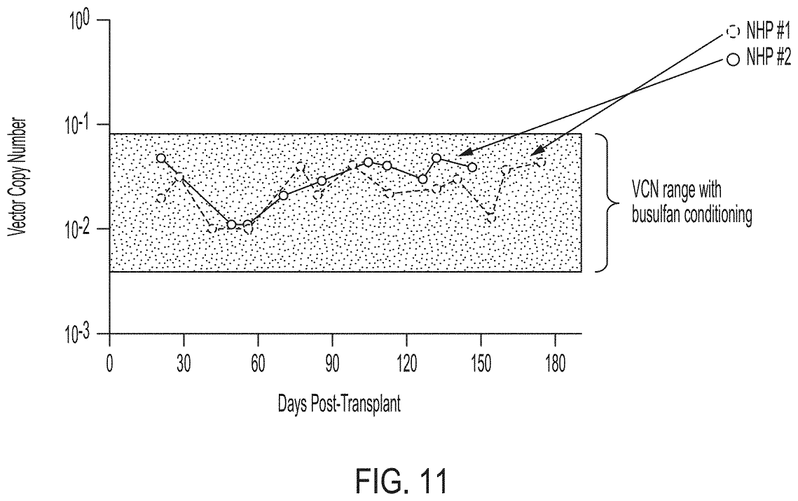

However, the peripheral granulocyte VCN of the transduced CD34+ cells used for the anti-CD117-ADC conditioned animals was stable over time and in the same range as that observed with the busulfan conditioned animals (see shaded region of FIG. 11). These data indicate that the conditioning with CD117-ADC was sufficient to enable engraftment of gene modified HSC at the same level as fully myeloablative busulfan.

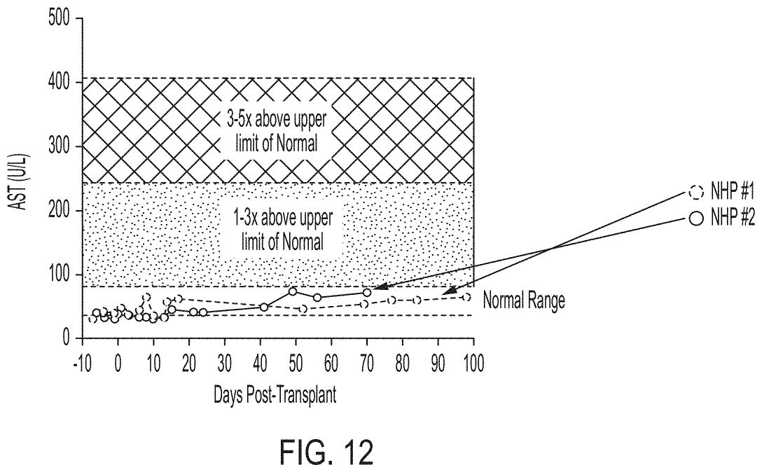

An advantageous aspect of these studies is that the CD117-ADC was well tolerated. The animals were observed to eat normally, with no observed gastroinstestinal (GI) side effects (Table 8), which is in contrast to animals treated with busulfan, which had loss of appetite (due to mucositis), weight loss, and diarrhea. No liver serum chemistry changes were observed (see, e.g., FIG. 12; no ADC-related changes outside the normal range were observed for aspartate aminotransferase (AST) alanine aminotransferase). In addition, no ADC-related changes outside the normal range were observed for ALT (data not shown), lactic acid dehydrogenase (LDH; data not shown), Albumin (data not shown), Creatinine (Cr; data not shown), blood urea nitrogen (BUN; data not shown), total bilirubin (TBIL; data not shown), alkaline phosphatase (ALP; data not shown), and Glucose (data not shown)), which is consistent with the absence of any observable behavioral abnormalities.

TABLE 8

| Busulfan Side Effect | Outcomes With CD117-ADC |
|---|---|
| Veno-occlusive Disease | Not observed |
| Wasting Syndrome | Not observed |
| Diarrhea | Not observed |
| Mucositis | Not observed |
| Seizures | Not observed |
| Emesis | Not observed |
| Pulmonary Fibrosis | Not observed |

The data shown herein demonstrate an anti-CD117 ADC that possesses potent activity on NHP CD34+ cells. This anti-CD117-ADC is fully myeloablative with a single dose in NHPs, has a favorable safety profile, spares the immune system and is cleared rapidly to allow for an appropriate timing of graft infusion. In a rhesus model of autologous gene modified HSCT, a single dose of the anti-CD117 ADC enables engraftment of gene modified HSC. These studies validate the use of an anti-CD117 ADC for targeted stem cell depletion prior to transplant and support its use as a new conditioning agent for auto-gene modified HSCT. Such a targeted approach for safer conditioning may improve the risk benefit profile for patients undergoing stem cell transplant and enable more patients to benefit from these potentially curative therapies including gene therapy.

TABLE 9
SEQUENCE SUMMARY

| Se- | | |
|---|---|---|
| quence | | |
| Identi- | | |
| fier | Description | Sequence |
| SEQ ID | CK6 CDR-H1 | SYWIG |
| NO: 1 | | |
| SEQ ID | CK6 CDR-H2 | IIYPGDSDTRYSPSFQG |
| NO: 2 | | |
| SEQ ID | CK6 CDR-H3 | HGRGYNGYEGAFDI |
| NO: 3 | | |
| SEQ ID | CK6 CDR-L1 | RASQGISSALA |
| NO: 4 | | |
| SEQ ID | CK6 CDR-L2 | DASSLES |
| NO: 5 | | |
| SEQ ID | CK6 CDR-L3 | CQQFNSYPLT |
| NO: 6 | | |
| SEQ ID | Consensus | EVQLVESGGGLVQPGGSLRLSCA |
| NO: 7 | human | ASGFTFSDYAMSWVRQAPGKGLE |
| | Ab Heavy | WVAVISENGSDTYYADSVKGRFT |
| | chain | ISRDDSKNTLYLQMNSLRAEDTA |
| | variable | VYYCARDRGGAVSYFDVWGQGTL |
| | domain | VTVSS |
| SEQ ID | Consensus | DIQMTQSPSSLSASVGDRVTITCR |
| NO: 8 | human | ASQDVSSYLAWYQQKPGKAPKLL |
| | Ab Light | IYAASSLESGVPSRFSGSGSGTDF |
| | chain | TLTISSLQPEDFATYYCQQYNSLP |
| | variable | YTFGQGTKVEIKRT |
| | domain | |
| SEQ ID | Ab67 Heavy | EVQLVESGGGLVQPGGSLRLSCA |
| NO: 9 | chain | ASGFTFSDADMDWVRQAPGKGL |
| | variable | EWVGRTRNKAGSYTTEYAASVKG |
| | region | RFTISRDDSKNSLYLQMNSLKTE |
| | {e.g., as | DTAVYYCAREPKYWIDFDLWGRG |
| | found | TLVTVSS |
| | in HC-67) | |
| | (CDRs in | |
| | bold) | |
| SEQ ID | Ab67 Light | DIQMTQSPSSLSASVGDRVTITCR |
| NO: 10 | chain | ASQSISSYLNWYQQKPGKAPKLLI |
| | variable | YAASSLQSGVPSRFSGSGSGTDFT |
| | region | LTISSLQPEDFATYYCQQSYIAPY |
| | {e.g., as | TFGGGTKVEIK |
| | found | |
| | in LC-67) | |
| | (CDRs in | |
| | bold) | |
| SEQ ID | Ab67 | FTFSDADMD |
| NO: 11 | CDR-H1 | |
| SEQ ID | Ab67 CDR-H2 | RTRNKAGSYTTEYAASVKG |
| NO: 12 | | |
| SEQ ID | Ab67 CDR-H3 | AREPKYWIDFDL |
| NO: 13 | | |
| SEQ ID | Ab67 CDR-L1 | RASQSISSYLN |
| NO: 14 | | |
| SEQ ID | Ab67 CDR-L2 | AASSLQS |
| NO: 15 | | |
| SEQ ID | Ab67 CDR-L3 | QQSYIAPYT |
| NO: 16 | | |
| SEQ ID | Ab67 | GAGGTGCAGCTGGTGGAGTCTG |
| NO: 17 | Heavy | GGGGAGGCTTGGTCCAGCCTGG |
| | chain | AGGGTCCCTGAGACTCTCCTGT |
| | variable | GCAGCCTCTGGATTCACCTTCAG |
| | region | TGACGCCGACATGGACTGGGTC |
| | (nucl) | CGCCAGGCTCCAGGGAAGGGGC |
| | | TGGAGTGGGTTGGCCGTACTAG |
| | | AAACAAAGCAGGAAGTTACACCA |
| | | CAGAATACGCCGCGTCTGTGAAA |
| | | GGCAGATTCACCATCTCAAGAGA |
| | | TGATTCAAAGAACTCACTGTATC |
| | | TGCAAATGAACAGCCTGAAAACC |
| | | GAGGACACGGCGGTGTACTACT |
| | | GCGCCAGAGAGCCIAAA1AC1G |
| | | GATCGACTTCGACCTATGGGGG |
| | | AGAGGTACCTTGGTCACCGTCTC |
| | | CTCA |
| SEQ ID | Ab67 Light | GACATCCAGATGACCCAGTCTCC |
| NO: 18 | chain | ATCCTCCCTGTCTGCATCTGTAG |
| | variable | GAGACAGAGTCACCATCACTTGC |
| | region | CGGGCAAGTCAGAGCATTAGCA |
| | (nucl) | GCTATTTAAATTGGTATCAGCAG |
| | | AAACCAGGGAAAGCCCCTAAGC |
| | | TCCTGATCTATGCTGCATCCAGT |
| | | TTGCAAAGTGGGGTCCCATCAAG |
| | | GTTCAGTGGCAGTGGATCTGGG |
| | | ACAGATTTCACTCTCACCATCAG |
| | | CAGTCTGCAACCTGAAGATTTTG |
| | | CAACTTACTACTGTCAGCAAAGC |
| | | TACATCGCCCCTTACACTTTTGG |
| | | CGGAGGGACCAAGGTTGAGATC |
| | | AAA |
| SEQ ID | Ab55 Heavy | QVQLVQSGAEVKKPGSSVKVSCK |
| NO: 19 | chain | ASGGTFRIYAISWVRQAPGQGLE |
| | variable | WMGGIIPDFGVANYAQKFQGRVTI |
| | region | TADESTSTAYMELSSLRSEDTAVY |
| | (e.g., | YCARGGLDTDEFDLWGRGTLVTV |
| | as found | SS |
| | in HC-55) | |
| | (CDRs in | |
| | bold) | |
| SEQ ID | Ab55 Light | DIQMTQSPSSLSASVGDRVTITC |
| NO: 20 | chain | RASQSINSYLNWYQQKPGKAPKL |
| | variable | LIYAASSLQSGVPSRFSGSGSGT |
| | region | DFTLTISSLQPEDFATYYC |
| | (e.g., | QQGVSDITFGGGTKVEIK |
| | as found | |
| | in LC-55) | |
| | (CDRs | |
| | in bold) | |
| SEQ ID | Ab55 CDR-H1 | GTFRIYAIS |
| NO: 21 | | |
| SEQ ID | Ab55 CDR-H2 | GIIPDFGVANYAQKFQG |
| NO: 22 | | |
| SEQ ID | Ab55 CDR-H3 | ARGGLDTDEFDL |
| NO: 23 | | |
| SEQ ID | Ab55 CDR-L1 | RASQSINSYLN |
| NO: 24 | | |
| SEQ ID | Ab55 CDR-L2 | AASSLQS |
| NO: 25 | | |
| SEQ ID | Ab55 CDR-L3 | QQGVSDIT |
| NO: 26 | | |
| SEQ ID | Ab55 Heavy | CAGGTGCAGCTGGTGCAGTCTG |
| NO: 27 | chain | GGGCTGAGGTGAAGAAGCCTGG |
| | variable | GTCCTCGGTGAAGGTCTCCTGC |
| | region | AAGGCTTCTGGAGGCACCTTCC |
| | (nucl) | GAATCTATGCTATCAGCTGGGTG |
| | | CGACAGGCCCCTGGACAAGGGC |
| | | TTGAGTGGATGGGAGGGATCAT |
| | | CCCTGACTTCGGTGTAGCAAACT |
| | | ACGCACAGAAGTTCCAGGGCAG |
| | | AGTCACGATTACCGCGGACGAAT |
| | | CCACGAGCACAGCCTACATGGA |
| | | GCTGAGCAGCCTGAGATCTGAG |
| | | GACACGGCGGTGTACTACTGCG |
| | | CCAGAGGTGGATTGGACACAGA |
| | | CGAGTTCGACCTATGGGGGAGA |
| | | GGTACCTTGGTCACCGTCTCCTC |
| | | A |
| SEQ ID | Ab55 Light | GACATCCAGATGACCCAGTCTCC |
| NO: 28 | chain | ATCCTCCCTGTCTGCATCTGTAG |
| | variable | GAGACAGAGTCACCATCACTTGC |
| | region | CGGGCAAGTCAGAGCATTAACA |
| | (nucl) | GCTATTTAAATTGGTATCAGCAG |
| | | AAACCAGGGAAAGCCCCTAAGC |
| | | TCCTGATCTATGCTGCATCCAGT |
| | | TTGCAAAGTGGGGTCCCATCAAG |
| | | GTTCAGTGGCAGTGGATCTGGG |
| | | ACAGATTTCACTCTCACCATCAG |
| | | CAGTCTGCAACCTGAAGATTTTG |
| | | CAACTTACTACTGTCAGCAAGGA |
| | | GTCAGTGACATCACTTTTGGCGG |
| | | AGGGACCAAGGTTGAGATCAAA |
| SEQ ID | Ab54 Heavy | QVQLVQSGAEVKKPGSSVKVSCK |
| NO: 29 | chain | ASGGTFSSYAISWVRQAPGQGLE |
| | variable | WMGGIIPIFGTANYAQKFQGRVTI |
| | region | TADESTSTAYMELSSLRSEDTAVY |
| | (e.g., | YCARGGLDTDEFDLWGRGTLVTV |
| | as found | SS |
| | in HC-54) | |
| | (CDRs in | |
| | bold) | |
| SEQ ID | Ab54 Light | DIQMTQSPSSLSASVGDRVTITC |
| NO: 30 | chain | RASQSINSYLNWYQQKPGKAPKL |
| | variable | LIYAASSLQSGVPSRFSGSGSG |
| | region | TDFTLTISSLQPEDFATYYC |
| | (e.g., | QQGVSDITFGGGTVEIK |
| | as found | |
| | in LC-54) | |
| | (CDRs in | |
| | bold) | |
| SEQ ID | Ab54 CDR-H1 | GTFSSYAIS |
| NO: 31 | | |
| SEQ ID | Ab54 CDR-H2 | GIIPIFGTANYAQKFQG |
| NO: 32 | | |
| SEQ ID | Ab54 CDR-H3 | ARGGLDTDEFDL |
| NO: 33 | | |
| SEQ ID | Ab54 CDR-L1 | RASQSINSYLN |
| NO: 34 | | |
| SEQ ID | Ab54 CDR-L2 | AASSLQS |
| NO: 35 | | |
| SEQ ID | Ab54 CDR-L3 | QQGVSDIT |
| NO: 36 | | |
| SEQ ID | Ab54 Heavy | CAGGTGCAGCTGGTGCAGTCTG |
| NO: 37 | chain | GGGCTGAGGTGAAGAAGCCTGG |
| | variable | GTCCTCGGTGAAGGTCTCCTGC |
| | region | AAGGCTTCTGGAGGCACCTTCA |
| | (nucl) | GCAGCTATGCTATCAGCTGGGT |
| | | GCGACAGGCCCCTGGACAAGGG |
| | | CTTGAGTGGATGGGAGGGATCA |
| | | TCCCTATCTTTGGTACAGCAAAC |
| | | TACGCACAGAAGTTCCAGGGCA |
| | | GAGTCACGATTACCGCGGACGA |
| | | ATCCACGAGCACAGCCTACATG |
| | | GAGCTGAGCAGCCTGAGATCTG |
| | | AGGACACGGCGGTGTACTACTG |
| | | CGCCAGAGGTGGATTGGACACA |
| | | GACGAGTTCGACCTATGGGGGA |
| | | GAGGTACCTTGGTCACCGTCTCC |
| | | TCA |
| SEQ ID | Ab54 Light | GACATCCAGATGACCCAGTCTCC |
| NO: 38 | chain | ATCCTCCCTGTCTGCATCTGTAG |
| | variable | GAGACAGAGTCACCATCACTTGC |
| | region | CGGGCAAGTCAGAGCATTAACA |
| | (nucl) | GCTATTTAAATTGGTATCAGCAG |
| | | AAACCAGGGAAAGCCCCTAAGC |
| | | TCCTGATCTATGCTGCATCCAGT |
| | | TTGCAAAGTGGGGTCCCATCAAG |
| | | GTTCAGTGGCAGTGGATCTGGG |
| | | ACAGATTTCACTCTCACCATCAG |
| | | CAGTCTGCAACCTGAAGATTTTG |
| | | CAACTTACTACTGTCAGCAAGGA |
| | | GTCAGTGACATCACTTTTGGCGG |
| | | AGGGACCAAGGTTGAGATCAAA |
| SEQ ID | Ab56 Heavy | QVQLVQSGAEVKKPGSSVKVSCK |
| NO: 39 | chain | ASGGTFSLYAISWVRQAPGQGLE |
| | variable | WMGGIIPAFGTANYAQKFQGRVTI |
| | region | TADESTSTAYMELSSLRSEDTAVY |
| | (e.g., as | YCARGGLDTDEFDLWGRGTLVTV |
| | found in | SS |
| | HC-56) | |
| | (CDRs in | |
| | bold) | |
| SEQ ID | Ab56 Light | DIQMTQSPSSLSASVGDRVTITC |
| NO: 40 | chain | RASQSINSYLNWYQQKPGKAPKL |
| | variable | LIYAASSLQSGVPSRFSGSGSGT |
| | region | DFTLTISSLQPEDFATYYC |
| | (e.g., | QQGVSDITFGGGTKVEIK |
| | as found | |
| | in LC-56) | |
| | (CDRs in | |
| | bold) | |
| SEQ ID | Ab56 CDR-H1 | GTFSLYAIS |
| NO: 41 | | |
| SEQ ID | Ab56 CDR-H2 | GIIPAFGTANYAQKFQG |
| NO: 42 | | |
| SEQ ID | Ab56 CDR-H3 | ARGGLDTDEFDL |
| NO: 43 | | |
| SEQ ID | Ab56 CDR-L1 | RASQSINSYLN |
| NO: 44 | | |
| SEQ ID | Ab56 CDR-L2 | AASSLQS |
| NO: 45 | | |
| SEQ ID | Ab56 CDR-L3 | QQGVSDIT |
| NO: 46 | | |
| SEQ ID | Ab56 Heavy | CAGGTGCAGCTGGTGCAGTCTG |
| NO: 47 | chain | GGGCTGAGGTGAAGAAGCCTGG |
| | variable | GTCCTCGGTGAAGGTCTCCTGC |
| | region | AAGGCTTCTGGAGGCACCTTCA |
| | (nucl) | GCCTCTATGCTATCTCCTGGGTG |
| | | CGACAGGCCCCTGGACAAGGGC |
| | | TTGAGTGGATGGGAGGGATCAT |
| | | CCCTGCCTTCGGTACCGCAAACT |
| | | ACGCACAGAAGTTCCAGGGCAG |
| | | AGTCACGATTACCGCGGACGAAT |
| | | CCACGAGCACAGCCTACATGGA |
| | | GCTGAGCAGCCTGAGATCTGAG |
| | | GACACGGCGGTGTACTACTGCG |
| | | CCAGAGGTGGATTGGACACAGA |
| | | CGAGTTCGACCTATGGGGGAGA |
| | | GGTACCTTGGTCACCGTCTCCT |
| | | CA |
| SEQ ID | Ab56 Light | GACATCCAGATGACCCAGTCTCC |
| NO: 48 | chain | ATCCTCCCTGTCTGCATCTGTAG |
| | variable | GAGACAGAGTCACCATCACTTGC |
| | region | CGGGCAAGTCAGAGCATTAACA |
| | (nucl) | GCTATTTAAATTGGTATCAGCAG |
| | | AAACCAGGGAAAGCCCCTAAGC |
| | | TCCTGATCTATGCTGCATCCAGT |
| | | TTGCAAAGTGGGGTCCCATCAAG |
| | | GTTCAGTGGCAGTGGATCTGGG |
| | | ACAGATTTCACTCTCACCATCAG |
| | | CAGTCTGCAACCTGAAGATTTTG |
| | | CAACTTACTACTGTCAGCAAGGA |
| | | GTCAGTGACATCACTTTTGGCGG |
| | | AGGGACCAAGGTTGAGATCAAA |
| SEQ ID | Ab57 Heavy | QVQLVQSGAEVKKPGSSVKVSCK |
| NO: 49 | chain | ASGGTFSLYAISWVRQAPGQGLE |
| | variable | WMGGIIPHFGLANYAQKFQGRVTI |
| | region | TADESTSTAYMELSSLRSEDTAVY |
| | (e.g., | YCARGGLDTDEFDLWGRGTLVTV |
| | as found | SS |
| | in HC-57) | |
| | (CDRs in | |
| | bold) | |
| SEQ ID | Ab57 Light | DIQMTQSPSSLSASVGDRVTITCR |
| NO: 50 | chain | ASQSINSYLNWYQQKPGKAPKLLI |
| | variable | YAASSLQSGVPSRFSGSGSGTDF |
| | region | TLTISSLQPEDFATYYCQQGVSD |
| | (e.g., | ITFGGGTKVEIK |
| | as found | |
| | in LC-57) | |
| | (CDRs in | |
| | bold) | |
| SEQ ID | Ab57 CDR-H1 | GTFSLYAIS |
| NO: 51 | | |
| SEQ ID | Ab57 CDR-H2 | GIIPHFGLANYAQKFQG |
| NO: 52 | | |
| SEQ ID | Ab57 CDR-H3 | ARGGLDTDEFDL |
| NO: 53 | | |
| SEQ ID | Ab57 CDR-L1 | RASQSINSYLN |
| NO: 54 | | |
| SEQ ID | Ab57 CDR-L2 | AASSLQS |
| NO: 55 | | |
| SEQ ID | Ab57 CDR-L3 | QQGVSDIT |
| NO: 56 | | |
| SEQ ID | Ab57 Heavy | CAGGTGCAGCTGGTGCAGTCTG |
| NO: 57 | chain | GGGCTGAGGTGAAGAAGCCTGG |
| | variable | GTCCTCGGTGAAGGTCTCCTGC |
| | region | AAGGCTTCTGGAGGCACCTTCTC |
| | (nucl) | CCTCTATGCTATCAGCTGGGTGC |
| | | GACAGGCCCCTGGACAAGGGCT |
| | | TGAGTGGATGGGAGGGATCATC |
| | | CCTCACTTCGGTCTCGCAAACTA |
| | | CGCACAGAAGTTCCAGGGCAGA |
| | | GTCACGATTACCGCGGACGAAT |
| | | CCACGAGCACAGCCTACATGGA |
| | | GCTGAGCAGCCTGAGATCTGAG |
| | | GACACGGCGGTGTACTACTGCG |
| | | CCAGAGGTGGATTGGACACAGA |
| | | CGAGTTCGACCTATGGGGGAGA |
| | | GGTACCTTGGTCACCGTCTCCTC |
| | | A |
| SEQ ID | Ab57 Light | GACATCCAGATGACCCAGTCTCC |
| NO: 58 | chain | ATCCTCCCTGTCTGCATCTGTAG |
| | variable | GAGACAGAGTCACCATCACTTGC |
| | region | CGGGCAAGTCAGAGCATTAACA |
| | (nucl) | GCTATTTAAATTGGTATCAGCAG |
| | | AAACCAGGGAAAGCCCCTAAGC |
| | | TCCTGATCTATGCTGCATCCAGT |
| | | TTGCAAAGTGGGGTCCCATCAAG |
| | | GTTCAGTGGCAGTGGATCTGGG |
| | | ACAGATTTCACTCTCACCATCAG |
| | | CAGTCTGCAACCTGAAGATTTTG |
| | | CAACTTACTACTGTCAGCAAGGA |
| | | GTCAGTGACATCACTTTTGGCGG |
| | | AGGGACCAAGGTTGAGATCAAA |
| SEQ ID | Ab58 Heavy | EVQLLESGGGLVQPGGSLRLSCA |
| NO: 59 | chain | ASGFTFSNYAMSWVRQAPGKGLE |
| | variable | WVSAISGSGGSTYYADSVKGRFTI |
| | region | SRDNSKNTLYLQMNSLRAEDTAV |
| | (e.g., | YYCAKGPPTYHTNYYYMDVWGK |
| | as found | GTTVTVSS |
| | in HC-58) | |
| | hIgG1 | |
| | backbone | |
| | (CDRs in | |
| | bold) | |
| SEQ ID | Ab58 Light | DIQMTQSPSSVSASVGDRVTITC |
| NO: 60 | chain | RASQGISSWLAWYQQKPGKAPKL |
| | variable | LIYASSLQSGVPSRFSGSGSGTD |
| | region | FTLTISSLQPEDFATYYCQQTN |
| | (e.g., | SFPYTFGGGTKVEIK |
| | as found | |
| | in LC-58) | |
| | (CDRs in | |
| | bold) | |
| SEQ ID | Ab58 CDR-H1 | FTFSNYAMS |
| NO: 61 | | |
| SEQ ID | Ab58 CDR-H2 | AISGSGGSTYYADSVKG |
| NO: 62 | | |
| SEQ ID | Ab58 CDR-H3 | AKGPPTYHTNYYYMDV |
| NO: 63 | | |
| SEQ ID | Ab58 CDR-L1 | RASQGISSWLA |
| NO: 64 | | |
| SEQ ID | Ab58 CDR-L2 | AASSLQS |
| NO: 65 | | |
| SEQ ID | Ab58 CDR-L3 | QQTNSFPYT |
| NO: 66 | | |
| SEQ ID | Ab58 Heavy | GAGGTGCAGCTGTTGGAGTCTG |
| NO: 67 | chain | GGGGAGGCTTGGTACAGCCTGG |
| | variable | GGGGTCCCTGAGACTCTCCTGT |
| | region | GCAGCCTCTGGATTCACCTTTAG |
| | (nucl) | CAATTATGCCATGAGCTGGGTCC |
| | | GCCAGGCTCCAGGGAAGGGGCT |
| | | GGAGTGGGTCTCAGCTATTAGTG |
| | | GTAGTGGTGGTAGCACATACTAC |
| | | GCAGACTCCGTGAAGGGCCGGT |
| | | TCACCATCTCCAGAGACAATTCC |
| | | AAGAACACGCTGTATCTGCAAAT |
| | | GAACAGCCTGAGAGCCGAGGAC |
| | | ACGGCGGTGTACTACTGCGCCA |
| | | AGGGCCCTCCTACATACCACACA |
| | | AACTACTACTACATGGACGTATG |
| | | GGGCAAGGGTACAACTGTCACC |
| | | GTCTCCTCA |
| SEQ ID | Ab58 Light | GACATCCAGATGACCCAGTCTCC |
| NO: 68 | chain | ATCTTCCGTGTCTGCATCTGTAG |
| | variable | GAGACAGAGTCACCATCACTTGT |
| | region | CGGGCGAGTCAGGGTATTAGCA |
| | (nucl) | GCTGGTTAGCCTGGTATCAGCA |
| | | GAAACCAGGGAAAGCCCCTAAG |
| | | CTCCTGATCTATGCTGCATCCAG |
| | | TTTGCAAAGTGGGGTCCCATCAA |
| | | GGTTCAGCGGCAGTGGATCTGG |
| | | GACAGATTTCACTCTCACCATCA |
| | | GCAGCCTGCAGCCTGAAGATTTT |
| | | GCAACTTATTACTGTCAGCAAAC |
| | | AAATAGTTTCCCTTACACTTTTGG |
| | | CGGAGGGACCAAGGTTGAGATC |
| | | AAA |
| SEQ ID | Ab61 Heavy | EVQLLESGGGLVQPGGSLRLSCA |
| NO: 69 | chain | ASGFTFSSYVMIWVRQAPGKGLE |
| | variable | WVSSISGDSVTTYYADSVKGRFTI |
| | region | SRDNSKNTLYLQMNSLRAEDTAV |
| | (e.g., | YYCAKGPPTYHTNYYYMDVWGK |
| | as found | GTTVTVSS |
| | in HC-61) | |
| | (CDRs in | |
| | bold) | |
| SEQ ID | Ab61 Light | DIQMTQSPSSVSASVGDRVTITC |
| NO: 70 | chain | RASQGISSWLAWYQQKPGKAPKL |
| | variable | LIYASSLQSGVPSRFSGSGSGTD |
| | region | FTLTISSLQPEDFATYYCQQT |
| | (e.g., | NSFPYTFGGGTKVEIK |
| | as found | |
| | in LC-61) | |
| | (CDRs in | |
| | bold) | |
| SEQ ID | Ab61 CDR-H1 | FTFSSYVMI |
| NO: 71 | | |
| SEQ ID | Ab61 CDR-H2 | SISGDSVTTYYADSVKG |
| NO: 72 | | |
| SEQ ID | Ab61 CDR-H3 | AKGPPTYHTNYYYMDV |
| NO: 73 | | |
| SEQ ID | Ab61 CDR-L1 | RASQGISSWLA |
| NO: 74 | | |
| SEQ ID | Ab61 CDR-L2 | AASSLQS |
| NO: 75 | | |
| SEQ ID | Ab61 CDR-L3 | QQTNSFPYT |
| NO: 76 | | |
| SEQ ID | Ab61 Heavy | GAGGTGCAGCTGTTGGAGTCTG |
| NO: 77 | chain | GGGGAGGCTTGGTACAGCCTGG |
| | variable | GGGGTCCCTGAGACTCTCCTGT |
| | region | GCAGCCTCTGGATTCACCTTTAG |
| | (nucl) | CAGCTATGTCATGATCTGGGTCC |
| | | GCCAGGCTCCAGGGAAGGGGCT |
| | | GGAGTGGGTCTCAAGCATTAGT |
| | | GGTGACAGCGTAACAACATACTA |
| | | CGCAGACTCCGTGAAGGGCCGG |
| | | TTCACCATCTCCAGAGACAATTC |
| | | CAAGAACACGCTGTATCTGCAAA |
| | | TGAACAGCCTGAGAGCCGAGGA |
| | | CACGGCGGTGTACTACTGCGCC |
| | | AAGGGCCCTCCTACATACCACAC |
| | | AAACTACTACTACATGGACGTAT |
| | | GGGGCAAGGGTACAACTGTCAC |
| | | CGTCTCCTCA |
| SEQ ID | Ab61 Light | GACATCCAGATGACCCAGTCTCC |
| NO: 78 | chain | ATCTTCCGTGTCTGCATCTGTAG |
| | variable | GAGACAGAGTCACCATCACTTGT |
| | region | CGGGCGAGTCAGGGTATTAGCA |
| | (nucl) | GCTGGTTAGCCTGGTATCAGCA |
| | | GAAACCAGGGAAAGCCCCTAAG |
| | | CTCCTGATCTATGCTGCATCCAG |
| | | TTTGCAAAGTGGGGTCCCATCAA |
| | | GGTTCAGCGGCAGTGGATCTGG |
| | | GACAGATTTCACTCTCACCATCA |
| | | GCAGCCTGCAGCCTGAAGATTTT |
| | | GCAACTTATTACTGTCAGCAAAC |
| | | AAATAGTTTCCCTTACACTTTTG |
| | | GCGGAGGGACCAAGGTTGAGATC |
| | | AAA |
| SEQ ID | Ab66 Heavy | EVQLVESGGGLVQPGGSLRLSCA |
| NO: 79 | chain | ASGFTFSDHYMDWVRQAPGKGL |
| | variable | EWVGRTRNKASSYTTEYAASVKG |
| | region | RFTISRDDSKNSLYLQMNSLKTED |
| | (e.g., | TAVYYCAREPKYWIDFDLWGRGT |
| | as found | LVTVSS |
| | in HC-66) | |
| | (CDRs in | |
| | bold) | |
| SEQ ID | Ab66 Light | DIQMTQSPSSLSASVGDRVTITCR |
| NO: 80 | chain | ASQSISSYLNWYQQKPGKAPKLLI |
| | variable | YAASSLQSGVPSRFSGSGSGTDF |
| | region | TLTISSLQPEDFATYYC |
| | (e.g., | QQSYIAPYTFGGGTKVEIK |
| | as found | |
| | in LC-66) | |
| | (CDRs in | |
| | bold) | |
| SEQ ID | Ab66 CDR-H1 | FTFSDHYMD |
| NO: 81 | | |
| SEQ ID | Ab66 CDR-H2 | RTRNKASSYTTEYAASVKG |
| NO: 82 | | |
| SEQ ID | Ab66 CDR-H3 | AREPKYWIDFDL |
| NO: 83 | | |
| SEQ ID | Ab66 CDR-L1 | RASQSISSYLN |
| NO: 84 | | |
| SEQ ID | Ab66 CDR-L2 | AASSLQS |
| NO: 85 | | |
| SEQ ID | Ab66 CDR-L3 | QQSYIAPYT |
| NO: 86 | | |
| SEQ ID | Ab66 Heavy | GAGGTGCAGCTGGTGGAGTCTG |
| NO: 87 | chain | GGGGAGGCTTGGTCCAGCCTGG |
| | variable | AGGGTCCCTGAGACTCTCCTGT |
| | region | GCAGCCTCTGGATTCACCTTCAG |
| | (nucl) | TGACCACTACATGGACTGGGTCC |
| | | GCCAGGCTCCAGGGAAGGGGCT |
| | | GGAGTGGGTTGGCCGTACTAGA |
| | | AACAAAGCTAGTAGTTACACCAC |
| | | AGAATACGCCGCGTCTGTGAAA |
| | | GGCAGATTCACCATCTCAAGAGA |
| | | TGATTCAAAGAACTCACTGTATC |
| | | TGCAAATGAACAGCCTGAAAACC |
| | | GAGGACACGGCGGTGTACTACT |
| | | GCGCCAGAGAGCCTAAATACTG |
| | | GATCGACTTCGACCTATGGGGG |
| | | AGAGGTACCTTGGTCACCGTCTC |
| | | CTCA |
| SEQ ID | Ab66 Light | GACATCCAGATGACCCAGTCTCC |
| NO: 88 | chain | ATCCTCCCTGTCTGCATCTGTAG |
| | variable | GAGACAGAGTCACCATCACTTGC |
| | region | CGGGCAAGTCAGAGCATTAGCA |
| | (nucl) | GCTATTTAAATTGGTATCAGCAG |
| | | AAACCAGGGAAAGCCCCTAAGC |
| | | TCCTGATCTATGCTGCATCCAGT |
| | | TTGCAAAGTGGGGTCCCATCAAG |
| | | GTTCAGTGGCAGTGGATCTGGG |
| | | ACAGATTTCACTCTCACCATCAG |
| | | CAGTCTGCAACCTGAAGATTTTG |
| | | CAACTTACTACTGTCAGCAAAGC |
| | | TACATCGCCCCTTACACTTTTGG |
| | | CGGAGGGACCAAGGTTGAGATC |
| | | AAA |
| SEQ ID | Ab68 Heavy | EVQLVESGGGLVQPGRSLRLSCT |
| NO: 89 | chain | ASGFTFSDHDMNWVRQAPGKGL |
| | variable | EWVGRTRNAAGSYTTEYAASVK |
| | region | GRFTISRDDSKNSLYLQMNSLKTE |
| | (e.g., | DTAVYYCAREPKYWIDFDLWGRG |
| | as found | TLVTVSS |
| | in HC-68) | |
| | (CDRs in | |
| | bold) | |
| SEQ ID | Ab68 Light | DIQMTQSPSSLSASVGDRVTITCR |
| NO: 90 | chain | ASQSISSYLNWYQQKPGKAPKLLI |
| | variable | YAASSLQSGVPSRFSGSGSGTDF |
| | region | TLTISSLQPEDFATYYC |
| | (e.g., | QQSYIAPYTFGGGTKVEIK |
| | as found | |
| | in LC-68) | |
| | (CDRs in | |
| | bold) | |
| SEQ ID | Ab68 CDR-H1 | FTFSDHDMN |
| NO: 91 | | |
| SEQ ID | Ab68 CDR-H2 | RTRNAAGSYTTEYAASVKG |
| NO: 92 | | |
| SEQ ID | Ab68 CDR-H3 | AREPKYWIDFDL |
| NO: 93 | | |
| SEQ ID | Ab68 CDR-L1 | RASQSISSYLN |
| NO: 94 | | |
| SEQ ID | Ab68 CDR-L2 | AASSLQS |
| NO: 95 | | |
| SEQ ID | Ab68 CDR-L3 | QQSYIAPYT |
| NO: 96 | | |
| SEQ ID | Ab68 Heavy | GAGGTGCAGCTGGTGGAGTCTG |
| NO: 97 | chain | GGGGAGGCTTGGTACAGCCAGG |
| | variable | GCGGTCCCTGAGACTCTCCTGTA |
| | region | CAGCTTCTGGATTCACCTTCAGT |
| | (nucl) | GACCACGACATGAACTGGGTCC |
| | | GCCAGGCTCCAGGGAAGGGGCT |
| | | GGAGTGGGTTGGCCGTACTAGA |
| | | AACGCCGCTGGAAGTTACACCA |
| | | CAGAATACGCCGCGTCTGTGAAA |
| | | GGCAGATTCACCATCTCAAGAGA |
| | | TGATTCAAAGAACTCACTGTATC |
| | | TGCAAATGAACAGCCTGAAAACC |
| | | GAGGACACGGCGGTGTACTACT |
| | | GCGCCAGAGAGCCTAAATACTG |
| | | GATCGACTTCGACCTATGGGGG |
| | | AGAGGTACCTTGGTCACCGTCTC |
| | | CTCA |
| SEQ ID | Ab68 Light | GACATCCAGATGACCCAGTCTCC |
| NO: 98 | chain | ATCCTCCCTGTCTGCATCTGTAG |
| | variable | GAGACAGAGTCACCATCACTTGC |
| | region | CGGGCAAGTCAGAGCATTAGCA |
| | (nucl) | GCTATTTAAATTGGTATCAGCAG |
| | | AAACCAGGGAAAGCCCCTAAGC |
| | | TCCTGATCTATGCTGCATCCAGT |
| | | TTGCAAAGTGGGGTCCCATCAAG |
| | | GTTCAGTGGCAGTGGATCTGGG |
| | | ACAGATTTCACTCTCACCATCAG |
| | | CAGTCTGCAACCTGAAGATTTTG |
| | | CAACTTACTACTGTCAGCAAAGC |
| | | TACATCGCCCCTTACACTTTTGG |
| | | CGGAGGGACCAAGGTTGAGATC |
| | | AAA |
| SEQ ID | Ab69 Heavy | EVQLVESGGGLVQPGGSLRLSCA |
| NO: 99 | chain | ASGFTFVDHDMDWVRQAPGKGL |
| | variable | EWVGRTRNKLGSYTTEYAASVKG |
| | region | RFTISRDDSKNSLYLQMNSLKTED |
| | (e.g., | TAVYYCAREPKYWIDFDLWGRGT |
| | as found | LVTVSS |
| | in HC-69) | |
| | (CDRs in | |
| | bold) | |
| SEQ ID | Ab69 Light | DIQMTQSPSSLSASVGDRVTITCR |
| NO: 100 | chain | ASQSISSYLNWYQQKPGKAPKLLI |
| | variable | YAASSLQSGVPSRFSGSGSGTDF |
| | region | TLTISSLQPEDFATYYCQQSYIAPY |
| | (e.g., | TFGGGTKVEIK |
| | as found | |
| | in LC-69) | |
| | (CDRs in | |
| | bold) | |
| SEQ ID | Ab69 CDR-H1 | FTFVDHDMD |
| NO: 101 | | |
| SEQ ID | Ab69 CDR-H2 | RTRNKLGSYTTEYAASVKG |
| NO: 102 | | |
| SEQ ID | Ab69 CDR-H3 | AREPKYWIDFDL |
| NO: 103 | | |
| SEQ ID | Ab69 CDR-L1 | RASQSISSYLN |
| NO: 104 | | |
| SEQ ID | Ab69 CDR-L2 | AASSLQS |
| NO: 105 | | |
| SEQ ID | Ab69 CDR-L3 | QQSYIAPYT |
| NO: 106 | | |
| SEQ ID | Ab69 Heavy | GAGGTGCAGCTGGTGGAGTCTG |
| NO: 107 | chain | GGGGAGGCTTGGTCCAGCCTGG |
| | variable | AGGGTCCCTGAGACTCTCCTGT |
| | region | GCAGCCTCTGGATTCACCTTCGT |
| | (nucl) | AGACCACGACATGGACTGGGTC |
| | | CGCCAGGCTCCAGGGAAGGGGC |
| | | TGGAGTGGGTTGGCCGTACTAG |
| | | AAACAAACTAGGAAGTTACACCA |
| | | CAGAATACGCCGCGTCTGTGAAA |
| | | GGCAGATTCACCATCTCAAGAGA |
| | | TGATTCAAAGAACTCACTGTATC |
| | | TGCAAATGAACAGCCTGAAAACC |
| | | GAGGACACGGCGGTGTACTACT |
| | | GCGCCAGAGAGCCTAAATACTG |
| | | GATCGACTTCGACCTATGGGGG |
| | | AGAGGTACCTTGGTCACCGTCTC |
| | | CTCA |
| SEQ ID | Ab69 Light | GACATCCAGATGACCCAGTCTCC |
| NO: 108 | chain | ATCCTCCCTGTCTGCATCTGTAG |
| | variable | GAGACAGAGTCACCATCACTTGC |
| | region | CGGGCAAGTCAGAGCATTAGCA |
| | (nucl) | GCTATTTAAATTGGTATCAGCAG |
| | | AAACCAGGGAAAGCCCCTAAGC |
| | | TCCTGATCTATGCTGCATCCAGT |
| | | TTGCAAAGTGGGGTCCCATCAAG |
| | | GTTCAGTGGCAGTGGATCTGGG |
| | | ACAGATTTCACTCTCACCATCAG |
| | | CAGTCTGCAACCTGAAGATTTTG |
| | | CAACTTACTACTGTCAGCAAAGC |
| | | TACATCGCCCCTTACACTTTTGG |
| | | CGGAGGGACCAAGGTTGAGATC |
| | | AAA |
| SEQ ID | Ab67 Light | DIQMTQSPSSLSASVGDRVTIT |
| NO: 109 | chain | CRASQSISSYLNWYQQKPGKA |
| | LC constant | PKLLIYAASSLQSGVPSRFSGS |
| | region | GSGTDFTLTISSLQPEDFATYY |
| | underlined | CQQSYIAPYTFGGGTKVEIKRT |
| | | VAAPSVFIFPPSDEQLKSGTAS |
| | | VVCLLNNFYPREAKVQWKVDN |
| | | ALQSGNSQESVTEQDSKDSTY |
| | | SLSSTLTLSKADYEKHKVYACE |
| | | VTHQGLSSPVTKSFNRGEC |
| SEQ ID | Ab67 Heavy | EVQLVESGGGLVQPGGSLRLS |
| NO: 110 | chain | CAASGFTFSDADMDWVRQAP |
| | HC constant | GKGLEWVGRTRNKAGSYTTEY |
| | region | AASVKGRFTISRDDSKNSLYLQ |
| | underlined | MNSLKTEDTAVYYCAREPKYW |
| | | IDFDLWGRGTLVTVSSASTKGP |
| | | SVFPLAPSSKSTSGGTAALGCL |
| | | VKDYFPEPVTVSWNSGALTSG |
| | | VHTFPAVLQSSGLYSLSSVVTV |
| | | PSSSLGTQTYICNVNHKPSNTK |
| | | VDKKVEPKSCDKTHTCPPCPA |
| | | PELLGGPSVFLFPPKPKDTLMI |
| | | SRTPEVTCVVVDVSHEDPEVK |
| | | FNWYVDGVEVHNAKTKPREEQ |
| | | YNSTYRVVSVLTVLHQDWLNG |
| | | KEYKCKVSNKALPAPIEKTISKA |
| | | KGQPREPQVYTLPPSRDELTK |
| | | NQVSLTCLVKGFYPSDIAVEWE |
| | | SNGQPENNYKTTPPVLDSDGS |
| | | FFLYSKLTVDKSRWQQGNVFS |
| | | CSVMHEALHNHYTQKSLSLSP |
| | | GK |
| SEQ ID | Ab67 Heavy | EVQLVESGGGLVQPGGSLRLS |
| NO: 111 | chain | CAASGFTFSDADMDWVRQAP |
| | (D265C)* | GKGLEWVGRTRNKAGSYTTEY |
| | HC constant | AASVKGRFTISRDDSKNSLYLQ |
| | region | MNSLKTEDTAVYYCAREPKYW |
| | underlined | IDFDLWGRGTLVTVSSASTKGP |
| | | SVFPLAPSSKSTSGGTAALGCL |
| | | VKDYFPEPVTVSWNSGALTSG |
| | | VHTFPAVLQSSGLYSLSSVVTV |
| | | PSSSLGTQTYICNVNHKPSNTK |
| | | VDKKVEPKSCDKTHTCPPCPA |
| | | PELLGGPSVFLFPPKPKDTLMI |
| | | SRTPEVTCVVVCVSHEDPEVK |
| | | FNWYVDGVEVHNAKTKPREEQ |
| | | YNSTYRVVSVLTVLHQDWLNG |
| | | KEYKCKVSNKALPAPIEKTISKA |
| | | KGQPREPQVYTLPPSRDELTK |
| | | NQVSLTCLVKGFYPSDIAVEWE |
| | | SNGQPENNYKTTPPVLDSDGS |
| | | FFLYSKLTVDKSRWQQGNVFS |
| | | CSVMHEALHNHYTQKSLSLSP |
| | | GK |
| SEQ ID | Ab67 Heavy | EVQLVESGGGLVQPGGSLRLS |
| NO: 112 | chain | CAASGFTFSDADMDWVRQAP |
| | (L234A/ | GKGLEWVGRTRNKAGSYTTEY |
| | L235A/ | AASVKGRFTISRDDSKNSLYLQ |
| | D265C)* | MNSLKTEDTAVYYCAREPKYW |
| | HC constant | IDFDLWGRGTLVTVSSASTKGP |
| | region | SVFPLAPSSKSTSGGTAALGCL |
| | underlined | VKDYFPEPVTVSWNSGALTSG |
| | | VHTFPAVLQSSGLYSLSSVVTV |
| | | PSSSLGTQTYICNVNHKPSNTK |
| | | VDKKVEPKSCDKTHTCPPCPA |
| | | PEAAGGPSVFLFPPKPKDTLMI |
| | | SRTPEVTCVVVCVSHEDPEVK |
| | | FNWYVDGVEVHNAKTKPREEQ |
| | | YNSTYRVVSVLTVLHQDWLNG |
| | | KEYKCKVSNKALPAPIEKTISKA |
| | | KGQPREPQVYTLPPSRDELTK |
| | | NQVSLTCLVKGFYPSDIAVEWE |
| | | SNGQPENNYKTTPPVLDSDGS |
| | | FFLYSKLTVDKSRWQQGNVFS |
| | | CSVMHEALHNHYTQKSLSLSP |
| | | GK |
| SEQ ID | Ab67 Heavy | EVQLVESGGGLVQPGGSLRLS |
| NO: 113 | chain | CAASGFTFSDADMDWVRQAP |
| | (D265C/ | GKGLEWVGRTRNKAGSYTTEY |
| | H435A)* | AASVKGRFTISRDDSKNSLYLQ |
| | HC constant | MNSLKTEDTAVYYCAREPKYW |
| | region | IDFDLWGRGTLVTVSSASTKGP |
| | underlined | SVFPLAPSSKSTSGGTAALGCL |
| | | VKDYFPEPVTVSWNSGALTSG |
| | | VHTFPAVLQSSGLYSLSSVVTV |
| | | PSSSLGTQTYICNVNHKPSNTK |
| | | VDKKVEPKSCDKTHTCPPCPA |
| | | PELLGGPSVFLFPPKPKDTLMI |
| | | SRTPEVTCVVVCVSHEDPEVK |
| | | FNWYVDGVEVHNAKTKPREEQ |
| | | YNSTYRVVSVLTVLHQDWLNGK |
| | | EYKCKVSNKALPAPIEKTISKA |
| | | KGQPREPQVYTLPPSRDELTK |
| | | NQVSLTCLVKGFYPSDIAVEWE |
| | | SNGQPENNYKTTPPVLDSDGS |
| | | FFLYSKLTVDKSRWQQGNVFS |
| | | CSVMHEALHNAYTQKSLSLSP |
| | | GK |
| SEQ ID | Ab67 Heavy | EVQLVESGGGLVQPGGSLRLS |
| NO: 114 | chain | CAASGFTFSDADMDWVRQAP |
| | (L234A/ | GKGLEWVGRTRNKAGSYTTEY |
| | L235A/ | AASVKGRFTISRDDSKNSLYLQ |
| | D265C/ | MNSLKTEDTAVYYCAREPKYW |
| | H435A)* | IDFDLWGRGTLVTVSSASTKGP |
| | HC constant | SVFPLAPSSKSTSGGTAALGCL |
| | region | VKDYFPEPVTVSWNSGALTSG |
| | underlined | VHTFPAVLQSSGLYSLSSVVTV |
| | | PSSSLGTQTYICNVNHKPSNTK |
| | | VDKKVEPKSCDKTHTCPPCPA |
| | | PEAAGGPSVFLFPPKPKDTLMI |
| | | SRTPEVTCVVVCVSHEDPEVK |
| | | FNWYVDGVEVHNAKTKPREEQ |
| | | YNSTYRVVSVLTVLHQDWLNGK |
| | | EYKCKVSNKALPAPIEKTISKA |
| | | KGQPREPQVYTLPPSRDELTK |
| | | NQVSLTCLVKGFYPSDIAVEWE |
| | | SNGQPENNYKTTPPVLDSDGS |
| | | FFLYSKLTVDKSRWQQGNVFS |
| | | CSVMHEALHNAYTQKSLSLSP |
| | | GK |
| SEQ ID | Ab55 Light | DIQMTQSPSSLSASVGDRVTITCR |
| NO: 115 | chain | ASQSINSYLNWYQQKPGKAPKLLI |
| | LC constant | YAASSLQSGVPSRFSGSGSGTDFT |
| | region | LTISSLQPEDFATYYCQQGVSDIT |
| | underlined | FGGGTKVEIKRTVAAPSVFIFPP |
| | | SDEQLKSGTASVVCLLNNFYP |
| | | REAKVQWKVDNALQSGNSQE |
| | | SVTEQDSKDSTYSLSSTLTLSK |
| | | ADYEKHKVYACEVTHQGLSSP |
| | | VTKSFNRGEC |
| SEQ ID | Ab55 Heavy | QVQLVQSGAEVKKPGSSVKVSCK |
| NO: 116 | chain | ASGGTFRIYAISWVRQAPGQGLE |
| | HC constant | WMGGIIPDFGVANYAQKFQGRVTI |
| | region | TADESTSTAYMELSSLRSEDTAVY |
| | underlined | YCARGGLDTDEFDLWGRGTLVTV |
| | | SSASTKGPSVFPLAPSSKSTSG |
| | | GTAALGCLVKDYFPEPVTVSW |
| | | NSGALTSGVHTFPAVLQSSGL |
| | | YSLSSVVTVPSSSLGTQTYICN |
| | | VNHKPSNTKVDKKVEPKSCDK |
| | | THTCPPCPAPELLGGPSVFLFP |
| | | PKPKDTLMISRTPEVTCVVVDV |
| | | SHEDPEVKFNWYVDGVEVHNA |
| | | KTKPREEQYNSTYRVVSVLTVL |
| | | HQDWLNGKEYKCKVSNKALPA |
| | | PIEKTISKAKGQPREPQVYTLP |
| | | PSRDELTKNQVSLTCLVKGFYP |
| | | SDIAVEWESNGQPENNYKTTP |
| | | PVLDSDGSFFLYSKLTVDKSR |
| | | WQQGNVFSCSVMHEALHNHY |
| | | TQKSLSLSPGK |
| SEQ ID | Ab55 Heavy | QVQLVQSGAEVKKPGSSVKVSCK |
| NO: 117 | chain | ASGGTFRIYAISWVRQAPGQGLE |
| | (D265C)* | WMGGIIPDFGVANYAQKFQGRVTI |
| | HC constant | TADESTSTAYMELSSLRSEDTAVY |
| | region | YCARGGLDTDEFDLWGRGTLVTV |
| | underlined | SSASTKGPSVFPLAPSSKSTSG |
| | | GTAALGCLVKDYFPEPVTVSW |
| | | NSGALTSGVHTFPAVLQSSGL |
| | | YSLSSVVTVPSSSLGTQTYICN |
| | | VNHKPSNTKVDKKVEPKSCDK |
| | | THTCPPCPAPELLGGPSVFLFP |
| | | PKPKDTLMISRTPEVTCVVVVC |
| | | SHEDPEVKFNWYVDGVEVHNA |
| | | KTKPREEQYNSTYRVVSVLTVL |
| | | HQDWLNGKEYKCKVSNKALPA |
| | | PIEKTISKAKGQPREPQVYTLP |
| | | PSRDELTKNQVSLTCLVKGFYP |
| | | SDIAVEWESNGQPENNYKTTP |
| | | PVLDSDGSFFLYSKLTVDKSR |
| | | WQQGNVFSCSVMHEALHNHY |
| | | TQKSLSLSPGK |
| SEQ ID | Ab55 Heavy | QVQLVQSGAEVKKPGSSVKVSCK |
| NO: 118 | chain | ASGGTFRIYAISWVRQAPGQGLE |
| | (L234A/ | WMGGIIPDFGVANYAQKFQGRVTI |
| | L235A / | TADESTSTAYMELSSLRSEDTAVY |
| | D265C)* | YCARGGLDTDEFDLWGRGTLVTV |
| | HC constant | SSASTKGPSVFPLAPSSKSTSG |
| | region | GTAALGCLVKDYFPEPVTVSW |
| | underlined | NSGALTSGVHTFPAVLQSSGL |
| | | YSLSSVVTVPSSSLGTQTYICN |
| | | VNHKPSNTKVDKKVEPKSCDK |
| | | THTCPPCPAPEAAGGPSVFLF |
| | | PPKPKDTLMISRTPEVTCVVVC |
| | | VSHEDPEVKFNWYVDGVEVHN |
| | | AKTKPREEQYNSTYRVVSVLT |
| | | VLHQDWLNGKEYKCKVSNKAL |
| | | PAPIEKTISKAKGQPREPQVYT |
| | | LPPSRDELTKNQVSLTCLVKGF |
| | | YPSDIAVEWESNGQPENNYKT |
| | | TPPVLDSDGSFFLYSKLTVDKS |
| | | RWQQGNVFSCSVMHEALHNH |
| | | YTQKSLSLSPGK |
| SEQ ID | Ab55 Heavy | QVQLVQSGAEVKKPGSSVKVSCK |
| NO: 119 | chain | ASGGTFRIYAISWVRQAPGQGLE |
| | (D265C/ | WMGGIIPDFGVANYAQKFQGRVTI |
| | H435A)* | TADESTSTAYMELSSLRSEDTAVY |
| | HC constant | YCARGGLDTDEFDLWGRGTLVTV |
| | region | SSASTKGPSVFPLAPSSKSTSG |
| | underlined | GTAALGCLVKDYFPEPVTVSW |
| | | NSGALTSGVHTFPAVLQSSGL |
| | | YSLSSVVTVPSSSLGTQTYICN |
| | | VNHKPSNTKVDKKVEPKSCDK |
| | | THTCPPCPAPELLGGPSVFLFP |
| | | PKPKDTLMISRTPEVTCVVVCV |
| | | SHEDPEVKFNWYVDGVEVHNA |
| | | KTKPREEQYNSTYRVVSVLTVL |
| | | HQDWLNGKEYKCKVSNKALPA |
| | | PIEKTISKAKGQPREPQVYTLP |
| | | PSRDELTKNQVSLTCLVKGFYP |
| | | SDIAVEWESNGQPENNYKTTP |
| | | PVLDSDGSFFLYSKLTVDKSR |
| | | WQQGNVFSCSVMHEALHNAY |
| | | TQKSLSLSPGK |
| SEQ ID | Ab55 Heavy | QVQLVQSGAEVKKPGSSVKVSCK |
| NO: 120 | chain | ASGGTFRIYAISWVRQAPGQGLE |
| | (L234A/ | WMGGIIPDFGVANYAQKFQGRVTI |
| | L235A/ | TADESTSTAYMELSSLRSEDTAVY |
| | D265C/ | YCARGGLDTDEFDLWGRGTLVTV |
| | H435A)* | SSASTKGPSVFPLAPSSKSTSG |
| | HC constant | GTAALGCLVKDYFPEPVTVSW |
| | region | NSGALTSGVHTFPAVLQSSGL |
| | underlined | YSLSSVVTVPSSSLGTQTYICN |
| | | VNHKPSNTKVDKKVEPKSCDK |
| | | THTCPPCPAPEAAGGPSVFLF |
| | | PPKPKDTLMISRTPEVTCVVVC |
| | | VSHEDPEVKFNWYVDGVEVHN |
| | | AKTKPREEQYNSTYRVVSVLT |
| | | VLHQDWLNGKEYKCKVSNKAL |
| | | PAPIEKTISKAKGQPREPQVYT |
| | | LPPSRDELTKNQVSLTCLVKGF |
| | | YPSDIAVEWESNGQPENNYKT |
| | | TPPVLDSDGSFFLYSKLTVDKS |
| | | RWQQGNVFSCSVMHEALHNA |
| | | YTQKSLSLSPGK |
| SEQ ID | Light chain | RTVAAPSVFIFPPSDEQLKSGT |
| NO: 121 | constant | ASVVCLLNNFYPREAKVQWKV |
| | region of | DNALQSGNSQESVTEQDSKDS |
| | LC-54, | TYSLSSTLTLSKADYEKHKVYA |
| | LC-55, | CEVTHQGLSSPVTKSFNRGEC |
| | LC-56, | |
| | LC-57, | |
| | LC-58, | |
| | LC-61, | |
| | LC-66, | |
| | LC-67, | |
| | LC-68, | |
| | LC-69 | |
| SEQ ID | Heavy chain | ASTKGPSVFPLAPSSKSTSGG |
| NO: 122 | constant | TAALGCLVKDYFPEPVTVSWN |
| | region of | SGALTSGVHTFPAVLQSSGLY |
| | WT | SLSSVVTVPSSSLGTQTYICNV |
| | | NHKPSNTKVDKKVEPKSCDKT |
| | | HTCPPCPAPELLGGPSVFLFP |
| | | PKPKDTLMISRTPEVTCVVVDV |
| | | SHEDPEVKFNWYVDGVEVHNA |
| | | KTKPREEQYNSTYRVVSVLTVL |
| | | HQDWLNGKEYKCKVSNKALPA |
| | | PIEKTISKAKGQPREPQVYTLP |
| | | PSRDELTKNQVSLTCLVKGFYP |
| | | SDIAVEWESNGQPENNYKTTP |
| | | PVLDSDGSFFLYSKLTVDKSR |
| | | WQQGNVFSCSVMHEALHNHY |
| | | TQKSLSLSPGK |
| SEQ ID | Heavy chain | ASTKGPSVFPLAPSSKSTSGG |
| NO: 123 | constant | TAALGCLVKDYFPEPVTVSWN |
| | region | SGALTSGVHTFPAVLQSSGLY |
| | (D265C)* | SLSSVVTVPSSSLGTQTYICNV |
| | | NHKPSNTKVDKKVEPKSCDKT |
| | | HTCPPCPAPELLGGPSVFLFPP |
| | | KPKDTLMISRTPEVTCVVVCVS |
| | | HEDPEVKFNWYVDGVEVHNAK |
| | | TKPREEQYNSTYRVVSVLTVLH |
| | | QDWLNGKEYKCKVSNKALPAP |
| | | IEKTISKAKGQPREPQVYTLPP |
| | | SRDELTKNQVSLTCLVKGFYPS |
| | | DIAVEWESNGQPENNYKTTPP |
| | | VLDSDGSFFLYSKLTVDKSRW |
| | | QQGNVFSCSVMHEALHNHYT |
| | | QKSLSLSPGK |
| SEQ ID | Heavy chain | ASTKGPSVFPLAPSSKSTSGG |
| NO: 124 | constant | TAALGCLVKDYFPEPVTVSWN |
| | region | SGALTSGVHTFPAVLQSSGLY |
| | (L234A/ | SLSSVVTVPSSSLGTQTYICNV |
| | L235A/ | NHKPSNTKVDKKVEPKSCDKT |
| | D265C)* | HTCPPCPAPEAAGGPSVFLFP |
| | | PKPKDTLMISRTPEVTCVVVCV |
| | | SHEDPEVKFNWYVDGVEVHNA |
| | | KTKPREEQYNSTYRVVSVLTVL |
| | | HQDWLNGKEYKCKVSNKALPA |
| | | PIEKTISKAKGQPREPQVYTLP |
| | | PSRDELTKNQVSLTCLVKGFYP |
| | | SDIAVEWESNGQPENNYKTTP |
| | | PVLDSDGSFFLYSKLTVDKSR |
| | | WQQGNVFSCSVMHEALHNHY |
| | | TQKSLSLSPGK |
| SEQ ID | Heavy chain | ASTKGPSVFPLAPSSKSTSGG |
| NO: 125 | constant | TAALGCLVKDYFPEPVTVSWN |
| | region | SGALTSGVHTFPAVLQSSGLY |
| | (H435A/ | SLSSVVTVPSSSLGTQTYICNV |
| | D265C)* | NHKPSNTKVDKKVEPKSCDKT |
| | | HTCPPCPAPELLGGPSVFLFPP |
| | | KPKDTLMISRTPEVTCVVVCVS |
| | | HEDPEVKFNWYVDGVEVHNAK |
| | | TKPREEQYNSTYRVVSVLTVLH |
| | | QDWLNGKEYKCKVSNKALPAP |
| | | IEKTISKAKGQPREPQVYTLPP |
| | | SRDELTKNQVSLTCLVKGFYPS |
| | | DIAVEWESNGQPENNYKTTPP |
| | | VLDSDGSFFLYSKLTVDKSRW |
| | | QQGNVFSCSVMHEALHNAYT |
| | | QKSLSLSPGK |
| SEQ ID | Heavy chain | ASTKGPSVFPLAPSSKSTSGG |
| NO: 126 | constant | TAALGCLVKDYFPEPVTVSWN |
| | region | SGALTSGVHTFPAVLQSSGLY |
| | (L234A/ | SLSSVVTVPSSSLGTQTYICNV |
| | L235A/ | NHKPSNTKVDKKVEPKSCDKT |
| | H435A/ | HTCPPCPAPEAAGGPSVFLFP |
| | D265C)* | PKPKDTLMISRTPEVTCVVVCV |
| | | SHEDPEVKFNWYVDGVEVHNA |
| | | KTKPREEQYNSTYRVVSVLTVL |
| | | HQDWLNGKEYKCKVSNKALPA |
| | | PIEKTISKAKGQPREPQVYTLP |
| | | PSRDELTKNQVSLTCLVKGFYP |
| | | SDIAVEWESNGQPENNYKTTP |
| | | PVLDSDGSFFLYSKLTVDKSR |
| | | WQQGNVFSCSVMHEALHNAY |
| | | TQKSLSLSPGK |
| SEQ ID | Consensus | GTF{S/R)(S/I/L) |
| NO: 127 | sequence | YAIS |
| | of | |
| | variable | |
| | heavy chain | |
| | CDR1 | |
| | (Abs 54-57) | |
| SEQ ID | Consensus | GIIP(I/D/A/H)FG |
| NO: 128 | sequence | (T/V/L)ANYAQKF |
| | of variable | QG |
| | heavy chain | |
| | CDR2 | |
| | (Abs 54-57) | |
| SEQ ID | Variable | ARGGLDTDEFDL |
| NO: 129 | heavy chain | |
| | CDR3 | |
| | (Abs 54-57) | |
| SEQ ID | Variable | RASQSINSYLN |
| NO: 130 | light chain | |
| | CDR1 | |
| | (Abs 54-57) | |
| SEQ ID | Variable | AASSLQS |
| NO: 131 | light chain | |
| | CDR2 | |
| | (Abs 54-57) | |
| SEQ ID | Variable | QQGVSDIT |
| NO: 132 | light chain | |
| | CDR3 | |
| | (Abs 54-57) | |
| SEQ ID | Consensus | FTFS(N/S)Y(A/V) |
| NO: 133 | sequence | M(S/I) |
| | of | |
| | variable | |
| | heavy chain | |
| | CDR1 | |
| | (Abs 58, 61) | |
| SEQ ID | Consensus | (A/S)ISG(S/D)(G/S) |
| NO: 134 | sequence of | (G/V)(S/T)TYYA |
| | variable | DSVKG |
| | heavy chain | |
| | CDR2 | |
| | (Abs 58, 61) | |
| SEQ ID | Variable | AKGPPTYHTNYYYMDV |
| NO: 135 | heavy chain | |
| | CDR3 | |
| | (Abs 58,61) | |
| SEQ ID | Variable | RASQGISSWLA |
| NO: 136 | light chain | |
| | CDR1 | |
| | (Abs 58, 61) | |
| SEQ ID | Variable | AASSLQS |
| NO: 137 | light chain | |
| | CDR2 | |
| | (Abs 58,61) | |
| SEQ ID | Variable | QQTNSFPYT |
| NO: 138 | light chain | |
| | CDR3 | |
| | (Abs 58,61) | |
| SEQ ID | Consensus | FTF(S/V)D(H/A) |
| NO: 139 | sequence of | (Y/D)M(D/N) |
| | variable | |
| | heavy chain | |
| | CDR1 | |
| | (Abs 66-69) | |
| SEQ ID | Consensus | RTRN(K/A)(A/L)(S/G) |
| NO: 140 | sequence of | SYTTEYAAS |
| | variable | VKG |
| | heavy | |
| | chain CDR2 | |
| | (Abs 66-69) | |
| SEQ ID | Variable | AREPKYWIDFDL |
| NO: 141 | heavy chain | |
| | CDR3 | |
| | (Abs 66-69) | |
| SEQ ID | Variable | RASQSISSYLN |
| NO: 142 | light chain | |
| | CDR1 | |
| | (Abs 66-69) | |
| SEQ ID | Variable | AASSLQS |
| NO: 143 | light chain | |
| | CDR2 | |
| | (Abs 66-69) | |
| SEQ ID | Variable | QQSYIAPYT |
| NO: 144 | light chain | |
| | CDR3 | |
| | (Abs 66-69) | |
| SEQ ID | Human CD117 | MRGARGAWDFLCVLLLLLRVQ |
| NO: 145 | (mast/ | TGSSQPSVSPGEPSPPSIHPG |
| | stem cell | KSDLIVRVGDEIRLLCTDPGFV |
| | growth factor | KWTFEILDETNENKQNEWITEK |
| | receptor Kit | AEATNTGKYTCTNKHGLSNSIY |
| | isoform 1 | VFVRDPAKLFLVDRSLYGKED |
| | precursor) | NDTLVRCPLTDPEVTNYSLK |
| | Protein | GCQGKPLPKDLRFIPDPKAG |
| | NCBI | IMIKSVKRAYHRLCLHCSVD |
| | Reference | QEGKSVLSEKFILKVRPAFK |
| | Sequence: | AVPVVSVSKASYLLREGEEF |
| | NP_0002 | TVTCTIKDVSSSVYSTWKRE |
| | 13.1 | NSQTKLQEKYNSWHHGDFNY |
| | | ERQATLTISSARVNDSGVFM |
| | | CYANNTFGSANVTTTLEVVD |
| | | KGFINIFPMINTTVFVNDGE |
| | | NVDLIVEYEAFPKPEHQQWI |
| | | YMNRTFTDKWEDYPKSENES |
| | | NIRYVSELHLTRLKGTEGGT |
| | | YTFLVSNSDVNAAIAFNVYV |
| | | NTKPEILTYDRLVNGMLQCV |
| | | AAGFPEPTIDWYFCPGTEQR |
| | | CSASVLPVDVQTLNSSGPPF |
| | | GKLVVQSSIDSSAFKHNGTV |
| | | ECKAYNDVGKTSAYFNFAFK |
| | | GNNKEQIHPHTLFTPLLIGF |
| | | VIVAGMMCIIVMILTYKYLQ |
| | | KPMYEVQWKVVEEINGNNYV |
| | | YIDPTQLPYDHKWEFPRNRL |
| | | SFGKTLGAGAFGKVVEATAY |
| | | GLIKSDAAMTVAVKMLKPSA |
| | | HLTEREALMSELKVLSYLGN |
| | | HMNIVNLLGACTIGGPTLVI |
| | | TEYCCYGDLLNFLRRKRDSF |
| | | ICSKQEDHAEAALYKNLLHS |
| | | KESSCSDSTNEYMDMKPGVS |
| | | YVVPTKADKRRSVRIGSYIE |
| | | RDVTPAIMEDDELALDLEDL |
| | | LSFSYQVAKGMAFLASKNCI |
| | | HRDLAARNILLTHGRITKIC |
| | | DFGLARDIKNDSNYVVKGNA |
| | | RLPVKWMAPESIFNCVYTFE |
| | | SDVWSYGIFLWELFSLGSSP |
| | | YPGMPVDSKFYKMIKEGFRM |
| | | LSPEHAPAEMYDIMKTCWDA |
| | | DPLKRPTFKQIVQLIEKQIS |
| | | ESTNHIYSNLANCSPNRQKP |
| | | VVDHSVRINSVGSTASSSQP |
| | | LLVHDDV |
| SEQ ID | Human CD117 | MRGARGAWDFLCVLLLLLRV |
| NO: 146 | (mast/stem | QTGSSQPSVSPGEPSPPSIH |
| | cell | PGKSDLIVRVGDEIRLLCTD |
| | growth | PGFVKWTFEILDETNENKQN |
| | factor | EWITEKAEATNTGKYTCTNK |
| | receptor | HGLSNSIY |
| | Kit isoform | VFVRDPAKLFLVDRSLYGKE |
| | 2 precursor) | DNDTLVRCPLTDPEVTNYSL |
| | Protein NCBI | KGCQGKPLPKDLRFIPDPKA |
| | Reference | GIMIKSVKRAYHRLCLHCSV |
| | Sequence: | DQEGKSVLSEKFILKVRPAF |
| | NP_00108 | KAVPVVSVSKASYLLREGEE |
| | 7241.1 | FTVTCTIKDVSSSVYSTWKR |
| | | ENSQTKLQEKYNSWHHGDFN |
| | | YERQATLTISSARVNDSGVF |
| | | MCYANNTFGSANVTTTLEVV |
| | | DKGFINIFPMINTTVFVNDG |
| | | ENVDLIVEYEAFPKPEHQQW |
| | | IYMNRTFTDKWEDYPKSENE |
| | | SNIRYVSELHLTRLKGTEGG |
| | | TYTFLVSNSDVNAAIAFNVY |
| | | VNTKPEILTYDRLVNGMLQC |
| | | VAAGFPEPTIDWYFCPGTEQ |
| | | RCSASVLPVDVQTLNSSGPP |
| | | FGKLVVQSSIDSSAFKHNGT |
| | | VECKAYNDVGKTSAYFNFAF |
| | | KEQIHPHTLFTPLLIGFVIV |
| | | AGMMCIIVMILTYKYLQKPM |
| | | YEVQWKVVEEINGNNYVYID |
| | | PTQLPYDHKWEFPRNRLSFG |
| | | KTLGAGAFGKVVEATAYGLI |
| | | KSDAAMTVAVKMLKPSAHLT |
| | | EREALMSELKVLSYLGNHMN |
| | | IVNLLGACTIGGPTLVITEY |
| | | CCYGDLLNFLRRKRDSFICS |
| | | KQEDHAEAALYKNLLHSKES |
| | | SCSDSTNEYMDMKPGVSYVV |
| | | PTKADKRRSVRIGSYIERDV |
| | | TPAIMEDDELALDLEDLLSF |
| | | SYQVAKGMAFLASKNCIHRD |
| | | LAARNILLTHGRITKICDFG |
| | | LARDIKNDSNYVVKGNARLP |
| | | VKWMAPESIFNCVYTFESDV |
| | | WSYGIFLWELFSLGSSPYPG |
| | | MPVDSKFYKMIKEGFRMLSP |
| | | EHAPAEMYDIMKTCWDADPL |
| | | KRPTFKQIVQLIEKQISEST |
| | | NHIYSNLANCSPNRQKPVVD |
| | | HSVRINSVGSTASSSQPLLV |
| | | HDDV |
| SEQ ID | Heavy chain | QVQLVQSGAAVKKPGESLKI |
| NO: 147 | variable | SCKGSGYRFTTYWIGWVRQM |
| | region of | PGKGLEWMGIIYPGDSDTRY |
| | HC-1 | SPSFQGQVTISAGKSISTAY |
| | | LQWSSLKASDTAMYYCARHG |
| | | RGYNGYEGAFDIWGQGTMVT |
| | | VSS |
| SEQ ID | Light chain | AIQLTQSPSSLSASVGDRVT |
| NO: 148 | variable | ITCRASQGVSSALAWYQQKP |
| | region of | GKAPKLLIYDASSLESGVPS |
| | LC-1 | RFSGSGSGTDFTLTISSLQP |
| | | EDFATYYCQQFNSYPLTFGG |
| | | GTKVEIK |
| SEQ ID | Heavy chain | QVQLVQSGAAVKKPGESLKI |
| NO: 147 | variable | SCKGSGYRFTTYWIGWVRQM |
| | region of | PGKGLEWMGIIYPGDSDTRY |
| | HC-2 | SPSFQGQVTISAGKSISTAY |
| | | LQWSSLKASDTAMYYCARHG |
| | | RGYNGYEGAFDIWGQGTMVT |
| | | VSS |
| SEQ ID | Light chain | DIQLTQSPSSLSASVGDRVT |
| NO: 149 | variable | ITCRASQGIRTDLGWYQQKP |
| | region of | GKAPKLLIYDASSLESGVPS |
| | LC-2 | RFSGSGSGTDFTLTISSLQP |
| | | EDFATYYCQQFNSYPLTFGG |
| | | GTKVEIK |
| SEQ ID | Heavy chain | QVQLVQSGAAVKKPGESLKI |
| NO: 147 | variable | SCKGSGYRFTTYWIGWVRQM |
| | region of H | PGKGLEWMGIIYPGDSDTRY |
| | C-3 | SPSFQGQVTISAGKSISTAY |
| | | LQWSSLKASDTAMYYCARHG |
| | | RGYNGYEGAFDIWGQGTMVT |
| | | VSS |
| SEQ ID | Light chain | AIRMTQSPSSLSASVGDRVT |
| NO: 150 | variable | ITCRASQGIRNDLAWYQQKP |
| | region of | GKTPKLLIYDASSLESGVPS |
| | LC-3 | RFSGSGSGTDFTLTISSLQP |
| | | EDFATYYCQQFNSYPLTFGG |
| | | GTKVEIK |
| SEQ ID | Heavy chain | QVQLVQSGAAVKKPGESLKI |
| NO: 147 | variable | SCKGSGYRFTTYWIGWVRQM |
| | region of | PGKGLEWMGIIYPGDSDTRY |
| | HC-4 | SPSFQGQVTISAGKSISTAY |
| | | LQWSSLKASDTAMYYCARHG |
| | | RGYNGYEGAFDIWGQGTMVT |
| | | VSS |
| SEQ ID | Light chain | AIQMTQSPSSLSASVGDRVT |
| NO: 151 | variable | ITCRASQGIRNDLGWYQQKP |
| | region of | GKAPKLLIYDASSLESGVPS |
| | LC-4 | RFSGSGSGTDFTLTISSLQP |
| | | EDFATYYCQQFNSYPLTFGG |
| | | GTKVDIK |
| SEQ ID | Heavy chain | QVQLVQSGAAVKKPGESLKI |
| NO: 147 | variable | SCKGSGYRFTTYWIGWVRQM |
| | region of | PGKGLEWMGIIYPGDSDTRY |
| | HC-5 | SPSFQGQVTISAGKSISTAY |
| | | LQWSSLKASDTAMYYCARHG |
| | | RGYNGYEGAFDIWGQGTMVT |
| | | VSS |
| SEQ ID | Light chain | NIQMTQSPSSLSASVGDRVT |
| NO: 152 | variable | ITCRASQAISDYLAWFQQKP |
| | region of | GKAPKLLIYDASNLETGVPS |
| | LC-5 | RFSGSGSGTDFTLTISSLQP |
| | | EDFATYYCQQLNSYPLTFGG |
| | | GTKVEIK |
| SEQ ID | Heavy chain | QVQLVQSGAAVKKPGESLKI |
| NO: 147 | variable | SCKGSGYRFTTYWIGWVRQM |
| | region of | PGKGLEWMGIIYPGDSDTRY |
| | HC-6 | SPSFQGQVTISAGKSISTAY |
| | | LQWSSLKASDTAMYYCARHG |
| | | RGYNGYEGAFDIWGQGTMVT |
| | | VSS |
| SEQ ID | Light chain | AIRMTQSPSSLSASVGDRVI |
| NO: 153 | variable | IACRASQGIGGALAWYQQKP |
| | region of | GNAPKVLVYDASTLESGVPS |
| | LC-6 | RFSGGGSGTDFTLTISSLQP |
| | | EDFATYYCQQFNSYPLTFGG |
| | | GTKLEIK |
| SEQ ID | Heavy chain | QVQLVQSGAAVKKPGESLKI |
| NO: 147 | variable | SCKGSGYRFTTYWIGWVRQM |
| | region of | PGKGLEWMGIIYPGDSDTRY |
| | HC-7 | SPSFQGQVTISAGKSISTAY |
| | | LQWSSLKASDTAMYYCARHG |
| | | RGYNGYEGAFDIWGQGTMVT |
| | | VSS |
| SEQ ID | Light chain | DIAMTQSPPSLSAFVGDRVT |
| NO: 154 | variable | ITCRASQGIISSLAWYQQKP |
| | region of | GKAPKLLIYDASSLESGVPS |
| | LC-7 | RFSGSGSGTDFTLTIRSLQP |
| | | EDFATYYCQQFNSYPLTFGG |
| | | GTKLEIK |
| SEQ ID | Heavy chain | QVQLVQSGAAVKKPGESLKI |
| NO: 147 | variable | SCKGSGYRFTTYWIGWVRQM |
| | region of | PGKGLEWMGIIYPGDSDTRY |
| | HC-8 | SPSFQGQVTISAGKSISTAY |
| | | LQWSSLKASDTAMYYCARHG |
| | | RGYNGYEGAFDIWGQGTMVT |
| | | VSS |
| SEQ ID | Light chain | DIQMTQSPSSLSASVGDRVT |
| NO: 155 | variable | ITCRASQGISSALAWYQQKA |
| | region of | GKAPKVLISDASSLESGVPS |
| | LC-8 | RFSGSGSGTDFTLSISSLQP |
| | | EDFATYYCQQFNGYPLTFGG |
| | | GTKVDIK |
| SEQ ID | Heavy chain | QVQLVQSGAAVKKPGESLKI |
| NO: 147 | variable | SCKGSGYRFTTYWIGWVRQM |
| | region of | PGKGLEWMGIIYPGDSDTRY |
| | HC-9 | SPSFQGQVTISAGKSISTAY |
| | amino acid | LQWSSLKASDTAMYYCARHG |
| | sequence | RGYNGYEGAFDIWGQGTMVT |
| | | VSS |
| SEQ ID | Light chain | AIRMTQSPSSLSASVGDRVT |
| NO: 156 | variable | ITCQASQGIRNDLGWYQQKP |
| | region of | GKAPKLLIYDASNLETGVPS |
| | LC-9 | RFSGSGSGTDFTFTISSLQP |
| | | EDIATYYCQQFNSYPLTFGG |
| | | GTKLEIK |
| SEQ ID | Heavy chain | QVQLVQSGAAVKKPGESLKI |
| NO: 147 | variable | SCKGSGYRFTTYWIGWVRQM |
| | region of | PGKGLEWMGIIYPGDSDTRY |
| | HC-10 | SPSFQGQVTISAGKSISTAY |
| | | LQWSSLKASDTAMYYCARHG |
| | | RGYNGYEGAFDIWGQGTMVT |
| | | VSS |
| SEQ ID | Light chain | NIQMTQSPSSLSTSVGDRVT |
| NO: 157 | variable | ITCRASQGIGTSLAWYQQKP |
| | region of | GKPPKLLIYDASSLESGVPS |
| | LC-10 | RLSGSGSGTDFTLTISSLQP |
| | | EDFATYYCQQSNSYPITFGQ |
| | | GTRLEIK |
| SEQ ID | Heavy chain | QVQLVQSGAAVKKPGESLKI |
| NO: 147 | variable | SCKGSGYRFTTYWIGWVRQM |
| | region of | PGKGLEWMGIIYPGDSDTRY |
| | HC-11 | SPSFQGQVTISAGKSISTAY |
| | | LQWSSLKASDTAMYYCARHG |
| | | RGYNGYEGAFDIWGQGTMVT |
| | | VSS |
| SEQ ID | Light chain | AIQLTQSPSSLSASVGDRVT |
| NO: 158 | variable | ITCRASQSIGDYLTWYQQKP |
| | region of | GKAPKVLIYGASSLQSGVPP |
| | LC-11 | RFSGSGSGTDFTLTVSSLQP |
| | | EDFATYYCQQLNSYPLTFGG |
| | | GTKLEIK |
| SEQ ID | Heavy chain | QVQLVQSGAAVKKPGESLKI |
| NO: 147 | variable | SCKGSGYRFTTYWIGWVRQM |
| | region of | PGKGLEWMGIIYPGDSDTRY |
| | HC-12 | SPSFQGQVTISAGKSISTAY |
| | | LQWSSLKASDTAMYYCARHG |
| | | RGYNGYEGAFDIWGQGTMVT |
| | | VSS |
| SEQ ID | Light chain | DIQLTQSPSSLSASVGDRVT |
| NO: 159 | variable | ITCRASQGVRSTLAWYQQKP |
| | region of | GKAPKLLIYDASILESGVPS |
| | LC-12 | RFSGSGSGTDFTLTISSLQP |
| | | EDFATYYCQQFNGYPLTFGQ |
| | | GTRLEIK |
| SEQ ID | Heavy chain | QVQLVQSGAAVKKPGESLKI |
| NO: 147 | variable | SCKGSGYRFTTYWIGWVRQM |
| | region of | PGKGLEWMGIIYPGDSDTRY |
| | HC-13 | SPSFQGQVTISAGKSISTAY |
| | | LQWSSLKASDTAMYYCARHG |
| | | RGYNGYEGAFDIWGQGTMVT |
| | | VSS |
| SEQ ID | Light chain | DIVMTQSPSSLSASVGDRVT |
| NO: 160 | variable | ITCRASQGIRNDLGWYQQKP |
| | region of | GKAPKLLIYDASSLESGVPS |
| | LC-13 | RFSGSGSGTDFTLTISSLQP |
| | | EDFATYYCQQFNSYPLTFGG |
| | | GTKLEIK |
| SEQ ID | Heavy chain | QVQLVQSGAAVKKPGESLKI |
| NO: 147 | variable | SCKGSGYRFTTYWIGWVRQM |
| | region of | PGKGLEWMGIIYPGDSDTRY |
| | HC-14 | SPSFQGQVTISAGKSISTAY |
| | | LQWSSLKASDTAMYYCARHG |
| | | RGYNGYEGAFDIWGQGTMVT |
| | | VSS |
| SEQ ID | Light chain | DIQLTQSPSSLSASVGDRVT |
| NO: 161 | variable | ITCRASQGISSFLAWYQQKP |
| | region of | GKAPKLLIYDASTLQSGVPS |
| | LC-14 | RFSGSASGTDFTLTISSLQP |
| | | EDFATYYCQQLNGYPLTFGG |
| | | GTKVEIK |
| SEQ ID | Heavy chain | QVQLVQSGAAVKKPGESLKI |
| NO: 147 | variable | SCKGSGYRFTTYWIGWVRQM |
| | region of | PGKGLEWMGIIYPGDSDTRY |
| | HC-15 | SPSFQGQVTISAGKSISTAY |
| | | LQWSSLKASDTAMYYCARHG |
| | | RGYNGYEGAFDIWGQGTMVT |
| | | VSS |
| SEQ ID | Light chain | AIQLTQSPSSLSASVGDRVT |
| NO: 162 | variable | ITCRASQGIGSALAWYQQKP |
| | region of | GIGPKLLIYDASTLESGVPA |
| | LC-15 | RFSGSGSRTDFTLTITSLQP |
| | | EDFATYYCQQFNGYPLTFGG |
| | | GTKLEIK |
| SEQ ID | Heavy chain | QVQLVQSGAAVKKPGESLKI |
| NO: 147 | variable | SCKGSGYRFTTYWIGWVRQM |
| | region of | PGKGLEWMGIIYPGDSDTRY |
| | HC-16 | SPSFQGQVTISAGKSISTAY |
| | | LQWSSLKASDTAMYYCARHG |
| | | RGYNGYEGAFDIWGQGTMVT |
| | | VSS |
| SEQ ID | Light chain | AIQLTQSPSSLSASVGDRVT |
| NO: 163 | variable | ITCRASQGITSALAWYQEKP |
| | region of | GKAPNLLIYDASSLESGVPS |
| | LC-16 | RFSGSGYGTDFTLTISSLQP |
| | | EDFATYYCQQLNSYPLTFGG |
| | | GTKVDIK |
| SEQ ID | Heavy chain | QIQLVQSGPELRKPGESVKI |
| NO: 164 | variable | SCKASGYTFTDYAMYWVKQA |
| | region of | PGKGLKWMGWINTYTGKPTY |
| | HC-17 | ADDFKGRFVFSLEASANTAN |
| | | LQISNLKNEDTATYFCARAR |
| | | GLVDDYVMDAWGQGTSVTVS |
| | | S |
| SEQ ID | Light chain | SYELIQPPSASVTLGNTVSL |
| NO: 165 | variable | TCVGDELSKRYAQWYQQKPD |
| | region of | KTIVSVIYKDSERPSGISDR |
| | LC-17 | FSGSSSGTTATLTIHGTLAE |
| | | DEADYYCLSTYSDDNLPVFG |
| | | GGTKLTVL |
| SEQ ID | Heavy chain | EVQLQQYGAELGKPGTSVRL |
| NO: 166 | variable | SCKVSGYNIRNTYIHWVNQR |
| | region of | PGEGLEWIGRIDPTNGNTIS |
| | HC-18 | AEKFKTKATLTADTSSHTAY |
| | | LQFSQLKSDDTAIYFCALNY |
| | | EGYADYWGQGVMVT GSS |
| SEQ ID | Light chain | DIQMTQSPSFLSASVGDRVT |
| NO: 167 | variable | INCKASQNINKYLNWYQQKV |
| | region of | GEAPKRLIFKTNSLQTGIPS |
| | LC-18 | RFSGSGSGTDYTLTISSLQT |
| | | EDVATYFCFQYNIGYTFGAG |
| | | TKVELK |
| SEQ ID | Heavy chain | EVQLQESGPGLVKPSQSLSL |
| NO: 168 | variable | TCSVTGYSISSNYRWNWIRK |
| | region of | FPGNKVEWMGYINSAGSTNY |
| | HC-19 | NPSLKSRISMTRDTSKNQFF |
| | | LQVNSVTTEDTATYYCARSL |
| | | RGYITDYSGFFDYWGQGVMV |
| | | TVSS |
| SEQ ID | Light chain | DIRMTQSPASLSASLGETVN |
| NO: 169 | variable | IECLASEDIFSDLAWYQQKP |
| | region of | GKSPQLLIYNANSLQNGVPS |
| | LC-19 | RFSGSGSGTRYSLKINSLQS |
| | | EDVATYFCQQYKNYPLTFGS |
| | | GTKLEIK |
| SEQ ID | Heavy chain | EVQLQQYGAELGKPGTSVRL |
| NO: 170 | variable | SCKLSGYKIRNTYIHWVNQR |
| | region of | PGKGLEWIGRIDPANGNTIY |
| | HC-20 | AEKFKSKVTLTADTSSNTAY |
| | | MQLSQLKSDDTALYFCAMNY |
| | | EGYEDYWGQGVMVTVSS |
| SEQ ID | Light chain | DIQMTQSPSFLSASVGDSVT |
| NO: 171 | variable | INCKASQNINKYLNWYQQKL |
| | region of | GEAPKRLIHKTDSLQTGIPS |
| | LC-20 | RFSGSGSGTDYTLTISSLQP |
| | | EDVATYFCFQYKSGFMFGAG |
| | | TKLELK |
| SEQ ID | Heavy chain | QIQLVQSGPELKKPGESVKI |
| NO: 172 | variable | SCKASGYTFTDYAVYWVIQA |
| | region of | PGKGLKWMGWINTYTGKPTY |
| | HC-21 | ADDFKGRFVFSLETSASTAN |
| | | LQISNLKNEDTATYFCARGA |
| | | GMTKDYVMDAWGRGVLVTVS |
| SEQ ID | Light chain | SYELIQPPSASVTLGNTVSL |
| NO: 173 | variable | TCVGDELSKRYAQWYQQKPD |
| | region of | KTIVSVIYKDSERPSDISDR |
| | LC-21 | FSGSSSGTTATLTIHGTLAE |
| | | DEADYYCLSTYSDDNLPVFG |
| | | GGTKLTVL |
| SEQ ID | Heavy chain | QVQLKESGPGLVQPSQTLSL |
| NO: 174 | variable | TCTVSGFSLTSYLVHWVRQPP |
| | region of | GKTLEWVGLMWNDGDTSYNS |
| | HC-22 | ALKSRLSISRDTSKSQVFLK |
| | | MHSLQAEDTATYYCARESNL |
| | | GFTYWGHGTLVTVSS |
| SEQ ID | Light chain | DIQMTQSPASLSASLEEIVT |
| NO: 175 | variable | ITCKASQGIDDDLSWYQQKP |
| | region of | GKSPQLLIYDVTRLADGVPS |
| | LC-22 | RFSGSRSGTQYSLKISRPQV |
| | | ADSGIYYCLQSYSTPYTFGA |
| | | GTKLELK |
| SEQ ID | Heavy chain | EVQLQQYGAELGKPGTSVRL |
| NO: 176 | variable | SCKVSGYNIRNTYIHWVHQR |
| | region of | PGEGLEWIGRIDPTNGNTIS |
| | HC-23 | AEKFKSKATLTADTSSNTAY |
| | | MQFSQLKSDDTAIYFCAMNY |
| | | EGYADYWGQGVMVTVSS |
| SEQ ID | Light chain | DIQMTQSPSFLSASVGDRLT |
| NO: 177 | variable | INCKASQNINKYLNWYQQKL |
| | region of | GEAPKRLIFKTNSLQTGIPS |
| | LC-23 | RFSGSGSGTDYTLTISSLQP |
| | | EDVATYFCFQYNIGFTFGAG |
| | | TKLELK |
| SEQ ID | Heavy chain | EVQLVESGGGLVQSGRSLKL |
| NO: 178 | variable | SCAASGFTVSDYYMAWVRQA |
| | region of | PTKGLEWVATINYDGSTTYH |
| | HC-24 | RDSVKGRFTISRDNAKSTLY |
| | | LQMDSLRSEDTATYYCARHG |
| | | DYGYHYGAYYFDYWGQGVMV |
| | | TVSS |
| SEQ ID | Light chain | DIVLTQSPALAVSLGQRATI |
| NO: 179 | variable | SCRASQTVSLSGYNLIHWYQ |
| | region of | QRTGQQPKLLIYRASNLAPG |
| | LC-24 | IPARFSGSGSGTDFTLTISP |
| | | VQSDDIATYYCQQSRESWTF |
| | | GGGTNLEMK |
| SEQ ID | Heavy chain | QIQLVQSGPELKKPGESVKI |
| NO: 180 | variable | SCKASGYTFTDYAIHWVKQA |
| | region of | PGQGLRWMAWINTETGKPTY |
| | HC-25 | ADDFKGRFVFSLEASASTAH |
| | | LQISNLKNEDTATFFCAGGS |
| | | HWFAYWGQGTLVTVSS |
| SEQ ID | Light chain | SYELIQPPSASVTLENTVSI |
| NO: 181 | variable | TCSGDELSNKYAHWYQQKPD |
| | region of | KTILEVIYNDSERPSGISDR |
| | LC-25 | FSGSSSGTTAILTIRDAQAE |
| | | DEADYYCLSTFSDDDLPIFG |
| | | GGTKLTVL |
| SEQ ID | Heavy chain | QIQLVQSGPELKKPGESVKI |
| NO: 172 | variable | SCKASGYTFTDYAVYWVIQA |
| | region of | PGKGLKWMGWINTYTGKPTY |
| | HC-26 | ADDFKGRFVFSLETSASTAN |
| | | LQISNLKNEDTATYFCARGA |
| | | GMTKDYVMDAWGRGVLVTVS |
| SEQ ID | Light chain | SYELIQPPSTSVTLGNTVSL |
| NO: 182 | variable | TCVGNELPKRYAYWFQQKPD |
| | region of | QSIVRLIYDDDRRPSGISDR |
| | LC-26 | FSGSSSGTTATLTIRDAQAE |
| | | DEAYYYCHSTYTDDKVPIFG |
| | | GGTKLTVL |
| SEQ ID | Heavy chain | EVQLVESGGGLVQPGRSMKL |
| NO: 183 | variable | SCKASGFTFSNYDMAWVRQA |
| | region of | PTRGLEWVASISYDGITAYY |
| | HC-27 | RDSVKGRFTISRENAKSTLY |
| | | LQLVSLRSEDTATYYCTTEG |
| | | GYVYSGPHYFDYWGQGVMVT |
| | | VSS |
| SEQ ID | Light chain | DIQMTQSPSSMSVSLGDTVT |
| NO: 184 | variable | ITCRASQDVGIFVNWFQQKP |
| | region of | GRSPRRMIYRATNLADGVPS |
| | LC-27 | RFSGSRSGSDYSLTISSLES |
| | | EDVADYHCLQYDEFPRTFGG |
| | | GTKLELK |
| SEQ ID | Heavy chain | EVQLQQYGAELGKPGTSVRL |
| NO: 185 | variable | SCKVSGYKIRNTYIHWVNQR |
| | region of | PGKGLEWIGRIDPANGNTIY |
| | HC-28 | AEKFKSKVTLTADTSSNTAY |
| | | MQLSQLKSDDTALYFCAMN |
| | | YEGYEDYWGQGV |
| | | MVTVSS |
| SEQ ID | Light chain | DIQMTQSPSFLSASVGDSVT |
| NO: 186 | variable | INCKASQNINKYLNWYQQKL |
| | region of | GEAPKRLIHKTNSLQPGFPS |
| | LC-28 | RFSGSGSGTDYTLTISSLQP |
| | | EDVAAYFCFQYNSGFTFGAG |
| | | TKLELK |
| SEQ ID | Heavy chain | QVQLVQSGAEVKKPGASVKV |
| NO: 187 | variable | SCKASGYTFTDYYIHWVRQA |
| | region of | PGQGLEWMGWMNPHSGDTGY |
| | HC-29 | AQKFQGRVTMTRDTSTSTVY |
| | | MELSSLRSEDTAVYYCARHG |
| | | RGYNGYEGAFDIWGQGTLVT |
| | | VSSAS |
| SEQ ID | Light chain | DIQMTQSPSSLSASVGDRVT |
| NO: 188 | variable | ITCRASQGIGNELGWYQQKP |
| | region of | GKAPKLLIYAASNLQSGVPS |
| | LC-29 | RFSGSGSGTDFTLTISSLQP |
| | | EDFATYYCQQYDNLPLTFGQ |
| | | GTKVEIK |
| SEQ ID | Heavy chain | QVQLVQSGAEVKKPGASVKV |
| NO: 189 | variable | SCKASGYTFTGYYLHWVRQA |
| | region of | PGQGLEWMGWINPNSGDTNY |
| | HC-30 | AQNFQGRVTMTRDTSTSTVY |
| | | MELSSLRSEDTAVYYCARHG |
| | | RGYNGYEGAFDIWGQGTLVT |
| | | VSSAS |
| SEQ ID | Light chain | DIQMTQSPSSLSASVGDRVT |
| NO: 190 | variable | ITCRASQGIRNDLGWYQQKP |
| | region of | GKAPKLLIYDASSLESGVPS |
| | LC-30 | RFSGSGSGTDFTLTISSLQP |
| | | EDFATYYCQQLNGYPLTFGG |
| | | GTKVEIK |
| SEQ ID | Heavy chain | QVQLVQSGAEVKKPGASVKV |
| NO: 191 | variable | SCKASGYTFTGYYLHWVRQA |
| | region of | PGQGLEWMGWINPNSGGTNY |
| | HC-31 | AQKFQGRVTMTRDTSTSTVY |
| | | MELSSLRSEDTAVYYCARHG |
| | | RGYEGYEGAFDIWGQGTLVT |
| | | VSSAS |
| SEQ ID | Light chain | DIQMTQSPSSLSASVGDRVT |
| NO: 192 | variable | ITCRASQGIRNDLGWYQQKP |
| | region of | GKAPKLLIYDASELETGVPS |
| | LC-31 | RFSGSGSGTDFTLTISSLQP |
| | | EDFATYYCQQLNGYPITFGQ |
| | | GTKVEIK |
| SEQ ID | Heavy chain | QVQLVQSGAEVKKPGASVKV |
| NO: 193 | variable | SCKASGYTFTSYYIHWVRQA |
| | region of | PGQGLEWMGWLNPSGGGTSY |
| | HC-32 | AQKFQGRVTMTRDTSTSTVY |
| | | MELSSLRSEDTAVYYCARHG |
| | | RGYDGYEGAFDIWGQGTLVT |
| | | VSSAS |
| SEQ ID | Light chain | DIQMTQSPSSLSASVGDRVT |
| NO: 194 | variable | ITCRASQGIRNDLGWYQQKP |
| | region of | GKAPKLLIYDASNLETGVPS |
| | LC-32 | RFSGSGSGTDFTLTISSLQP |
| | | EDFATYYCQQLNGYPLTFGG |
| | | GTKVEIK |
| SEQ ID | Heavy chain | QVQLVQSGAEVKKPGASVKV |
| NO: 195 | variable | SCKASGYTFSTYYMHWVRQA |
| | region of | PGQGLEWMGIINPSGGSTSY |
| | HC-33 | AQKFQGRVTMTRDTSTSTVY |
| | | MKLSSLRSEDTAVYYCARHG |
| | | RGYEGYEGAFDIWGQGTLVT |
| | | VSSAS |
| SEQ ID | Light chain | DIQMTQSPSSLSASVGDRVT |
| NO: 196 | variable | ITCRASQGIRDDLGWYQQKP |
| | region of | GKAPKLLIYDASNLETGVPS |
| | LC-33 | RFSGSGSGTDFTLTISSLQP |
| | | EDFATYYCQQANGFPLTFGG |
| | | GTKVEIK |
| SEQ ID | Heavy chain | QVQLVQSGAEVKKPGASVKV |
| NO: 197 | variable | SCKASGYTFTGYYIHWVRQA |
| | region of | PGQGLEWMGIINPSGGNTNY |
| | HC-34 | AQNFQGRVTMTRDTSTSTVY |
| | | MELSSLRSEDTAVYYCARHG |
| | | RGYNAYEGAFDIWGQGTLVT |
| | | VSSAS |
| SEQ ID | Light chain | DIQMTQSPSSLSASVGDRVT |
| NO: 198 | variable | ITCRASQGIRNDLGWYQQKP |
| | region of | GKAPKLLIYDASNLETGVPS |
| | LC-34 | RFSGSGSGTDFTLTISSLQP |
| | | EDFATYYCQQVNGYPLTFGG |
| | | GTKVEIK |
| SEQ ID | Heavy chain | QVQLVQSGAEVKKPGASVKV |
| NO: 199 | variable | SCKASGGTFSSYAISWVRQA |
| | region of | PGQGLEWMGVINPTVGGANY |
| | HC-35 | AQKFQGRVTMTRDTSTSTVY |
| | | MELSSLRSEDTAVYYCARHG |
| | | RGYNEYEGAFDIWGQGTLVT |
| | | VSSAS |
| SEQ ID | Light chain | DIQMTQSPSSLSASVGDRVT |
| NO: 200 | variable | ITCQASQDISDYLNWYQQKP |
| | region of | GKAPKLLIYDASNLETGVPS |
| | LC-35 | RFSGSGSGTDFTLTISSLQP |
| | | EDFATYYCQQGNSFPLTFGG |
| | | GTKLEIK |
| SEQ ID | Heavy chain | QVQLVQSGAEVKKLGASVKV |
| NO: 201 | variable | SCKASGYTFSSYYMHWVRQA |
| | region of | PGQGLEWMGVINPNGAGTNF |
| | HC-36 | AQKFQGRVTMTRDTSTSTVY |
| | | MELSSLRSEDTAVYYCARHG |
| | | RGYEGYEGAFDIWGQGTLVT |
| | | VSSAS |
| SEQ ID | Light chain | DIQMTQSPSSLSASVGDRVT |
| NO: 190 | variable | ITCRASQGIRNDLGWYQQKP |
| | region of | GKAPKLLIYDASSLESGVPS |
| | LC-36 | RFSGSGSGTDFTLTISSLQP |
| | | EDFATYYCQQLNGYPLTFGG |
| | | GTKVEIK |
| SEQ ID | Heavy chain | QVQLVQSGAEVKKPGASVKV |
| NO: 202 | variable | SCKASGYTFTTYYMHWVRQA |
| | region of | PGQGLEWMGWINPTGGGTNY |
| | HC-37 | AQNFQGRVTMTRDTSTSTVY |
| | | MELSSLRSEDTAVYYCARHG |
| | | RGYEGYEGAFDIWGQGTLVT |
| | | VSSAS |
| SEQ ID | Light chain | DIQMTQSPSSLSASVGDRVT |
| NO: 203 | variable | ITCRASQGIRNDVSWYQQKP |
| | region of | GKAPKLLIYDASNLETGVPS |
| | LC-37 | RFSGSGSGTDFTLTISSLQP |
| | | EDFATYYCQQLSGYPITFGQ |
| | | GTKLEIK |
| SEQ ID | Heavy chain | QVQLVQSGAEVKKPGASVKV |
| NO: 204 | variable | SCKASGYTFTSYYIHWVRQA |
| | region of | PGQGLEWMGMINPSGGSTNY |
| | HC-38 | AQKFQGRVTMTRDTSTSTVY |
| | | MELSSLRSEDTAVYYCARHG |
| | | RGYNDYEGAFDIWGQGTLVT |
| | | VSSAS |
| SEQ ID | Light chain | DIQMTQSPSSLSASVGDRVT |
| NO: 205 | variable | ITCRASQSISDWLAWYQQKP |
| | region of | GKAPKLLIYEASNLEGGVPS |
| | LC-38 | RFSGSGSGTDFTLTISSLQP |
| | | EDFATYYCQQANSFPYTFGQ |
| | | GTKVEIK |
| SEQ ID | Heavy chain | QVQLVQSGAEVKKPGASVKV |
| NO: 206 | variable | SCKASGYIFSAYYIHWVRQA |
| | region of | PGQGLEWMGIINPSGGSTRY |
| | HC-39 | AQKFQGRVTMTRDTSTSTVY |
| | | MELSSLRSEDTAVYYCARHG |
| | | RGYGGYEGAFDIWDQGTLVT |
| | | VSSAS |
| SEQ ID | Light chain | DIQMTQSPSSLSASVGDRVT |
| NO: 207 | variable | ITCRASQGIGDYVAWYQQKP |
| | region of | GKAPKLLIYDASNLETGVPS |
| | LC-39 | RFSGSGSGTDFTLTISSLQP |
| | | EDFATYYCQQLNGYPITFGQ |
| | | GTRLEIK |
| SEQ ID | Heavy chain | EVQLVQSGAEVKKPGESLKI |
| NO: 208 | variable | SCKGSGYRFTSYWIGWVRQM |
| | region of | PGKGLEWMGIIYPDDSDTRY |
| | HC-40 | SPSFQGQVTISVDKSNSTAY |
| | | LQWSSLKASDTAMYYCARHG |
| | | RGYNGYEGAFDIWGQGTLVT |
| | | VSSAS |
| SEQ ID | Light chain | DIQMTQSPSSLSASVGDRVT |
| NO: 209 | variable | ITCRASQGISSYLAWYQQKP |
| | region of | GKAPKLLIYDASNLETGVPS |
| | LC-40 | RFSGSGSGTYFTLTISSLQP |
| | | EDFATYYCQQGASFPITFGQ |
| | | GTKVEIK |
| SEQ ID | Heavy chain | EVQLVQSGAEVKKPGESLKI |
| NO: 210 | variable | SCKGSGSSFPNSWIAWVRQM |
| | region of | PGKGLEWMGIIYPSDSDTRY |
| | HC-41 | SPSFQGQVTISADKSISTAY |
| | | LQWSSLEASDTAMYYCARHG |
| | | RGYNGYEGAFDIWGQGTLVT |
| | | VSSAS |
| SEQ ID | Light chain | DIQMTQSPSSLSASVGDRVT |
| NO: 211 | variable | ITCRASQGIRNYLAWYQQKP |
| | region of | GKAPKLLIYDASSLQSGVPS |
| | LC-41 | RFSGSGSGTDFTLTISSLQP |
| | | EDFATYYCQQLNSYPLTFGG |
| | | GTKVEIK |
| SEQ ID | Heavy chain | EVQLVQSGAEVKKPGESLKI |
| NO: 212 | variable | SCKGSGYSFDSYWIGWVRQM |
| | region of | PGKGLEWMGIMYPGDSDTRY |
| | HC-42 | SPSFQGQVTISADKSISTAY |
| | | LQWSSLKASDTAMYYCARHG |
| | | RGYNAYEGAFDIWGQGTLVT |
| | | VSSAS |
| SEQ ID | Light chain | DIQMTQSPSSLSASVGDRVT |
| NO: 213 | variable | ITCRASQSINNWLAWYQQKP |
| | region of | GKAPKLLIYDAFILQSGVPS |
| | LC-42 | RFSGSGSGTDFTLTISSLQP |
| | | EDFATYYCLQLNSYPLTFGP |
| | | GTKVDIK |
| SEQ ID | Heavy chain | EVQLVQSGAEVKKPGESLKI |
| NO: 214 | variable | SCKGSGYSFTNWIAWVRQMP |
| | region of | GKGLEWMGIIYPGDSETRYS |
| | HC-43 | PSFQGQVTISADKSISTAYL |
| | | QWSSLKASDTAMYYCARHGR |
| | | GYYGYEGAFDIWGQGTLVTV |
| | | SSAS |
| SEQ ID | Light chain | DIQMTQSPSSLSASVGDRVT |
| NO: 215 | variable | ITCRASQGISDNLNWYQQKP |
| | region of | GKAPKLLIYDASNLETGVPS |
| | LC-43 | RFSGSGSGTDFTLTISSLQP |
| | | EDFATYYCQQAISFPLTFGQ |
| | | GTKVEIK |
| SEQ ID | Heavy chain | EVQLVQSGAEVKKPGESLKI |
| NO: 216 | variable | SCKGSGYNFTSYWIGWVRQM |
| | region of | PGKGLEWMGVIYPDDSETRY |
| | HC-44 | SPSFQGQVTISADKSISTAY |
| | | LQWSSLKASDTAMYYCARHG |
| | | RGYNGYEGAFDIWGQGTLVT |
| | | VSSAS |
| SEQ ID | Light chain | DIQMTQSPSSLSASVGDRVT |
| NO: 217 | variable | ITCRASRDIRDDLGWYQQKP |
| | region of | GKAPKLLIYDASNLETGVPS |
| | LC-44 | RFSGSGSGTDFTLTISSLQP |
| | | EDFATYYCQQANSFPLTFGG |
| | | GTKVEIK |
| SEQ ID | Heavy chain | EVQLVQSGAEVKKPGESLKI |
| NO: 218 | variable | SCKGSGYTFNTYIGWVRQMP |
| | region of | GKGLEWMGIIYPGDSGTRYS |
| | HC-45 | PSFQGQVTISADKAISTAYL |
| | | QWSSLKASDTAMYYCARHSR |
| | | GYNGYEGAFDIWGQGTLVTV |
| | | SSAS |
| SEQ ID | Light chain | DIQMTQSPSSLSASVGDRVT |
| NO: 219 | variable | ITCRASQGISNYLAWYQQKP |
| | region of | GKAPKLLIYDASNLETGVPS |
| | LC-45 | RFSGSGSGTDFTLTISSLQP |
| | | EDFATYYCQQANSFPVTFGQ |
| | | GTKVEIK |
| SEQ ID | Heavy chain | EVQLVQSGAEVKKPGESLKI |
| NO: 220 | variable | SCKGSGYNFTTYWIGWVRQM |
| | region of | PGKGLEWMGIIHPADSDTRY |
| | HC-46 | NPSFQGQVTISADKSISTAY |
| | | LQWSSLKASDTAMYYCARHG |
| | | RGYNGYEGAFDIWGQGTLVT |
| | | VSSAS |
| SEQ ID | Light chain | DIQMTQSPSSLSASVGDRVT |
| NO: 221 | variable | ITCRVSQGISSYLAWYQQKP |
| | region of | GKAPKLLIYDASNLETGVPS |
| | LC-46 | RFSGSGSGTDFTLTISSLQP |
| | | EDFATYYCQQANSFPLTFGG |
| | | GTKVEIK |
| SEQ ID | Heavy chain | EVQLVQSGAEVKKPGESLKI |
| NO: 222 | variable | SCKGSGYRFSNYWIAWVRQM |
| | region of | PGKGLEWMGIIYPDNSDTRY |
| | HC-47 | SPSFQGQVTISADKSISTAY |
| | | LQWSSLKASDTAMYYCARHG |
| | | RGYDGYEGAFDIWGQGTLVT |
| | | VSSAS |
| SEQ ID | Light chain | DIQMTQSPSSLSASVGDRVT |
| NO: 223 | variable | ITCRASQGIRSDLAWYQQKP |
| | region of | GKAPKLLIYGASSLQSGVPS |
| | LC-47 | RFSGSGSGTDFTLTISSLQP |
| | | EDFATYYCQQANSFPLSFGQ |
| | | GTKVEIK |
| SEQ ID | Heavy chain | EVQLVQSGAEVKKPGESLKI |
| NO: 224 | variable | SCKGSGYRFASYWIGWVRQM |
| | region of | PGKGLEWMGITYPGDSETRY |
| | HC-48 | NPSQGQVTISADKSISTAYL |
| | | QWSSLKASDTAMYYCARHGR |
| | | GYGGYEGAFDIWGQGTLVTV |
| | | SSAS |
| SEQ ID | Light chain | DIQMTQSPSSLSASVGDRVT |
| NO: 225 | variable | ITCRASQGIRNDLGWYQQKP |
| | region of | GKAPKLLIYDASNLETGVPS |
| | LC-48 | RFSGSGSGTDFTLTISSLQP |
| | | EDFATYYCQQANSFPLTFGG |
| | | GTKVEIK |
| SEQ ID | Heavy chain | EVQLVQSGAEVKKPGESLKI |
| NO: 226 | variable | SCKGSGYSFTSYWIGWVRQM |
| | region of | PGKGLEWMGIIYPGDSDTRY |
| | HC-49 | SPSFQGQVTISADKSISTAY |
| | | LQWSSLKASDTAMYYCARHG |
| | | RGYNGYEGAFDIWGQGTLVT |
| | | VSSAS |
| SEQ ID | Light chain | DIQMTQSPSSLSASVGDRVT |
| NO: 227 | variable | ITCRASQSISNWLAWYQQKP |
| | region of | GKAPKLLIYDASNLETGVPS |
| | LC-49 | RFSGSGSGTDFTLTISSLQP |
| | | EDFATYYCQQTNSFPLTFGQ |
| | | GTRLEIK |
| SEQ ID | Heavy chain | QVQLVQSGAAVKKPGESLKI |
| NO: 147 | variable | SCKGSGYRFTTYWIGWVRQM |
| | region of | PGKGLEWMGIIYPGDSDTRY |
| | HC-74 | SPSFQGQVTISAGKSISTAY |
| | | LQWSSLKASDTAMYYCARHG |
| | | RGYNGYEGAFDIWGQGTMVT |
| | | VSS |
| SEQ ID | Light chain | DIQLTQSPSSLSASVGDRVT |
| NO: 228 | variable | ITCRASQGVISALAWYQQKP |
| | region of | GKAPKLLIYDASSLESGVPS |
| | LC-74 | RFSGSGSGTDFTLTISSLQP |
| | | EDFATYYCQQFNSYPLTFGG |
| | | GTKVEIK |
| SEQ ID | Heavy chain | QVQLVQSGAAVKKPGESLKI |
| NO: 147 | variable | SCKGSGYRFTTYWIGWVRQM |
| | region of | PGKGLEWMGIIYPGDSDTRY |
| | HC-75 | SPSFQGQVTISAGKSISTAY |
| | | LQWSSLKASDTAMYYCARHG |
| | | RGYNGYEGAFDIWGQGTMVT |
| | | VSS |
| SEQ ID | Light chain | DIQLTQSPSSLSASVGDRVT |
| NO: 229 | variable | ITCRASQGIRSALAWYQQKP |
| | region of | GKAPKLLIYDASSLESGVPS |
| | LC-75 | RFSGSGSGTDFTLTISSLQP |
| | | EDFATYYCQQFNSYPLTFGG |
| | | GTKVEIK |
| SEQ ID | Heavy chain | QVQLVQSGAAVKKPGESLKI |
| NO: 147 | variable | SCKGSGYRFTTYWIGWVRQM |
| | region of | PGKGLEWMGIIYPGDSDTRY |
| | HC-76 | SPSFQGQVTISAGKSISTAY |
| | | LQWSSLKASDTAMYYCARHG |
| | | RGYNGYEGAFDIWGQGTMVT |
| | | VSS |
| SEQ ID | Light chain | DIQLTQSPSSLSASVGDRVT |
| NO: 230 | variable | ITCRASQGVGSALAWYQQKP |
| | region of | GKAPKLLIYDASSLESGVPS |
| | LC-76 | RFSGSGSGTDFTLTISSLQP |
| | | EDFATYYCQQFNSYPLTFGG |
| | | GTKVEIK |
| SEQ ID | Heavy chain | QVQLVQSGAAVKKPGESLKI |
| NO: 147 | variable | SCKGSGYRFTTYWIGWVRQM |
| | region of | PGKGLEWMGIIYPGDSDTRY |
| | HC-77 | SPSFQGQVTISAGKSISTAY |
| | | LQWSSLKASDTAMYYCARHG |
| | | RGYNGYEGAFDIWGQGTMVT |
| | | VSS |
| SEQ ID | Light chain | DIQLTQSPSSLSASVGDRVT |
| NO: 231 | variable | ITCRASQGVISALAWYQQKP |
| | region of | GKAPKLLIYDASILESGVPS |
| | LC-77 | RFSGSGSGTDFTLTISSLQP |
| | | EDFATYYCQQFNSYPLTFGG |
| | | GTKVEIK |
| SEQ ID | Heavy chain | QVQLVQSGAAVKKPGESLKI |
| NO: 147 | variable | SCKGSGYRFTTYWIGWVRQM |
| | region of | PGKGLEWMGIIYPGDSDTRY |
| | HC-78 | SPSFQGQVTISAGKSISTAY |
| | | LQWSSLKASDTAMYYCARHG |
| | | RGYNGYEGAFDIWGQGTMVT |
| | | VSS |
| SEQ ID | Light chain | DIQLTQSPSSLSASVGDRVT |
| NO: 232 | variable | ITCRASQGIRSALAWYQQKP |
| | region of | GKAPKLLIYDASILESGVPS |
| | LC-78 | RFSGSGSGTDFTLTISSLQP |
| | | EDFATYYCQQFNSYPLTFGG |
| | | GTKVEIK |
| SEQ ID | Heavy chain | QVQLVQSGAAVKKPGESLKI |
| NO: 147 | variable | SCKGSGYRFTTYWIGWVRQM |
| | region of | PGKGLEWMGIIYPGDSDTRY |
| | HC-79 | SPSFQGQVTISAGKSISTAY |
| | | LQWSSLKASDTAMYYCARHG |
| | | RGYNGYEGAFDIWGQGTMVT |
| | | VSS |
| SEQ ID | Light chain | DIQLTQSPSSLSASVGDRVT |
| NO: 233 | variable | ITCRASQGVGSALAWYQQKP |
| | region of | GKAPKLLIYDASILESGVPS |
| | LC-79 | RFSGSGSGTDFTLTISSLQP |
| | | EDFATYYCQQFNSYPLTFGG |
| | | GTKVEIK |
| SEQ ID | Heavy chain | QVQLVQSGAAVKKPGESLKI |
| NO: 147 | variable | SCKGSGYRFTTYWIGWVRQM |
| | region of | PGKGLEWMGIIYPGDSDTRY |
| | HC-80 | SPSFQGQVTISAGKSISTAY |
| | | LQWSSLKASDTAMYYCARHG |
| | | RGYNGYEGAFDIWGQGTMVT |
| | | VSS |
| SEQ ID | Light chain | DIQLTQSPSSLSASVGDRVT |
| NO: 234 | variable | ITCRASQGISSALAWYQQKP |
| | region of | GKAPKLLIYDASILESGVPS |
| | LC-80 | RFSGSGSGTDFTLTISSLQP |
| | | EDFATYYCQQFNSYPLTFGG |
| | | GTKVEIK |
| SEQ ID | Heavy chain | QVQLVQSGAAVKKPGESLKI |
| NO: 147 | variable | SCKGSGYRFTTYWIGWVRQM |
| | region of | PGKGLEWMGIIYPGDSDTRY |
| | HC-81 | SPSFQGQVTISAGKSISTAY |
| | | LQWSSLKASDTAMYYCARHG |
| | | RGYNGYEGAFDIWGQGTMVT |
| | | VSS |
| SEQ ID | Light chain | DIQLTQSPSSLSASVGDRVT |
| NO: 235 | variable | ITCRASQGVISALAWYQQKP |
| | region of | GKAPKLLIYDASTLESGVPS |
| | LC-81 | RFSGSGSGTDFTLTISSLQP |
| | | EDFATYYCQQFNSYPLTFGG |
| | | GTKVEIK |
| SEQ ID | Heavy chain | QVQLVQSGAAVKKPGESLKI |
| NO: 147 | variable | SCKGSGYRFTTYWIGWVRQM |
| | region of | PGKGLEWMGIIYPGDSDTRY |
| | HC-82 | SPSFQGQVTISAGKSISTAY |
| | | LQWSSLKASDTAMYYCARHG |
| | | RGYNGYEGAFDIWGQGTMVT |
| | | VSS |
| SEQ ID | Light chain | DIQLTQSPSSLSASVGDRVT |
| NO: 236 | variable | ITCRASQGIRSALAWYQQKP |
| | region of | GKAPKLLIYDASTLESGVPS |
| | LC-82 | RFSGSGSGTDFTLTISSLQP |
| | | EDFATYYCQQFNSYPLTFGG |
| | | GTKVEIK |
| SEQ ID | Heavy chain | QVQLVQSGAAVKKPGESLKI |
| NO: 147 | variable | SCKGSGYRFTTYWIGWVRQM |
| | region of | PGKGLEWMGIIYPGDSDTRY |
| | HC-83 | SPSFQGQVTISAGKSISTAY |
| | | LQWSSLKASDTAMYYCARHG |
| | | RGYNGYEGAFDIWGQGTMVT |
| | | VSS |
| SEQ ID | Light chain | DIQLTQSPSSLSASVGDRVT |
| NO: 237 | variable | ITCRASQGVGSALAWYQQKP |
| | region of | GKAPKLLIYDASTLESGVPS |
| | LC-83 | RFSGSGSGTDFTLTISSLQP |
| | | EDFATYYCQQFNSYPLTFGG |
| | | GTKVEIK |
| SEQ ID | Heavy chain | QVQLVQSGAAVKKPGESLKI |
| NO: 147 | variable | SCKGSGYRFTTYWIGWVRQM |
| | region of | PGKGLEWMGIIYPGDSDTRY |
| | HC-84 | SPSFQGQVTISAGKSISTAY |
| | | LQWSSLKASDTAMYYCARHG |
| | | RGYNGYEGAFDIWGQGTMVT |
| | | VSS |
| SEQ ID | Light chain | DIQLTQSPSSLSASVGDRVT |
| NO: 237 | variable | ITCRASQGVGSALAWYQQKP |
| | region of | GKAPKLLIYDASTLESGVPS |
| | LC-84 | RFSGSGSGTDFTLTISSLQP |
| | | EDFATYYCQQFNSYPLTFGG |
| | | GTKVEIK |
| SEQ ID | Heavy chain | EVQLVQSGAEVKKPGESLKI |
| NO: 238 | variable | SCKGSGYRFTTSWIGWVRQM |
| | region of | PGKGLEWMGIIYPGDSDTRY |
| | HC-245 | SPSFQGQVTISADKSISTAY |
| | | LQWSSLKASDTAMYYCARHG |
| | | LGYNGYEGAFDIWGQGTLVT |
| | | VSS |
| SEQ ID | Light chain | DIQMTQSPSSLSASVGDRVT |
| NO: 239 | variable | ITCRASQGIGSALAWYQQKP |
| | region of | GKAPKLLIYDASTLESGVPS |
| | LC-245 | RFSGSGSGTDFTLTISSLQP |
| | | EDFATYYCQQFNGYPLTFGQ |
| | | GTRLEIK |
| SEQ ID | Heavy chain | QVQLVQSGAAVKKPGESLKI |
| NO: 147 | variable | SCKGSGYRFTTYWIGWVRQM |
| | region of | PGKGLEWMGIIYPGDSDTRY |
| | HC-246 | SPSFQGQVTISAGKSISTAY |
| | | LQWSSLKASDTAMYYCARHG |
| | | RGYNGYEGAFDIWGQGTMVT |
| | | VSS |
| SEQ ID | Light chain | DIQMTQSPSSLSASVGDRVT |
| NO: 239 | variable | ITCRASQGIGSALAWYQQKP |
| | region of | GKAPKLLIYDASTLESGVPS |
| | LC-246 | RFSGSGSGTDFTLTISSLQP |
| | | EDFATYYCQQFNGYPLTFGQ |
| | | GTRLEIK |
| SEQ ID | Heavy chain | QVQLVQSGAAVKKPGESLKI |
| NO: 147 | variable | SCKGSGYRFTTYWIGWVRQM |
| | region of | PGKGLEWMGIIYPGDSDTRY |
| | HC-247 | SPSFQGQVTISAGKSISTAY |
| | | LQWSSLKASDTAMYYCARHG |
| | | RGYNGYEGAFDIWGQGTMVT |
| | | VSS |
| SEQ ID | Light chain | DIQMTQSPSSLSASVGDRVT |
| NO: 240 | variable | ITCRASRGISDYLAWYQQKP |
| | region of | GKAPKLLIYDASNLETGVPS |
| | LC-247 | RFSGSGSGTDFTLTISSLQP |
| | | EDFATYYCQQANSFPITFGQ |
| | | GTRLEIK |
| SEQ ID | Heavy chain | EVQLVQSGAEVKKPGESLKI |
| NO: 238 | variable | SCKGSGYRFTTSWIGWVRQM |
| | region of | PGKGLEWMGIIYPGDSDTRY |
| | HC-248 | SPSFQGQVTISADKSISTAY |
| | | LQWSSLKASDTAMYYCARHG |
| | | LGYNGYEGAFDIWGQGTLVT |
| | | VSS |
| SEQ ID | Light chain | DIQMTQSPSSLSASVGDRVT |
| NO: 241 | variable | ITCRASQGIGSALAWYQQKP |
| | region of | GKAPKLLIYDASTLESGVPS |
| | LC-248 | RFSGSGSGTDFTLTISSLQP |
| | | EDFATYYCQQLNGYPLTFGQ |
| | | GTRLEIK |
| SEQ ID | Heavy chain | EVQLVQSGAEVKKPGESLKI |
| NO: 238 | variable | SCKGSGYRFTTSWIGWVRQM |
| | region of | PGKGLEWMGIIYPGDSDTRY |
| | HC-249 | SPSFQGQVTISADKSISTAY |
| | | LQWSSLKASDTAMYYCARHG |
| | | LGYNGYEGAFDIWGQGTLVT |
| | | VSS |
| SEQ ID | Light chain | DIQMTQSPSSLSASVGDRVT |
| NO: 242 | variable | ITCRASQGIGSALAWYQQKP |
| | region of | GKAPKLLIYDASNLETGVPS |
| | LC-249 | RFSGSGSGTDFTLTISSLQP |
| | | EDFATYYCQQLNGYPLTFGQ |
| | | GTRLEIK |
| SEQ ID | Heavy chain | EVQLVQSGAEVKKPGESLKI |
| NO: 243 | variable | SCKGSGYSFTNYWIGWVRQM |
| | region of | PGKGLEWMAIINPRDSDTRY |
| | Ab 85 | RPSFQGQVTISADKSISTAY |
| | | LQWSSLKASDTAMYYCARHG |
| | | RGYEGYEGAFDIWGQGTLVT |
| | | VSS |
| SEQ ID | Light chain | DIQMTQSPSSLSASVGDRVT |
| NO: 244 | variable | ITCRSSQGIRSDLGWYQQKP |
| | region of | GKAPKLLIYDASNLETGVPS |
| | Ab 85 | RFSGSGSGTDFTLTISSLQP |
| | | EDFATYYCQQANGFPLTFGG |
| | | GTKVEIK |
| SEQ ID | Ab85 CDR-H1 | NYWIG |
| NO: 245 | | |
| SEQ ID | Ab85 CDR-H2 | IINPRDSDTRYRPSFQG |
| NO: 246 | | |
| SEQ ID | Ab85 CDR-H3 | HGRGYEGYEGAFDI |
| NO: 247 | | |
| SEQ ID | Ab85 CDR-L1 | RSSQGIRSDLG |
| NO: 248 | | |
| SEQ ID | Ab85 CDR-L2 | DASNLET |
| NO: 249 | Ab249 CDR-L2 | |
| SEQ ID | Ab85 CDR-L3 | QQANGFPLT |
| NO: 250 | | |
| SEQ ID | Heavy chain | EVQLVQSGAEVKKPGESLKI |
| NO: 251 | variable | SCKGSGYSFTNYWIGWVRQM |
| | region | PGKGLEWMGIIYPGDSDIRY |
| | of Ab 86 | SPSLQGQVTISVDTSTSTAY |
| | | LQWNSLKPSDTAMYYCARHG |
| | | RGYNGYEGAFDIWGQGTLVT |
| | | VSS |
| SEQ ID | Light chain | DIQMTQSPSSLSASVGDRVT |
| NO: 252 | variable | ITCRASQGIGDSLAWYQQKP |
| | region | GKAPKLLIYDASNLETGVPS |
| | of Ab 86 | RFSGSGSGTDFTLTISSLQP |
| | | EDFATYYCQQLNGYPITFGQ |
| | | GTKVEIK |
| SEQ ID | Ab86 CDR-H1 | NYWIG |
| NO: 245 | | |
| SEQ ID | Ab86 CDR-H2 | IIYPGDSDIRYSPSLQG |
| NO: 253 | | |
| SEQ ID | Ab86 CDR-H3 | HGRGYNGYEGAFDI |
| NO:3 | | |
| SEQ ID | Ab86 CDR-L1 | RASQGIGDSLA |
| NO: 254 | | |
| SEQ ID | Ab86 CDR-L2 | DASNLET |
| NO: 249 | | |
| SEQ ID | Ab86 CDR-L3 | QQLNGYPIT |
| NO: 255 | | |
| SEQ ID | Heavy chain | EVQLVQSGAEVKKPGESLKI |
| NO: 243 | variable | SCKGSGYSFTNYWIGWVRQM |
| | region of | PGKGLEWMAIINPRDSDTRY |
| | Ab 87 | RPSFQGQVTISADKSISTAY |
| | | LQWSSLKASDTAMYYCARHG |
| | | RGYEGYEGAFDIWGQGTLVT |
| | | VSS |
| SEQ ID | Light chain | DIQMTQSPSSLSASVGDRVT |
| NO: 256 | variable | ITCRASQGIRNDLGWYQQKP |
| | region of | GKAPKLLIYDASSLESGVPS |
| | Ab 87 | RFSGSGSGTDFTLTISSLQP |
| | | EDFATYYCQQLNGYPITFGQ |
| | | GTKVEIK |
| SEQ ID | Ab87 CDR-H1 | NYWIG |
| NO: 245 | | |
| SEQ ID | Ab87 CDR-H2 | IINPRDSDTRYRPSFQG |
| NO: 246 | | |
| SEQ ID | Ab87 CDR-H3 | HGRGYEGYEGAFDI |
| NO: 247 | | |
| SEQ ID | Ab87 CDR-L1 | RASQGIRNDLG |
| NO: 257 | | |
| SEQ ID | Ab87 CDR-L2 | DASSLES |
| NO:5 | | |
| SEQ ID | Ab87 CDR-L3 | QQLNGYPIT |
| NO: 255 | | |
| SEQ ID | Heavy chain | EVQLVQSGAEVKKPGESLKI |
| NO: 258 | variable | SCKGSGYSFTNYWIGWVRQM |
| | region of | PGKGLEWMGIIYPGDSLTRY |
| | Ab 88 | SPSFQGQVTISADKSISTAY |
| | | LQWSSLKASDTAMYYCARHG |
| | | RGYNGYEGAFDIWGQGTLVT |
| | | VSS |
| SEQ ID | Light chain | DIQMTQSPSSLSASVGDRVT |
| NO: 256 | variable | ITCRASQGIRNDLGWYQQKP |
| | region of | GKAPKLLIYDASSLESGVPS |
| | Ab 88 | RFSGSGSGTDFTLTISSLQP |
| | | EDFATYYCQQLNGYPITFGQ |
| | | GTKVEIK |
| SEQ ID | Ab88 CDR-H1 | NYWIG |
| NO: 245 | | |
| SEQ ID | Ab88 CDR-H2 | IIYPGDSLTRYSPSFQG |
| NO: 259 | | |
| SEQ ID | Ab88 CDR-H3 | HGRGYNGYEGAFDI |
| NO:3 | | |
| SEQ ID | Ab88 CDR-L1 | RASQGIRNDLG |
| NO: 257 | | |
| SEQ ID | Ab88 CDR-L2 | DASSLES |
| NO:5 | | |
| SEQ ID | Ab88 CDR-L3 | QQLNGYPIT |
| NO: 255 | | |
| SEQ ID | Heavy chain | EVQLVQSGAEVKKPGESLKI |
| NO: 260 | variable | SCKGSGYSFTNYWIGWVRQM |
| | region of | PGKGLEWMGIIYPGDSDTRY |
| | Ab89 | SPSFQGQVTISADKSISTAY |
| | | LQWSSLKASDTAMYYCARHG |
| | | RGYNGYEGAFDIWGQGTLVT |
| | | VSS |
| SEQ ID | Light chain | DIQMTQSPSSLSASVGDRVT |
| NO: 252 | variable | ITCRASQGIGDSLAWYQQKP |
| | region of | GKAPKLLIYDASNLETGVPS |
| | Ab89 | RFSGSGSGTDFTLTISSLQP |
| | | EDFATYYCQQLNGYPITFGQ |
| | | GTKVEIK |
| SEQ ID | Ab89 CDR-H1 | NYWIG |
| NO: 245 | | |
| SEQ ID | Ab89 CDR-H2 | IIYPGDSDTRYSPSFQG |
| NO:2 | | |
| SEQ ID | Ab89 CDR-H3 | HGRGYNGYEGAFDI |
| NO:3 | | |
| SEQ ID | Ab89 CDR-L1 | RASQGIGDSLA |
| NO: 254 | | |
| SEQ ID | Ab89 CDR-L2 | DASNLET |
| NO: 249 | | |
| SEQ ID | Ab89 CDR-L3 | QQLNGYPIT |
| NO: 255 | | |
| SEQ ID | Heavy chain | QVQLVQSGAAVKKPGESLKI |
| NO: 261 | variable | SCKGSGYRFTSYWIGWVRQM |
| | region | PGKGLEWMGIIYPGDSDTRY |
| | amino | SPSFQGQVTISAGKSISTAY |
| | acid | LQWSSLKASDTAMYYCARHG |
| | sequence | RGYNGYEGAFDIWGQGTMVT |
| | of CK6 | VSS |
| SEQ ID | Light chain | AIQLTQSPSSLSASVGDRVT |
| NO: 262 | variable | ITCRASQGISSALAWYQQKP |
| | region | GKAPKLLIYDASSLESGVPS |
| | amino | RFSGSGSGTDFTLTISSLQP |
| | acid | EDFATYYCQQFNSYPLTFGG |
| | sequence | GTKVEIK |
| | of CK6 | |
| SEQ ID | Ab77 CDR-H1 | TYWIG |
| NO: 263 | | |
| SEQ ID | Ab77 CDR-H2 | IIYPGDSDTRYSPSFQG |
| N0:2 | | |
| SEQ ID | Ab77 CDR-H3 | HGRGYNGYEGAFDI |
| N0:3 | | |
| SEQ ID | Ab77 CDR-L1 | RASQGVISALA |
| NO: 264 | | |
| SEQ ID | Ab77 CDR-L2 | DASILES |
| NO: 265 | | |
| SEQ ID | Ab77 CDR-L3 | QQFNSYPLT |
| NO: 266 | | |
| SEQ ID | Ab79 CDR-H1 | TYWIG |
| NO: 263 | | |
| SEQ ID | Ab79 CDR-H2 | IIYPGDSDTRYSPSFQG |
| NO:2 | | |
| SEQ ID | Ab79 CDR-H3 | HGRGYNGYEGAFDI |
| NO:3 | | |
| SEQ ID | Ab79 CDR-L1 | RASQGVGSALA |
| NO: 267 | | |
| SEQ ID | Ab79 CDR-L2 | DASILES |
| NO: 265 | | |
| SEQ ID | Ab79 CDR-L3 | QQFNSYPLT |
| NO: 266 | | |
| SEQ ID | Ab81 CDR-H1 | TYWIG |
| NO: 263 | | |
| SEQ ID | Ab81 CDR-H2 | IIYPGDSDTRYSPSFQG |
| NO:2 | | |
| SEQ ID | Ab81 CDR-H3 | HGRGYNGYEGAFDI |
| NO:3 | | |
| SEQ ID | Ab81 CDR-L1 | RASQGVISALA |
| NO: 264 | | |
| SEQ ID | Ab81 CDR-L2 | DASTLES |
| NO: 268 | | |
| SEQ ID | Ab81 CDR-L3 | QQFNSYPLT |
| NO: 266 | | |
| SEQ ID | Heavy chain | ASTKGPSVFPLAPSSKSTSG |
| NO: 269 | constant | GTAALGCLVKDYFPEPVTVS |
| | region | WNSGALTSGVHTFPAVLQSS |
| | (Wild type | GLYSLSSVVTVPSSSLGTQT |
| | (WT)) | YICNVNHKPSNTKVDKKVEP |
| | | KSCDKTHTCPPCPAPELLGG |
| | | PSVFLFPPKPKDTLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSRDE |
| | | LTKNQVSLTCLVKGFYPSDI |
| | | AVEWESNGQPENNYKTTPPV |
| | | LDSDGSFFLYSKLTVDKSRW |
| | | QQGNVFSCSVMHEALHNHYT |
| | | QKSLSLSPGK |
| SEQ ID | Heavy chain | ASTKGPSVFPLAPSSKSTSG |
| NO: 270 | constant | GTAALGCLVKDYFPEPVTVS |
| | region with | WNSGALTSGVHTFPAVLQSS |
| | L234A, L235A | GLYSLSSVVTVPSSSLGTQT |
| | (LALA) | YICNVNHKPSNTKVDKKVEP |
| | mutations | KSCDKTHTCPPCPAPEAAGG |
| | (mutations | PSVFLFPPKPKDTLMISRTP |
| | in bold)* | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSRDE |
| | | LTKNQVSLTCLVKGFYPSDI |
| | | AVEWESNGQPENNYKTTPPV |
| | | LDSDGSFFLYSKLTVDKSRW |
| | | QQGNVFSCSVMHEALHNHYT |
| | | QKSLSLSPGK |
| SEQ ID | Heavy chain | ASTKGPSVFPLAPSSKSTSG |
| NO: 271 | constant | GTAALGCLVKDYFPEPVTVS |
| | region | WNSGALTSGVHTFPAVLQSS |
| | with D265C | GLYSLSSVVTVPSSSLGTQT |
| | mutation | YICNVNHKPSNTKVDKKVEP |
| | (mutation | KSCDKTHTCPPCPAPELLGG |
| | in bold)* | PSVFLFPPKPKDTLMISRTP |
| | | EVTCVVVCVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSRDE |
| | | LTKNQVSLTCLVKGFYPSDI |
| | | AVEWESNGQPENNYKTTPPV |
| | | LDSDGSFFLYSKLTVDKSRW |
| | | QQGNVFSCSVMHEALHNHYT |
| | | QKSLSLSPGK |
| SEQ ID | Heavy chain | ASTKGPSVFPLAPSSKSTSG |
| NO: 272 | constant | GTAALGCLVKDYFPEPVTVS |
| | region with | WNSGALTSGVHTFPAVLQSS |
| | H435A | GLYSLSSVVTVPSSSLGTQT |
| | mutation | YICNVNHKPSNTKVDKKVEP |
| | (mutation | KSCDKTHTCPPCPAPELLGG |
| | in bold)* | PSVFLFPPKPKDTLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSRDE |
| | | LTKNQVSLTCLVKGFYPSDI |
| | | AVEWESNGQPENNYKTTPPV |
| | | LDSDGSFFLYSKLTVDKSRW |
| | | QQGNVFSCSVMHEALHNAYT |
| | | QKSLSLSPGK |
| SEQ ID | Heavy chain | ASTKGPSVFPLAPSSKSTSG |
| NO: 273 | Constant | GTAALGCLVKDYFPEPVTVS |
| | region: | WNSGALTSGVHTFPAVLQSS |
| | modified | GLYSLSSVVTVPSSSLGTQT |
| | Fc region | YICNVNHKPSNTKVDKKVEP |
| | with L234A, | KSCDKTHTCPPCPAPEAAGG |
| | L235A, | PSVFLFPPKPKDTLMISRTP |
| | D265C | EVTCVVVCVSHEDPEVKFNW |
| | mutations | YVDGVEVHNAKTKPREEQYN |
| | (mutations | STYRVVSVLTVLHQDWLNGK |
| | in bold)* | EYKCKVSNKALPAPIEKTIS |
| | | KAKGQPREPQVYTLPPSRDE |
| | | LTKNQVSLTCLVKGFYPSDI |
| | | AVEWESNGQPENNYKTTPPV |
| | | LDSDGSFFLYSKLTVDKSRW |
| | | QQGNVFSCSVMHEALHNHYT |
| | | QKSLSLSPGK |
| SEQ ID | Heavy chain | ASTKGPSVFPLAPSSKSTSG |
| NO: 274 | constant | GTAALGCLVKDYFPEPVTVS |
| | region: | WNSGALTSGVHTFPAVLQSS |
| | modified | GLYSLSSVVTVPSSSLGTQT |
| | Fc region | YICNVNHKPSNTKVDKKVEP |
| | with L234A, | KSCDKTHTCPPCPAPEAAGG |
| | L235A, | PSVFLFPPKPKDTLMISRTP |
| | D265C, | EVTCVVVCVSHEDPEVKFNW |
| | H435A | YVDGVEVHNAKTKPREEQYN |
| | mutations | STYRVVSVLTVLHQDWLNGK |
| | (mutations | EYKCKVSNKALPAPIEKTIS |
| | in bold)* | KAKGQPREPQVYTLPPSRDE |
| | | LTKNQVSLTCLVKGFYPSDI |
| | | AVEWESNGQPENNYKTTPPV |
| | | LDSDGSFFLYSKLTVDKSRW |
| | | QQGNVFSCSVMHEALHNAYT |
| | | QKSLSLSPGK |
| SEQ ID | Ab85 full | EVQLVQSGAEVKKPGESLKI |
| NO: 275 | length | SCKGSGYSFTNYWIGWVRQM |
| | heavy chain | PGKGLEWMAIINPRDSDTRY |
| | sequence; | RPSFQGQVTISADKSISTAY |
| | constant | LQWSSLKASDTAMYYCARHG |
| | region | RGYEGYEGAFDIWGQGTLVT |
| | underlined | VSSASTKGPSVFPLAPSSKS |
| | | TSGGTAALGCLVKDYFPEPV |
| | | TVSWNSGALTSGVHTFPAVL |
| | | QSSGLYSLSSVVTVPSSSLG |
| | | TQTYICNVNHKPSNTKVDKK |
| | | VEPKSCDKTHTCPPCPAPEL |
| | | LGGPSVFLFPPKPKDTLMIS |
| | | RTPEVTCVVVDVSHEDPEVK |
| | | FNWYVDGVEVHNAKTKPREE |
| | | QYNSTYRVVSVLTVLHQDWL |
| | | NGKEYKCKVSNKALPAPIEK |
| | | TISKAKGQPREPQVYTLPPS |
| | | RDELTKNQVSLTCLVKGFYP |
| | | SDIAVEWESNGQPENNYKTT |
| | | PPVLDSDGSFFLYSKLTVDK |
| | | SRWQQGNVFSCSVMHEALHN |
| | | HYTQKSLSLSPGK |
| SEQ ID | Ab85 full | EVQLVQSGAEVKKPGESLKI |
| NO: 276 | length | SCKGSGYSFTNYWIGWVRQM |
| | heavy chain | PGKGLEWMAIINPRDSDTRY |
| | sequence; | RPSFQGQVTISADKSISTAY |
| | constant | LQWSSLKASDTAMYYCARHG |
| | region | RGYEGYEGAFDIWGQGTLVT |
| | underlined; | VSSASTKGPSVFPLAPSSKS |
| | modified | TSGGTAALGCLVKDYFPEPV |
| | Fc region | TVSWNSGALTSGVHTFPAVL |
| | with L234A, | QSSGLYSLSSVVTVPSSSLG |
| | L235A | TQTYICNVNHKPSNTKVDKK |
| | mutations | VEPKSCDKTHTCPPCPAPEA |
| | (mutations | AGGPSVFLFPPKPKDTLMIS |
| | in bold)* | RTPEVTCVVVDVSHEDPEVK |
| | | FNWYVDGVEVHNAKTKPREE |
| | | QYNSTYRVVSVLTVLHQDWL |
| | | NGKEYKCKVSNKALPAPIEK |
| | | TISKAKGQPREPQVYTLPPS |
| | | RDELTKNQVSLTCLVKGFYP |
| | | SDIAVEWESNGQPENNYKTT |
| | | PPVLDSDGSFFLYSKLTVDK |
| | | SRWQQGNVFSCSVMHEALHN |
| | | HYTQKSLSLSPGK |
| SEQ ID | Ab85 full | EVQLVQSGAEVKKPGESLKI |
| NO: 277 | length | SCKGSGYSFTNYWIGWVRQM |
| | heavy chain | PGKGLEWMAIINPRDSDTRY |
| | sequence: | RPSFQGQVTISADKSISTAY |
| | constant | LQWSSLKASDTAMYYCARHG |
| | region | RGYEGYEGAFDIWGQGTLVT |
| | underlined; | VSSASTKGPSVFPLAPSSKS |
| | modified | TSGGTAALGCLVKDYFPEPV |
| | Fc region | TVSWNSGALTSGVHTFPAVL |
| | with | QSSGLYSLSSVVTVPSSSLG |
| | L234A, | TQTYICNVNHKPSNTKVDKK |
| | L235A, | VEPKSCDKTHTCPPCPAPEA |
| | D265C | AGGPSVFLFPPKPKDTLMIS |
| | mutations | RTPEVTCVVVCVSHEDPEVK |
| | (mutations | FNWYVDGVEVHNAKTKPREE |
| | in bold)* | QYNSTYRVVSVLTVLHQDWL |
| | | NGKEYKCKVSNKALPAPIEK |
| | | TISKAKGQPREPQVYTLPPS |
| | | RDELTKNQVSLTCLVKGFYP |
| | | SDIAVEWESNGQPENNYKTT |
| | | PPVLDSDGSFFLYSKLTVDK |
| | | SRWQQGNVFSCSVMHEALHN |
| | | HYTQKSLSLSPGK |
| SEQ ID | Ab85 full | EVQLVQSGAEVKKPGESLKI |
| NO: 278 | length | SCKGSGYSFTNYWIGWVRQM |
| | heavy | PGKGLEWMAIINPRDSDTRY |
| | chain | RPSFQGQVTISADKSISTAY |
| | sequence | LQWSSLKASDTAMYYCARHG |
| | (LALA- | RGYEGYEGAFDIWGQGTLVT |
| | D265C- | VSSASTKGPSVFPLAPSSKS |
| | H435A | TSGGTAALGCLVKDYFPEPV |
| | mutant); | TVSWNSGALTSGVHTFPAVL |
| | constant | QSSGLYSLSSVVTVPSSSLG |
| | region | TQTYICNVNHKPSNTKVDKK |
| | underlined | VEPKSCDKTHTCPPCPAPEA |
| | | AGGPSVFLFPPKPKDTLMIS |
| | | RTPEVTCVVVCVSHEDPEVK |
| | | FNWYVDGVEVHNAKTKPREE |
| | | QYNSTYRVVSVLTVLHQDWL |
| | | NGKEYKCKVSNKALPAPIEK |
| | | TISKAKGQPREPQVYTLPPS |
| | | RDELTKNQVSLTCLVKGFYP |
| | | SDIAVEWESNGQPENNYKTT |
| | | PPVLDSDGSFFLYSKLTVDK |
| | | SRWQQGNVFSCSVMHEALHN |
| | | AYTQKSLSLSPGK |
| SEQ ID | Ab249 full | EVQLVQSGAEVKKPGESLKI |
| NO: 279 | length | SCKGSGYRFTTSWIGWVRQM |
| | heavy | PGKGLEWMGIIYPGDSDTRY |
| | chain | SPSFQGQVTISADKSISTAY |
| | sequence; | LQWSSLKASDTAMYYCARHG |
| | constant | LGYNGYEGAFDIWGQGTLVT |
| | region | VSSASTKGPSVFPLAPSSKS |
| | underlined | TSGGTAALGCLVKDYFPEPV |
| | | TVSWNSGALTSGVHTFPAVL |
| | | QSSGLYSLSSVVTVPSSSLG |
| | | TQTYICNVNHKPSNTKVDKK |
| | | VEPKSCDKTHTCPPCPAPEL |
| | | LGGPSVFLFPPKPKDTLMIS |
| | | RTPEVTCVVVDVSHEDPEVK |
| | | FNWYVDGVEVHNAKTKPREE |
| | | QYNSTYRVVSVLTVLHQDWL |
| | | NGKEYKCKVSNKALPAPIEK |
| | | TISKAKGQPREPQVYTLPPS |
| | | RDELTKNQVSLTCLVKGFYP |
| | | SDIAVEWESNGQPENNYKTT |
| | | PPVLDSDGSFFLYSKLTVDK |
| | | SRWQQGNVFSCSVMHEALHN |
| | | HYTQKSLSLSPGK |
| SEQ ID | Ab249 full | EVQLVQSGAEVKKPGESLKI |
| NO: 280 | length | SCKGSGYRFTTSWIGWVRQM |
| | heavy chain | PGKGLEWMGIIYPGDSDTRY |
| | sequence; | SPSFQGQVTISADKSISTAY |
| | constant | LQWSSLKASDTAMYYCARHG |
| | region | LGYNGYEGAFDIWGQGTLVT |
| | underlined | VSSASTKGPSVFPLAPSSKS |
| | (LALA | TSGGTAALGCLVKDYFPEPV |
| | mutations)* | TVSWNSGALTSGVHTFPAVL |
| | | QSSGLYSLSSVVTVPSSSLG |
| | | TQTYICNVNHKPSNTKVDKK |
| | | VEPKSCDKTHTCPPCPAPEA |
| | | AGGPSVFLFPPKPKDTLMIS |
| | | RTPEVTCVVVDVSHEDPEVK |
| | | FNWYVDGVEVHNAKTKPREE |
| | | QYNSTYRVVSVLTVLHQDWL |
| | | NGKEYKCKVSNKALPAPIEK |
| | | TISKAKGQPREPQVYTLPPS |
| | | RDELTKNQVSLTCLVKGFYP |
| | | SDIAVEWESNGQPENNYKTT |
| | | PPVLDSDGSFFLYSKLTVDK |
| | | SRWQQGNVFSCSVMHEALHN |
| | | HYTQKSLSLSPGK |
| SEQ ID | Ab249 full | EVQLVQSGAEVKKPGESLKI |
| NO: 281 | length | SCKGSGYRFTTSWIGWVRQM |
| | heavy chain | PGKGLEWMGIIYPGDSDTRY |
| | sequence; | SPSFQGQVTISADKSISTAY |
| | constant | LQWSSLKASDTAMYYCARHG |
| | region | LGYNGYEGAFDIWGQGTLVT |
| | underlined | VSSASTKGPSVFPLAPSSKS |
| | (LALA- | TSGGTAALGCLVKDYFPEPV |
| | D265C | TVSWNSGALTSGVHTFPAVL |
| | mutations)* | QSSGLYSLSSVVTVPSSSLG |
| | | TQTYICNVNHKPSNTKVDKK |
| | | VEPKSCDKTHTCPPCPAPEA |
| | | AGGPSVFLFPPKPKDTLMIS |
| | | RTPEVTCVVVCVSHEDPEVK |
| | | FNWYVDGVEVHNAKTKPREE |
| | | QYNSTYRVVSVLTVLHQDWL |
| | | NGKEYKCKVSNKALPAPIEK |
| | | TISKAKGQPREPQVYTLPPS |
| | | RDELTKNQVSLTCLVKGFYP |
| | | SDIAVEWESNGQPENNYKTT |
| | | PPVLDSDGSFFLYSKLTVDK |
| | | SRWQQGNVFSCSVMHEALHN |
| | | HYTQKSLSLSPGK |
| SEQ ID | Ab249 full | EVQLVQSGAEVKKPGESLKI |
| NO: 282 | length | SCKGSGYRFTTSWIGWVRQM |
| | heavy chain | PGKGLEWMGIIYPGDSDTRY |
| | sequence; | SPSFQGQVTISADKSISTAY |
| | constant | LQWSSLKASDTAMYYCARHG |
| | region | LGYNGYEGAFDIWGQGTLVT |
| | underlined; | VSSASTKGPSVFPLAPSSKS |
| | (LALA- | TSGGTAALGCLVKDYFPEPV |
| | D265C- | TVSWNSGALTSGVHTFPAVL |
| | H435A | QSSGLYSLSSVVTVPSSSLG |
| | mutations)* | TQTYICNVNHKPSNTKVDKK |
| | | VEPKSCDKTHTCPPCPAPEA |
| | | AGGPSVFLFPPKPKDTLMIS |
| | | RTPEVTCVVVCVSHEDPEVK |
| | | FNWYVDGVEVHNAKTKPREE |
| | | QYNSTYRVVSVLTVLHQDWL |
| | | NGKEYKCKVSNKALPAPIEK |
| | | TISKAKGQPREPQVYTLPPS |
| | | RDELTKNQVSLTCLVKGFYP |
| | | SDIAVEWESNGQPENNYKTT |
| | | PPVLDSDGSFFLYSKLTVDK |
| | | SRWQQGNVFSCSVMHEALHN |
| | | AYTQKSLSLSPGK |
| SEQ ID | Light chain | RTVAAPSVFIFPPSDEQLKS |
| NO: 283 | constant | GTASVVCLLNNFYPREAKVQ |
| | region | WKVDNALQSGNSQESVTEQD |
| | | SKDSTYSLSSTLTLSKADYE |
| | | KHKVYACEVTHQGLSSPVTK |
| | | SFNRGEC |
| SEQ ID | Ab85 full | DIQMTQSPSSLSASVGDRVT |
| NO: 284 | length light | ITCRSSQGIRSDLGWYQQKP |
| | chain; | GKAPKLLIYDASNLETGVPS |
| | constant | RFSGSGSGTDFTLTISSLQP |
| | region | EDFATYYCQQANGFPLTFGG |
| | underlined | GTKVEIKRTVAAPSVFIFPP |
| | | SDEQLKSGTASVVCLLNNFY |
| | | PREAKVQWKVDNALQSGNSQ |
| | | ESVTEQDSKDSTYSLSSTLT |
| | | LSKADYEKHKVYACEVTHQG |
| | | LSSPVTKSFNRGEC |
| SEQ ID | Ab249 light | DIQMTQSPSSLSASVGDRVT |
| NO: 285 | chain; | ITCRASQGIGSALAWYQQKP |
| | constant | GKAPKLLIYDASNLETGVPS |
| | region | RFSGSGSGTDFTLTISSLQP |
| | underlined | EDFATYYCQQLNGYPLTFGQ |
| | | GTRLEIKRTVAAPSVFIFPP |
| | | SDEQLKSGTASVVCLLNNFY |
| | | PREAKVQWKVDNALQSGNSQ |
| | | ESVTEQDSKDSTYSLSSTLT |
| | | LSKADYEKHKVYACEVTHQG |
| | | LSSPVTKSFNRGEC |
| SEQ ID | Ab249 HC-CDR1 | TSWIG |
| NO: 286 | | |
| SEQ ID | Ab249 HC-CDR3 | HGLGYNGYEGAFDI |
| NO: 287 | | |
| SEQ ID | Ab249 LC-CDR1 | RASQGIGSALA |
| NO: 288 | | |
| SEQ ID | Ab249 LC-CDR3 | CQQLNGYPLT |
| NO: 289 | | |
| SEQ ID | Shiga-like | KEFTLDFSTAKTYVDSLNVI |
| NO: 290 | toxin 1 | RSAIGTPLQTISSGGTSLLM |
| | subunit A | IDSGSGDNLFAVDVRGIDPE |
| | (SLT-1A) | EGRFNNLRLIVERNNLYVTG |
| | | FVNRTNNVFYRFADFSHVTF |
| | | PGTTAVTLSGDSSYTTLQRV |
| | | AGISRTGMQINRHSLTTSYL |
| | | DLMSHSGTSLTQSVARAMLR |
| | | FVTVTAEALRFRQIQRGFRT |
| | | TLDDLSGRSYVMTAEDVDLT |
| | | LNWGRLSSVLPDYHGQDSVR |
| | | VGRISFGSINAILGSVALIL |
| | | NCHHHASRVARMASDEFPSM |
| | | CPADGRVRGITHNKILWDSS |
| | | TLGAILMRRTISS |
| SEQ ID | Shiga toxin | KEFTLDFSTAKTYVDSLNVI |
| NO: 291 | subunit A | RSAIGTPLQTISSGGTSLLM |
| | (StxA) | IDSGTGDNLFAVDVRGIDPE |
| | | EGRFNNLRLIVERNNLYVTG |
| | | FVNRTNNVFYRFADFSHVTF |
| | | PGTTAVTLSGDSSYTTLQRV |
| | | AGISRTGMQINRHSLTTSYL |
| | | DLMSHSGTSLTQSVARAMLR |
| | | FVTVTAEALRFRQIQRGFRT |
| | | TLDDLSGRSYVMTAEDVDLT |
| | | LNWGRLSSVLPDYHGQDSVR |
| | | VGRISFGSINAILGSVALIL |
| | | NCHHHASRVARMASDEFPSM |
| | | CPADGRVRGITHNKILWDSS |
| | | TLGAILMRRTISS |
| SEQ ID | Shiga-like | DEFTVDFSSQKSYVDSLNSI |
| NO: 292 | toxin 2 | RSAISTPLGNISQGGVSVSV |
| | subunit A | INHVLGGNYISLNVRGLDPY |
| | (SLT-2A) | SERFNHLRLIMERNNLYVAG |
| | | FINTETNIFYRFSDFSHISV |
| | | PDVITVSMTTDSSYSSLQRI |
| | | ADLERTGMQIGRHSLVGSYL |
| | | DLMEFRGRSMTRASSRAMLR |
| | | FVTVIAEALRFRQIQRGFRP |
| | | ALSEASPLYTMTAQDVDLTL |
| | | NWGRISNVLPEYRGEEGVRI |
| | | GRISFNSLSAILGSVAVILN |
| | | CHSTGSYSVRSVSQKQKTEC |
| | | QIVGDRAAIKVNNVLWEANT |
| | | IAALLNRKPQDLTEPNQ |
| SEQ ID | CD2 (isoform 1) | MSFPCKFVASFLLIFNVSSK |
| NO: 293 | (NP_001758.2) | GAVSKEITNALETWGALGQD |
| | | INLDIPSFQMSDDIDDIKWE |
| | | KTSDKKKIAQFRKEKETFKE |
| | | KDTYKLFKNGTLKIKHLKTD |
| | | DQDIYKVSIYDTKGKNVLEK |
| | | IFDLKIQERVSKPKISWTCI |
| | | NTTLTCEVMNGTDPELNLYQ |
| | | DGKHLKLSQRVITHKWTTSL |
| | | SAKFKCTAGNKVSKESSVEP |
| | | VSCPEKGLDIYLIIGICGGG |
| | | SLLMVFVALLVFYITKRKKQ |
| | | RSRRNDEELETRAHRVATEE |
| | | RGRKPHQIPASTPQNPATSQ |
| | | HPPPPPGHRSQAPSHRPPPP |
| | | GHRVQHQPQKRPPAPSGTQV |
| | | HQQKGPPLPRPRVQPKPPHG |
| | | AAENSLSPSSN |
| SEQ ID | Anti-CD2 | EYYMY |
| NO: 294 | antibody | |
| | CDR-H1 | |
| SEQ ID | Anti-CD2 | RIDPEDGSIDYVEKFKK |
| NO: 295 | antibody | |
| | CDR-H2 | |
| SEQ ID | Anti-CD2 | GKFNYRFAY |
| NO: 296 | antibody | |
| | CDR-H3 | |
| SEQ ID | Anti-CD2 | RSSQSLLHSSGNTYLN |
| NO: 297 | antibody | |
| | CDR-L1 | |
| SEQ ID | Anti-CD2 | LVSKLES |
| NO: 298 | antibody | |
| | CDR-L2 | |
| SEQ ID | Anti-CD2 | MQFTHYPYT |
| NO: 299 | antibody | |
| | CDR-L3 | |
| SEQ ID | Anti-CD2 | QVQLVQSGAEVKKPGASVKV |
| NO: 300 | antibody | SCKASGYTFTEYYMYWVRQA |
| | Heavy chain | PGQGLELMGRIDPEDGSIDY |
| | variable | VEKFKKKVTLTADTSSSTAY |
| | region | MELSSLTSDDTAVYYCARGK |
| | | FNYRFAYWGQGTLVTVSS |
| SEQ ID | Anti-CD2 | DVVMTQSPPSLLVTLGQPAS |
| NO: 301 | antibody | ISCRSSQSLLHSSGNTYLNW |
| | Light chain | LLQRPGQSPQPLIYLVSKLE |
| | variable | SGVPDRFSGSGSGTDFTLKI |
| | region | SGVEAEDVGVYYCMQFTHYP |
| | | YTFGQGTKLEIK |
| SEQ ID | Anti-CD2 | GFTFSSY |
| NO: 302 | antibody | |
| | CDR-H1 | |
| SEQ ID | Anti-CD2 | SGGGF |
| NO: 303 | antibody | |
| | CDR-H2 | |
| SEQ ID | Anti-CD2 | SSYGEIMDY |
| NO: 304 | antibody | |
| | CDR-H3 | |
| SEQ ID | Anti-CD2 | RASQRIGTSIH |
| NO: 305 | antibody | |
| | CDR-L1 | |
| SEQ ID | Anti-CD2 | YASESIS |
| NO: 306 | antibody | |
| | CDR-L2 | |
| SEQ ID | Anti-CD2 | QQSHGWPFTF |
| NO: 307 | antibody | |
| | CDR-L3 | |
| SEQ ID | Anti-CD2 | EVKLVESGGGLVKPGGSLKL |
| NO: 308 | antibody | SCAASGFTFSSYDMSWVRQT |
| | Heavy chain | PEKRLEWVASISGGGFLYYL |
| | variable | DSVKGRFTISRDNARNILYL |
| | region | HMTSLRSEDTAMYYCARSSY |
| | | GEIMDYWGQGTSVTVSS |
| SEQ ID | Anti-CD2 | DILLTQSPAILSVSPGERVS |
| NO: 309 | antibody | FSCRASQRIGTSIHWYQQRT |
| | Light chain | TGSPRLLIKYASESISGIPS |
| | variable | RFSGSGSGTDFTLSINSVES |
| | region | EDVADYYCQQSHGWPFTFGG |
| | | GTKLEIE |
| SEQ ID | Anti-CD2 | SSYGELMDY |
| NO: 310 | antibody | |
| | CDR-H3 | |
| SEQ ID | Anti-CD2 | EVKLVESGGGLVKPGGSLKL |
| NO: 311 | antibody | SCAASGFTFSSYDMSWVRQT |
| | Heavy chain | PEKRLEWVASISGGGFLYYL |
| | variable | DSVKGRFTISRDNARNILYL |
| | region | HMTSLRSEDTAMYYCARSSY |
| | | GELMDYWGQGTSVTVSS |
| SEQ ID | CD5 (isoform 1) | MPMGSLQPLATLYLLGMLVA |
| NO: 312 | (NP 055022.2) | SCLGRLSWYDPDFQARLTRS |
| | | NSKCQGQLEVYLKDGWHMVC |
| | | SQSWGRSSKQWEDPSQASKV |
| | | CQRLNCGVPLSLGPFLVTYT |
| | | PQSSIICYGQLGSFSNCSHS |
| | | RNDMCHSLGLTCLEPQKTTP |
| | | PTTRPPPTTTPEPTAPPRLQ |
| | | LVAQSGGQHCAGVVEFYSGS |
| | | LGGTISYEAQDKTQDLENFL |
| | | CNNLQCGSFLKHLPETEAGR |
| | | AQDPGEPREHQPLPIQWKIQ |
| | | NSSCTSLEHCFRKIKPQKSG |
| | | RVLALLCSGFQPKVQSRLVG |
| | | GSSICEGTVEVRQGAQWAAL |
| | | CDSSSARSSLRWEEVCREQQ |
| | | CGSVNSYRVLDAGDPTSRGL |
| | | FCPHQKLSQCHELWERNSYC |
| | | KKVFVTCQDPNPAGLAAGTV |
| | | ASIILALVLLVVLLVVCGPL |
| | | AYKKLVKKFRQKKQRQWIGP |
| | | TGMNQNMSFHRNHTATVRSH |
| | | AENPTASHVDNEYSQPPRNS |
| | | HLSAYPALEGALHRSSMQPD |
| | | NSSDSDYDLHGAQRL |
| SEQ ID | Ab5D7 Heavy | QVTLKESGPVLVKPTETLTL |
| NO: 313 | chain | TCTFSGFSLSTSGMGVGWIR |
| | variable | QAPGKGLEWVAHIWWDDDVY |
| | region | YNPSLKSRLTITKDASKDQV |
| | | SLKLSSVTAADTAVYYCVRR |
| | | RATGTGFDYWGQGTLVTVSS |
| SEQ ID | Ab5D7 Light | NIVMTQSPSSLSASVGDRVT |
| NO: 314 | chain | ITCQASQDVGTAVAWYQQKP |
| | variable | DQSPKLLIYWTSTRHTGVPD |
| | region | RFTGSGSGTDFTLTISSLQP |
| | | EDIATYFCHQYNSYNTFGSG |
| | | TKLEIK |
| SEQ ID | Ab5D7 VH CDR1 | FSLSTSGMG |
| NO: 315 | | |
| SEQ ID | Ab5D7 VH CDR2 | WWDDD |
| NO: 316 | | |
| SEQ ID | Ab5D7 VH CDR3 | RRATGTGFDY |
| NO: 317 | | |
| SEQ ID | Ab5D7 VL CDR1 | QDVGTA |
| NO: 318 | | |
| SEQ ID | Ab5D7 VL CDR2 | WTSTRHT |
| NO: 319 | | |
| SEQ ID | Ab5D7 VL CDR3 | YNSYNT |
| NO: 320 | | |

*Fc residues are numbered according to the EU index in Kabat et al.

### OTHER EMBODIMENTS

All publications, patents, and patent applications mentioned in this specification are incorporated herein by reference to the same extent as if each independent publication or patent application was specifically and individually indicated to be incorporated by reference.

While the present disclosure has been described in connection with specific embodiments thereof, it will be understood that it is capable of further modifications and this application is intended to cover any variations, uses, or adaptations of the present disclosure following, in general, the principles of the present disclosure and including such departures from the present disclosure that come within known or customary practice within the art to which the present disclosure pertains and may be applied to the essential features hereinbefore set forth, and follows in the scope of the claims.

Other embodiments are within the claims.

1. A method of administering genetically modified stem cells to a human subject in need thereof, the method comprising:
a) administering to the human subject an antibody-drug conjugate (ADC) that binds to a cell surface molecule expressed on hematopoietic stem cells (HSC) and/or immune cells, thereby depleting HSCs and/or immune cells from the human subject; and
b) administering to the human subject a transplant comprising a population of genetically modified stem cells.

2. A method of treating a human subject with genetically modified cells, the method comprising administering a transplant comprising a population of genetically modified stem cells to the human subject in need thereof, wherein the human subject has received a conditioning treatment comprising an antibody-drug conjugate (ADC) that binds to a cell surface molecule expressed on hematopoietic stem cells (HSC) and/or immune cells.

3. The method of claim 1, wherein the genetically modified stem cells are autologous stem cells, allogeneic stem cells, or HSCs.

4-5. (canceled)

6. The method of claim 1, wherein the subject is suffering from cancer, a hemoglobinopathy disorder, myelodysplastic disorder, immunodeficiency disorder, or a metabolic disorder.

7. The method of claim 6, wherein:
the hemoglobinopathy disorder is selected from any one or more of sickle cell anemia, thalassemia, Fanconi anemia, aplastic anemia, or Wiskott-Aldrich syndrome;
the immunodeficiency disorder is a congenital immunodeficiency or an acquired immunodeficiency;
the metabolic disorder is selected from any one or more of a glycogen storage disease, mucopolysaccharidosis, Gaucher's Disease, Hurlers Disease, sphingolipidoses, globoid cell leukodystrophy, or metachromatic leukodystrophy; or
the cancer is selected from any one or more of leukemia, lymphoma, multiple myeloma, or neuroblastoma.

8. (canceled)

9. The method of claim 6, wherein:
the acquired immunodeficiency is human immunodeficiency virus or acquired immune deficiency syndrome (AIDS); or
the cancer is a hematological cancer.

10-12. (canceled)

13. The method of claim 9, wherein the hematological cancer is acute myeloid leukemia, acute lymphoid leukemia, chronic myeloid leukemia, chronic lymphoid leukemia, or multiple myeloma.

14. The method of claim 1, wherein the subject is suffering from a disorder selected from any one or more of an adenosine deaminase deficiency, severe combined immunodeficiency, hyper immunoglobulin M syndrome, Chediak-Higashi disease, hereditary lymphohistiocytosis, osteopetrosis, osteogenesis imperfecta, storage diseases, thalassemia major, systemic sclerosis, systemic lupus erythematosus, multiple sclerosis, or juvenile rheumatoid arthritis.

15. The method of claim 1, wherein the subject is suffering from an autoimmune disorder.

16. The method of claim 15, wherein the autoimmune disorder is selected from any one or more of multiple sclerosis, human systemic lupus, rheumatoid arthritis, inflammatory bowel disease, treating psoriasis, Type 1 diabetes mellitus, acute disseminated encephalomyelitis, Addison's disease, alopecia universalis, ankylosing spondylitisis, antiphospholipid antibody syndrome, aplastic anemia, autoimmune hemolytic anemia, autoimmune hepatitis, autoimmune inner ear disease, autoimmune lymphoproliferative syndrome, autoimmune oophoritis, Balo disease, Behcet's disease, bullous pemphigoid, cardiomyopathy, Chagas' disease, chronic fatigue immune dysfunction syndrome, chronic inflammatory demyelinating polyneuropathy, Crohn's disease, cicatrical pemphigoid, coeliac sprue-dermatitis herpetiformis, cold agglutinin disease, CREST syndrome, Degos disease, discoid lupus, dysautonomia, endometriosis, essential mixed cryoglobulinemia, fibromyalgia-fibromyositis, Goodpasture's syndrome, Grave's disease, Guillain-Barre syndrome, Hashimoto's thyroiditis, Hidradenitis suppurativa, idiopathic and/or acute thrombocytopenic purpura, idiopathic pulmonary fibrosis, IgA neuropathy, interstitial cystitis, juvenile arthritis, Kawasaki's disease, lichen planus, Lyme disease, Meniere disease, mixed connective tissue disease, myasthenia gravis, neuromyotonia, opsoclonus myoclonus syndrome, optic neuritis, Ord's thyroiditis, pemphigus vulgaris, pernicious anemia, polychondritis, polymyositis and dermatomyositis, primary biliary cirrhosis, polyarteritis nodosa, polyglandular syndromes, polymyalgia rheumatica, primary agammaglobulinemia, Raynaud phenomenon, Reiter's syndrome, rheumatic fever, sarcoidosis, scleroderma, Sjögren's syndrome, stiff person syndrome, Takayasu's arteritis, temporal arteritis, ulcerative colitis, uveitis, vasculitis, vitiligo, vulvodynia, chronic granulomatosis disease, or Wegener's granulomatosis.

17. The method of claim 1, wherein the population of stem cells has been genetically modified to alter a target gene.

18. The method of claim 17, wherein the target gene is selected from one or more of beta-globin, gamma-globin, adenosine deaminase, arylsulfatase A, WASp gene, phagocyte NADPH oxidase, galatosyla ceramidase, beta-galactosidase, beta-hexosaminidase, alpha-L iduronidase, ATM serine/threonine kinase, Ribosome maturation protein SBDS, or CCR5.

19. The method of claim 1, wherein the transplant comprising a population of genetically modified stem cells has been modified using a gene editing system.

20. The method of claim 19, wherein the gene editing system is a CRISPR/Cas system.

21. The method of claim 1, wherein the ADC comprises an antibody or antigen-binding fragment thereof that binds one or more cell surface molecule selected from CD2, CD5, CD7, CDwl2, CD13, CD15, CD19, CD21, CD22, CD29, CD30, CD33, CD34, CD36, CD38, CD40, CD41, CD42a, CD42b, CD42c, CD42d, CD43, CD45, CD45RA, CD45RB, CD45RC, CD45RO, CD48, CD49b, CD49d, CD49e, CD49f, CD50, CD53, CD55, CD64a, CD68, CD71, CD72, CD73, CD81, CD82, CD85A, CD85K, CD90, CD99, CD104, CD105, CD109, CD110, CD111, CD112, CD114, CD115, CD117, CD123, CD124, CD126, CD127, CD130, CD131, CD133, CD135, CD137, CD138, CD151, CD157, CD162, CD164, CD168, CD172a, CD173, CD174, CD175, CD175s, CD176, CD183, CD191, CD200, CD201, CD205, CD217, CD220, CD221, CD222, CD223, CD224, CD225, CD226, CD227, CD228, CD229, CD230, CD235a, CD235b, CD236, CD236R, CD238, CD240, CD242, CD243, CD277, CD292, CDw293, CD295, CD298, CD309, CD318, CD324, CD325, CD338, CD344, CD349, or CD350.

22. The method of claim 1, wherein the ADC comprises an antibody or antigen-binding fragment thereof that binds CD117.

23. The method of claim 1, wherein the ADC is administered in an amount sufficient to deplete a population of CD117+ cells in the subject.

24. The method of claim 22, wherein the CD117 is GNNK+ CD117.

25. The method of claim 22, wherein the anti-CD117 antibody or antigen-binding fragment thereof comprises:
(a) a heavy chain variable region comprising a CDR1 domain comprising the amino acid sequence as set forth in SEQ ID NO: 31, a CDR2 domain comprising the amino acid sequence as set forth in SEQ ID NO:32, and a CDR3 domain comprising the amino acid sequence as set forth in SEQ ID NO: 33; and a light chain variable region comprising a CDR1 domain comprising the amino acid sequence as set forth in SEQ ID NO: 34, a CDR2 domain comprising the amino acid sequence as set forth in SEQ ID NO:35, and a CDR3 domain comprising the amino acid sequence as set forth in SEQ ID NO: 36;
(b) a heavy chain variable region comprising a CDR1 domain comprising the amino acid sequence as set forth in SEQ ID NO: 21, a CDR2 domain comprising the amino acid sequence as set forth in SEQ ID NO:22, and a CDR3 domain comprising the amino acid sequence as set forth in SEQ ID NO: 23; and a light chain variable region comprising a CDR1 domain comprising the amino acid sequence as set forth in SEQ ID NO: 24, a CDR2 domain comprising the amino acid sequence as set forth in SEQ ID NO:25, and a CDR3 domain comprising the amino acid sequence as set forth in SEQ ID NO: 26;
(c) a heavy chain variable region comprising a CDR1 domain comprising the amino acid sequence as set forth in SEQ ID NO: 41, a CDR2 domain comprising the amino acid sequence as set forth in SEQ ID NO:42, and a CDR3 domain comprising the amino acid sequence as set forth in SEQ ID NO: 43; and a light chain variable region comprising a CDR1 domain comprising the amino acid sequence as set forth in SEQ ID NO: 44, a CDR2 domain comprising the amino acid sequence as set forth in SEQ ID NO:45, and a CDR3 domain comprising the amino acid sequence as set forth in SEQ ID NO: 46;
(d) a heavy chain variable region comprising a CDR1 domain comprising the amino acid sequence as set forth in SEQ ID NO: 51, a CDR2 domain comprising the amino acid sequence as set forth in SEQ ID NO:52, and a CDR3 domain comprising the amino acid sequence as set forth in SEQ ID NO: 53; and a light chain variable region comprising a CDR1 domain comprising the amino acid sequence as set forth in SEQ ID NO: 54, a CDR2 domain comprising the amino acid sequence as set forth in SEQ ID NO:55, and a CDR3 domain comprising the amino acid sequence as set forth in SEQ ID NO: 56;
(e) a heavy chain variable region comprising a CDR1 domain comprising the amino acid sequence as set forth in SEQ ID NO: 61, a CDR2 domain comprising the amino acid sequence as set forth in SEQ ID NO:62, and a CDR3 domain comprising the amino acid sequence as set forth in SEQ ID NO: 63; and a light chain variable region comprising a CDR1 domain comprising the amino acid sequence as set forth in SEQ ID NO: 64, a CDR2 domain comprising the amino acid sequence as set forth in SEQ ID NO:65, and a CDR3 domain comprising the amino acid sequence as set forth in SEQ ID NO: 66;
(f) a heavy chain variable region comprising a CDR1 domain comprising the amino acid sequence as set forth in SEQ ID NO: 71, a CDR2 domain comprising the amino acid sequence as set forth in SEQ ID NO:72, and a CDR3 domain comprising the amino acid sequence as set forth in SEQ ID NO: 73; and a light chain variable region comprising a CDR1 domain comprising the amino acid sequence as set forth in SEQ ID NO: 74, a CDR2 domain comprising the amino acid sequence as set forth in SEQ ID NO:75, and a CDR3 domain comprising the amino acid sequence as set forth in SEQ ID NO: 76;
(g) a heavy chain variable region comprising a CDR1 domain comprising the amino acid sequence as set forth in SEQ ID NO: 81, a CDR2 domain comprising the amino acid sequence as set forth in SEQ ID NO:82, and a CDR3 domain comprising the amino acid sequence as set forth in SEQ ID NO: 83; and a light chain variable region comprising a CDR1 domain comprising the amino acid sequence as set forth in SEQ ID NO: 84, a CDR2 domain comprising the amino acid sequence as set forth in SEQ ID NO:85, and a CDR3 domain comprising the amino acid sequence as set forth in SEQ ID NO: 86;
(h) a heavy chain variable region comprising a CDR1 domain comprising the amino acid sequence as set forth in SEQ ID NO: 11, a CDR2 domain comprising the amino acid sequence as set forth in SEQ ID NO:12, and a CDR3 domain comprising the amino acid sequence as set forth in SEQ ID NO: 13; and a light chain variable region comprising a CDR1 domain comprising the amino acid sequence as set forth in SEQ ID NO: 14, a CDR2 domain comprising the amino acid sequence as set forth in SEQ ID NO:15, and a CDR3 domain comprising the amino acid sequence as set forth in SEQ ID NO: 16;
(i) a heavy chain variable region comprising a CDR1 domain comprising the amino acid sequence as set forth in SEQ ID NO: 91, a CDR2 domain comprising the amino acid sequence as set forth in SEQ ID NO:92, and a CDR3 domain comprising the amino acid sequence as set forth in SEQ ID NO: 93; and a light chain variable region comprising a CDR1 domain comprising the amino acid sequence as set forth in SEQ ID NO: 94, a CDR2 domain comprising the amino acid sequence as set forth in SEQ ID NO:95, and a CDR3 domain comprising the amino acid sequence as set forth in SEQ ID NO: 96;
(j) a heavy chain variable region comprising a CDR1 domain comprising the amino acid sequence as set forth in SEQ ID NO: 101, a CDR2 domain comprising the amino acid sequence as set forth in SEQ ID NO:102, and a CDR3 domain comprising the amino acid sequence as set forth in SEQ ID NO: 103; and a light chain variable region comprising a CDR1 domain comprising the amino acid sequence as set forth in SEQ ID NO: 104, a CDR2 domain comprising the amino acid sequence as set forth in SEQ ID NO:105, and a CDR3 domain comprising the amino acid sequence as set forth in SEQ ID NO: 106; or
(k) a heavy chain variable region comprising a CDR1 domain comprising the amino acid sequence as set forth in SEQ ID NO: 245, a CDR2 domain comprising the amino acid sequence as set forth in SEQ ID NO:246, and a CDR3 domain comprising the amino acid sequence as set forth in SEQ ID NO: 247; and a light chain variable region comprising a CDR1 domain comprising the amino acid sequence as set forth in SEQ ID NO: 248, a CDR2 domain comprising the amino acid sequence as set forth in SEQ ID NO:249, and a CDR3 domain comprising the amino acid sequence as set forth in SEQ ID NO: 250.

26. The method of claim 22, wherein the anti-CD117 antibody or antigen-binding fragment thereof comprises a heavy chain variable region comprising a CDR1 domain comprising the amino acid sequence as set forth in SEQ ID NO: 127, a CDR2 domain comprising the amino acid sequence as set forth in SEQ ID NO:128, and a CDR3 domain comprising the amino acid sequence as set forth in SEQ ID NO: 129; and comprising a light chain variable region comprising a CDR1 domain comprising the amino acid sequence as set forth in SEQ ID NO: 130, a CDR2 domain comprising the amino acid sequence as set forth in SEQ ID NO:131, and a CDR3 domain comprising the amino acid sequence as set forth in SEQ ID NO: 132.

27. The method of claim 22, wherein the anti-CD117 antibody or antigen-binding fragment thereof comprises:
(a) a heavy chain variable region comprising a CDR1 domain comprising the amino acid sequence as set forth in SEQ ID NO: 133, a CDR2 domain comprising the amino acid sequence as set forth in SEQ ID NO:134, and a CDR3 domain comprising the amino acid sequence as set forth in SEQ ID NO: 135; and comprising a light chain variable region comprising a CDR1 domain comprising the amino acid sequence as set forth in SEQ ID NO: 136, a CDR2 domain comprising the amino acid sequence as set forth in SEQ ID NO:137, and a CDR3 domain comprising the amino acid sequence as set forth in SEQ ID NO: 138; or
b) a heavy chain variable region comprising a CDR1 domain comprising the amino acid sequence as set forth in SEQ ID NO: 139, a CDR2 domain comprising the amino acid sequence as set forth in SEQ ID NO:140, and a CDR3 domain comprising the amino acid sequence as set forth in SEQ ID NO: 141; and comprising a light chain variable region comprising a CDR1 domain comprising the amino acid sequence as set forth in SEQ ID NO: 142, a CDR2 domain comprising the amino acid sequence as set forth in SEQ ID NO:143, and a CDR3 domain comprising the amino acid sequence as set forth in SEQ ID NO: 144.

28. The method of claim 22, wherein the anti-CD117 antibody or antigen-binding fragment thereof comprises:
(a) a heavy chain variable region comprising the amino acid sequence as set forth in SEQ ID NO: 29, and a light chain variable region comprising the amino acid sequence as set forth in SEQ ID NO: 30;
(b) a heavy chain variable region comprising the amino acid sequence as set forth in SEQ ID NO: 19, and a light chain variable region comprising the amino acid sequence as set forth in SEQ ID NO: 20;
(c) a heavy chain variable region comprising the amino acid sequence as set forth in SEQ ID NO: 39, and a light chain variable region comprising the amino acid sequence as set forth in SEQ ID NO: 40;
(d) a heavy chain variable region comprising the amino acid sequence as set forth in SEQ ID NO: 49, and a light chain variable region comprising the amino acid sequence as set forth in SEQ ID NO: 50;
(e) a heavy chain variable region comprising the amino acid sequence as set forth in SEQ ID NO: 59, and a light chain variable region comprising the amino acid sequence as set forth in SEQ ID NO: 60;
(f) a heavy chain variable region comprising the amino acid sequence as set forth in SEQ ID NO: 69, and a light chain variable region comprising the amino acid sequence as set forth in SEQ ID NO: 70;
(g) a heavy chain variable region comprising the amino acid sequence as set forth in SEQ ID NO: 79, and a light chain variable region comprising the amino acid sequence as set forth in SEQ ID NO: 80;
(h) a heavy chain variable region comprising the amino acid sequence as set forth in SEQ ID NO: 9, and a light chain variable region comprising the amino acid sequence as set forth in SEQ ID NO: 10;
(i) a heavy chain variable region comprising the amino acid sequence as set forth in SEQ ID NO: 89, and a light chain variable region comprising the amino acid sequence as set forth in SEQ ID NO: 90;
(j) a heavy chain variable region comprising the amino acid sequence as set forth in SEQ ID NO: 99, and a light chain variable region comprising the amino acid sequence as set forth in SEQ ID NO: 100; or
(k) a heavy chain variable region comprising the amino acid sequence as set forth in SEQ ID NO: 243, and a light chain variable region comprising the amino acid sequence as set forth in SEQ ID NO: 244.

29. The method of claim 22, wherein the anti-CD117 antibody, or antigen binding fragment thereof has a dissociation rate (K_{OFF}) of 1×10⁻² to 1×10⁻³, 1×10⁻³ to 1×10⁻⁴, 1×10⁻⁵ to 1×10⁻⁶, 1×10⁻⁶ to 1×10⁻⁷ or 1×10⁻⁷ to 1×10⁻⁸as measured by bio-layer interferometry (BLI).

30. The method of claim 22, wherein the antibody or antigen-binding fragment thereof binds CD117 with a K_{D} of about 100 nM or less, about 90 nM or less, about 80 nM or less, about 70 nM or less, about 60 nM or less, about 50 nM or less, about 40 nM or less, about 30 nM or less, about 20 nM or less, about 10 nM or less, about 8 nM or less, about 6 nM or less, about 4 nM or less, about 2 nM or less, about 1 nM or less as determined by a Bio-Layer Interferometry (BLI) assay.

31. The method of claim 21, wherein the antibody or antigen-binding fragment thereof is human.

32. The method of claim 21, wherein the antibody or antigen-binding fragment thereof is an intact antibody.

33. The method of claim 21, wherein the antibody or antigen-binding fragment thereof is an IgG.

34. The method of claim 21, wherein the antibody or antigen-binding fragment thereof is an IgG1 or an IgG4.

35. The method of claim 21, wherein the antibody or antigen-binding fragment thereof is a monoclonal antibody.

36. The method of claim 21, wherein the antibody or antigen-binding fragment thereof comprises a heavy chain constant region having an amino acid sequence as set forth as SEQ ID NO: 122 and/or a light chain constant region comprising an amino acid sequence as set forth in SEQ ID NO: 121.

37. The method of claim 21, wherein the antibody or antigen-binding fragment thereof comprises an Fc region comprising at least one amino acid substitution selected from the group consisting of D265C, H435A, L234A, and L235A (numbering according to the EU index).

38. The method of claim 37, wherein the Fc region comprises amino acid substitutions D265C, L234A, and L235A (numbering according to the EU index).

39. The method of claim 22, wherein the anti-CD117 antibody or antigen-binding fragment thereof comprises;
a light chain comprising an amino acid sequence as set forth in SEQ ID NO: 109, and a heavy chain comprising an amino acid sequence selected from the group consisting of SEQ ID NO: 110, SEQ ID NO: 111, SEQ ID NO: 112, SEQ ID NO: 113, and SEQ ID NO: 114;
a light chain comprising an amino acid sequence as set forth in SEQ ID NO: 115, and a heavy chain comprising an amino acid sequence selected from the group consisting of SEQ ID NO: 116, SEQ ID NO: 117, SEQ ID NO: 118, SEQ ID NO: 119, and SEQ ID NO: 120; or
a light chain comprising an amino acid sequence as set forth in SEQ ID NO: 284, and a heavy chain comprising an amino acid sequence selected from the group consisting of SEQ ID NO: 275, SEQ ID NO: 276, SEQ ID NO: 277, and SEQ ID NO: 278.

40-41. (canceled)

42. The method of claim 22, wherein the anti-CD117 antibody or antigen-binding fragment thereof comprises
a heavy chain comprising an HC-CDR1, an HC-CDR2, and an HC-CDR3 or a variable region sequence from the heavy chain variable region of Ab55, Ab54, Ab56, Ab57, Ab58, Ab61, Ab66, Ab67, Ab68, Ab69, Ab85, Ab86, Ab87, Ab88, Ab89, Ab77, Ab79, Ab81, Ab85, or Ab249, and a light chain comprising an LC-CDR1, an LC-CDR2, and an LC-CDR3 or a variable region sequence from the light chain variable region of Ab55, Ab54, Ab56, Ab57, Ab58, Ab61, Ab66, Ab67, Ab68, Ab69, Ab85, Ab86, Ab87, Ab88, Ab89, Ab77, Ab79, Ab81, Ab85, or Ab249, or
a heavy chain comprising an HC-CDR1, an HC-CDR2, and an HC-CDR3 or a variable region from the heavy chain variable region amino acid sequence of SEQ ID NO: 147, 164, 166, 168, 170, 172, 174, 176, 178, 180, 183, 185, 187, 189, 191, 193, 195, 197, 199, 201, 202, 204, 206, 208, 210, 212, 214, 216, 218, 220, 222, 224, 226, 238, or 243, and a light chain comprising an LC-CDR1, an LC-CDR2, and an LC-CDR3 or a variable region from the light chain variable region amino acid sequence of SEQ ID NO: 148, 149, 150, 151, 152, 153, 154, 155, 156, 157, 158, 159, 160, 161, 162, 163, 165, 167, 169, 171, 173, 175, 177, 179, 181, 182, 184, 186, 188, 190, 192, 194, 196, 198, 200, 203, 205, 207, 209, 211, 213, 215, 217, 219, 221, 223, 225, 227, 228, 229, 230, 231, 232, 233, 234, 235, 236, 237, 239, 240, 241, 242, or 244.

43. The method of claim 1, wherein the ADC is represented by the formula Ab-(Z-L-Cy)ₙ, wherein:
Ab is the antibody or antigen-binding fragment thereof,
L is a linker;
Z is a chemical moiety formed by a coupling reaction between a reactive substituent present on L and a reactive substituent present within the antibody or antigen-binding fragment thereof,
Cy is a cytotoxin selected from the group consisting of an amatoxin, pseudomonas exotoxin A, deBouganin, diphtheria toxin, saporin, maytansine, a maytansinoid, a pyrrolobenzodiazepine, a pyrrolobenzodiazepine dimer, an indolinobenzodiazepine, an indolinobenzodiazepine dimer, a calicheamicin, an auristatin, and an anthracycline; and
n is an integer from about 1 to about 20, which represents the average number of cytotoxins per antibody.
